(19) United States
(12) Patent Application Publication
(10) Pub. No.: US 2010/0184815 A1
(43) Pub. Date: Jul. 22, 2010

(54) AGONISTS OF PEROXISOME PROLIFERATOR ACTIVATED RECEPTOR-ALPHA

(76) Inventors: Gary W. Luehr, Hayward, CA (US); Arathi Sundaram, Fremont, CA (US); Priya Jaishankar, Hayward, CA (US); Chhaya Bhakta, San Ramon, CA (US); Pascal Druzgala, Santa Rosa, CA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/641,954

(22) Filed: Dec. 18, 2009

Related U.S. Application Data
(60) Provisional application No. 61/139,016, filed on Dec. 19, 2008.

(51) Int. Cl. A61K 31/4196; C07D 249/12; C07D 249/04; C07D 231/12; A61K 31/4192; A61K 31/415; A61P 9/10

(52) U.S. Cl. 514/383; 548/263.2; 548/255; 548/373.1; 514/359; 514/406

(57) ABSTRACT

Disclosed are compounds of the formula:

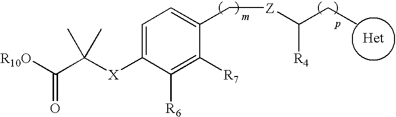

And pharmaceutically acceptable salts thereof, wherein the variables are as defined herein, which are useful in activating PPARα and in treating atherosclerosis hypercholesterolemia, primary hypercholesterolemia or mixed dyslipidemia, hypertriglyceridemia, Frederickson Types IV and V hyperlipidemia. Pharmaceutical compositions, intermediates useful in the preparation of the compounds of formula I and methods of making the compounds of formula I are also disclosed.

### BACKGROUND OF INVENTION

1. Field of the Invention

The present invention disclosed novel potent compounds which selectively activates Peroxisome Proliferator-Activated Receptor Alpha.

2. Technical Background

Peroxisome Proliferator Activated Receptors (PPARs) are members of the nuclear receptor superfamily that are ligand activated transcription factors. Three subtypes of PPARs have been identified (PPARα, PPARγ, PPARβ/δ) with each regulating tissue specific biological expression. (J. Med. Chem. 2000, 43, 527-550). Once activated with a natural ligand PPARs bind to retinoic acid receptors (RXR's) to form a dimer. (Nature, 1992, 358, 771-774). The PPAR-RXR dimer then binds to the Peroxisome Proliferator Response Element (PPRE) to initiate transcription or transrepression which results in a biological response. These PPRE's are seen in promoter region in a variety of target genes whose respective proteins are involved in lipid homeostasis. (Curr. Opin. Lipidol. 2001, 12, 245-254; Cell 1999, 97, 508).

PPARα is mainly expressed in the liver, heart, and muscle. Activation of PPARα by its natural ligand, fatty acids, leads to transrepression of apolipoprotein C-III (Apo C-III) and initiation of transcription for lipoprotein lipase (LPL), apolipoprotein A-I (Apo A-I), and apolipoprotein A-II (Apo A-II). The down regulation of Apo C-III and up regulation of LPL results in a reduction of triglycerides. Activation of Apo A-I and Apo A-II results in an increase of HDL (Circulation, 1998, 98, 2088-2093). Both reduced triglycerides and increased HDL are important in combating atherosclerosis hypercholesterolemia, primary hypercholesterolemia or mixed dyslipidemia, hypertriglyceridemia, Frederickson Types IV and V hyperlipidemia and other maladies.

Atherosclerosis is a slowly progressive disease of the arteries in which foam cells (macrophages filled with cholesterol containing lipid particles) is deposited on the inside of the arterial walls. As plaque builds up and arteries are narrowed, proper blood flow is hindered giving rise to hypertension and angina. This can then lead to congestive heart failure, heart attack, or stroke. Atherosclerosis also results in high blood pressure (hypertension) as the blood is forced through the narrowed arteries, which increases the risk of stroke and heart attack. The current treatment options include aspirin, statins, ACEI's, AIIRA's, and fibrates.

Aspirin is the most popular antiplatelet agent because of its low cost and physician confidence in prescribing it. New task force recommendations for primary prevention of cardiovascular events support the use of aspirin in high-risk patients; thus, it is unlikely to be supplanted by other antiplatelet agents. Clopidogrel (Sanofi-Synthélabo/Bristol-Myers Squibb's Plavix), however, continues to account for the majority of the sales in this class.

Statins (HMG-CoA reductase inhibitors) are the second-most-prescribed class in atherosclerosis. They are expected to gain a more prominent position in this market as more subclinical patients are treated aggressively to prevent cardiovascular events. Further, clinical patients will moderately increase their statin use as physicians adhere more strictly to guidelines, which call for lower target lipid levels. The most widely used agents in this class are atorvastatin (Warner-Lambert/Pfizer's Lipitor), simvastatin (Merck's Zocor), and pravastatin (Bristol-Myers Squibb's Pravachol).

ACEIs—As the Heart Outcomes Prevention Evaluation (HOPE) results are incorporated into medical practice and guidelines, angiotensin-converting enzyme inhibitors (ACEIs) are expected to gain ground as treatments solely for atherosclerosis. The American Heart Association/American College of Cardiology (AHA/ACC) has already recommended using these drugs in high-risk primary prevention and in secondary prevention, regardless of underlying hypertension.

AIIRAs—Like ACEIs, angiotensin II receptor antagonists (AIIRAs) may also be used for vascular protection if clinical trials demonstrate efficacy similar to that of ACEIs. (At present, AIIRAs are given only to patients who cannot tolerate ACEIs.) Physicians, however, will resist adding more pills to an already complex regimen and may not readily follow these recommendations until combined medications are available.

Ligands for the PPAR-α receptor include fatty acids and fibrates. Fibrates have been shown to reduce coronary heart disease (CHD) in both primary and secondary prevention monotherapy clinical trials. Fibrates available in the United States are clofibrate, gemfibrozil, and fenofibrate. Changes in potency provide similar effects at lower dosages: clofibrate is usually administered at 1000 mg BID, gemfibrozil at 600 mg BID, and fenofibrate at 200 mg/d in a single dose. Other fibrates available in other countries are bezafibrate and ciprofibrate. Concerns about the long-term safety of fibrates arose with publication of the results of the World Health Organization Cooperative Trial in 1978 and 1980, which showed an increase in all-cause mortality with clofibrate. In this primary-prevention trial, not only did clofibrate patients have more deaths from ischemic heart disease and stroke, but they also had more deaths from cancer. Since that time, however, the Helsinki Heart Study, published in 1987, showed no increase in adverse effects with gemfibrozil. The fibrates primarily affect triglyceride metabolism and consequently have the greatest effects on triglyceride and high-density lipoprotein cholesterol (HDL-C) levels.

Combination lipid-altering drug therapy is often indicated for patients who (1) are unable to achieve treatment goals with single lipid-altering drug treatment, (2) may be at risk for intolerance, toxicity, or adverse drug interactions with a higher dose of a single lipid-altering drug, or (3) may benefit from the combined use of two or more lipid-altering drugs with complementary mechanisms of actions and complementary effects upon reducing atherosclerotic CHD risk. However, current PPAR agonists used in combination with statin therapy can cause dangerous side effects, including liver toxicity, gallstones, and rhabdomyolosis (muscle wasting). Compounds of the instant invention aim to improve upon current PPAR agonists' safety profiles via, for example, a shorter half-life, very little escape to other tissues, and elimination in part or primarily through esterase metabolism. There is a significant unmet medical need for safer PPAR agonists with the efficacy of, for example, fenofibrate, for use either as monotherapy or in conjunction with statin therapy. With respect to combination therapy, the compounds, compositions and salts of the present invention aim to mitigate or eliminate drug-drug interactions with statins (as well as other drugs).

### SUMMARY OF THE INVENTION

Compounds of the formula:

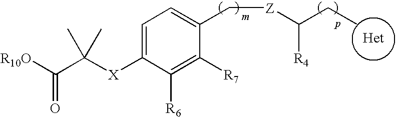

or pharmaceutically acceptable salts thereof, wherein

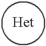

is pyrazolyl, triazolyl, or triazolonyl, each of which is substituted with 1, 2, or 3 groups that are independently aryl, arylalkyl, oxo, or C₁-C₁₀ alkyl, where the aryl portions of the aryl and arylalkyl groups are unsubstituted or substituted with 1, 2, or 3 groups that are independently C₁-C₆ alkyl optionally substituted with 1, 2, or 3 groups that are independently C₁-C₄ alkoxy, C₁-C₄ haloalkyl, or C₁-C₄ haloalkoxy; C₁-C₆ alkoxy; C₁-C₆ thioalkoxy; halogen; C₁-C₄ haloalkyl; C₁-C₄ haloalkoxy; C₁-C₄ thiohaloalkoxy; or —CO₂—C₁-C₆ alkyl where the alkyl group is optionally substituted with one or more halogens;
m is 0, 1, or 2;
n is 0, 1, or 2;
p is 0, 1, or 2;
R₄ is H; C₁-C₈ alkyl or C₁-C₄ haloalkyl;
R₆ is H, C₁-C₄ alkyl or C₁-C₄ alkoxy;
R₇ is H, C₁-C₄ alkyl or C₁-C₄ alkoxy;
R₁₀ is H, C₁-C₆ alkyl, allyl, or —C₁-C₂ alkyl-phenyl;
X is O, S, SO, or SO₂; and
Z is —C(O)O—.

A disclosed herein is a pharmaceutical composition comprising a compound of Formula 1 and at least one pharmaceutically acceptable solvent, carrier, excipient, diluent or glidant.

Further disclosed herein is a method of treating atherosclerosis hypercholesterolemia, primary hypercholesterolemia or mixed dyslipidemia, hypertriglyceridemia, Frederickson Types IV or V hyperlipidemia, the method comprising administering a therapeutically effective amount of a compound or salt of Formula 1 to a patient in need of such treatment.

Also disclosed herein is a method of activating PPARα, the method comprising administering a therapeutically effective amount of a compound or salt of Formula 1 to a patient in need of such treatment.

### DETAILED DESCRIPTION OF THE EMBODIMENTS OF THE INVENTION

As described above, provided herein are compounds of Formula 1.

In one aspect, provided are compounds of Formula 1-1, i.e., compounds of Formula 1 wherein

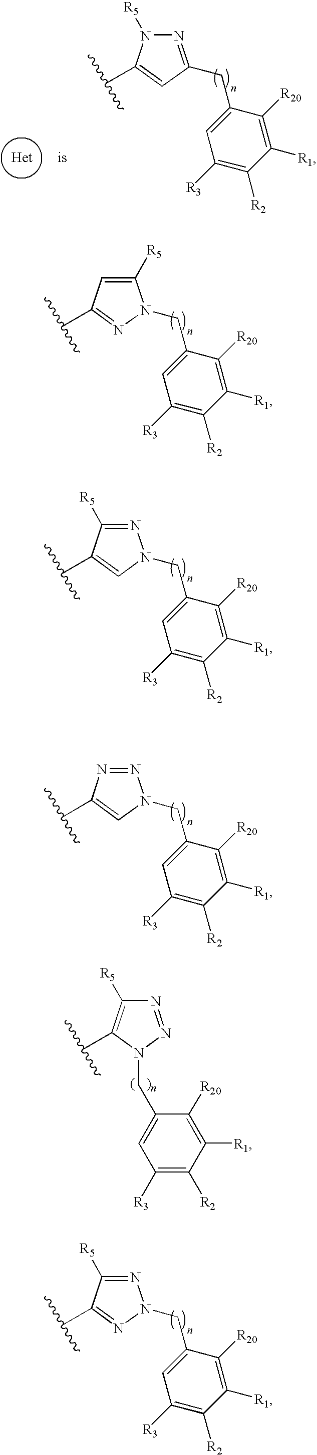

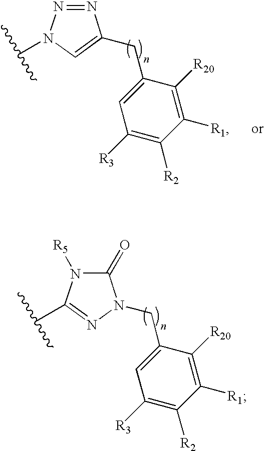

where
R₁ is H, halogen, C₁-C₆ alkyl, C₁-C₄ haloalkyl;
R₂ is H, C₁-C₆ alkyl optionally substituted with 1, 2, or 3 groups that are independently halo, C₁-C₄ alkoxy, C₁-C₄ haloalkyl, or C₁-C₄ haloalkoxy; C₁-C₆ alkoxy; C₁-C₆ thioalkoxy; halogen; C₁-C₄ haloalkyl; C₁-C₄ haloalkoxy; C₁-C₄ thiohaloalkoxy; —CO₂—C₁-C₆ alkyl where the alkyl group is optionally substituted with one or more halogens;
R₃ is H, halogen, C₁-C₆ alkyl, C₁-C₄ haloalkyl;
or R₂ and R₃ and the carbons to which they are attached form a phenyl ring or a cyclohexyl ring, each of which is optionally substituted with up to four groups that are independently halogen, C₁-C₄ alkyl, or C₁-C₄ alkoxy;
R₅ is H, C₁-C₁₀ alkyl, C₁-C₄ haloalkyl, C₃-C₆ cycloalkyl, aryl, aryl C₁-C₄ alkyl, wherein the aryl portions of the aryl and arylalkyl groups are unsubstituted or substituted with 1, 2, or 3 groups that are independently C₁-C₄ alkyl, C₁-C₄ alkoxy, halogen, C₁-C₄ haloalkyl, or C₁-C₄ haloalkoxy; and
R₂₀ is H, halogen, C₁-C₆ alkyl, or C₁-C₆ alkoxy;
with the proviso that when

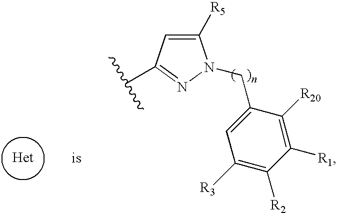

R₅ is not methyl.

In a further aspect, provided are compounds of Formula 2, i.e., compounds of Formula 1-1, having the formula.

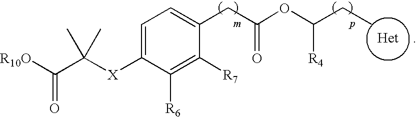

In a further aspect, provided are compounds of Formula 2-1, i.e., compounds of Formula 2, wherein
R₆ is H, methyl, ethyl, methoxy, or ethoxy; and
R₇ is H, methyl, ethyl, methoxy, or ethoxy.

In another aspect, provided are compounds of Formula 2-2, i.e., compounds of Formula 2, wherein R₁₀ is H, methyl, allyl or benzyl; and m is 0 or 1. In one embodiment, R₁₀ is H or methyl. In another embodiment, R₁₀ is H.

In another aspect, provided are compounds of Formula 2-3, i.e., compounds of Formula 2, wherein
R₄ is H, C₁-C₆ alkyl, or C₁-C₂ haloalkyl; and
p is 0 or 1.

In another aspect, provided are compounds of Formula 2-4, i.e., compounds of Formula 2, wherein
R₁ is H, halo, C₁-C₄ alkyl, or C₁-C₂ haloalkyl;
R₂ is H, C₁-C₄ alkyl, C₁-C₄ alkoxy, halo, C₁-C₄ thioalkoxy, C₁-C₂ haloalkyl; C₁-C₂ haloalkoxy; C₁-C₂ thiohaloalkoxy; —CO₂—C₁-C₄ alkyl where the alkyl group is optionally substituted with one or more halogens;
R₃ is H, halo, C₁-C₄ alkyl, or C₁-C₂ haloalkyl; and
R₂₀ is halogen, C₁-C₆ alkyl, or C₁-C₆ alkoxy.

In still another aspect, provided are compounds of Formula 2-5, i.e., compounds of Formula 2, wherein
R₅ is H, C₁-C₂ haloalkyl, C₁-C₈ alkyl, C₃-C₆ cycloalkyl, phenyl, benzyl, or phenethyl, wherein the phenyl portions of the aforementioned groups are unsubstituted or substituted with 1, 2, or 3 groups that are independently C₁-C₄ alkyl, C₁-C₄ alkoxy, halogen, C₁-C₂ haloalkyl, or C₁-C₂ haloalkoxy.

In still another aspect, provided are compounds of Formula 2-6, i.e., compounds of Formula 2, wherein
m is 0 or 1;
p is 0 or 1;
R₁ is H, halo, C₁-C₄ alkyl, or C₁-C₂ haloalkyl;
R₂ is H, C₁-C₄ alkyl, C₁-C₄ alkoxy, halo, C₁-C₄ thioalkoxy, C₁-C₂ haloalkyl; C₁-C₂ haloalkoxy; C₁-C₂ thiohaloalkoxy; —CO₂—C₁-C₄ alkyl where the alkyl group is optionally substituted with one or more halogens;
R₃ is H, halo, C₁-C₄ alkyl, or C₁-C₂ haloalkyl;
R₄ is H, C₁-C₆ alkyl, or C₁-C₂ haloalkyl;
R₅ is H, C₁-C₂ haloalkyl, C₁-C₈ alkyl, C₃-C₆ cycloalkyl, phenyl, benzyl, or phenethyl, wherein the phenyl portions of the aforementioned groups are unsubstituted or substituted with 1, 2, or 3 groups that are independently C₁-C₄ alkyl, C₁-C₄ alkoxy, halogen, C₁-C₂ haloalkyl, or C₁-C₂ haloalkoxy;
R₆ is H, methyl, ethyl, methoxy, or ethoxy;
R₇ is H, methyl, ethyl, methoxy, or ethoxy;
R₁₀ is H, methyl, allyl or benzyl; and
R₂₀ is halogen, C₁-C₆ alkyl, or C₁-C₆ alkoxy.

In still another aspect, provided are compounds of Formula 3, i.e., compounds of Formula 2-6, having the formula:

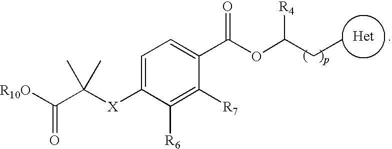

In another aspect, provided are compounds of Formula 3-1, i.e., compounds of Formula 3, wherein

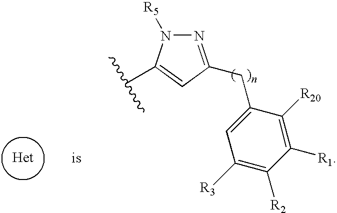

In another aspect, provided are compounds of Formula 3-2, i.e., compounds of Formula 3-1, wherein R₁ is H, F, tert-butyl, CF₃, or methyl.

In yet another aspect, provided are compounds of Formula 3-3, i.e., compounds of Formula 3-1, wherein R₂ is H, C₁-C₄ alkyl (for example, tert-butyl, methyl, ethyl or isopropyl) methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

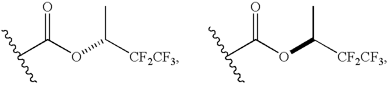

### or —C(CF₃)₂F.

In another aspect, provided are compounds of Formula 3-4, i.e., compounds of Formula 3-1, wherein R₃ is H, F, tert-butyl, CF₃, or methyl.

In still another aspect, provided are compounds of Formula 3-5, i.e., compounds of Formula 3-1, wherein R₄ is H, methyl, ethyl, propyl, CCl₃, CF₃, or hexyl.

In still another aspect, provided are compounds of Formula 3-6, i.e., compounds of Formula 3-1, wherein R₂₀ is H or F.

In still another aspect, provided are compounds of Formula 3-7, i.e., compounds of Formula 3-1, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 3-8, i.e., compounds of Formula 3-1, wherein
R₁ is H, F, tert-butyl, CF₃, methyl;
R₂ is H, C₁-C₄ alkyl (for example, tert-butyl, methyl, ethyl or isopropyl), methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

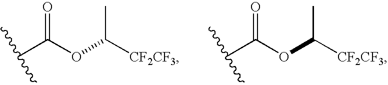

### or —C(CF₃)₂F;

R₃ is H, F, tert-butyl, CF₃, methyl;
R₄ is H, methyl, ethyl, propyl, CCl₃, CF₃, or hexyl; and
R₂₀ is H or F.

In still another aspect, provided are compounds of Formula 3-9, i.e., compounds of Formula 3-8, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 3-10, i.e., compounds of Formula 3-9, wherein R₂₀ is F.

In a further aspect, provided are compounds of Formula 3-11, i.e., compounds of Formula 3-10, wherein R₁, R₂, and R₃ are H.

In a further aspect, provided are compounds of Formula 3-12, i.e., compounds of Formula 3-10, wherein R₁ and R₃ are H.

In a further aspect, provided are compounds of Formula 3-13, i.e., compounds of Formula 3-10, wherein R₁ is F or methyl; and R₃ is H.

In a further aspect, provided are compounds of Formula 3-14, i.e., compounds of Formula 3-10, wherein R₁ is CF₃, F or methyl; and R₃ is CF₃, F or methyl. In one embodiment, at least one of R₁ and R₃ is CF₃. In one embodiment, R₁ and R₃ are CF₃.

In a further aspect, provided are compounds of Formula 3-15, i.e., compounds according to any one of Formulas 3-12, 3-13, or 3-14, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

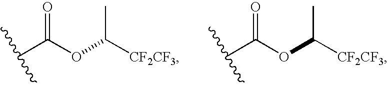

### or —C(CF₃)₂F.

In a further aspect, provided are compounds of Formula 3-16, i.e., compounds of Formula 3-15, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 3-17, i.e., compounds of Formula 3-15, wherein R₂ is methoxy, thiomethoxy, —SCF₃, —OCF₃, or —C(CF₃)₂OCH₃.

In a further aspect, provided are compounds of Formula 3-18, i.e., compounds of Formula 3-15, wherein R₂ is —CO₂Me, —CO₂Et, —CO₂CH₂CH₂CF₃, or —CO₂C(CF₃)₂CH₃.

In a further aspect, provided are compounds of Formula 3-19, i.e., compounds of Formula 3-15, wherein R₂ is

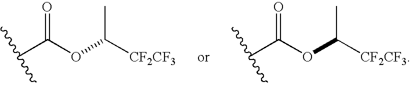

In a further aspect, provided are compounds of Formula 3-20, i.e., compounds of Formula 3-15, wherein R₂ is —C(CF₃)₂F.

In a further aspect, provided are compounds of Formula 3-21, i.e., compounds according to any one of Formulas 3-10, 3-11, 3-12, 3-13, 3-14, 3-15, 3-16, 3-17, 3-18, 3-19 or 3-20, wherein R₅ is H.

In a further aspect, provided are compounds of Formula 3-22, i.e., compounds according to any one of Formulas 3-10, 3-11, 3-12, 3-13, 3-14, 3-15, 3-16, 3-17, 3-18, 3-19 or 3-20, wherein R₅ is methyl, propyl, phenyl, hexyl, octyl, heptyl, pentyl, or ethyl.

In an aspect, provided are compounds of Formula 3-23, i.e., compounds according to any one of Formulas 3-10, 3-11, 3-12, 3-13, 3-14, 3-15, 3-16, 3-17, 3-18, 3-19 or 3-20, wherein R₅ is CF₃.

In a further aspect, provided are compounds of Formula 3-24, i.e., compounds according to any one of Formulas 3-10, 3-11, 3-12, 3-13, 3-14, 3-15, 3-16, 3-17, 3-18, 3-19 or 3-20, wherein R₅ is cyclopentyl.

In another aspect, provided are compounds of Formula 3-25, i.e., compounds according to any one of Formulas 3-10, 3-11, 3-12, 3-13, 3-14, 3-15, 3-16, 3-17, 3-18, 3-19 or 3-20, wherein R₅ is 2-fluorobenzyl, or benzyl.

In still another aspect, provided are compounds of Formula 3-26, i.e., compounds of Formula 3-9, wherein R₂₀ is H.

In a further aspect, provided are compounds of Formula 3-27, i.e., compounds of Formula 3-26, wherein R₁, R₂, and R₃ are H.

In a further aspect, provided are compounds of Formula 3-28, i.e., compounds of Formula 3-26, wherein R₁ and R₃ are H.

In a further aspect, provided are compounds of Formula 3-29, i.e., compounds of Formula 3-26, wherein R₁ is F or methyl; and R₃ is H.

In a further aspect, provided are compounds of Formula 3-30, i.e., compounds of Formula 3-26, wherein R₁ is F or methyl; and R₃ is F or methyl.

In a further aspect, provided are compounds of Formula 3-31, i.e., compounds according to any one of Formulas 3-28, 3-29, or 3-30, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

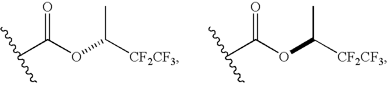

### or —C(CF₃)₂F.

In a further aspect, provided are compounds of Formula 3-32, i.e., compounds of Formula 3-31, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 3-33, i.e., compounds of Formula 3-31, wherein R₂ is methoxy, thiomethoxy, —SCF₃, —OCF₃, or —C(CF₃)₂OCH₃.

In a further aspect, provided are compounds of Formula 3-34, i.e., compounds of Formula 3-31, wherein R₂ is —CO₂Me, —CO₂Et, —CO₂CH₂CH₂CF₃, or —CO₂C(CF₃)₂CH₃.

In a further aspect, provided are compounds of Formula 3-35, i.e., compounds of Formula 3-31, wherein R₂ is

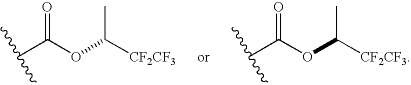

In a further aspect, provided are compounds of Formula 3-36, i.e., compounds of Formula 3-31, wherein R₂ is —C(CF₃)₂F.

In a still further aspect, provided are compounds of Formula 3-37, i.e., compounds according to any one of Formulas 3-26, 3-27, 3-28, 3-29, 3-30, 3-31, 3-32, 3-33, 3-34, 3-35 or 3-36, wherein R₅ is H.

In a further aspect, provided are compounds of Formula 3-38, i.e., compounds according to any one of Formulas 3-26, 3-27, 3-28, 3-29, 3-30, 3-31, 3-32, 3-33, 3-34, 3-35 or 3-36, wherein R₅ is methyl, propyl, phenyl, hexyl, octyl, heptyl, pentyl, or ethyl.

In an aspect, provided are compounds of Formula 3-39, i.e., compounds according to any one of Formulas 3-26, 3-27, 3-28, 3-29, 3-30, 3-31, 3-32, 3-33, 3-34, 3-35 or 3-36, wherein R₅ is CF₃.

In a further aspect, provided are compounds of Formula 3-40, i.e., compounds according to any one of Formulas 3-26, 3-27, 3-28, 3-29, 3-30, 3-31, 3-32, 3-33, 3-34, 3-35 or 3-36, wherein R₅ is cyclopentyl.

In another aspect, provided are compounds of Formula 3-41, i.e., compounds according to any one of Formulas 3-26, 3-27, 3-28, 3-29, 3-30, 3-31, 3-32, 3-33, 3-34, 3-35 or 3-36, wherein R₅ is 2-fluorobenzyl, or benzyl.

In another aspect, provided are compounds of Formula 3-42, i.e., compounds according to any one of Formulas 3-1 up to and including 3-41 wherein n is 0.

In still another aspect, provided are compounds of Formula 3-43, i.e., compounds according to any one of Formulas 3-1 up to and including 3-41 wherein n is 1.

In another aspect, provided are compounds of Formula 3-44, i.e., compounds according to either of Formulas 3-42 or 3-43, wherein R₆ and R₇ are H.

In another aspect, provided are compounds of Formula 3-45, i.e., compounds according to either of Formulas 3-42 or 3-43, wherein R₆ is OCH₃ and R₇ is H.

In yet another aspect, provided are compounds of Formula 3-46, i.e., compounds according to either of Formulas 3-42 or 3-43, wherein R₆ is H and R₇ is OCH₃.

In another aspect, provided are compounds of Formula 3-47, i.e., compounds according to either of Formulas 3-42 or 3-43, wherein R₆ and R₇ are OCH₃.

In another aspect, provided are compounds of Formula 3-48, i.e., compounds according to any one of Formulas 3-42 or 3-43, wherein R₆ and R₇ are independently H or methyl. In one embodiment, R₆ is H and R₇ is methyl. In another embodiment, R₆ is methyl and R₇ is H.

In another aspect, provided are compounds of Formula 3-49, i.e., compounds according to any one of Formulas 3-44, 3-45, 3-46, 3-47 or 3-48, wherein R₄ is H.

In another aspect, provided are compounds of Formula 3-50, i.e., compounds according to any one of Formulas 3-44, 3-45, 3-46, 3-47 or 3-48, wherein R₄ is methyl, ethyl, propyl, or hexyl. In one embodiment, R₄ is methyl. In another embodiment, R₄ is hexyl.

In another aspect, provided are compounds of Formula 3-51, i.e., compounds according to any one of Formulas 3-44, 3-45, 3-46, 3-47 or 3-48, wherein R₄ is CCl₃, CF₃. In one embodiment, R₄ is CF₃.

In another aspect, provided are compounds of Formula 3-52, i.e., compounds according to any one of Formulas 3 up to and including 3-51, wherein X is O.

In another aspect, provided are compounds of Formula 3-53, i.e., compounds according to any one of Formulas 3 up to and including 3-51, wherein X is S.

In an aspect, provided are compounds of Formula 3-54, i.e., compounds according to any one of Formulas 3 up to and including 3-53, wherein p is 0.

In an aspect, provided are compounds of Formula 3-55, i.e., compounds according to any one of Formulas 3 up to and including 3-53, wherein p is 1.

In an aspect, provided are compounds of Formula 3-56, i.e., compounds according to any one of Formulas 3 up to and including 3-54, having the following formula:

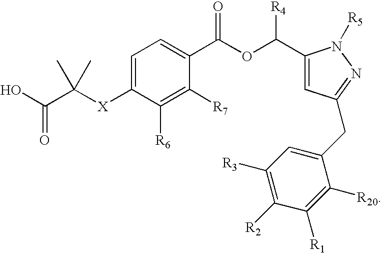

In another aspect, provided are compounds of Formula 4, i.e., compounds of Formula 3, wherein

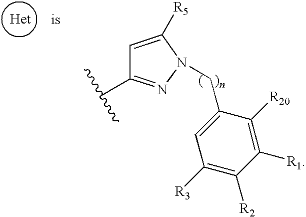

In another aspect, provided are compounds of Formula 4-2, i.e., compounds of Formula 4, wherein R₁ is H, F, tert-butyl, CF₃, or methyl.

In yet another aspect, provided are compounds of Formula 4-3, i.e., compounds of Formula 4, wherein R₂ is H, C₁-C₄ alkyl (for example, tert-butyl, methyl, ethyl or isopropyl) methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

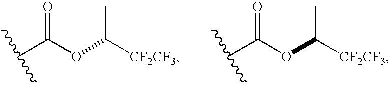

### or —C(CF₃)₂F.

In another aspect, provided are compounds of Formula 4-4, i.e., compounds of Formula 4, wherein R₃ is H, F, tert-butyl, CF₃, or methyl.

In still another aspect, provided are compounds of Formula 4-5, i.e., compounds of Formula 4, wherein R₄ is H, methyl, ethyl, propyl, CCl₃, CF₃, or hexyl.

In still another aspect, provided are compounds of Formula 4-6, i.e., compounds of Formula 4, wherein R₂₀ is H or F.

In still another aspect, provided are compounds of Formula 4-7, i.e., compounds of Formula 4, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 4-8, i.e., compounds of Formula 4, wherein
R₁ is H, F, tert-butyl, CF₃, methyl;
R₂ is H, C₁-C₄ alkyl (for example, tert-butyl, methyl, ethyl or isopropyl), methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

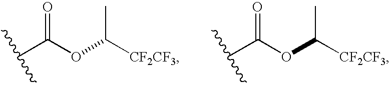

### or —C(CF₃)₂F;

R₃ is H, F, tert-butyl, CF₃, methyl;
R₄ is H, methyl, ethyl, propyl, CCl₃, CF₃, or hexyl; and
R₂₀ is H or F.

In still another aspect, provided are compounds of Formula 4-9, i.e., compounds of Formula 4-8, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 4-10, i.e., compounds of Formula 4-9, wherein R₂₀ is F.

In a further aspect, provided are compounds of Formula 4-11, i.e., compounds of Formula 4-10, wherein R₁, R₂, and R₃ are H.

In a further aspect, provided are compounds of Formula 4-12, i.e., compounds of Formula 4-10, wherein R₁ and R₃ are H.

In a further aspect, provided are compounds of Formula 4-13, i.e., compounds of Formula 4-10, wherein R₁ is F or methyl; and R₃ is H.

In a further aspect, provided are compounds of Formula 4-14, i.e., compounds of Formula 4-10, wherein R₁ is CF₃, F or methyl; and R₃ is CF₃, F or methyl. In one embodiment, at least one of R₁ and R₃ is CF₃. In one embodiment, R₁ and R₃ are CF₃.

In a further aspect, provided are compounds of Formula 4-15, i.e., compounds according to any one of Formulas 4-12, 4-13, or 4-14, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

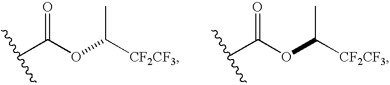

### or —C(CF₃)₂F.

In a further aspect, provided are compounds of Formula 4-16, i.e., compounds of Formula 4-15, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 4-17, i.e., compounds of Formula 4-15, wherein R₂ is methoxy, thiomethoxy, —SCF₃, —OCF₃, or —C(CF₃)₂OCH₃.

In a further aspect, provided are compounds of Formula 4-18, i.e., compounds of Formula 4-15, wherein R₂ is —CO₂Me, —CO₂Et, —CO₂CH₂CH₂CF₃, or —CO₂C(CF₃)₂CH₃.

In a further aspect, provided are compounds of Formula 4-19, i.e., compounds of Formula 4-15, wherein R₂ is

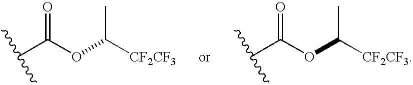

In a further aspect, provided are compounds of Formula 4-20, i.e., compounds of Formula 4-15, wherein R₂ is —C(CF₃)₂F.

In a further aspect, provided are compounds of Formula 4-21, i.e., compounds according to any one of Formulas 4-10, 4-11, 4-12, 4-13, 4-14, 4-15, 4-16, 4-17, 4-18, 4-19 or 4-20, wherein R₅ is H.

In a further aspect, provided are compounds of Formula 4-22, i.e., compounds according to any one of Formulas 4-10, 4-11, 4-12, 4-13, 4-14, 4-15, 4-16, 4-17, 4-18, 4-19 or 4-20, wherein R₅ is methyl, propyl, phenyl, hexyl, octyl, heptyl, pentyl, or ethyl.

In an aspect, provided are compounds of Formula 4-23, i.e., compounds according to any one of Formulas 4-10, 4-11, 4-12, 4-13, 4-14, 4-15, 4-16, 4-17, 4-18, 4-19 or 4-20, wherein R₅ is CF₃.

In a further aspect, provided are compounds of Formula 4-24, i.e., compounds according to any one of Formulas 4-10, 4-11, 4-12, 4-13, 4-14, 4-15, 4-16, 4-17, 4-18, 4-19 or 4-20, wherein R₅ is cyclopentyl.

In another aspect, provided are compounds of Formula 4-25, i.e., compounds according to any one of Formulas 4-10, 4-11, 4-12, 4-13, 4-14, 4-15, 4-16, 4-17, 4-18, 4-19 or 4-20, wherein R₅ is 2-fluorobenzyl, or benzyl.

In still another aspect, provided are compounds of Formula 4-26, i.e., compounds of Formula 4-9, wherein R₂₀ is H.

In a further aspect, provided are compounds of Formula 4-27, i.e., compounds of Formula 4-26, wherein R₁, R₂, and R₃ are H.

In a further aspect, provided are compounds of Formula 4-28, i.e., compounds of Formula 4-26, wherein R₁ and R₃ are H.

In a further aspect, provided are compounds of Formula 4-29, i.e., compounds of Formula 4-26, wherein R₁ is F or methyl; and R₃ is H.

In a further aspect, provided are compounds of Formula 4-30, i.e., compounds of Formula 4-26, wherein R₁ is F or methyl; and R₃ is F or methyl.

In a further aspect, provided are compounds of Formula 4-31, i.e., compounds according to any one of Formulas 4-28, 4-29, or 4-30, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

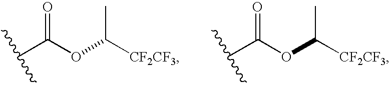

### or —C(CF₃)₂F.

In a further aspect, provided are compounds of Formula 4-32, i.e., compounds of Formula 4-31, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 4-33, i.e., compounds of Formula 4-31, wherein R₂ is methoxy, thiomethoxy, —SCF₃, —OCF₃, or —C(CF₃)₂OCH₃.

In a further aspect, provided are compounds of Formula 4-34, i.e., compounds of Formula 4-31, wherein R₂ is —CO₂Me, —CO₂Et, —CO₂CH₂CH₂CF₃, or —CO₂C(CF₃)₂CH₃.

In a further aspect, provided are compounds of Formula 4-35, i.e., compounds of Formula 4-31, wherein R₂ is

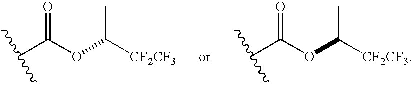

In a further aspect, provided are compounds of Formula 4-36, i.e., compounds of Formula 4-31, wherein R₂ is —C(CF₃)₂F.

In a still further aspect, provided are compounds of Formula 4-37, i.e., compounds according to any one of Formulas 4-26, 4-27, 4-28, 4-29, 4-30, 4-31, 4-32, 4-33, 4-34, 4-35 or 4-36, wherein R₅ is H.

In a further aspect, provided are compounds of Formula 4-38, i.e., compounds according to any one of Formulas 4-26, 4-27, 4-28, 4-29, 4-30, 4-31, 4-32, 4-33, 4-34, 4-35 or 4-36, wherein R₅ is methyl, propyl, phenyl, hexyl, octyl, heptyl, pentyl, or ethyl.

In an aspect, provided are compounds of Formula 4-39, i.e., compounds according to any one of Formulas 4-26, 4-27, 4-28, 4-29, 4-30, 4-31, 4-32, 4-33, 4-34, 4-35 or 4-36, wherein R₅ is CF₃.

In a further aspect, provided are compounds of Formula 4-40, i.e., compounds according to any one of Formulas 4-26, 4-27, 4-28, 4-29, 4-30, 4-31, 4-32, 4-33, 4-34, 4-35 or 4-36, wherein R₅ is cyclopentyl.

In another aspect, provided are compounds of Formula 4-41, i.e., compounds according to any one of Formulas 4-26, 4-27, 4-28, 4-29, 4-30, 4-31, 4-32, 4-33, 4-34, 4-35 or 4-36, wherein R₅ is 2-fluorobenzyl, or benzyl.

In another aspect, provided are compounds of Formula 4-42, i.e., compounds according to any one of Formulas 4 up to and including 4-41 wherein n is 0.

In still another aspect, provided are compounds of Formula 4-43, i.e., compounds according to any one of Formulas 4 up to and including 4-41 wherein n is 1.

In another aspect, provided are compounds of Formula 4-44, i.e., compounds according to either of Formulas 4-42 or 4-43, wherein R₆ and R₇ are H.

In another aspect, provided are compounds of Formula 4-45, i.e., compounds according to either of Formulas 4-42 or 4-43, wherein R₆ is OCH₃ and R₇ is H.

In yet another aspect, provided are compounds of Formula 4-46, i.e., compounds according to either of Formulas 4-42 or 4-43, wherein R₆ is H and R₇ is OCH₃.

In another aspect, provided are compounds of Formula 4-47, i.e., compounds according to either of Formulas 4-42 or 4-43, wherein R₆ and R₇ are OCH₃.

In another aspect, provided are compounds of Formula 4-48, i.e., compounds according to any one of Formulas 4-42 or 4-43, wherein R₆ and R₇ are independently H or methyl. In one embodiment, R₆ is H and R₇ is methyl. In another embodiment, R₆ is methyl and R₇ is H.

In another aspect, provided are compounds of Formula 4-49, i.e., compounds according to any one of Formulas 4-44, 4-45, 4-46, 4-47 or 4-48, wherein R₄ is H.

In another aspect, provided are compounds of Formula 4-50, i.e., compounds according to any one of Formulas 4-44, 4-45, 4-46, 4-47 or 4-48, wherein R₄ is methyl, ethyl, propyl, or hexyl. In one embodiment, R₄ is methyl. In another embodiment, R₄ is hexyl.

In another aspect, provided are compounds of Formula 4-51, i.e., compounds according to any one of Formulas 4-44, 4-45, 4-46, 4-47 or 4-48, wherein R₄ is CCl₃, CF₃. In one embodiment, R₄ is CF₃.

In another aspect, provided are compounds of Formula 4-52, i.e., compounds according to any one of Formulas 4 up to and including 4-51, wherein X is O.

In another aspect, provided are compounds of Formula 4-53, i.e., compounds according to any one of Formulas 4 up to and including 4-51, wherein X is S.

In an aspect, provided are compounds of Formula 4-54, i.e., compounds according to any one of Formulas 4 up to and including 4-53, wherein p is 0.

In an aspect, provided are compounds of Formula 4-55, i.e., compounds according to any one of Formulas 4 up to and including 4-53, wherein p is 1.

In an aspect, provided are compounds of Formula 4-56, i.e., compounds according to any one of Formulas 4 up to and including 4-54, having the following formula:

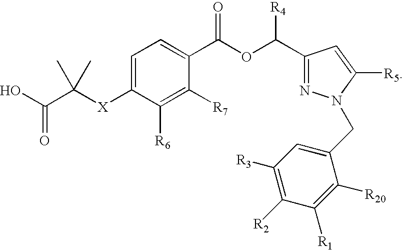

In another aspect, provided are compounds of Formula 5, i.e., compounds of Formula 3, wherein

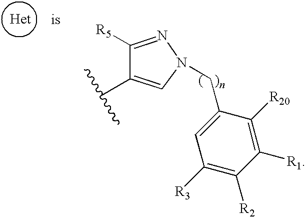

In another aspect, provided are compounds of Formula 5-2, i.e., compounds of Formula 5, wherein R₁ is H, F, tert-butyl, CF₃, or methyl.

In yet another aspect, provided are compounds of Formula 5-3, i.e., compounds of Formula 5, wherein R₂ is H, C₁-C₄ alkyl (for example, tert-butyl, methyl, ethyl or isopropyl) methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

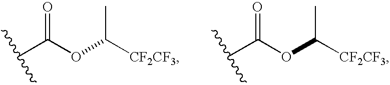

### or —C(CF₃)₂F.

In another aspect, provided are compounds of Formula 5-4, i.e., compounds of Formula 5, wherein R₃ is H, F, tert-butyl, CF₃, or methyl.

In still another aspect, provided are compounds of Formula 5-5, i.e., compounds of Formula 5, wherein R₄ is H, methyl, ethyl, propyl, CCl₃, CF₃, or hexyl.

In still another aspect, provided are compounds of Formula 5-6, i.e., compounds of Formula 5, wherein R₂₀ is H or F.

In still another aspect, provided are compounds of Formula 5-7, i.e., compounds of Formula 5, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 5-8, i.e., compounds of Formula 5, wherein
R₁ is H, F, tert-butyl, CF₃, methyl;
R₂ is H, C₁-C₄ alkyl (for example, tert-butyl, methyl, ethyl or isopropyl), methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

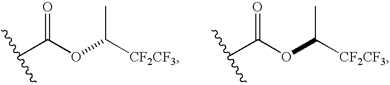

### or —C(CF₃)₂F;

R₃ is H, F, tert-butyl, CF₃, methyl;
R₄ is H, methyl, ethyl, propyl, CCl₃, CF₃, or hexyl; and
R₂₀ is H or F.

In still another aspect, provided are compounds of Formula 5-9, i.e., compounds of Formula 5-8, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 5-10, i.e., compounds of Formula 5-9, wherein R₂₀ is F.

In a further aspect, provided are compounds of Formula 5-11, i.e., compounds of Formula 5-10, wherein R₁, R₂, and R₃ are H.

In a further aspect, provided are compounds of Formula 5-12, i.e., compounds of Formula 5-10, wherein R₁ and R₃ are H.

In a further aspect, provided are compounds of Formula 5-13, i.e., compounds of Formula 5-10, wherein R₁ is F or methyl; and R₃ is H.

In a further aspect, provided are compounds of Formula 5-14, i.e., compounds of Formula 5-10, wherein R₁ is CF₃, F or methyl; and R₃ is CF₃, F or methyl. In one embodiment, at least one of R₁ and R₃ is CF₃. In one embodiment, R₁ and R₃ are CF₃.

In a further aspect, provided are compounds of Formula 5-15, i.e., compounds according to any one of Formulas 5-12, 5-13, or 5-14, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

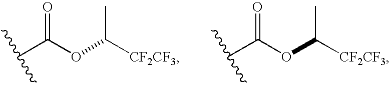

### or —C(CF₃)₂F.

In a further aspect, provided are compounds of Formula 5-16, i.e., compounds of Formula 5-15, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 5-17, i.e., compounds of Formula 5-15, wherein R₂ is methoxy, thiomethoxy, —SCF₃, —OCF₃, or —C(CF₃)₂OCH₃.

In a further aspect, provided are compounds of Formula 5-18, i.e., compounds of Formula 5-15, wherein R₂ is —CO₂Me, —CO₂Et, —CO₂CH₂CH₂CF₃, or —CO₂C(CF₃)₂CH₃.

In a further aspect, provided are compounds of Formula 5-19, i.e., compounds of Formula 5-15, wherein R₂ is

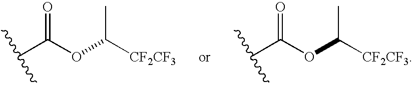

In a further aspect, provided are compounds of Formula 5-20, i.e., compounds of Formula 5-15, wherein R₂ is —C(CF₃)₂F.

In a further aspect, provided are compounds of Formula 5-21, i.e., compounds according to any one of Formulas 5-10, 5-11, 5-12, 5-13, 5-14, 5-15, 5-16, 5-17, 5-18, 5-19 or 5-20, wherein R₅ is H.

In a further aspect, provided are compounds of Formula 5-22, i.e., compounds according to any one of Formulas 5-10, 5-11, 5-12, 5-13, 5-14, 5-15, 5-16, 5-17, 5-18, 5-19 or 5-20, wherein R₅ is methyl, propyl, phenyl, hexyl, octyl, heptyl, pentyl, or ethyl.

In an aspect, provided are compounds of Formula 5-23, i.e., compounds according to any one of Formulas 5-10, 5-11, 5-12, 5-13, 5-14, 5-15, 5-16, 5-17, 5-18, 5-19 or 5-20, wherein R₅ is CF₃.

In a further aspect, provided are compounds of Formula 5-24, i.e., compounds according to any one of Formulas 5-10, 5-11, 5-12, 5-13, 5-14, 5-15, 5-16, 5-17, 5-18, 5-19 or 5-20, wherein R₅ is cyclopentyl.

In another aspect, provided are compounds of Formula 5-25, i.e., compounds according to any one of Formulas 5-10, 5-11, 5-12, 5-13, 5-14, 5-15, 5-16, 5-17, 5-18, 5-19 or 5-20, wherein R₅ is 2-fluorobenzyl, or benzyl.

In still another aspect, provided are compounds of Formula 5-26, i.e., compounds of Formula 5-9, wherein R₂₀ is H.

In a further aspect, provided are compounds of Formula 5-27, i.e., compounds of Formula 5-26, wherein R₁, R₂, and R₃ are H.

In a further aspect, provided are compounds of Formula 5-28, i.e., compounds of Formula 5-26, wherein R₁ and R₃ are H.

In a further aspect, provided are compounds of Formula 5-29, i.e., compounds of Formula 5-26, wherein R₁ is F or methyl; and R₃ is H.

In a further aspect, provided are compounds of Formula 5-30, i.e., compounds of Formula 5-26, wherein R₁ is F or methyl; and R₃ is F or methyl.

In a further aspect, provided are compounds of Formula 5-31, i.e., compounds according to any one of Formulas 5-28, 5-29, or 5-30, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

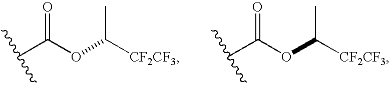

### or —C(CF₃)₂F.

In a further aspect, provided are compounds of Formula 5-32, i.e., compounds of Formula 5-31, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 5-33, i.e., compounds of Formula 5-31, wherein R₂ is methoxy, thiomethoxy, —SCF₃, —OCF₃, or —C(CF₃)₂OCH₃.

In a further aspect, provided are compounds of Formula 5-34, i.e., compounds of Formula 5-31, wherein R₂ is —CO₂Me, —CO₂Et, —CO₂CH₂CH₂CF₃, or —CO₂C(CF₃)₂CH₃.

In a further aspect, provided are compounds of Formula 5-35, i.e., compounds of Formula 5-31, wherein R₂ is

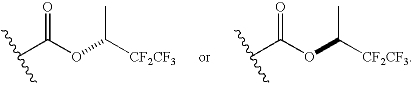

In a further aspect, provided are compounds of Formula 5-36, i.e., compounds of Formula 5-31, wherein R₂ is —C(CF₃)₂F.

In a still further aspect, provided are compounds of Formula 5-37, i.e., compounds according to any one of Formulas 5-26, 5-27, 5-28, 5-29, 5-30, 5-31, 5-32, 5-33, 5-34, 5-35 or 5-36, wherein R₅ is H.

In a further aspect, provided are compounds of Formula 5-38, i.e., compounds according to any one of Formulas 5-26, 5-27, 5-28, 5-29, 5-30, 5-31, 5-32, 5-33, 5-34, 5-35 or 5-36, wherein R₅ is methyl, propyl, phenyl, hexyl, octyl, heptyl, pentyl, or ethyl.

In an aspect, provided are compounds of Formula 5-39, i.e., compounds according to any one of Formulas 5-26, 5-27, 5-28, 5-29, 5-30, 5-31, 5-32, 5-33, 5-34, 5-35 or 5-36, wherein R₅ is CF₃.

In a further aspect, provided are compounds of Formula 5-40, i.e., compounds according to any one of Formulas 5-26, 5-27, 5-28, 5-29, 5-30, 5-31, 5-32, 5-33, 5-34, 5-35 or 5-36, wherein R₅ is cyclopentyl.

In another aspect, provided are compounds of Formula 5-41, i.e., compounds according to any one of Formulas 5-26, 5-27, 5-28, 5-29, 5-30, 5-31, 5-32, 5-33, 5-34, 5-35 or 5-36, wherein R₅ is 2-fluorobenzyl, or benzyl.

In another aspect, provided are compounds of Formula 5-42, i.e., compounds according to any one of Formulas 5 up to and including 5-41 wherein n is 0.

In still another aspect, provided are compounds of Formula 5-43, i.e., compounds according to any one of Formulas 5 up to and including 5-41 wherein n is 1.

In another aspect, provided are compounds of Formula 5-44, i.e., compounds according to either of Formulas 5-42 or 5-43, wherein R₆ and R₇ are H.

In another aspect, provided are compounds of Formula 5-45, i.e., compounds according to either of Formulas 5-42 or 5-43, wherein R₆ is OCH₃ and R₇ is H.

In yet another aspect, provided are compounds of Formula 5-46, i.e., compounds according to either of Formulas 5-42 or 5-43, wherein R₆ is H and R₇ is OCH₃.

In another aspect, provided are compounds of Formula 5-47, i.e., compounds according to either of Formulas 5-42 or 5-43, wherein R₆ and R₇ are OCH₃.

In another aspect, provided are compounds of Formula 5-48, i.e., compounds according to any one of Formulas 5-42 or 5-43, wherein R₆ and R₇ are independently H or methyl. In one embodiment, R₆ is H and R₇ is methyl. In another embodiment, R₆ is methyl and R₇ is H.

In another aspect, provided are compounds of Formula 5-49, i.e., compounds according to any one of Formulas 5-44, 5-45, 5-46, 5-47 or 5-48, wherein R₄ is H.

In another aspect, provided are compounds of Formula 5-50, i.e., compounds according to any one of Formulas 5-44, 5-45, 5-46, 5-47 or 5-48, wherein R₄ is methyl, ethyl, propyl, or hexyl. In one embodiment, R₄ is methyl. In another embodiment, R₄ is hexyl.

In another aspect, provided are compounds of Formula 5-51, i.e., compounds according to any one of Formulas 5-44, 5-45, 5-46, 5-47 or 5-48, wherein R₄ is CCl₃, CF₃. In one embodiment, R₄ is CF₃.

In another aspect, provided are compounds of Formula 5-52, i.e., compounds according to any one of Formulas 5 up to and including 5-51, wherein X is O.

In another aspect, provided are compounds of Formula 5-53, i.e., compounds according to any one of Formulas 5 up to and including 5-51, wherein X is S.

In an aspect, provided are compounds of Formula 5-54, i.e., compounds according to any one of Formulas 5 up to and including 5-53, wherein p is 0.

In an aspect, provided are compounds of Formula 5-55, i.e., compounds according to any one of Formulas 5 up to and including 5-53, wherein p is 1.

In an aspect, provided are compounds of Formula 5-56, i.e., compounds according to any one of Formulas 5 up to and including 5-54, having the following formula:

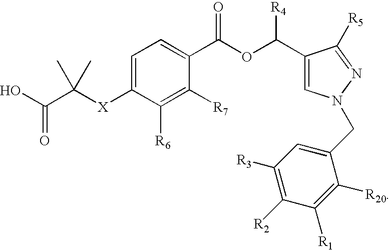

In another aspect, provided are compounds of Formula 6, i.e., compounds of Formula 3, wherein

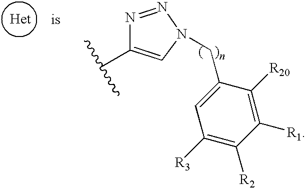

In another aspect, provided are compounds of Formula 6-2, i.e., compounds of Formula 6, wherein R₁ is H, F, tert-butyl, CF₃, or methyl.

In yet another aspect, provided are compounds of Formula 6-3, i.e., compounds of Formula 6, wherein R₂ is H, C₁-C₄ alkyl (for example, tert-butyl, methyl, ethyl or isopropyl) methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

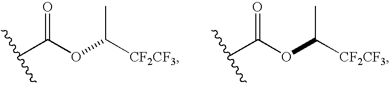

### or —C(CF₃)₂F.

In another aspect, provided are compounds of Formula 6-4, i.e., compounds of Formula 6, wherein R₃ is H, F, tert-butyl, CF₃, or methyl.

In still another aspect, provided are compounds of Formula 6-5, i.e., compounds of Formula 6, wherein R₄ is H, methyl, ethyl, propyl, CCl₃, CF₃, or hexyl.

In still another aspect, provided are compounds of Formula 6-6, i.e., compounds of Formula 6, wherein R₂₀ is H or F.

In still another aspect, provided are compounds of Formula 6-7, i.e., compounds of Formula 6, wherein R₃ is H, F, tert-butyl, CF₃, or methyl; and R₄ is H, methyl, ethyl, propyl, CCl₃, CF₃, or hexyl.

In still another aspect, provided are compounds of Formula 6-8, i.e., compounds of Formula 6, wherein
R₁ is H, F, tert-butyl, CF₃, methyl;
R₂ is H, C₁-C₄ alkyl (for example, tert-butyl, methyl, ethyl or isopropyl), methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

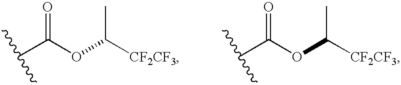

### or —C(CF₃)₂F;

R₃ is H, F, tert-butyl, CF₃, methyl;
R₄ is H, methyl, ethyl, propyl, CCl₃, CF₃, or hexyl; and
R₂₀ is H or F.

In still another aspect, provided are compounds of Formula 6-10, i.e., compounds of Formula 6-8, wherein R₂₀ is F.

In a further aspect, provided are compounds of Formula 6-11, i.e., compounds of Formula 6-10, wherein R₁, R₂, and R₃ are H.

In a further aspect, provided are compounds of Formula 6-12, i.e., compounds of Formula 6-10, wherein R₁ and R₃ are H.

In a further aspect, provided are compounds of Formula 6-13, i.e., compounds of Formula 6-10, wherein R₁ is F or methyl; and R₃ is H.

In a further aspect, provided are compounds of Formula 6-14, i.e., compounds of Formula 6-10, wherein R₁ is CF₃, F or methyl; and R₃ is CF₃, F or methyl. In one embodiment, at least one of R₁ and R₃ is CF₃. In one embodiment, R₁ and R₃ are CF₃.

In a further aspect, provided are compounds of Formula 6-15, i.e., compounds according to any one of Formulas 6-12, 6-13, or 6-14, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

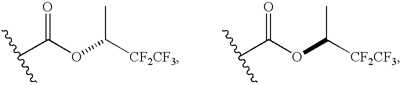

### or —C(CF₃)₂F.

In a further aspect, provided are compounds of Formula 6-16, i.e., compounds of Formula 6-15, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 6-17, i.e., compounds of Formula 6-15, wherein R₂ is methoxy, thiomethoxy, —SCF₃, —OCF₃, or —C(CF₃)₂OCH₃.

In a further aspect, provided are compounds of Formula 6-18, i.e., compounds of Formula 6-15, wherein R₂ is —CO₂Me, —CO₂Et, —CO₂CH₂CH₂CF₃, or —CO₂C(CF₃)₂CH₃.

In a further aspect, provided are compounds of Formula 6-19, i.e., compounds of Formula 6-15, wherein R₂ is

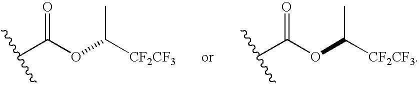

In a further aspect, provided are compounds of Formula 6-20, i.e., compounds of Formula 6-15, wherein R₂ is —C(CF₃)₂F.

In still another aspect, provided are compounds of Formula 6-26, i.e., compounds of Formula 6-8, wherein R₂₀ is H.

In a further aspect, provided are compounds of Formula 6-27, i.e., compounds of Formula 6-26, wherein R₁, R₂, and R₃ are H.

In a further aspect, provided are compounds of Formula 6-28, i.e., compounds of Formula 6-26, wherein R₁ and R₃ are H.

In a further aspect, provided are compounds of Formula 6-29, i.e., compounds of Formula 6-26, wherein R₁ is F or methyl; and R₃ is H.

In a further aspect, provided are compounds of Formula 6-30, i.e., compounds of Formula 6-26, wherein R₁ is F or methyl; and R₃ is F or methyl.

In a further aspect, provided are compounds of Formula 6-31, i.e., compounds according to any one of Formulas 6-28, 6-29, or 6-30, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

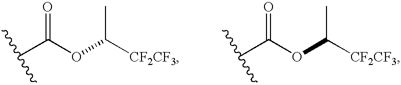

### or —C(CF₃)₂F.

In a further aspect, provided are compounds of Formula 6-32, i.e., compounds of Formula 6-31, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 6-33, i.e., compounds of Formula 6-31, wherein R₂ is methoxy, thiomethoxy, —SCF₃, —OCF₃, or —C(CF₃)₂OCH₃.

In a further aspect, provided are compounds of Formula 6-34, i.e., compounds of Formula 6-31, wherein R₂ is —CO₂Me, —CO₂Et, —CO₂CH₂CH₂CF₃, or —CO₂C(CF₃)₂CH₃.

In a further aspect, provided are compounds of Formula 6-35, i.e., compounds of Formula 6-31, wherein R₂ is

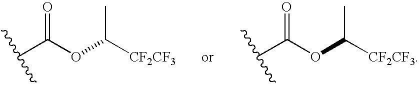

In a further aspect, provided are compounds of Formula 6-36, i.e., compounds of Formula 6-31, wherein R₂ is —C(CF₃)₂F.

In another aspect, provided are compounds of Formula 6-42, i.e., compounds according to any one of Formulas 6 up to and including 6-36 wherein n is 0.

In still another aspect, provided are compounds of Formula 6-43, i.e., compounds according to any one of Formulas 6 up to and including 6-36 wherein n is 1.

In another aspect, provided are compounds of Formula 6-44, i.e., compounds according to either of Formulas 6-42 or 6-43, wherein R₆ and R₇ are H.

In another aspect, provided are compounds of Formula 6-45, i.e., compounds according to either of Formulas 6-42 or 6-43, wherein R₆ is OCH₃ and R₇ is H.

In yet another aspect, provided are compounds of Formula 6-46, i.e., compounds according to either of Formulas 6-42 or 6-43, wherein R₆ is H and R₇ is OCH₃.

In another aspect, provided are compounds of Formula 6-47, i.e., compounds according to either of Formulas 6-42 or 6-43, wherein R₆ and R₇ are OCH₃.

In another aspect, provided are compounds of Formula 6-48, i.e., compounds according to any one of Formulas 6-42 or 6-43, wherein R₆ and R₇ are independently H or methyl. In one embodiment, R₆ is H and R₇ is methyl. In another embodiment, R₆ is methyl and R₇ is H.

In another aspect, provided are compounds of Formula 6-49, i.e., compounds according to any one of Formulas 6-44, 6-45, 6-46, 6-47 or 6-48, wherein R₄ is H.

In another aspect, provided are compounds of Formula 6-50, i.e., compounds according to any one of Formulas 6-44, 6-45, 6-46, 6-47 or 6-48, wherein R₄ is methyl, ethyl, propyl, or hexyl. In one embodiment, R₄ is methyl. In another embodiment, R₄ is hexyl.

In another aspect, provided are compounds of Formula 6-51, i.e., compounds according to any one of Formulas 6-44, 6-45, 6-46, 6-47 or 6-48, wherein R₄ is CCl₃, CF₃. In one embodiment, R₄ is CF₃.

In another aspect, provided are compounds of Formula 6-52, i.e., compounds according to any one of Formulas 6 up to and including 6-51, wherein X is O.

In another aspect, provided are compounds of Formula 6-53, i.e., compounds according to any one of Formulas 6 up to and including 6-51, wherein X is S.

In an aspect, provided are compounds of Formula 6-54, i.e., compounds according to any one of Formulas 6 up to and including 6-53, wherein p is 0.

In an aspect, provided are compounds of Formula 6-55, i.e., compounds according to any one of Formulas 6 up to and including 6-53, wherein p is 1.

In an aspect, provided are compounds of Formula 6-56, i.e., a compound wherein X is S, n is 1, R₁ is H, R₂ is methyl, R₃ is H, R₄ is (R) or (S) methyl, R₆ is H, R₇ is H, R₂₀ is H, and p is 0.

In an aspect, provided are compounds of Formula 6-57, i.e., a compound wherein X is S, n is 1, R₁ is H, R₂ is CF₃, R₃ is H, R₄ is H, R₆ is H, R₇ is H, R₂₀ is H, and p is 1.

In an aspect, provided are compounds of Formula 6-58, i.e., a compound wherein X is S, n is 1, R₁ is H, R₂ is CF₃, R₃ is H, R₄ is (R) or (S) methyl, R₆ is H, R₇ is H, R₂₀ is H, and p is 1.

In an aspect, provided are compounds of Formula 6-59, i.e., compounds according to any one of Formulas 6 up to and including 6-54 and 6-56, having the following formula:

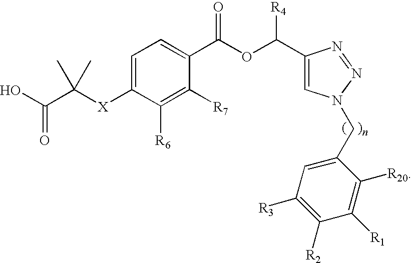

In another aspect, provided are compounds of Formula 7, i.e., compounds of Formula 3, wherein

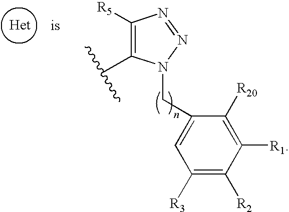

In another aspect, provided are compounds of Formula 7-2, i.e., compounds of Formula 7, wherein R₁ is H, F, tert-butyl, CF₃, or methyl.

In yet another aspect, provided are compounds of Formula 7-3, i.e., compounds of Formula 7, wherein R₂ is H, C₁-C₄ alkyl (for example, tert-butyl, methyl, ethyl or isopropyl) methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

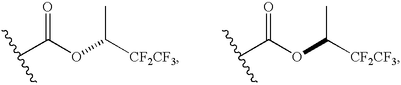

### or —C(CF₃)₂F.

In another aspect, provided are compounds of Formula 7-4, i.e., compounds of Formula 7, wherein R₃ is H, F, tert-butyl, CF₃, or methyl.

In still another aspect, provided are compounds of Formula 7-5, i.e., compounds of Formula 7, wherein R₄ is H, methyl, ethyl, propyl, CCl₃, CF₃, or hexyl.

In still another aspect, provided are compounds of Formula 7-6, i.e., compounds of Formula 7, wherein R₂₀ is H or F.

In still another aspect, provided are compounds of Formula 7-7, i.e., compounds of Formula 7, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 7-8, i.e., compounds of Formula 7, wherein
R₁ is H, F, tert-butyl, CF₃, methyl;
R₂ is H, C₁-C₄ alkyl (for example, tert-butyl, methyl, ethyl or isopropyl), methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

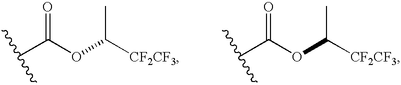

### or —C(CF₃)₂F;

R₃ is H, F, tert-butyl, CF₃, methyl;
R₄ is H, methyl, ethyl, propyl, CCl₃, CF₃, or hexyl; and
R₂₀ is H or F.

In still another aspect, provided are compounds of Formula 7-9, i.e., compounds of Formula 7-8, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 7-10, i.e., compounds of Formula 7-9, wherein R₂₀ is F.

In a further aspect, provided are compounds of Formula 7-11, i.e., compounds of Formula 7-10, wherein R₁, R₂, and R₃ are H.

In a further aspect, provided are compounds of Formula 7-12, i.e., compounds of Formula 7-10, wherein R₁ and R₃ are H.

In a further aspect, provided are compounds of Formula 7-13, i.e., compounds of Formula 7-10, wherein R₁ is F or methyl; and R₃ is H.

In a further aspect, provided are compounds of Formula 7-14, i.e., compounds of Formula 7-10, wherein R₁ is CF₃, F or methyl; and R₃ is CF₃, F or methyl. In one embodiment, at least one of R₁ and R₃ is CF₃. In one embodiment, R₁ and R₃ are CF₃.

In a further aspect, provided are compounds of Formula 7-15, i.e., compounds according to any one of Formulas 7-12, 7-13, or 7-14, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

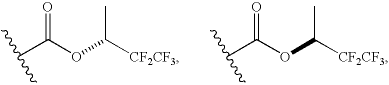

### or —C(CF₃)₂F.

In a further aspect, provided are compounds of Formula 7-16, i.e., compounds of Formula 7-15, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 7-17, i.e., compounds of Formula 7-15, wherein R₂ is methoxy, thiomethoxy, —SCF₃, —OCF₃, or —C(CF₃)₂OCH₃.

In a further aspect, provided are compounds of Formula 7-18, i.e., compounds of Formula 7-15, wherein R₂ is —CO₂Me, —CO₂Et, —CO₂CH₂CH₂CF₃, or —CO₂C(CF₃)₂CH₃.

In a further aspect, provided are compounds of Formula 7-19, i.e., compounds of Formula 7-15, wherein R₂ is

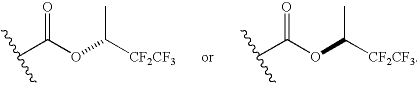

In a further aspect, provided are compounds of Formula 7-20, i.e., compounds of Formula 7-15, wherein R₂ is —C(CF₃)₂F.

In a further aspect, provided are compounds of Formula 7-21, i.e., compounds according to any one of Formulas 7-10, 7-11, 7-12, 7-13, 7-14, 7-15, 7-16, 7-17, 7-18, 7-19 or 7-20, wherein R₅ is H.

In a further aspect, provided are compounds of Formula 7-22, i.e., compounds according to any one of Formulas 7-10, 7-11, 7-12, 7-13, 7-14, 7-15, 7-16, 7-17, 7-18, 7-19 or 7-20, wherein R₅ is methyl, propyl, phenyl, hexyl, octyl, heptyl, pentyl, or ethyl.

In an aspect, provided are compounds of Formula 7-23, i.e., compounds according to any one of Formulas 7-10, 7-11, 7-12, 7-13, 7-14, 7-15, 7-16, 7-17, 7-18, 7-19 or 7-20, wherein R₅ is CF₃.

In a further aspect, provided are compounds of Formula 7-24, i.e., compounds according to any one of Formulas 7-10, 7-11, 7-12, 7-13, 7-14, 7-15, 7-16, 7-17, 7-18, 7-19 or 7-20, wherein R₅ is cyclopentyl.

In another aspect, provided are compounds of Formula 7-25, i.e., compounds according to any one of Formulas 7-10, 7-11, 7-12, 7-13, 7-14, 7-15, 7-16, 7-17, 7-18, 7-19 or 7-20, wherein R₅ is 2-fluorobenzyl, or benzyl.

In still another aspect, provided are compounds of Formula 7-26, i.e., compounds of Formula 7-9, wherein R₂₀ is H.

In a further aspect, provided are compounds of Formula 7-27, i.e., compounds of Formula 7-26, wherein R₁, R₂, and R₃ are H.

In a further aspect, provided are compounds of Formula 7-28, i.e., compounds of Formula 7-26, wherein R₁ and R₃ are H.

In a further aspect, provided are compounds of Formula 7-29, i.e., compounds of Formula 7-26, wherein R₁ is F or methyl; and R₃ is H.

In a further aspect, provided are compounds of Formula 7-30, i.e., compounds of Formula 7-26, wherein R₁ is F or methyl; and R₃ is F or methyl.

In a further aspect, provided are compounds of Formula 7-31, i.e., compounds according to any one of Formulas 7-28, 7-29, or 7-30, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

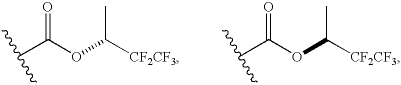

### or —C(CF₃)₂F.

In a further aspect, provided are compounds of Formula 7-32, i.e., compounds of Formula 7-31, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 7-33, i.e., compounds of Formula 7-31, wherein R₂ is methoxy, thiomethoxy, —SCF₃, —OCF₃, or —C(CF₃)₂OCH₃.

In a further aspect, provided are compounds of Formula 7-34, i.e., compounds of Formula 7-31, wherein R₂ is —CO₂Me, —CO₂Et, —CO₂CH₂CH₂CF₃, or —CO₂C(CF₃)₂CH₃.

In a further aspect, provided are compounds of Formula 7-35, i.e., compounds of Formula 7-31, wherein R₂ is

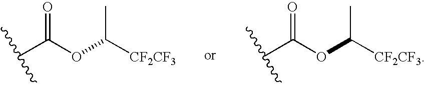

In a further aspect, provided are compounds of Formula 7-36, i.e., compounds of Formula 7-31, wherein R₂ is —C(CF₃)₂F.

In a still further aspect, provided are compounds of Formula 7-37, i.e., compounds according to any one of Formulas 7-26, 7-27, 7-28, 7-29, 7-30, 7-31, 7-32, 7-33, 7-34, 7-35 or 7-36, wherein R₅ is H.

In a further aspect, provided are compounds of Formula 7-38, i.e., compounds according to any one of Formulas 7-26, 7-27, 7-28, 7-29, 7-30, 7-31, 7-32, 7-33, 7-34, 7-35 or 7-36, wherein R₅ is methyl, propyl, phenyl, hexyl, octyl, heptyl, pentyl, or ethyl.

In an aspect, provided are compounds of Formula 7-39, i.e., compounds according to any one of Formulas 7-26, 7-27, 7-28, 7-29, 7-30, 7-31, 7-32, 7-33, 7-34, 7-35 or 7-36, wherein R₅ is CF₃.

In a further aspect, provided are compounds of Formula 7-40, i.e., compounds according to any one of Formulas 7-26, 7-27, 7-28, 7-29, 7-30, 7-31, 7-32, 7-33, 7-34, 7-35 or 7-36, wherein R₅ is cyclopentyl.

In another aspect, provided are compounds of Formula 7-41, i.e., compounds according to any one of Formulas 7-26, 7-27, 7-28, 7-29, 7-30, 7-31, 7-32, 7-33, 7-34, 7-35 or 7-36, wherein R₅ is 2-fluorobenzyl, or benzyl.

In another aspect, provided are compounds of Formula 7-42, i.e., compounds according to any one of Formulas 7 up to and including 7-41 wherein n is 0.

In still another aspect, provided are compounds of Formula 7-43, i.e., compounds according to any one of Formulas 7 up to and including 7-41 wherein n is 1.

In another aspect, provided are compounds of Formula 7-44, i.e., compounds according to either of Formulas 7-42 or 7-43, wherein R₆ and R₇ are H.

In another aspect, provided are compounds of Formula 7-45, i.e., compounds according to either of Formulas 7-42 or 7-43, wherein R₆ is OCH₃ and R₇ is H.

In yet another aspect, provided are compounds of Formula 7-46, i.e., compounds according to either of Formulas 7-42 or 7-43, wherein R₆ is H and R₇ is OCH₃.

In another aspect, provided are compounds of Formula 7-47, i.e., compounds according to either of Formulas 7-42 or 7-43, wherein R₆ and R₇ are OCH₃.

In another aspect, provided are compounds of Formula 7-48, i.e., compounds according to any one of Formulas 7-42 or 7-43, wherein R₆ and R₇ are independently H or methyl. In one embodiment, R₆ is H and R₇ is methyl. In another embodiment, R₆ is methyl and R₇ is H.

In another aspect, provided are compounds of Formula 7-49, i.e., compounds according to any one of Formulas 7-44, 7-45, 7-46, 7-47 or 7-48, wherein R₄ is H.

In another aspect, provided are compounds of Formula 7-50, i.e., compounds according to any one of Formulas 7-44, 7-45, 7-46, 7-47 or 7-48, wherein R₄ is methyl, ethyl, propyl, or hexyl. In one embodiment, R₄ is methyl. In another embodiment, R₄ is hexyl.

In another aspect, provided are compounds of Formula 7-51, i.e., compounds according to any one of Formulas 7-44, 7-45, 7-46, 7-47 or 7-48, wherein R₄ is CCl₃, CF₃. In one embodiment, R₄ is CF₃.

In another aspect, provided are compounds of Formula 7-52, i.e., compounds according to any one of Formulas 7 up to and including 7-51, wherein X is O.

In another aspect, provided are compounds of Formula 7-53, i.e., compounds according to any one of Formulas 7 up to and including 7-51, wherein X is S.

In an aspect, provided are compounds of Formula 7-54, i.e., compounds according to any one of Formulas 7 up to and including 7-53, wherein p is 0.

In an aspect, provided are compounds of Formula 7-55, i.e., compounds according to any one of Formulas 7 up to and including 7-53, wherein p is 1.

In an aspect, provided are compounds of Formula 7-56, i.e., compounds according to any one of Formulas 7 up to and including 7-54, having the following formula:

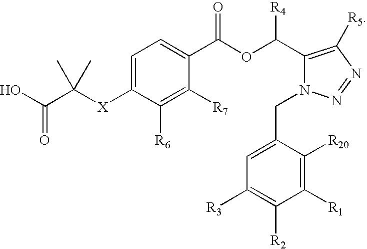

In another aspect, provided are compounds of Formula 8, i.e., compounds of Formula 3, wherein

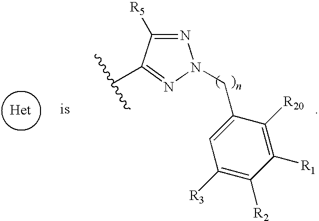

In another aspect, provided are compounds of Formula 8-2, i.e., compounds of Formula 8, wherein R₁ is H, F, tert-butyl, CF₃, or methyl.

In yet another aspect, provided are compounds of Formula 8-3, i.e., compounds of Formula 8, wherein R₂ is H, C₁-C₄ alkyl (for example, tert-butyl, methyl, ethyl or isopropyl) methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

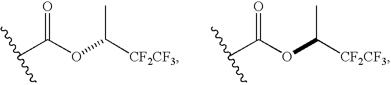

### or —C(CF₃)₂F.

In another aspect, provided are compounds of Formula 8-4, i.e., compounds of Formula 8, wherein R₃ is H, F, tert-butyl, CF₃, or methyl.

In still another aspect, provided are compounds of Formula 8-5, i.e., compounds of Formula 8, wherein R₄ is H, methyl, ethyl, propyl, CCl₃, CF₃, or hexyl.

In still another aspect, provided are compounds of Formula 8-6, i.e., compounds of Formula 8, wherein R₂₀ is H or F.

In still another aspect, provided are compounds of Formula 8-7, i.e., compounds of Formula 8, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 8-8, i.e., compounds of Formula 8, wherein
R₁ is H, F, tert-butyl, CF₃, methyl;
R₂ is H, C₁-C₄ alkyl (for example, tert-butyl, methyl, ethyl or isopropyl), methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

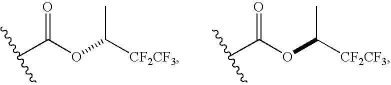

### or —C(CF₃)₂F;

R₃ is H, F, tert-butyl, CF₃, methyl;
R₄ is H, methyl, ethyl, propyl, CCl₃, CF₃, or hexyl; and
R₂₀ is H or F.

In still another aspect, provided are compounds of Formula 8-9, i.e., compounds of Formula 8-8, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 8-10, i.e., compounds of Formula 8-9, wherein R₂₀ is F.

In a further aspect, provided are compounds of Formula 8-11, i.e., compounds of Formula 8-10, wherein R₁, R₂, and R₃ are H.

In a further aspect, provided are compounds of Formula 8-12, i.e., compounds of Formula 8-10, wherein R₁ and R₃ are H.

In a further aspect, provided are compounds of Formula 8-13, i.e., compounds of Formula 8-10, wherein R₁ is F or methyl; and R₃ is H.

In a further aspect, provided are compounds of Formula 8-14, i.e., compounds of Formula 8-10, wherein R₁ is CF₃, F or methyl; and R₃ is CF₃, F or methyl. In one embodiment, at least one of R₁ and R₃ is CF₃. In one embodiment, R₁ and R₃ are CF₃.

In a further aspect, provided are compounds of Formula 8-15, i.e., compounds according to any one of Formulas 8-12, 8-13, or 8-14, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

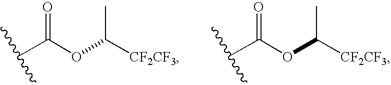

### or —C(CF₃)₂F.

In a further aspect, provided are compounds of Formula 8-16, i.e., compounds of Formula 8-15, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 8-17, i.e., compounds of Formula 8-15, wherein R₂ is methoxy, thiomethoxy, —SCF₃, —OCF₃, or —C(CF₃)₂OCH₃.

In a further aspect, provided are compounds of Formula 8-18, i.e., compounds of Formula 8-15, wherein R₂ is —CO₂Me, —CO₂Et, —CO₂CH₂CH₂CF₃, or —CO₂C(CF₃)₂CH₃.

In a further aspect, provided are compounds of Formula 8-19, i.e., compounds of Formula 8-15, wherein R₂ is

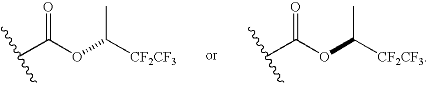

In a further aspect, provided are compounds of Formula 8-20, i.e., compounds of Formula 8-15, wherein R₂ is —C(CF₃)₂F.

In a further aspect, provided are compounds of Formula 8-21, i.e., compounds according to any one of Formulas 8-10, 8-11, 8-12, 8-13, 8-14, 8-15, 8-16, 8-17, 8-18, 8-19 or 8-20, wherein R₅ is H.

In a further aspect, provided are compounds of Formula 8-22, i.e., compounds according to any one of Formulas 8-10, 8-11, 8-12, 8-13, 8-14, 8-15, 8-16, 8-17, 8-18, 8-19 or 8-20, wherein R₅ is methyl, propyl, phenyl, hexyl, octyl, heptyl, pentyl, or ethyl.

In an aspect, provided are compounds of Formula 8-23, i.e., compounds according to any one of Formulas 8-10, 8-11, 8-12, 8-13, 8-14, 8-15, 8-16, 8-17, 8-18, 8-19 or 8-20, wherein R₅ is CF₃.

In a further aspect, provided are compounds of Formula 8-24, i.e., compounds according to any one of Formulas 8-10, 8-11, 8-12, 8-13, 8-14, 8-15, 8-16, 8-17, 8-18, 8-19 or 8-20, wherein R₅ is cyclopentyl.

In another aspect, provided are compounds of Formula 8-25, i.e., compounds according to any one of Formulas 8-10, 8-11, 8-12, 8-13, 8-14, 8-15, 8-16, 8-17, 8-18, 8-19 or 8-20, wherein R₅ is 2-fluorobenzyl, or benzyl.

In still another aspect, provided are compounds of Formula 8-26, i.e., compounds of Formula 8-9, wherein R₂₀ is H.

In a further aspect, provided are compounds of Formula 8-27, i.e., compounds of Formula 8-26, wherein R₁, R₂, and R₃ are H.

In a further aspect, provided are compounds of Formula 8-28, i.e., compounds of Formula 8-26, wherein R₁ and R₃ are H.

In a further aspect, provided are compounds of Formula 8-29, i.e., compounds of Formula 8-26, wherein R₁ is F or methyl; and R₃ is H.

In a further aspect, provided are compounds of Formula 8-30, i.e., compounds of Formula 8-26, wherein R₁ is F or methyl; and R₃ is F or methyl.

In a further aspect, provided are compounds of Formula 8-31, i.e., compounds according to any one of Formulas 8-28, 8-29, or 8-30, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

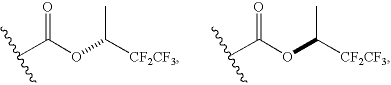

### or —C(CF₃)₂F.

In a further aspect, provided are compounds of Formula 8-32, i.e., compounds of Formula 8-31, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 8-33, i.e., compounds of Formula 8-31, wherein R₂ is methoxy, thiomethoxy, —SCF₃, —OCF₃, or —C(CF₃)₂OCH₃.

In a further aspect, provided are compounds of Formula 8-34, i.e., compounds of Formula 8-31, wherein R₂ is —CO₂Me, —CO₂Et, —CO₂CH₂CH₂CF₃, or —CO₂C(CF₃)₂CH₃.

In a further aspect, provided are compounds of Formula 8-35, i.e., compounds of Formula 8-31, wherein R₂ is

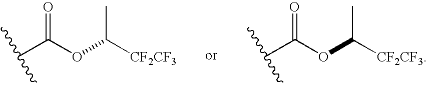

In a further aspect, provided are compounds of Formula 8-36, i.e., compounds of Formula 8-31, wherein R₂ is —C(CF₃)₂F.

In a still further aspect, provided are compounds of Formula 8-37, i.e., compounds according to any one of Formulas 8-26, 8-27, 8-28, 8-29, 8-30, 8-31, 8-32, 8-33, 8-34, 8-35 or 8-36, wherein R₅ is H.

In a further aspect, provided are compounds of Formula 8-38, i.e., compounds according to any one of Formulas 8-26, 8-27, 8-28, 8-29, 8-30, 8-31, 8-32, 8-33, 8-34, 8-35 or 8-36, wherein R₅ is methyl, propyl, phenyl, hexyl, octyl, heptyl, pentyl, or ethyl.

In an aspect, provided are compounds of Formula 8-39, i.e., compounds according to any one of Formulas 8-26, 8-27, 8-28, 8-29, 8-30, 8-31, 8-32, 8-33, 8-34, 8-35 or 8-36, wherein R₅ is CF₃.

In a further aspect, provided are compounds of Formula 8-40, i.e., compounds according to any one of Formulas 8-26, 8-27, 8-28, 8-29, 8-30, 8-31, 8-32, 8-33, 8-34, 8-35 or 8-36, wherein R₅ is cyclopentyl.

In another aspect, provided are compounds of Formula 8-41, i.e., compounds according to any one of Formulas 8-26, 8-27, 8-28, 8-29, 8-30, 8-31, 8-32, 8-33, 8-34, 8-35 or 8-36, wherein R₅ is 2-fluorobenzyl, or benzyl.

In another aspect, provided are compounds of Formula 8-42, i.e., compounds according to any one of Formulas 8 up to and including 8-41 wherein n is 0.

In still another aspect, provided are compounds of Formula 8-43, i.e., compounds according to any one of Formulas 8 up to and including 8-41 wherein n is 1.

In another aspect, provided are compounds of Formula 8-44, i.e., compounds according to either of Formulas 8-42 or 8-43, wherein R₆ and R₇ are H.

In another aspect, provided are compounds of Formula 8-45, i.e., compounds according to either of Formulas 8-42 or 8-43, wherein R₆ is OCH₃ and R₇ is H.

In yet another aspect, provided are compounds of Formula 8-46, i.e., compounds according to either of Formulas 8-42 or 8-43, wherein R₆ is H and R₇ is OCH₃.

In another aspect, provided are compounds of Formula 8-47, i.e., compounds according to either of Formulas 8-42 or 8-43, wherein R₆ and R₇ are OCH₃.

In another aspect, provided are compounds of Formula 8-48, i.e., compounds according to any one of Formulas 8-42 or 8-43, wherein R₆ and R₇ are independently H or methyl. In one embodiment, R₆ is H and R₇ is methyl. In another embodiment, R₆ is methyl and R₇ is H.

In another aspect, provided are compounds of Formula 8-49, i.e., compounds according to any one of Formulas 8-44, 8-45, 8-46, 8-47 or 8-48, wherein R₄ is H.

In another aspect, provided are compounds of Formula 8-50, i.e., compounds according to any one of Formulas 8-44, 8-45, 8-46, 8-47 or 8-48, wherein R₄ is methyl, ethyl, propyl, or hexyl. In one embodiment, R₄ is methyl. In another embodiment, R₄ is hexyl.

In another aspect, provided are compounds of Formula 8-51, i.e., compounds according to any one of Formulas 8-44, 8-45, 8-46, 8-47 or 8-48, wherein R₄ is CCl₃, CF₃. In one embodiment, R₄ is CF₃.

In another aspect, provided are compounds of Formula 8-52, i.e., compounds according to any one of Formulas 8 up to and including 8-51, wherein X is O.

In another aspect, provided are compounds of Formula 8-53, i.e., compounds according to any one of Formulas 8 up to and including 8-51, wherein X is S.

In an aspect, provided are compounds of Formula 8-54, i.e., compounds according to any one of Formulas 8 up to and including 8-53, wherein p is 0.

In an aspect, provided are compounds of Formula 8-55, i.e., compounds according to any one of Formulas 8 up to and including 8-53, wherein p is 1.

In an aspect, provided are compounds of Formula 8-56, i.e., compounds according to any one of Formulas 8 up to and including 8-54, having the following formula:

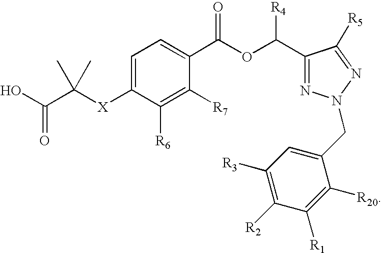

In another aspect, provided are compounds of Formula 9, i.e., compounds of Formula 3, wherein

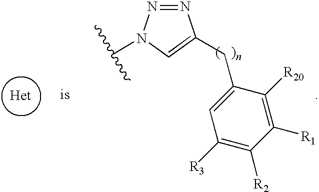

In another aspect, provided are compounds of Formula 9-2, i.e., compounds of Formula 9, wherein R₁ is H, F, tert-butyl, CF₃, or methyl.

In yet another aspect, provided are compounds of Formula 9-3, i.e., compounds of Formula 9, wherein R₂ is H, C₁-C₄ alkyl (for example, tert-butyl, methyl, ethyl or isopropyl) methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

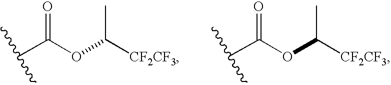

### or —C(CF₃)₂F.

In another aspect, provided are compounds of Formula 9-4, i.e., compounds of Formula 9, wherein R₃ is H, F, tert-butyl, CF₃, or methyl.

In still another aspect, provided are compounds of Formula 9-5, i.e., compounds of Formula 9, wherein R₄ is H, methyl, ethyl, propyl, CCl₃, CF₃, or hexyl.

In still another aspect, provided are compounds of Formula 9-6, i.e., compounds of Formula 9, wherein R₂₀ is H or F.

In still another aspect, provided are compounds of Formula 9-7, i.e., compounds of Formula 9, wherein R₃ is H, F, tert-butyl, CF₃, or methyl; and R₄ is H, methyl, ethyl, propyl, CCl₃, CF₃, or hexyl.

In still another aspect, provided are compounds of Formula 9-8, i.e., compounds of Formula 9, wherein
R₁ is H, F, tert-butyl, CF₃, methyl;
R₂ is H, C₁-C₄ alkyl (for example, tert-butyl, methyl, ethyl or isopropyl), methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

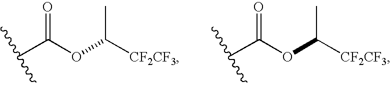

### or —C(CF₃)₂F;

R₃ is H, F, tert-butyl, CF₃, methyl;
R₄ is H, methyl, ethyl, propyl, CCl₃, CF₃, or hexyl; and
R₂₀ is H or F.

In still another aspect, provided are compounds of Formula 9-10, i.e., compounds of Formula 9-8, wherein R₂₀ is F.

In a further aspect, provided are compounds of Formula 9-11, i.e., compounds of Formula 9-10, wherein R₁, R₂, and R₃ are H.

In a further aspect, provided are compounds of Formula 9-12, i.e., compounds of Formula 9-10, wherein R₁ and R₃ are H.

In a further aspect, provided are compounds of Formula 9-13, i.e., compounds of Formula 9-10, wherein R₁ is F or methyl; and R₃ is H.

In a further aspect, provided are compounds of Formula 9-14, i.e., compounds of Formula 9-10, wherein R₁ is CF₃, F or methyl; and R₃ is CF₃, F or methyl. In one embodiment, at least one of R₁ and R₃ is CF₃. In one embodiment, R₁ and R₃ are CF₃.

In a further aspect, provided are compounds of Formula 9-15, i.e., compounds according to any one of Formulas 9-12, 9-13, or 9-14, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

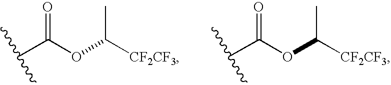

### or —C(CF₃)₂F.

In a further aspect, provided are compounds of Formula 9-16, i.e., compounds of Formula 9-15, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 9-17, i.e., compounds of Formula 9-15, wherein R₂ is methoxy, thiomethoxy, —SCF₃, —OCF₃, or —C(CF₃)₂OCH₃.

In a further aspect, provided are compounds of Formula 9-18, i.e., compounds of Formula 9-15, wherein R₂ is —CO₂Me, —CO₂Et, —CO₂CH₂CH₂CF₃, or —CO₂C(CF₃)₂CH₃.

In a further aspect, provided are compounds of Formula 9-19, i.e., compounds of Formula 9-15, wherein R₂ is

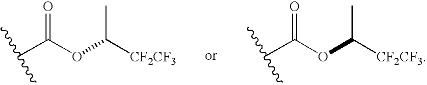

In a further aspect, provided are compounds of Formula 9-20, i.e., compounds of Formula 9-15, wherein R₂ is —C(CF₃)₂F.

In still another aspect, provided are compounds of Formula 9-26, i.e., compounds of Formula 9-8, wherein R₂₀ is H.

In a further aspect, provided are compounds of Formula 9-27, i.e., compounds of Formula 9-26, wherein R₁, R₂, and R₃ are H.

In a further aspect, provided are compounds of Formula 9-28, i.e., compounds of Formula 9-26, wherein R₁ and R₃ are H.

In a further aspect, provided are compounds of Formula 9-29, i.e., compounds of Formula 9-26, wherein R₁ is F or methyl; and R₃ is H.

In a further aspect, provided are compounds of Formula 9-30, i.e., compounds of Formula 9-26, wherein R₁ is F or methyl; and R₃ is F or methyl.

In a further aspect, provided are compounds of Formula 9-31, i.e., compounds according to any one of Formulas 9-28, 9-29, or 9-30, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

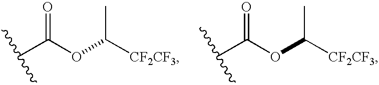

### or —C(CF₃)₂F.

In a further aspect, provided are compounds of Formula 9-32, i.e., compounds of Formula 9-31, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 9-33, i.e., compounds of Formula 9-31, wherein R₂ is methoxy, thiomethoxy, —SCF₃, —OCF₃, or —C(CF₃)₂OCH₃.

In a further aspect, provided are compounds of Formula 9-34, i.e., compounds of Formula 9-31, wherein R₂ is —CO₂Me, —CO₂Et, —CO₂CH₂CH₂CF₃, or —CO₂C(CF₃)₂CH₃.

In a further aspect, provided are compounds of Formula 9-35, i.e., compounds of Formula 9-31, wherein R₂ is

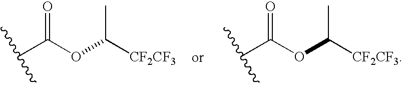

In a further aspect, provided are compounds of Formula 9-36, i.e., compounds of Formula 9-31, wherein R₂ is —C(CF₃)₂F.

In another aspect, provided are compounds of Formula 9-42, i.e., compounds according to any one of Formulas 9 up to and including 9-36 wherein n is 0.

In still another aspect, provided are compounds of Formula 9-43, i.e., compounds according to any one of Formulas 9 up to and including 9-36 wherein n is 1.

In another aspect, provided are compounds of Formula 9-44, i.e., compounds according to either of Formulas 9-42 or 9-43, wherein R₆ and R₇ are H.

In another aspect, provided are compounds of Formula 9-45, i.e., compounds according to either of Formulas 9-42 or 9-43, wherein R₆ is OCH₃ and R₇ is H.

In yet another aspect, provided are compounds of Formula 9-46, i.e., compounds according to either of Formulas 9-42 or 9-43, wherein R₆ is H and R₇ is OCH₃.

In another aspect, provided are compounds of Formula 9-47, i.e., compounds according to either of Formulas 9-42 or 9-43, wherein R₆ and R₇ are OCH₃.

In another aspect, provided are compounds of Formula 9-48, i.e., compounds according to any one of Formulas 9-42 or 9-43, wherein R₆ and R₇ are independently H or methyl. In one embodiment, R₆ is H and R₇ is methyl. In another embodiment, R₆ is methyl and R₇ is H.

In another aspect, provided are compounds of Formula 9-49, i.e., compounds according to any one of Formulas 9-44, 9-45, 9-46, 9-47 or 9-48, wherein R₄ is H.

In another aspect, provided are compounds of Formula 9-50, i.e., compounds according to any one of Formulas 9-44, 9-45, 9-46, 9-47 or 9-48, wherein R₄ is methyl, ethyl, propyl, or hexyl. In one embodiment, R₄ is methyl. In another embodiment, R₄ is hexyl.

In another aspect, provided are compounds of Formula 9-51, i.e., compounds according to any one of Formulas 9-44, 9-45, 9-46, 9-47 or 9-48, wherein R₄ is CCl₃, CF₃. In one embodiment, R₄ is CF₃.

In another aspect, provided are compounds of Formula 9-52, i.e., compounds according to any one of Formulas 9 up to and including 9-51, wherein X is O.

In another aspect, provided are compounds of Formula 9-53, i.e., compounds according to any one of Formulas 9 up to and including 9-51, wherein X is S.

In an aspect, provided are compounds of Formula 9-54, i.e., compounds according to any one of Formulas 9 up to and including 9-53, wherein p is 0.

In an aspect, provided are compounds of Formula 9-55, i.e., compounds according to any one of Formulas 9 up to and including 9-53, wherein p is 1.

In an aspect, provided are compounds of Formula 9-56, i.e., compounds according to any one of Formulas 9 up to and including 9-54, having the following formula:

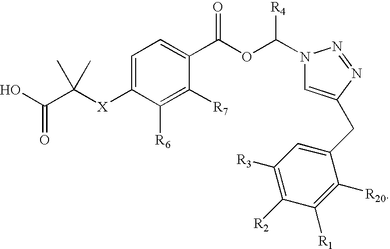

In another aspect, provided are compounds of Formula 10, i.e., compounds of Formula 3, wherein

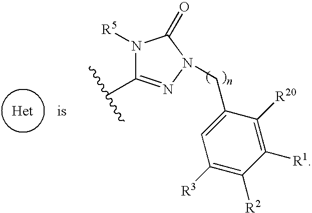

In another aspect, provided are compounds of Formula 10-2, i.e., compounds of Formula 10, wherein R₁ is H, F, tert-butyl, CF₃, or methyl.

In yet another aspect, provided are compounds of Formula 10-3, i.e., compounds of Formula 10, wherein R₂ is H, C₁-C₄ alkyl (for example, tert-butyl, methyl, ethyl or isopropyl) methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

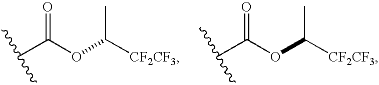

### or —C(CF₃)₂F.

In another aspect, provided are compounds of Formula 10-4, i.e., compounds of Formula 10, wherein R₃ is H, F, tert-butyl, CF₃, or methyl.

In still another aspect, provided are compounds of Formula 10-5, i.e., compounds of Formula 10, wherein R₄ is H, methyl, ethyl, propyl, CCl₃, CF₃, or hexyl.

In still another aspect, provided are compounds of Formula 10-6, i.e., compounds of Formula 10, wherein R₂₀ is H or F.

In still another aspect, provided are compounds of Formula 10-7, i.e., compounds of Formula 10, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 10-8, i.e., compounds of Formula 10, wherein
R₁ is H, F, tert-butyl, CF₃, methyl;
R₂ is H, C₁-C₄ alkyl (for example, tert-butyl, methyl, ethyl or isopropyl), methoxy, CF₃, phenyl, thiomethoxy, F, Cl, Br, OCF₃, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

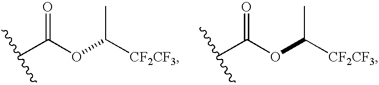

### or —C(CF₃)₂F;

R₃ is H, F, tert-butyl, CF₃, methyl;
R₄ is H, methyl, ethyl, propyl, CCl₃, CF₃, or hexyl; and
R₂₀ is H or F.

In still another aspect, provided are compounds of Formula 10-9, i.e., compounds of Formula 10-8, wherein R₅ is H, methyl, CF₃, propyl, phenyl, hexyl, octyl, cyclopentyl, 2-fluorobenzyl, benzyl, heptyl, pentyl, or ethyl.

In still another aspect, provided are compounds of Formula 10-10, i.e., compounds of Formula 10-9, wherein R₂₀ is F.

In a further aspect, provided are compounds of Formula 10-11, i.e., compounds of Formula 10-10, wherein R₁, R₂, and R₃ are H.

In a further aspect, provided are compounds of Formula 10-12, i.e., compounds of Formula 10-10, wherein R₁ and R₃ are H.

In a further aspect, provided are compounds of Formula 10-13, i.e., compounds of Formula 10-10, wherein R₁ is F or methyl; and R₃ is H.

In a further aspect, provided are compounds of Formula 10-14, i.e., compounds of Formula 10-10, wherein R₁ is CF₃, F or methyl; and R₃ is CF₃, F or methyl. In one embodiment, at least one of R₁ and R₃ is CF₃. In one embodiment, R₁ and R₃ are CF₃.

In a further aspect, provided are compounds of Formula 10-15, i.e., compounds according to any one of Formulas 13-12, 13-13, or 13-14, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

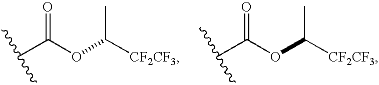

### or —C(CF₃)₂F.

In a further aspect, provided are compounds of Formula 10-16, i.e., compounds of Formula 10-15, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 10-17, i.e., compounds of Formula 10-15, wherein R₂ is methoxy, thiomethoxy, —SCF₃, —OCF₃, or —C(CF₃)₂OCH₃.

In a further aspect, provided are compounds of Formula 10-18, i.e., compounds of Formula 10-15, wherein R₂ is —CO₂Me, —CO₂Et, —CO₂CH₂CH₂CF₃, or —CO₂C(CF₃)₂CH₃.

In a further aspect, provided are compounds of Formula 10-19, i.e., compounds of Formula 10-15, wherein R₂ is

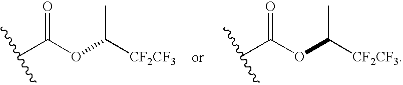

In a further aspect, provided are compounds of Formula 10-20, i.e., compounds of Formula 10-15, wherein R₂ is —C(CF₃)₂F.

In a further aspect, provided are compounds of Formula 10-21, i.e., compounds according to any one of Formulas 13-10, 13-11, 13-12, 13-13, 13-14, 13-15, 13-16, 13-17, 13-18, 13-19 or 13-20, wherein R₅ is H.

In a further aspect, provided are compounds of Formula 10-22, i.e., compounds according to any one of Formulas 13-10, 13-11, 13-12, 13-13, 13-14, 13-15, 13-16, 13-17, 13-18, 13-19 or 13-20, wherein R₅ is methyl, propyl, phenyl, hexyl, octyl, heptyl, pentyl, or ethyl.

In an aspect, provided are compounds of Formula 10-23, i.e., compounds according to any one of Formulas 13-10, 13-11, 13-12, 13-13, 13-14, 13-15, 13-16, 13-17, 13-18, 13-19 or 13-20, wherein R₅ is CF₃.

In a further aspect, provided are compounds of Formula 10-24, i.e., compounds according to any one of Formulas 13-10, 13-11, 13-12, 13-13, 13-14, 13-15, 13-16, 13-17, 13-18, 13-19 or 13-20, wherein R₅ is cyclopentyl.

In another aspect, provided are compounds of Formula 10-25, i.e., compounds according to any one of Formulas 13-10, 13-11, 13-12, 13-13, 13-14, 13-15, 13-16, 13-17, 13-18, 13-19 or 13-20, wherein R₅ is 2-fluorobenzyl, or benzyl.

In still another aspect, provided are compounds of Formula 10-26, i.e., compounds of Formula 10-9, wherein R₂₀ is H.

In a further aspect, provided are compounds of Formula 10-27, i.e., compounds of Formula 10-26, wherein R₁, R₂, and R₃ are H.

In a further aspect, provided are compounds of Formula 10-28, i.e., compounds of Formula 10-26, wherein R₁ and R₃ are H.

In a further aspect, provided are compounds of Formula 10-29, i.e., compounds of Formula 10-26, wherein R₁ is F or methyl; and R₃ is H.

In a further aspect, provided are compounds of Formula 10-30, i.e., compounds of Formula 10-26, wherein R₁ is F or methyl; and R₃ is F or methyl.

In a further aspect, provided are compounds of Formula 10-31, i.e., compounds according to any one of Formulas 13-28, 13-29, or 13-30, wherein R₂ is H, C₁-C₄ alkyl (for example, tert-butyl, methyl, ethyl or isopropyl), methoxy, CF₃, phenyl, thiomethoxy, F, Cl, Br, —SCF₃, —OCF₃, —C(CF₃)₂OCH₃, phenyl, —CO₂Me, —CO₂Et, —CO₂CH₂CH₂CF₃, —CO₂C(CF₃)₂CH₃,

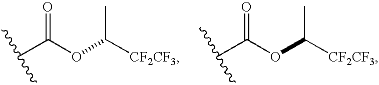

### or —C(CF₃)₂F.

In a further aspect, provided are compounds of Formula 10-32, i.e., compounds of Formula 10-31, wherein R₂ is H or C₁-C₄ alkyl. In one embodiment, R₂ is H, tert-butyl, methyl, ethyl or isopropyl. In one embodiment, R₂ is H. In another embodiment, R₂ is tert-butyl, methyl, ethyl or isopropyl.

In a further aspect, provided are compounds of Formula 10-33, i.e., compounds of Formula 10-31, wherein R₂ is methoxy, thiomethoxy, —SCF₃, —OCF₃, or —C(CF₃)₂OCH₃.

In a further aspect, provided are compounds of Formula 10-34, i.e., compounds of Formula 10-31, wherein R₂ is —CO₂Me, —CO₂Et, —CO₂CH₂CH₂CF₃, or —CO₂C(CF₃)₂CH₃.

In a further aspect, provided are compounds of Formula 10-35, i.e., compounds of Formula 10-31, wherein R₂ is

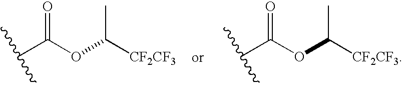

In a further aspect, provided are compounds of Formula 10-36, i.e., compounds of Formula 10-31, wherein R₂ is —C(CF₃)₂F.

In a still further aspect, provided are compounds of Formula 10-37, i.e., compounds according to any one of Formulas 13-26, 13-27, 13-28, 13-29, 13-30, 13-31, 13-32, 13-33, 13-34, 13-35 or 13-36, wherein R₅ is H.

In a further aspect, provided are compounds of Formula 10-38, i.e., compounds according to any one of Formulas 13-26, 13-27, 13-28, 13-29, 13-30, 13-31, 13-32, 13-33, 13-34, 13-35 or 13-36, wherein R₅ is methyl, propyl, phenyl, hexyl, octyl, heptyl, pentyl, or ethyl.

In an aspect, provided are compounds of Formula 10-39, i.e., compounds according to any one of Formulas 13-26, 13-27, 13-28, 13-29, 13-30, 13-31, 13-32, 13-33, 13-34, 13-35 or 13-36, wherein R₅ is CF₃.

In a further aspect, provided are compounds of Formula 10-40, i.e., compounds according to any one of Formulas 13-26, 13-27, 13-28, 13-29, 13-30, 13-31, 13-32, 13-33, 13-34, 13-35 or 13-36, wherein R₅ is cyclopentyl.

In another aspect, provided are compounds of Formula 10-41, i.e., compounds according to any one of Formulas 13-26, 13-27, 13-28, 13-29, 13-30, 13-31, 13-32, 13-33, 13-34, 13-35 or 13-36, wherein R₅ is 2-fluorobenzyl, or benzyl.

In another aspect, provided are compounds of Formula 10-42, i.e., compounds according to any one of Formulas 13 up to and including 13-41 wherein n is 0.

In still another aspect, provided are compounds of Formula 10-43, i.e., compounds according to any one of Formulas 13 up to and including 13-41 wherein n is 1.

In another aspect, provided are compounds of Formula 10-44, i.e., compounds according to either of Formulas 13-42 or 13-43, wherein R₆ and R₇ are H.

In another aspect, provided are compounds of Formula 10-45, i.e., compounds according to either of Formulas 13-42 or 13-43, wherein R₆ is OCH₃ and R₇ is H.

In yet another aspect, provided are compounds of Formula 10-46, i.e., compounds according to either of Formulas 13-42 or 13-43, wherein R₆ is H and R₇ is OCH₃.

In another aspect, provided are compounds of Formula 10-47, i.e., compounds according to either of Formulas 13-42 or 13-43, wherein R₆ and R₇ are OCH₃.

In another aspect, provided are compounds of Formula 10-48, i.e., compounds according to any one of Formulas 13-42 or 13-43, wherein R₆ and R₇ are independently H or methyl. In one embodiment, R₆ is H and R₇ is methyl. In another embodiment, R₆ is methyl and R₇ is H.

In another aspect, provided are compounds of Formula 10-49, i.e., compounds according to any one of Formulas 13-44, 13-45, 13-46, 13-47 or 13-48, wherein R₄ is H.

In another aspect, provided are compounds of Formula 10-50, i.e., compounds according to any one of Formulas 13-44, 13-45, 13-46, 13-47 or 13-48, wherein R₄ is methyl, ethyl, propyl, or hexyl. In one embodiment, R₄ is methyl. In another embodiment, R₄ is hexyl.

In another aspect, provided are compounds of Formula 10-51, i.e., compounds according to any one of Formulas 13-44, 13-45, 13-46, 13-47 or 13-48, wherein R₄ is CCl₃, CF₃. In one embodiment, R₄ is CF₃.

In another aspect, provided are compounds of Formula 10-52, i.e., compounds according to any one of Formulas 13 up to and including 13-51, wherein X is O.

In another aspect, provided are compounds of Formula 10-53, i.e., compounds according to any one of Formulas 13 up to and including 13-51, wherein X is S.

In an aspect, provided are compounds of Formula 10-54, i.e., compounds according to any one of Formulas 13 up to and including 13-53, wherein p is 0.

In an aspect, provided are compounds of Formula 10-55, i.e., compounds according to any one of Formulas 13 up to and including 13-53, wherein p is 1.

In an aspect, provided are compounds of Formula 10-56, i.e., a compound wherein X is S, n is 1, p is 0, R₁ is H, R₂ is methyl, R₃ is H, R₄ is (R) or (S) methyl, R₅ is propyl, R₆ is H, R₇ is H, R₂₀ is H.

In an aspect, provided are compounds of Formula 10-57, i.e., a compound wherein X is S, n is 1, p is 0, R₁ is H, R₂ is methyl, R₃ is H, R₄ is (R) methyl, R₅ is propyl, R₆ is H, R₇ is H, R₂₀ is H.

In an aspect, provided are compounds of Formula 10-58, i.e., a compound wherein X is S, n is 1, p is 0, R₁ is H, R₂ is methyl, R₃ is H, R₄ is (S) methyl, R₅ is propyl, R₆ is H, R₇ is H, R₂₀ is H.

In an aspect, provided are compounds of Formula 10-56, i.e., compounds according to any one of Formulas 13 up to and including 13-54, 13-56, 13-57 and 13-58, having the following formula:

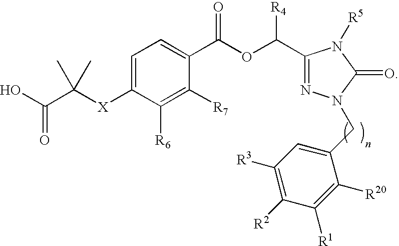

### Definitions

For Example:

The term “pharmaceutically acceptable salts” or “a pharmaceutically acceptable salt thereof” refer to salts prepared from pharmaceutically acceptable non-toxic acids or bases including inorganic acids and bases and organic acids and bases. Since the compound of the present invention is basic, salts may be prepared from pharmaceutically acceptable non-toxic acids. Suitable pharmaceutically acceptable acid addition salts for the compound of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic, and the like. Preferred acid addition salts are the chloride and sulfate salts and the salts of di- and tri-carboxylic acids, for example, tartrate, citrate, maleate, succinate, and the like.

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

By “alkyl” is meant a straight or branched, non-cyclic, hydrocarbon. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, heptyl and octyl. “C₁-C₆ alkyl” denotes straight or branched, non-cyclic, alkyl groups having 1-6 carbon atoms. Likewise, “C₁-C₄ alkyl” denotes straight or branched, non-cyclic, alkyl groups having 1-4 carbon atoms.

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl, naphthyl, and anthracenyl. More preferred aryl groups are phenyl and naphthyl. Most preferred is phenyl.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

The disclosures in this application of all articles and references, including patents, are incorporated herein by reference.

### Chemical Name

### No.

1 2-methyl-2-(4-(((1-methyl-3-phenyl-1H-pyrazol-5-yl)methoxy)carbonyl)phenylthio)propanoic acid
2 2-methyl-2-(4-(((1-methyl-3-phenyl-1H-pyrazol-5-yl)methoxy)carbonyl)phenoxy)propanoic acid
3 2-methyl-2-(4-(((1-(4-methylbenzyl)-1H-pyrazol-4-yl)methoxy)carbonyl)phenylthio)propanoic acid
4 2-methyl-2-(4-(((1-(4-methylbenzyl)-3-(trifluoromethyl)-1H-pyrazol-4-yl)methoxy)carbonyl)phenoxy)propanoic acid
5 2-methyl-2-(4-(((1-(4-methylbenzyl)-3-(trifluoromethyl)-1H-pyrazol-4-yl)methoxy)carbonyl)phenylthio)propanoic acid
6 2-(4-(((1-(4-tert-butylbenzyl)-1H-pyrazol-4-yl)methoxy)carbonyl)phenylthio)-2-methylpropanoic acid
7 2-(4-(((1-(4-tert-butylbenzyl)-1H-pyrazol-4-yl)methoxy)carbonyl)phenoxy)-2-methylpropanoic acid
8 2-(4-(((1-(4-methoxybenzyl)-1H-pyrazol-4-yl)methoxy)carbonyl)phenylthio)-2-methylpropanoic acid
9 2-(4-(((1-(4-methoxybenzyl)-1H-pyrazol-4-yl)methoxy)carbonyl)phenoxy)-2-methylpropanoic acid
10 2-methyl-2-(4-(((1-(4-(trifluoromethyl)benzyl)-1H-pyrazol-4-yl)methoxy)carbonyl)phenylthio)propanoic acid
11 2-methyl-2-(4-(((1-(4-(trifluoromethyl)benzyl)-1H-pyrazol-4-yl)methoxy)carbonyl)phenoxy)propanoic acid
12 2-methyl-2-(4-(((3-propyl-1-(4-(trifluoromethyl)benzyl)-1H-pyrazol-4-yl)methoxy)carbonyl)phenylthio)propanoic acid
13 2-methyl-2-(4-(((3-propyl-1-(4-(trifluoromethyl)benzyl)-1H-pyrazol-4-yl)methoxy)carbonyl)phenoxy)propanoic acid
14 2-methyl-2-(4-(((1-propyl-3-p-tolyl-1H-pyrazol-5-yl)methoxy)carbonyl)phenylthio)propanoic acid
15 2-methyl-2-(4-(((1-propyl-3-p-tolyl-1H-pyrazol-5-yl)methoxy)carbonyl)phenoxy)propanoic acid
16 2-methyl-2-(4-(((3-phenyl-1-propyl-1H-pyrazol-5-yl)methoxy)carbonyl)phenylthio)propanoic acid
17 2-methyl-2-(4-(((3-phenyl-1-p-tolyl-1H-pyrazol-4-yl)methoxy)carbonyl)phenoxy)propanoic acid
18 2-methyl-2-(4-(((1-(4-(trifluoromethyl)phenyl)-1H-pyrazol-4-yl)methoxy)carbonyl)phenylthio)propanoic acid
19 2-methyl-2-(4-(((1-(4-(trifluoromethyl)phenyl)-1H-pyrazol-4-yl)methoxy)carbonyl)phenoxy)propanoic acid
20 2-methyl-2-(4-(((1-(4-(methylthio)benzyl)-1H-pyrazol-4-yl)methoxy)carbonyl)phenylthio)propanoic acid
21 2-methyl-2-(4-(((1-(4-(methylthio)benzyl)-1H-pyrazol-4-yl)methoxy)carbonyl)phenoxy)propanoic acid
22 2-(4-(((1-(biphenyl-4-ylmethyl)-1H-pyrazol-4-yl)methoxy)carbonyl)phenylthio)-2-methylpropanoic acid
23 2-(4-(((1-(biphenyl-4-ylmethyl)-1H-pyrazol-4-yl)methoxy)carbonyl)phenoxy)-2-methylpropanoic acid
24 2-(4-(((1-(3,4-difluorobenzyl)-1H-pyrazol-4-yl)methoxy)carbonyl)phenylthio)-2-methylpropanoic acid
25 2-(4-(((1-(3,4-difluorobenzyl)-1H-pyrazol-4-yl)methoxy)carbonyl)phenoxy)-2-methylpropanoic acid
26 2-methyl-2-(4-(((1-(naphthalen-2-ylmethyl)-1H-pyrazol-4-yl)methoxy)carbonyl)phenylthio)propanoic acid
27 2-methyl-2-(4-(((1-(naphthalen-2-ylmethyl)-1H-pyrazol-4-yl)methoxy)carbonyl)phenoxy)propanoic acid
28 2-(4-(((1-(3,4-di-tert-butylbenzyl)-1H-pyrazol-4-yl)methoxy)carbonyl)phenoxy)-2-methylpropanoic acid
29 2-methyl-2-(4-(((1-((5,5,8,8-tetramethyl-5,6,7,8-tetrahydronaphthalen-2-yl)methyl)-1H-pyrazol-4-yl)methoxy)carbonyl)phenylthio)propanoic acid
30 2-methyl-2-(4-(((1-(3-(trifluoromethyl)benzyl)-1H-pyrazol-4-yl)methoxy)carbonyl)phenylthio)propanoic acid
31 2-methyl-2-(4-(((1-(3-(trifluoromethyl)benzyl)-1H-pyrazol-4-yl)methoxy)carbonyl)phenoxy)propanoic acid
32 2-methyl-2-(4-(((1-(4-(trifluoromethoxy)benzyl)-1H-pyrazol-4-yl)methoxy)carbonyl)phenylthio)propanoic acid
35 2-methyl-2-(4-(2-oxo-2-((1-(4-(trifluoromethyl)phenyl)-1H-pyrazol-4-yl)methoxy)ethyl)phenoxy)propanoic acid
36 (R)-2-methyl-2-(4-((1-(3-(4-methylbenzyl)-1-propyl-1H-pyrazol-5-yl)ethoxy)carbonyl)phenoxy)propanoic acid
37 (S)-2-methyl-2-(4-((1-(3-(4-methylbenzyl)-1-propyl-1H-pyrazol-5-yl)ethoxy)carbonyl)phenoxy)propanoic acid
38 (R)-2-methyl-2-(4-((2,2,2-trifluoro-1-(3-(4-methylbenzyl)-1-propyl-1H-pyrazol-5-yl)ethoxy)carbonyl)phenoxy)propanoic acid
39 (S)-2-methyl-2-(4-((2,2,2-trifluoro-1-(3-(4-methylbenzyl)-1-propyl-1H-pyrazol-5-yl)ethoxy)carbonyl)phenoxy)propanoic acid
45 (R)-2-methyl-2-(4-((1-(3-(4-methylbenzyl)-1-propyl-1H-pyrazol-5-yl)ethoxy)carbonyl)phenylthio)propanoic acid
46 (S)-2-methyl-2-(4-((1-(3-(4-methylbenzyl)-1-propyl-1H-pyrazol-5-yl)ethoxy)carbonyl)phenylthio)propanoic acid
47 (R)-2-methyl-2-(4-((2,2,2-trifluoro-1-(3-(4-methylbenzyl)-1-propyl-1H-pyrazol-5-yl)ethoxy)carbonyl)phenylthio)propanoic acid
48 (S)-2-methyl-2-(4-((2,2,2-trifluoro-1-(3-(4-methylbenzyl)-1-propyl-1H-pyrazol-5-yl)ethoxy)carbonyl)phenylthio)propanoic acid
54 4-(1-Carboxy-1-methyl-ethoxy)-benzoic acid 5-(4-methyl-benzyl)-2-propyl-2H-pyrazol-3-ylmethyl ester
55 4-(1-Carboxy-1-methyl-ethylsulfanyl)-benzoic acid 5-(4-methyl-benzyl)-2-propyl-2H-pyrazol-3-ylmethyl ester
56 2-(4-(((1-(4-isopropylphenyl)-1H-1,2,3-triazol-4-yl)methoxy)carbonyl)phenylthio)-2-methylpropanoic acid
57 2-methyl-2-(4-(((1-(4-(trifluoromethyl)phenyl)-1H-1,2,3-triazol-4-yl)methoxy)carbonyl)phenylthio)propanoic acid
58 2-methyl-2-(4-(((1-(4-(trifluoromethyl)phenyl)-1H-1,2,3-triazol-4-yl)methoxy)carbonyl)phenoxy)propanoic acid
59 2-(4-(((1-(4-isopropylphenyl)-1H-1,2,3-triazol-5-yl)methoxy)carbonyl)phenylthio)-2-methylpropanoic acid
60 2-methyl-2-(4-(((1-(4-methylbenzyl)-1H-1,2,3-triazol-4-yl)methoxy)carbonyl)phenylthio)propanoic acid
61 2-methyl-2-(4-(((1-(4-methylbenzyl)-1H-1,2,3-triazol-4-yl)methoxy)carbonyl)phenoxy)propanoic acid
62 2-methyl-2-(4-(((1-(4-methylbenzyl)-1H-1,2,3-triazol-5-yl)methoxy)carbonyl)phenylthio)propanoic acid
63 2-methyl-2-(4-(((1-(4-methylbenzyl)-1H-1,2,3-triazol-5-yl)methoxy)carbonyl)phenoxy)propanoic acid
64 2-methyl-2-(4-(((1-(4-(trifluoromethyl)benzyl)-1H-1,2,3-triazol-4-yl)methoxy)carbonyl)phenoxy)propanoic acid
65 2-methyl-2-(4-(((5-methyl-2-phenyl-2H-1,2,3-triazol-4-yl)methoxy)carbonyl)phenylthio)propanoic acid
66 2-methyl-2-(4-(((5-methyl-2-phenyl-2H-1,2,3-triazol-4-yl)methoxy)carbonyl)phenoxy)propanoic acid
67 2-methyl-2-(4-(((1-(4-(trifluoromethyl)benzyl)-1H-1,2,3-triazol-4-yl)methoxy)carbonyl)phenylthio)propanoic acid
68 2-(4-(((1-(4-methoxybenzyl)-1H-1,2,3-triazol-4-yl)methoxy)carbonyl)phenylthio)-2-methylpropanoic acid
69 2-(4-(((1-(4-tert-butylbenzyl)-1H-1,2,3-triazol-4-yl)methoxy)carbonyl)phenylthio)-2-methylpropanoic acid
70 2-(4-(((1-(4-tert-butylbenzyl)-1H-1,2,3-triazol-4-yl)methoxy)carbonyl)phenoxy)-2-methylpropanoic acid
71 2-methyl-2-(4-(((1-(4-(trifluoromethylthio)benzyl)-1H-1,2,3-triazol-4-yl)methoxy)carbonyl)phenylthio)propanoic acid
72 2-methyl-2-(4-(((1-(4-(trifluoromethylthio)benzyl)-1H-1,2,3-triazol-4-yl)methoxy)carbonyl)phenoxy)propanoic acid
73 2-methyl-2-(4-(((1-(4-(trifluoromethoxy)benzyl)-1H-1,2,3-triazol-4-yl)methoxy)carbonyl)phenylthio)propanoic acid
74 2-methyl-2-(4-(((1-(4-(trifluoromethoxy)benzyl)-1H-1,2,3-triazol-4-yl)methoxy)carbonyl)phenoxy)propanoic acid
75 2-(4-(((1-(4-(1,1,1,3,3,3-hexafluoro-2-methoxypropan-2-yl)benzyl)-1H-1,2,3-triazol-4-yl)methoxy)carbonyl)phenylthio)-2-methylpropanoic acid
76 2-(4-(((1-(4-(1,1,1,3,3,3-hexafluoro-2-methoxypropan-2-yl)benzyl)-1H-1,2,3-triazol-4-yl)methoxy)carbonyl)phenoxy)-2-methylpropanoic acid
77 2-(4-(((1-benzyl-1H-1,2,3-triazol-4-yl)methoxy)carbonyl)phenylthio)-2-methylpropanoic acid
78 2-(4-(((1-benzyl-1H-1,2,3-triazol-4-yl)methoxy)carbonyl)phenoxy)-2-methylpropanoic acid
79 2-(3-methoxy-4-(((1-(4-(trifluoromethyl)benzyl)-1H-1,2,3-triazol-4-yl)methoxy)carbonyl)phenoxy)-2-methylpropanoic acid
80 2-methyl-2-(3-methyl-4-(((1-(4-(trifluoromethyl)benzyl)-1H-1,2,3-triazol-4-yl)methoxy)carbonyl)phenoxy)propanoic acid
81 2-methyl-2-(4-((1-(1-(4-methylbenzyl)-1H-1,2,3-triazol-4-yl)ethoxy)carbonyl)phenylthio)propanoic acid
82 2-methyl-2-(4-((1-(1-(4-methylbenzyl)-1H-1,2,3-triazol-5-yl)ethoxy)carbonyl)phenylthio)propanoic acid
83 2-methyl-2-(4-((1-(1-(4-methylbenzyl)-1H-1,2,3-triazol-4-yl)heptyloxy)carbonyl)phenylthio)propanoic acid
84 2-methyl-2-(4-((1-(1-(4-methylbenzyl)-1H-1,2,3-triazol-4-yl)heptyloxy)carbonyl)phenoxy)propanoic acid
85 (S)-2-methyl-2-(4-((1-(1-(4-methylbenzyl)-1H-1,2,3-triazol-4-yl)ethoxy)carbonyl)phenylthio)propanoic acid
86 (S)-2-methyl-2-(4-((1-(1-(4-methylbenzyl)-1H-1,2,3-triazol-4-yl)ethoxy)carbonyl)phenoxy)propanoic acid
87 (R)-2-methyl-2-(4-((1-(1-(4-methylbenzyl)-1H-1,2,3-triazol-4-yl)ethoxy)carbonyl)phenylthio)propanoic acid
88 (R)-2-methyl-2-(4-((1-(1-(4-methylbenzyl)-1H-1,2,3-triazol-4-yl)ethoxy)carbonyl)phenoxy)propanoic acid
89 2-(4-((2-(4-(4-tert-butylphenyl)-1H-1,2,3-triazol-1-yl)ethoxy)carbonyl)phenylthio)-2-methylpropanoic acid
90 2-(4-((2-(4-(4-tert-butylphenyl)-1H-1,2,3-triazol-1-yl)ethoxy)carbonyl)phenoxy)-2-methylpropanoic acid
91 2-methyl-2-(4-((2-(1-(4-(trifluoromethyl)benzyl)-1H-1,2,3-triazol-4-yl)ethoxy)carbonyl)phenylthio)propanoic acid
92 2-methyl-2-(4-((2-(1-(4-(trifluoromethyl)benzyl)-1H-1,2,3-triazol-4-yl)ethoxy)carbonyl)phenoxy)propanoic acid
93 2-methyl-2-(4-(((1-(4-methylphenethyl)-1H-1,2,3-triazol-4-yl)methoxy)carbonyl)phenylthio)propanoic acid
94 2-methyl-2-(4-(((1-(4-methylphenethyl)-1H-1,2,3-triazol-4-yl)methoxy)carbonyl)phenoxy)propanoic acid
95 (R)-2-methyl-2-(4-((1-(1-(4-(trifluoromethyl)benzyl)-1H-1,2,3-triazol-4-yl)ethoxy)carbonyl)phenylthio)propanoic acid
96 (R)-2-methyl-2-(4-((1-(1-(4-(trifluoromethyl)benzyl)-1H-1,2,3-triazol-4-yl)ethoxy)carbonyl)phenoxy)propanoic acid
97 (S)-2-methyl-2-(4-((1-(1-(4-(trifluoromethyl)benzyl)-1H-1,2,3-triazol-4-yl)ethoxy)carbonyl)phenylthio)propanoic acid
98 (S)-2-methyl-2-(4-((1-(1-(4-(trifluoromethyl)benzyl)-1H-1,2,3-triazol-4-yl)ethoxy)carbonyl)phenoxy)propanoic acid
99 2-(2-methoxy-4-(((1-(4-methylbenzyl)-1H-1,2,3-triazol-4-yl)methoxy)carbonyl)phenoxy)-2-methylpropanoic acid
100 2-(4-(((1-(4-tert-butylbenzyl)-1H-1,2,3-triazol-4-yl)methoxy)carbonyl)-2-methoxyphenoxy)-2-methylpropanoic acid
101 2-(2-methoxy-4-(((1-(4-(trifluoromethyl)benzyl)-1H-1,2,3-triazol-4-yl)methoxy)carbonyl)phenoxy)-2-methylpropanoic acid
102 2-methyl-2-(4-(((1-(4-(trifluoromethyl)benzyl)-1H-1,2,3-triazol-4-yl)methoxy)carbonyl)phenylthio)propanoic acid
103 2-(4-(((1-(4-methoxybenzyl)-1H-1,2,3-triazol-4-yl)methoxy)carbonyl)phenoxy)-2-methylpropanoic acid
104 2-(3-methoxy-4-(((1-(4-(trifluoromethyl)benzyl)-1H-1,2,3-triazol-4-yl)methoxy)carbonyl)phenoxy)-2-methylpropanoic acid
105 (R)-2-(4-((1-(1-(4-tert-butylbenzyl)-1H-1,2,3-triazol-4-yl)propan-2-yloxy)carbonyl)phenylthio)-2-methylpropanoic acid
106 (S)-2-(4-((3-(1-(4-tert-butylbenzyl)-1H-1,2,3-triazol-4-yl)-1,1,1-trifluoropropan-2-yloxy)carbonyl)phenylthio)-2-methylpropanoic acid
107 (R)-2-(4-((3-(1-(4-tert-butylbenzyl)-1H-1,2,3-triazol-4-yl)-1,1,1-trifluoropropan-2-yloxy)carbonyl)phenylthio)-2-methylpropanoic acid
112 (R)-2-methyl-2-(4-((1-(1-(4-(trifluoromethyl)benzyl)-1H-1,2,3-triazol-4-yl)propan-2-yloxy)carbonyl)phenylthio)propanoic acid
113 (S)-2-methyl-2-(4-((1-(1-(4-(trifluoromethyl)benzyl)-1H-1,2,3-triazol-4-yl)propan-2-yloxy)carbonyl)phenylthio)propanoic acid
114 (R)-2-methyl-2-(4-((1,1,1-trifluoro-3-(1-(4-(trifluoromethyl)benzyl)-1H-1,2,3-triazol-4-yl)propan-2-yloxy)carbonyl)phenylthio)propanoic acid
115 (S)-2-methyl-2-(4-((1,1,1-trifluoro-3-(1-(4-(trifluoromethyl)benzyl)-1H-1,2,3-triazol-4-yl)propan-2-yloxy)carbonyl)phenylthio)propanoic acid
121 (S)-2-methyl-2-(4-((2,2,2-trifluoro-1-(1-(4-methylbenzyl)-1H-1,2,3-triazol-4-yl)ethoxy)carbonyl)phenylthio)propanoic acid
122 (R)-2-methyl-2-(4-((2,2,2-trifluoro-1-(1-(4-methylbenzyl)-1H-1,2,3-triazol-4-yl)ethoxy)carbonyl)phenylthio)propanoic acid
127 (R)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid
128 (S)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid
133 4-(1-Carboxy-1-methyl-ethylsulfanyl)-benzoic acid (S)-2-[1-(4-tert-butyl-benzyl)-1H-1,2,3-triazol-4-yl]-1-methyl-ethyl ester
134 4-(1-Carboxy-1-methyl-ethylsulfanyl)-benzoic acid 1-methyl-2-[1-(4-trifluoromethyl-benzyl)-1H-1,2,3-triazol-4-yl]-ethyl ester
135 4-(1-Carboxy-1-methyl-ethylsulfanyl)-benzoic acid (R)-1-[1-(4-tert-butyl-benzyl)-1H-1,2,3-triazol-4-yl]-ethyl ester
136 4-(1-Carboxy-1-methyl-ethylsulfanyl)-benzoic acid (R)-1-[1-(4-trifluoromethylsulfanyl-benzyl)-1H-1,2,3-triazol-4-yl]-ethyl ester
137 4-(1-Carboxy-1-methyl-ethylsulfanyl)-benzoic acid (R)-1-[1-(4-trifluoromethoxy-benzyl)-1H-1,2,3-triazol-4-yl]-ethyl ester
138 4-(1-Carboxy-1-methyl-ethylsulfanyl)-benzoic acid (R)-2-[1-(4-tert-butyl-benzyl)-1H-1,2,3-triazol-4-yl]-1-methyl-ethyl ester
139 4-(1-Carboxy-1-methyl-ethylsulfanyl)-benzoic acid (R)-1-[1-(3-trifluoromethyl-benzyl)-1H-1,2,3-triazol-4-yl]-ethyl ester
140 4-(1-Carboxy-1-methyl-ethylsulfanyl)-benzoic acid (R)-1-[1-(3,5-bis-trifluoromethyl-benzyl)-1H-1,2,3-triazol-4-yl]-ethyl ester
141 4-(1-Carboxy-1-methyl-ethylsulfanyl)-benzoic acid (R)-1-[1-(3,5-difluoro-benzyl)-1H-1,2,3-triazol-4-yl]-ethyl ester
142 4-(1-Carboxy-1-methyl-ethoxy)-3-methyl-benzoic acid 1-(4-trifluoromethyl-benzyl)-1H-1,2,3-triazol-4-ylmethyl ester
143 2-methyl-2-(4-(2-oxo-2-((1-(4-(trifluoromethyl)benzyl)-1H-1,2,3-triazol-4-yl)methoxy)ethyl)phenoxy)propanoic acid
144 4-(1-Carboxy-1-methyl-ethylsulfanyl)-benzoic acid 2-[1-(4-tert-butyl-benzyl)-1H-1,2,3-triazol-4-yl]-ethyl ester
145 4-(1-Carboxy-1-methyl-ethylsulfanyl)-benzoic acid 2-[1-(4-methyl-benzyl)-1H-1,2,3-triazol-4-yl]-ethyl ester
146 4-(1-Carboxy-1-methyl-ethylsulfanyl)-benzoic acid (R)-1-methyl-2-[1-(4-trifluoromethyl-benzyl)-1H-1,2,3-triazol-4-yl]-ethyl ester
147 4-(1-Carboxy-1-methyl-ethylsulfanyl)-benzoic acid (S)-1-methyl-2-[1-(4-trifluoromethyl-benzyl)-1H-1,2,3-triazol-4-yl]-ethyl ester
148 2-methyl-2-(4-(((1-(4-methylbenzyl)-5-oxo-4-propyl-4,5-dihydro-1H-1,2,4-triazol-3-yl)methoxy)carbonyl)phenylthio)propanoic acid
149 2-methyl-2-(4-(((1-(4-methylbenzyl)-5-oxo-4-propyl-4,5-dihydro-1H-1,2,4-triazol-3-yl)methoxy)carbonyl)phenoxy)propanoic acid
150 2-(4-(((1-(4-tert-butylbenzyl)-5-oxo-4-propyl-4,5-dihydro-1H-1,2,4-triazol-3-yl)methoxy)carbonyl)phenoxy)-2-methylpropanoic acid
151 2-(4-(((4-hexyl-1-(4-methylbenzyl)-5-oxo-4,5-dihydro-1H-1,2,4-triazol-3-yl)methoxy)carbonyl)phenoxy)-2-methylpropanoic acid
152 2-methyl-2-(4-(((1-(4-methylbenzyl)-4-octyl-5-oxo-4,5-dihydro-1H-1,2,4-triazol-3-yl)methoxy)carbonyl)phenoxy)propanoic acid
153 2-(4-(((4-cyclopentyl-1-(4-methylbenzyl)-5-oxo-4,5-dihydro-1H-1,2,4-triazol-3-yl)methoxy)carbonyl)phenoxy)-2-methylpropanoic acid
154 2-(4-(((4-benzyl-1-(4-methylbenzyl)-5-oxo-4,5-dihydro-1H-1,2,4-triazol-3-yl)methoxy)carbonyl)phenoxy)-2-methylpropanoic acid
155 2-(4-(((4-(2-fluorobenzyl)-1-(4-methylbenzyl)-5-oxo-4,5-dihydro-1H-1,2,4-triazol-3-yl)methoxy)carbonyl)phenoxy)-2-methylpropanoic acid
156 2-methyl-2-(4-(((5-oxo-1-phenethyl-4-propyl-4,5-dihydro-1H-1,2,4-triazol-3-yl)methoxy)carbonyl)phenoxy)propanoic acid
157 2-(4-(((1-(4-fluorobenzyl)-5-oxo-4-propyl-4,5-dihydro-1H-1,2,4-triazol-3-yl)methoxy)carbonyl)phenoxy)-2-methylpropanoic acid
158 2-(4-(((1-(2-fluorobenzyl)-5-oxo-4-propyl-4,5-dihydro-1H-1,2,4-triazol-3-yl)methoxy)carbonyl)phenoxy)-2-methylpropanoic acid
159 2-(4-(((1-(4-tert-butylbenzyl)-5-oxo-4-propyl-4,5-dihydro-1H-1,2,4-triazol-3-yl)methoxy)carbonyl)phenylthio)-2-methylpropanoic acid
160 2-(4-(((4-hexyl-1-(4-methylbenzyl)-5-oxo-4,5-dihydro-1H-1,2,4-triazol-3-yl)methoxy)carbonyl)phenylthio)-2-methylpropanoic acid
161 2-methyl-2-(4-(((1-(4-methylbenzyl)-4-octyl-5-oxo-4,5-dihydro-1H-1,2,4-triazol-3-yl)methoxy)carbonyl)phenylthio)propanoic acid
162 2-(4-(((1-(3,4-dimethylbenzyl)-5-oxo-4-propyl-4,5-dihydro-1H-1,2,4-triazol-3-yl)methoxy)carbonyl)phenylthio)-2-methylpropanoic acid
163 2-(4-(((1-(biphenyl-4-ylmethyl)-5-oxo-4-propyl-4,5-dihydro-1H-1,2,4-triazol-3-yl)methoxy)carbonyl)phenylthio)-2-methylpropanoic acid
164 2-(4-(((1-(4-bromobenzyl)-5-oxo-4-propyl-4,5-dihydro-1H-1,2,4-triazol-3-yl)methoxy)carbonyl)phenylthio)-2-methylpropanoic acid
165 2-(4-(((1-(4-fluorobenzyl)-5-oxo-4-propyl-4,5-dihydro-1H-1,2,4-triazol-3-yl)methoxy)carbonyl)phenylthio)-2-methylpropanoic acid
166 2-(4-(((1-(2,4-difluorobenzyl)-5-oxo-4-propyl-4,5-dihydro-1H-1,2,4-triazol-3-yl)methoxy)carbonyl)phenoxy)-2-methylpropanoic acid
167 2-methyl-2-(4-(((5-oxo-4-propyl-1-(4-(trifluoromethyl)benzyl)-4,5-dihydro-1H-1,2,4-triazol-3-yl)methoxy)carbonyl)phenoxy)propanoic acid
168 2-methyl-2-(4-(((5-oxo-4-propyl-1-(4-(trifluoromethoxy)benzyl)-4,5-dihydro-1H-1,2,4-triazol-3-yl)methoxy)carbonyl)phenoxy)propanoic acid
169 2-(4-(((1-(4-(methoxycarbonyl)benzyl)-5-oxo-4-propyl-4,5-dihydro-1H-1,2,4-triazol-3-yl)methoxy)carbonyl)phenoxy)-2-methylpropanoic acid
170 2-(4-(((1-(4-(ethoxycarbonyl)benzyl)-5-oxo-4-propyl-4,5-dihydro-1H-1,2,4-triazol-3-yl)methoxy)carbonyl)phenoxy)-2-methylpropanoic acid
171 2-methyl-2-(4-(((5-oxo-4-propyl-1-(4-((3,3,3-trifluoropropoxy)carbonyl)benzyl)-4,5-dihydro-1H-1,2,4-triazol-3-yl)methoxy)carbonyl)phenoxy)propanoic acid
172 2-(4-(((1-(4-((1,1,1,3,3,3-hexafluoro-2-methylpropan-2-yloxy)carbonyl)benzyl)-5-oxo-4-propyl-4,5-dihydro-1H-1,2,4-triazol-3-yl)methoxy)carbonyl)phenoxy)-2-methylpropanoic acid
173 (S)-2-methyl-2-(4-(((5-oxo-1-(4-((3,3,4,4,4-pentafluorobutan-2-yloxy)carbonyl)benzyl)-4-propyl-4,5-dihydro-1H-1,2,4-triazol-3-yl)methoxy)carbonyl)phenoxy)propanoic acid
174 (R)-2-methyl-2-(4-(((5-oxo-1-(4-((3,3,4,4,4-pentafluorobutan-2-yloxy)carbonyl)benzyl)-4-propyl-4,5-dihydro-1H-1,2,4-triazol-3-yl)methoxy)carbonyl)phenoxy)propanoic acid
175 2-methyl-2-(4-(((5-oxo-4-propyl-1-(4-(trifluoromethyl)benzyl)-4,5-dihydro-1H-1,2,4-triazol-3-yl)methoxy)carbonyl)phenylthio)propanoic acid
176 2-methyl-2-(4-(((5-oxo-4-propyl-1-(3-(trifluoromethyl)benzyl)-4,5-dihydro-1H-1,2,4-triazol-3-yl)methoxy)carbonyl)phenylthio)propanoic acid
177 2-(4-(((1-(3,5-bis(trifluoromethyl)benzyl)-5-oxo-4-propyl-4,5-dihydro-1H-1,2,4-triazol-3-yl)methoxy)carbonyl)phenylthio)-2-methylpropanoic acid
178 2-methyl-2-(4-(((5-oxo-4-propyl-1-(4-(trifluoromethoxy)benzyl)-4,5-dihydro-1H-1,2,4-triazol-3-yl)methoxy)carbonyl)phenylthio)propanoic acid
179 2-methyl-2-(4-(((5-oxo-4-propyl-1-(4-(trifluoromethylthio)benzyl)-4,5-dihydro-1H-1,2,4-triazol-3-yl)methoxy)carbonyl)phenylthio)propanoic acid
180 2-(4-(((1-(4-tert-butylbenzyl)-5-oxo-4-propyl-4,5-dihydro-1H-1,2,4-triazol-3-yl)methoxy)carbonyl)-2-methoxyphenoxy)-2-methylpropanoic acid
181 2-(2-methoxy-4-(((1-(4-methylbenzyl)-5-oxo-4-propyl-4,5-dihydro-1H-1,2,4-triazol-3-yl)methoxy)carbonyl)phenoxy)-2-methylpropanoic acid
182 2-(3-methoxy-4-(((1-(4-methylbenzyl)-5-oxo-4-propyl-4,5-dihydro-1H-1,2,4-triazol-3-yl)methoxy)carbonyl)phenoxy)-2-methylpropanoic acid
183 2-methyl-2-(3-methyl-4-(((1-(4-methylbenzyl)-5-oxo-4-propyl-4,5-dihydro-1H-1,2,4-triazol-3-yl)methoxy)carbonyl)phenoxy)propanoic acid
184 2-(4-(((4-hexyl-1-(4-methylbenzyl)-5-oxo-4,5-dihydro-1H-1,2,4-triazol-3-yl)methoxy)carbonyl)-2-methoxyphenoxy)-2-methylpropanoic acid
185 2-(4-(((1-(4-tert-butylbenzyl)-4-hexyl-5-oxo-4,5-dihydro-1H-1,2,4-triazol-3-yl)methoxy)carbonyl)-2-methoxyphenoxy)-2-methylpropanoic acid
186 2-(2-methoxy-4-(((1-(4-methylbenzyl)-4-octyl-5-oxo-4,5-dihydro-1H-1,2,4-triazol-3-yl)methoxy)carbonyl)phenoxy)-2-methylpropanoic acid
187 2-(4-(((1-(4-tert-butylbenzyl)-4-hexyl-5-oxo-4,5-dihydro-1H-1,2,4-triazol-3-yl)methoxy)carbonyl)phenylthio)-2-methylpropanoic acid
188 2-(4-(((1-(4-tert-butylbenzyl)-4-hexyl-5-oxo-4,5-dihydro-1H-1,2,4-triazol-3-yl)methoxy)carbonyl)phenoxy)-2-methylpropanoic acid
189 2-(4-(((1-(4-tert-butylbenzyl)-4-heptyl-5-oxo-4,5-dihydro-1H-1,2,4-triazol-3-yl)methoxy)carbonyl)phenylthio)-2-methylpropanoic acid
190 2-(4-(((1-(4-tert-butylbenzyl)-4-heptyl-5-oxo-4,5-dihydro-1H-1,2,4-triazol-3-yl)methoxy)carbonyl)phenoxy)-2-methylpropanoic acid
191 2-(4-(((4-heptyl-1-(4-methylbenzyl)-5-oxo-4,5-dihydro-1H-1,2,4-triazol-3-yl)methoxy)carbonyl)phenylthio)-2-methylpropanoic acid
192 2-(4-(((4-heptyl-1-(4-methylbenzyl)-5-oxo-4,5-dihydro-1H-1,2,4-triazol-3-yl)methoxy)carbonyl)phenoxy)-2-methylpropanoic acid
193 2-(4-(((4-ethyl-1-(4-methylbenzyl)-5-oxo-4,5-dihydro-1H-1,2,4-triazol-3-yl)methoxy)carbonyl)phenylthio)-2-methylpropanoic acid
194 2-(4-(((4-ethyl-1-(4-methylbenzyl)-5-oxo-4,5-dihydro-1H-1,2,4-triazol-3-yl)methoxy)carbonyl)phenoxy)-2-methylpropanoic acid
195 2-(4-(((1-(4-tert-butylbenzyl)-4-ethyl-5-oxo-4,5-dihydro-1H-1,2,4-triazol-3-yl)methoxy)carbonyl)phenylthio)-2-methylpropanoic acid
196 2-(4-(((1-(4-tert-butylbenzyl)-4-ethyl-5-oxo-4,5-dihydro-1H-1,2,4-triazol-3-yl)methoxy)carbonyl)phenoxy)-2-methylpropanoic acid
197 2-(4-(((4-ethyl-5-oxo-1-(4-(trifluoromethyl)benzyl)-4,5-dihydro-1H-1,2,4-triazol-3-yl)methoxy)carbonyl)phenylthio)-2-methylpropanoic acid
198 2-(4-(((4-ethyl-5-oxo-1-(4-(trifluoromethyl)benzyl)-4,5-dihydro-1H-1,2,4-triazol-3-yl)methoxy)carbonyl)phenoxy)-2-methylpropanoic acid
199 2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid
200 2-methyl-2-(4-((1-(1-(4-methylbenzyl)-5-oxo-4-propyl-4,5-dihydro-1H-1,2,4-triazol-3-yl)ethoxy)carbonyl)phenoxy)propanoic acid
201 2-(4-(((1-(4-(1,1,1,3,3,3-hexafluoro-2-methoxypropan-2-yl)benzyl)-4-hexyl-5-oxo-4,5-dihydro-1H-1,2,4-triazol-3-yl)methoxy)carbonyl)phenylthio)-2-methylpropanoic acid
202 2-(4-(((1-(4-(1,1,1,3,3,3-hexafluoro-2-methoxypropan-2-yl)benzyl)-4-hexyl-5-oxo-4,5-dihydro-1H-1,2,4-triazol-3-yl)methoxy)carbonyl)phenoxy)-2-methylpropanoic acid
203 2-(4-(((4-hexyl-5-oxo-1-(4-(perfluoropropan-2-yl)benzyl)-4,5-dihydro-1H-1,2,4-triazol-3-yl)methoxy)carbonyl)phenylthio)-2-methylpropanoic acid
204 2-(4-(((4-hexyl-5-oxo-1-(4-(perfluoropropan-2-yl)benzyl)-4,5-dihydro-1H-1,2,4-triazol-3-yl)methoxy)carbonyl)phenoxy)-2-methylpropanoic acid
205 2-methyl-2-(4-(((5-oxo-4-propyl-1-(3-(trifluoromethyl)benzyl)-4,5-dihydro-1H-1,2,4-triazol-3-yl)methoxy)carbonyl)phenoxy)propanoic acid
206 2-(4-(((1-(3,5-bis(trifluoromethyl)benzyl)-5-oxo-4-propyl-4,5-dihydro-1H-1,2,4-triazol-3-yl)methoxy)carbonyl)phenoxy)-2-methylpropanoic acid
207 2-(4-(((1-(3,5-difluorobenzyl)-5-oxo-4-propyl-4,5-dihydro-1H-1,2,4-triazol-3-yl)methoxy)carbonyl)phenoxy)-2-methylpropanoic acid
208 2-(4-(((1-(biphenyl-4-ylmethyl)-5-oxo-4-propyl-4,5-dihydro-1H-1,2,4-triazol-3-yl)methoxy)carbonyl)phenoxy)-2-methylpropanoic acid
209 2-(4-(((1-(3,4-dimethylbenzyl)-5-oxo-4-propyl-4,5-dihydro-1H-1,2,4-triazol-3-yl)methoxy)carbonyl)phenoxy)-2-methylpropanoic acid
210 2-methyl-2-(4-(((5-oxo-4-propyl-1-(4-(trifluoromethylthio)benzyl)-4,5-dihydro-1H-1,2,4-triazol-3-yl)methoxy)carbonyl)phenoxy)propanoic acid
211 2-(4-(((1-(4-bromobenzyl)-5-oxo-4-propyl-4,5-dihydro-1H-1,2,4-triazol-3-yl)methoxy)carbonyl)phenoxy)-2-methylpropanoic acid
212 2-(4-(((1-(4-tert-butylbenzyl)-4-heptyl-5-oxo-4,5-dihydro-1H-1,2,4-triazol-3-yl)methoxy)carbonyl)-2-methoxyphenoxy)-2-methylpropanoic acid
213 2-methyl-2-(4-(((1-(4-methylbenzyl)-5-oxo-4,5-dihydro-1H-1,2,4-triazol-3-yl)methoxy)carbonyl)phenoxy)propanoic acid
214 2-(4-(((1-(4-tert-butylbenzyl)-5-oxo-4,5-dihydro-1H-1,2,4-triazol-3-yl)methoxy)carbonyl)phenoxy)-2-methylpropanoic acid
215 2-methyl-2-(4-(((1-(4-methylbenzyl)-5-oxo-4,5-dihydro-1H-1,2,4-triazol-3-yl)methoxy)carbonyl)phenylthio)propanoic acid
216 2-(4-(((1-(4-tert-butylbenzyl)-5-oxo-4,5-dihydro-1H-1,2,4-triazol-3-yl)methoxy)carbonyl)phenylthio)-2-methylpropanoic acid
217 (4-heptyl-1-(4-methylbenzyl)-5-oxo-4,5-dihydro-1H-1,2,4-triazol-3-yl)methyl 4-(1-ethoxy-2-methyl-1-oxopropan-2-ylthio)benzoate
218 2-(4-(((1-(3,5-difluorobenzyl)-5-oxo-4-propyl-4,5-dihydro-1H-1,2,4-triazol-3-yl)methoxy)carbonyl)phenylthio)-2-methylpropanoic acid
219 2-methyl-2-(4-(((1-(4-methylbenzyl)-5-oxo-4,5-dihydro-1H-1,2,4-triazol-3-yl)methoxy)carbonyl)phenoxy)propanoic acid
220 2-(4-(((1-(4-tert-butylbenzyl)-5-oxo-4,5-dihydro-1H-1,2,4-triazol-3-yl)methoxy)carbonyl)phenoxy)-2-methylpropanoic acid
221 2-methyl-2-(4-(((4-methyl-1-(4-methylbenzyl)-5-oxo-4,5-dihydro-1H-1,2,4-triazol-3-yl)methoxy)carbonyl)phenoxy)propanoic acid
222 2-(4-(((1-(4-tert-butylbenzyl)-4-methyl-5-oxo-4,5-dihydro-1H-1,2,4-triazol-3-yl)methoxy)carbonyl)phenoxy)-2-methylpropanoic acid
223 2-methyl-2-(4-(((1-(4-methylbenzyl)-5-oxo-4,5-dihydro-1H-1,2,4-triazol-3-yl)methoxy)carbonyl)phenylthio)propanoic acid
224 2-(4-(((1-(4-tert-butylbenzyl)-5-oxo-4,5-dihydro-1H-1,2,4-triazol-3-yl)methoxy)carbonyl)phenylthio)-2-methylpropanoic acid
225 2-methyl-2-(4-(((4-methyl-1-(4-methylbenzyl)-5-oxo-4,5-dihydro-1H-1,2,4-triazol-3-yl)methoxy)carbonyl)phenylthio)propanoic acid
226 2-(4-(((1-(4-tert-butylbenzyl)-4-methyl-5-oxo-4,5-dihydro-1H-1,2,4-triazol-3-yl)methoxy)carbonyl)phenylthio)-2-methylpropanoic acid
227 2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
228 2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
233 (R)-2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
236 (S)-2-methyl-2-(4-((1-(1-(4-methylbenzyl)-5-oxo-4,5-dihydro-1H-1,2,4-triazol-3-yl)ethoxy)carbonyl)phenylthio)propanoic acid
237 (R)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
240 (S)-2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
241 (S)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
242 (R)-2-methyl-2-(4-((1-(1-(4-methylbenzyl)-5-oxo-4,5-dihydro-1H-1,2,4-triazol-3-yl)ethoxy)carbonyl)phenylthio)propanoic acid
248 4-(1-Carboxy-1-methyl-ethylsulfanyl)-benzoic acid (S)-1-[1-(4-methyl-benzyl)-5-oxo-4-propyl-4,5-dihydro-1H-1,2,4-triazol-3-yl]-ethyl ester
249 4-(1-Carboxy-1-methyl-ethylsulfanyl)-benzoic acid (R)-1-[1-(4-methyl-benzyl)-5-oxo-4-propyl-4,5-dihydro-1H-1,2,4-triazol-3-yl]-ethyl ester
250 4-(1-Carboxy-1-methyl-ethoxy)-benzoic acid (S)-1-[1-(4-methyl-benzyl)-5-oxo-4-propyl-4,5-dihydro-1H-1,2,4-triazol-3-yl]-ethyl ester
251 4-(1-Carboxy-1-methyl-ethoxy)-benzoic acid (R)-1-[1-(4-methyl-benzyl)-5-oxo-4-propyl-4,5-dihydro-1H-1,2,4-triazol-3-yl]-ethyl ester
252 4-(1-Carboxy-1-methyl-ethylsulfanyl)-benzoic acid 1-[1-(4-tert-butyl-benzyl)-5-oxo-4-propyl-4,5-dihydro-1H-1,2,4-triazol-3-yl]-2,2,2-trifluoro-ethyl ester
253 4-(1-Carboxy-1-methyl-ethylsulfanyl)-benzoic acid (S)-1-[1-(4-tert-butyl-benzyl)-5-oxo-4-propyl-4,5-dihydro-1H-1,2,4-triazol-3-yl]-ethyl ester
254 4-(1-Carboxy-1-methyl-ethylsulfanyl)-benzoic acid (R)-1-[5-oxo-4-propyl-1-(4-trifluoromethyl-benzyl)-4,5-dihydro-1H-1,2,4-triazol-3-yl]-ethyl ester
255 4-(1-Carboxy-1-methyl-ethylsulfanyl)-benzoic acid (R)-1-[1-(4-tert-butyl-benzyl)-5-oxo-4-propyl-4,5-dihydro-1H-1,2,4-triazol-3-yl]-ethyl ester
256 4-(1-Carboxy-1-methyl-ethoxy)-benzoic acid (S)-1-[5-oxo-4-propyl-1-(4-trifluoromethyl-benzyl)-4,5-dihydro-1H-1,2,4-triazol-3-yl]-ethyl ester
4-(1-Carboxy-1-methyl-ethoxy)-benzoic acid (S)-1-[1-(4-tert-butyl-benzyl)-5-oxo-4-propyl-4,5-dihydro-1H-1,2,4-triazol-3-yl]-ethyl ester
258 4-(1-Carboxy-1-methyl-ethoxy)-benzoic acid (R)-1-[5-oxo-4-propyl-1-(4-trifluoromethyl-benzyl)-4,5-dihydro-1H-1,2,4-triazol-3-yl]-ethyl ester
259 4-(1-Carboxy-1-methyl-ethoxy)-benzoic acid (R)-1-[1-(4-tert-butyl-benzyl)-5-oxo-4-propyl-4,5-dihydro-1H-1,2,4-triazol-3-yl]-ethyl ester
260 4-(1-Carboxy-1-methyl-ethylsulfanyl)-benzoic acid (S)-1-[5-oxo-4-propyl-1-(4-trifluoromethyl-benzyl)-4,5-dihydro-1H-1,2,4-triazol-3-yl]-ethyl ester
261 4-(1-Carboxy-1-methyl-ethylsulfanyl)-benzoic acid 1-(4-methyl-benzyl)-5-oxo-4,5-dihydro-1H-1,2,4-triazol-3-ylmethyl ester
262 4-Carboxymethylsulfanyl-benzoic acid 1-(4-tert-butyl-benzyl)-4-hexyl-5-oxo-4,5-dihydro-1H-1,2,4-triazol-3-ylmethyl ester
263 4-(1-Carboxy-1-methyl-ethoxy)-benzoic acid 1-[4-heptyl-1-(4-methyl-benzyl)-5-oxo-4,5-dihydro-1H-1,2,4-triazol-3-yl]-ethyl ester
264 4-(1-Carboxy-1-methyl-ethylsulfanyl)-benzoic acid 1-[4-heptyl-1-(4-methyl-benzyl)-5-oxo-4,5-dihydro-1H-1,2,4-triazol-3-yl]-ethyl ester
265 4-(1-Carboxy-1-methyl-ethoxy)-3-methyl-benzoic acid 1-(4-methyl-benzyl)-5-oxo-4-propyl-4,5-dihydro-1H-1,2,4-triazol-3-ylmethyl ester
266 4-(1-Carboxy-1-methyl-ethoxy)-3-methyl-benzoic acid 1-(4-tert-butyl-benzyl)-5-oxo-4-propyl-4,5-dihydro-1H-1,2,4-triazol-3-ylmethyl ester

The preceding compound names, as well as the compound names in the examples below, were generated using either ChemDraw Ultra version 8.03, which is available from Cambridgesoft Corporation, ACD Namepro software, version 6.0, or the AutoNom plugin for MDL Draw 2.1.

Another aspect of the invention relates to methods of treating various disorders related to affector binding PPARα. Exemplary indications for each of the compounds, salts and compositions recited herein include atherosclerosis hypercholesterolemia, dyslipidemias, e.g., primary hyperlipidemia, mixed dyslipidemia, hypertriglyceridemia, the Fredrickson Hyperlipidemia Classifications (e.g., Type II-V) and the like. Other indications may include inflammatory conditions such as ulcerative colitis, psoriasis, chronic obstructive pulmonary disease (COPD) and the like.

“Combination therapy” (or “co-therapy”) includes the administration of a compound of the invention and at least a second agent as part of a specific treatment regimen intended to provide the beneficial effect from the co-action of these therapeutic agents. One example is the combination of a compound of the invention with a statin, a present advantage being marked decrease or elimination of drug-drug interactions (DDI) between them compared to the marked increase in the incidence DDI (e.g., rhabdomyolysis) with combinations of current fibrates and statins. The beneficial effect of the combination includes, but is not limited to, pharmacokinetic or pharmacodynamic co-action resulting from the combination of therapeutic agents. Combinations of the compounds of the present invention and the other active agents may be administered together in a single combination or separately. Where separate administration is employed, the administration of one element may be prior to, concurrent with, or subsequent to the administration of other agents. Administration of these therapeutic agents in combination typically is carried out over a defined time period (usually minutes, hours, days or weeks depending upon the combination selected). In one embodiment, “combination therapy” encompasses the administration of two or more of these therapeutic agents as part of separate monotherapy regimens that incidentally and arbitrarily result in the combinations of the present invention. In another embodiment, “combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues.

The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also embraces the administration of the therapeutic agents as described above in further combination with other biologically active ingredients and non-drug therapies (e.g., diet, surgery, radiation treatment, or a medical device). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

“Treating”, includes any effect, e.g., lessening, reducing, modulating, or eliminating, that results in the improvement of the condition, disease, disorder, etc. “Treating”, includes any effect, e.g., lessening, reducing, modulating, or eliminating, that results in the improvement of the condition, disease, disorder, etc. “Treating” or “treatment” of a disease state includes: (1) preventing the disease state, i.e., causing the clinical symptoms of the disease state not to develop in a subject that may be exposed to or predisposed to the disease state, but does not yet experience or display symptoms of the disease state; (2) inhibiting the disease state, i.e., arresting the development of the disease state or its clinical symptoms; or (3) relieving the disease state, i.e., causing temporary or permanent regression of the disease state or its clinical symptoms. “Disease state” means any disease, condition, symptom, or indication.

Dosage rates and routes of administration of the disclosed compounds are similar to those already used in the art and known to the skilled artisan (see, for example, Physicians' Desk Reference, 54th Ed., Medical Economics Company, Montvale, N.J., 2000).

The compounds of general Formulae 1-25 of the invention may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of general Formulae 1-25 and a pharmaceutically acceptable carrier. One or more compounds of general Formulae 1-25 may be present in association with one or more non-toxic pharmaceutically acceptable carriers and/or diluents and/or adjuvants, and if desired other active ingredients, e.g., other antidepressant or antipsychotic drugs. The pharmaceutical compositions containing compounds of general Formulae 1-25 may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds of general Formulae 1-25 may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formulae 1-25 may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The formulations may also be applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

The invention is illustrated further by the following examples, which are not to be construed as limiting the invention in scope or spirit to the specific procedures described in them.

### Example 1

General Triazolone Synthesis. Reactions were performed in dry solvents under an atmosphere of nitrogen unless otherwise specified, and were followed by thin-layer chromatography (TLC) on Analtech (0.25 mm) glass-packed precoated silica gel plates, which were visualized by short wave UV light or in an iodine chamber. The term “standard work-up” refers to addition of water to the reaction mixture, extraction with EtOAc (3×), washing the combined organic layers successively with water and brine, drying over anhydrous Na₂SO₄, filtering and concentrating on a Buchi R-114 rotary evaporator. Chromatographic separations were performed on silica gel columns (Aldrich Silica Gel 70-230 mesh, 60 A) or on a Gilson liquid handler using a reverse phase Polaris C18 column (5μ, 100×212). ¹H NMR spectra were recorded on a Nicolet/GE NT 300 spectrometer.

### Example 2

### Compound 149

### Sodium 2-methyl-2-(4-(((1-(4-methylbenzyl)-5-oxo-4-propyl-4,5-dihydro-1H-1,2,4-triazol-3-yl)methoxy)carbonyl)phenoxy)propanoate

### Preparation of methyl 4-(1-tert-butoxy-2-methyl-1-oxopropan-2-yloxy)benzoate

To a solution of methyl 4-hydroxybenzoate (5.0 g, 32.9 mmol) and potassium carbonate (4.91 g, 35.5 mmol) in DMF (40 mL) was added tert-butyl 2-bromo-2-methylpropanoate (7.4 mL, 39.7 mmol, d=1.196). The reaction was stirred at 75° C. for 16 hours. The reaction was cooled and subjected to a standard work-up. The organic layer was concentrated to a colorless oil (4.42 g, 15.01 mmol). ¹H NMR (DMSO-d₆): δ 1.32 (s, 9H), 1.52(s, 6H), 3.76 (s, 3H), 6.81 (td, 2H, J=3.6 Hz, J=8.4 Hz), 7.84 (td, 2H, J=2.8 Hz, J=8.8 Hz).

### Preparation of 4-(1-tert-butoxy-2-methyl-1-oxopropan-2-yloxy)benzoic acid (Acid B)

To methyl 4-(1-tert-butoxy-2-methyl-1-oxopropan-2-yloxy)benzoate (4.42 g, 15.01 mmol) in MeOH (40 mL) was added KOH (20 mL, 2M, 40 mmol) and THF (10 mL). The reaction was allowed to stir for 16 hours before HCl (1.2 M) was added until a precipitate stopped forming The solid was filtered and washed with hexane and dried on the hi vac to an off white solid. ¹H NMR (DMSO-d₆): δ 1.32 (s, 9H), 1.52 (s, 6H), 6.80 (d, 2H, J=8.8 Hz), 7.82 (d, 2H, J=9.2 Hz) 12.4 (br, 1H).

### Preparation of methyl 2-(trityloxy)acetate

To Hunig's base (4.8 mL, 27.5 mmol, d=0.742) in DCM (25 mL) was added methyl glycolate (1.4 mL, 18.4 mmol, d=1.184) and then trityl chloride (5.15 g, 18.4 mmol). The reaction was allowed to stir at room temperature for 48 hours before it was quenched with saturated NaHCO₃. The aqueous layer was extracted twice more with dichloromethane. The combined organics were washed with brine and dried over Na₂SO₄, filtered and concentrated. The desired product was purified via silica gel and the product was eluded with 10% ethyl acetate/hexanes. The fractions were pooled and concentrated to yield a yellow oil (3.83 g). ¹H NMR (DMSO-d₆): δ 3.53 (s, 3H), 3.69(s, 2H), 7.32 (m, 15H).

### Preparation of 2-(trityloxy)acetohydrazide

To methyl 2-(trityloxy)acetate (3.16 g, 9.51 mmol) in MeOH (50 mL) was added hydrazine-monohydrate (2.3 mL, 47.4 mmol, d=1.032). The reaction was allowed to stir for 16 hours at room temperature before it was concentrated and dried to a white powder (3.1 g). ¹H NMR (DMSO-d₆): δ 3.42 (s, 2H), 4.29 (brs, 2H), 7.32 (m, 15H), 9.12 (s, 1H).

### Preparation of N-propyl-2-(2-(trityloxy)acetyl)hydrazinecarboxamide

To 2-(trityloxy)acetohydrazide (1.37 g, 4.1 mmol) in THF (30 mL) was added propyl isocyanate (0.39 mL, 4.1 mmol, d=0.896). The reaction was allowed to stir at room temperature for 16 hours before it was concentrated in vacuo to a white powder (1.72 g). ¹H NMR (DMSO-d₆): δ 0.82 (t, 3H, J=8.0 Hz), 1.37 (m, 2H), 2.95 (q, 2H, J=7.2 Hz, J=15.2 Hz), 3.50 (s, 2H), 6.33 (t, 1H, J=6.4 Hz), 7.28 (m, 5H), 7.31 (m, 5H), 7.45 (m, 5H), 7.67 (s 1H), 9.45 (s, 1H).

### Preparation of 4-propyl-3-(trityloxymethyl)-1H-1,2,4-triazol-5(4H)-one

To N-propyl-2-(2-(trityloxy)acetyl)hydrazinecarboxamide (0.637 g, 1.5 mmol) in MeOH (20 mL) was added KOH (1.10 g, 19.6 mmol). This mixture was refluxed for 16 hours before it was subjected to a standard workup. The product was purified over silica gel by eluding with 1:1 ethylacetate:hexanes. The pooled fractions were concentrated to a white solid (0.311 g). ¹H NMR (DMSO-d₆): δ 0.66 (t, 3H, J=8.0 Hz), 1.41 (sex, 2H), 3.32 (t, 2H, J=8.0 Hz), 3.89 (s, 2H), 7.35 (m, 15H), 11.68 (s, 1H).

### Preparation of 1-(4-methylbenzyl)-4-propyl-3-(trityloxymethyl)-1H-1,2,4-triazol-5(4H)-one

To 4-propyl-3-(trityloxymethyl)-1H-1,2,4-triazol-5(4H)-one (0.30 g, 0.75 mmol), 4 methyl benzyl bromide (0.219 g, 1.18 mmol) and potassium carbonate (0.59 g, 4.27 mmol) was added DMF (9 mL). The reaction was heated to 50° C. for 16 hours before water was slowly added. The mixture was allowed to stir for 30 minutes before the solid was filtered and washed with minimal amount of hexanes. The filter cake was dried to a white solid (0.301 g, 0.597 mmol). ¹H NMR (DMSO-d₆): δ 0.68 (t, 3H, J=8.8 Hz), 1.44 (m, 2H), 2.27 (s, 3H), 3.39 (t, 2H, J=8.0 Hz), 3.95 (s, 2H), 4.82 (s, 2H), 6.33 (t, 1H, J=6.4 Hz), 7.36 (m, 15H), 11.69 (s, 1H).

### Preparation of 3-(hydroxymethyl)-1-(4-methylbenzyl)-4-propyl-1H-1,2,4-triazol-5(4H)-one

To 1-(4-methylbenzyl)-4-propyl-3-(trityloxymethyl)-1H-1,2,4-triazol-5(4H)-one (0.30 g, 0.59 mmol) in dichloromethane (10 mL) was added TFA (2 mL). The reaction was stirred for 1 hour before it was concentrated and purified over silica gel. The product was eluded with 1:3 hexanes:ethyl acetate and concentrated to an oil (71 mgs). ¹H NMR (DMSO-d₆): δ 0.85 (t, 3H, J=7.2 Hz), 1.65 (sex, 2H), 2.28 (s, 3H), 3.60 (t, 2H, J=7.2 Hz), 4.32 (s, 2H), 4.80 (s, 2H), 5.60 (brs, 1H), 6.33 (td, 2H, J=2.0 Hz, J=8.4 Hz), 7.34 (td, 2H, J=2.4 Hz, J=8.4 Hz).

### Preparation of (1-(4-methylbenzyl)-5-oxo-4-propyl-4,5-dihydro-1H-1,2,4-triazol-3-yl)methyl 4-(1-tert-butoxy-2-methyl-1-oxopropan-2-yloxy)benzoate

To 3-(hydroxymethyl)-1-(4-methylbenzyl)-4-propyl-1H-1,2,4-triazol-5(4H)-one (70 mgs, 0.27 mmol) and 4-(1-tert-butoxy-2-methyl-1-oxopropan-2-yloxy)benzoic acid (Acid B) (75 mgs, 0.27 mmol) was added EDC (52 mgs, 0.27 mmol), DMAP (33 mgs, 0.27 mmol), and DMF (6 mL). The reaction was allowed to stir for 16 hours before being subjected to a standard work-up. The product was purified over silica gel and eluded with 35% ethyl acetate in hexanes. The appropriate fractions were pooled and concentrated to an oil (137 mgs). ¹H NMR (DMSO-d₆): δ 0.80 (t, 3H, J=7.6 Hz), 1.34 (s, 9H), 1.56 (s, 6H), 1.60 (m 2H), 2.26 (s, 3H), 4.84 (s, 2H), 5.21 (s, 2H), 6.85 (d, 2H, J=6.4 Hz), 7.13 (s, 4H), 7.87 (d, 2H, J=9.2 Hz).

### Preparation of sodium 2-methyl-2-(4-(((1-(4-methylbenzyl)-5-oxo-4-propyl-4,5-dihydro-1H-1,2,4-triazol-3-yl)methoxy)carbonyl)phenoxy)propanol

To (1-(4-methylbenzyl)-5-oxo-4-propyl-4,5-dihydro-1H-1,2,4-triazol-3-yl)methyl 4-(1-tert-butoxy-2-methyl-1-oxopropan-2-yloxy)benzoate (137 mgs, 0.26 mmol) dissolved in DCM (7 mL) was added TFA (3 mL). The reaction was allowed to stir for 1 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (53 mgs). A solution of white powder (21.2 mgs, 0.0455 mmol) acetonitrile (2 mL) and NaHCO₃ (45.5 uL, 1N, 0.0455 mmol) was frozen and lyophilized to a white powder. ¹H NMR (DMSO-d₆): δ 0.81 (t, 3H, J=7.6 Hz), 1.55 (s, 6H), 1.63 (m 2H), 2.26 (s, 3H), 3.63 (t, 2H, J=7.6 Hz), 4.85 (s, 2H), 5.20 (s, 2H), 6.84 (td, 2H, J=1.6 Hz, J=9.2 Hz), 7.14 (s, 4H), 7.87 (td, 2H, J=2.4 Hz, J=9.2 Hz).

### Example 3

### Compound 150

### Sodium 2-(4-(((1-(4-tert-butylbenzyl)-5-oxo-4-propyl-4,5-dihydro-1H-1,2,4-triazol-3-yl)methoxy)carbonyl)phenoxy)-2-methylpropanoate

### Preparation of 1-(4-tert-butylbenzyl)-4-propyl-3-(trityloxymethyl)-1H-1,2,4-triazol-5(4H)-one

To 4-propyl-3-(trityloxymethyl)-1H-1,2,4-triazol-5(4H)-one (0.30 g, 0.75 mmol), 4 t-butyl benzyl bromide (220 uL, 1.2 mmol, d=1.236) and potassium carbonate (0.6 g, 4.35 mmol) was added DMF (10 mL). The reaction was heated to 50° C. for 16 hours before being cooled and water slowly added. The mixture was allowed to stir for 30 minutes before the solid was filtered and washed with minimal amount of hexanes. The filter cake was dried to a white solid (0.335 g). ¹H NMR (DMSO-d₆): δ 0.68 (t, 3H, J=7.2 Hz), 1.26 (s, 9H), 1.45 (m, 2H), 3.95 (s, 2H), 4.83 (s, 2H), 7.36 (m, 15H).

### Preparation of 1-(4-tert-butylbenzyl)-3-(hydroxymethyl)-4-propyl-1H-1,2,4-triazol-5(4H)-one

To 1-(4-tert-butylbenzyl)-4-propyl-3-(trityloxymethyl)-1H-1,2,4-triazol-5(4H)-one (0.335 g, 0.614 mmol) in dichloromethane (5 mL) was added TFA (3 mL). The reaction was stirred for 10 min. before it was concentrated and purified over silica gel. The product was eluded with 1:3 hexanes:ethyl acetate and concentrated to an oil (106 mgs). ¹H NMR (DMSO-d₆): δ 0.85 (t, 3H, J=7.2 Hz), 1.25 (s, 9H), 1.65 (sex, 2H), 3.60 (t, 2H, J=7.2 Hz), 4.32 (s, 2H), 4.80 (s, 2H), 5.54 (brs, 1H), 7.15 (td, 2H, J=2.0 Hz, J=8.4 Hz), 7.34 (td, 2H, J=2.4 Hz, J=8.4 Hz).

### Preparation of sodium 2-(4-(((1-(4-tert-butylbenzyl)-5-oxo-4-propyl-4,5-dihydro-1H-1,2,4-triazol-3-yl)methoxy)carbonyl)phenoxy)-2-methylpropanoate

To 1-(4-tert-butylbenzyl)-3-(hydroxymethyl)-4-propyl-1H-1,2,4-triazol-5(4H)-one (89 mgs, 0.29 mmol) and 4-(1-tert-butoxy-2-methyl-1-oxopropan-2-yloxy)benzoic acid (82 mgs, 0.29 mmol) was added EDC (56 mgs, 0.29 mmol), DMAP (36 mgs, 0.29 mmol), and DMF (3 mL). The reaction was allowed to stir for 16 hours before it was subjected to a standard work-up and concentrated to an oil, which was dissolved in DCM (5 mL) before TFA (2 mL) was added. The reaction was allowed to stir for 1 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (95.7 mgs), which was taken up into a solution with acetonitrile (2 mL) and NaHCO₃ (0.188 mL, 1N, 0.188 mmol). This mixture was frozen and lyophilized to a white powder (96 mgs). ¹H NMR (DMSO-d₆): δ 0.82 (t, 3H, J=7.2 Hz), 1.25 (s, 18H), 1.39 (s, 6H), 1.62 (sex, 2H), 3.63 (t, 2H, J=7.6 Hz), 4.85 (s, 2H), 5.19 (s, 2H), 6.85 (td, 2H, J=2.0 Hz, J=8.8 Hz), 7.17 (d, 2H, J=8.8 Hz), 7.35 (td, 2H, J=2.4 Hz, J=8.8 Hz), 7.74 (td, 2H, J=2.4 Hz, J=9.2 Hz).

### Example 4

### Compound 155

### Sodium 2-(4-(((4-(2-fluorobenzyl)-1-(4-methylbenzyl)-5-oxo-4,5-dihydro-1H-1,2,4-triazol-3-yl)methoxy)carbonyl)phenoxy)-2-methylpropanoate

### Preparation of N-(2-fluorobenzyl)-2-(2-(trityloxy)acetyl)hydrazinecarboxamide

To 2-(2-(trityloxy)acetyl)hydrazinecarboxamide (1.0 g, 3.0 mmol) in THF (10 mL) was added 2-fluorobenzyl isocyanate (0.384 mL, 3.0 mmol, d=1.18). The reaction was allowed to stir at room temperature for 16 hours before it was concentrated to a solid (1.49 g). ¹H NMR (DMSO-d₆): δ 3.51 (s, 2H), 4.25 (d, 2H, J=5.6 Hz), 7.27 (m, 19H), 7.93 (s, 1H), 9.59 (s, 1H).

### Preparation of 4-(2-fluorobenzyl)-3-(trityloxymethyl)-1H-1,2,4-triazol-5(4H)-one

To N-(2-fluorobenzyl)-2-(2-(trityloxy)acetyl)hydrazinecarboxamide (1.49 g, 3.1 mmol) in MeOH (30 mL) was added KOH (2.25 g, 40.2 mmol). This mixture was refluxed for 16 hours before HCl (1.2M) was added. The mixture was then subjected to a standard work-up. The organics were filtered, concentrated, and dried to an oil. ¹H NMR (DMSO-d₆): δ 3.83 (s, 2H), 4.75 (s, 2H), 7.28 (m, 19H), 11.92 (s, 1H).

### Preparation of 4-(2-fluorobenzyl)-1-(4-methylbenzyl)-3-(trityloxymethyl)-1H-1,2,4-triazol-5(4H)-one

To 4-(2-fluorobenzyl)-3-(trityloxymethyl)-1H-1,2,4-triazol-5(4H)-one (0.465 g, 1.0 mmol), 4 methyl benzyl bromide (0.296 uL, 1.6 mmol) and potassium carbonate (0.787 g, 5.70 mmol) were added DMF (9 mL). The reaction was heated to 50° C. for 48 hours before it was cooled and subjected to a standard work-up. The organics were filtered and concentrated to an oil. ¹H NMR (DMSO-d₆): δ 2.28 (s, 3H), 3.87 (s, 2H), 4.80 (s, 2H), 4.88 (s, 2H), 7.36 (m, 23H).

### Preparation of 4-(2-fluorobenzyl)-3-(hydroxymethyl)-1-(4-methylbenzyl)-1H-1,2,4-triazol-5(4H)-one

To 4-(2-fluorobenzyl)-1-(4-methylbenzyl)-3-(trityloxymethyl)-1H-1,2,4-triazol-5(4H)-one (0.569 g, 1.0 mmol) in dichloromethane (10 mL) was added TFA (0.5 mL). The reaction was stirred for 10 min. before it was concentrated and purified over silica gel. The product was eluded with 2:3 hexanes:ethyl acetate and concentrated to an oil (129 mgs). ¹H NMR (DMSO-d₆): δ 2.27 (s, 3H), 4.26 (s, 2H), 4.83 (s, 2H), 4.96 (s, 2H), 5.54 (brs, 1H), 7.12 (s, 4H), 7.18 (m, 3H), 7.35 (m, 1H).

### Preparation of sodium 2-(((4(4-(2-fluorobenzyl)-1-(4-methylbenzyl)-5-oxo-4,5-dihydro-1H-1,2,4-triazol-3-yl)methoxy)carbonyl)phenoxy)-2-methylpropanoate

To 4-(2-fluorobenzyl)-3-(hydroxymethyl)-1-(4-methylbenzyl)-1H-1,2,4-triazol-5(4H)-one (64.5 mgs, 0.197 mmol) and 4-(1-tert-butoxy-2-methyl-1-oxopropan-2-yloxy)benzoic acid (Acid B) (55 mgs, 0.197 mmol) was added EDC (38 mgs, 0.197 mmol), DMAP (24 mgs, 0.197 mmol), and DCM (10 mL). The reaction was allowed to stir for 16 hours before it was subjected to a standard work-up and concentrated to an oil, which was dissolved in DCM (6 mL) before TFA (1 mL) was added. The reaction was allowed to stir for 0.5 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (27.7 mgs), which were taken up into a solution with acetonitrile (2 mL) and NaHCO₃ (0.0519 mL, 1N, 0.052 mmol). This mixture was frozen and lyophilized to a white powder. ¹H NMR (DMSO-d₆): δ 1.38 (s, 6H), 2.28 (s, 3H), 4.91 (s, 2H), 5.01 (s, 2H), 5.12 (s, 2H), 6.69 (td, 2H, J=2.0 Hz, J=8.8 Hz), 7.11 (m, 2H), 7.18 (d, 2H, J=6.0 Hz), 7.32 (td, 2H, J=2.0 Hz, J=9.2 Hz).

### Example 5

### Compound 152

### Sodium 2-methyl-2-(4-(((1-(4-methylbenzyl)-4-octyl-5-oxo-4,5-dihydro-1H-1,2,4-triazol-3-yl)methoxy)carbonyl)phenoxy)propanoate

### Preparation of N-octyl-2-(2-(trityloxy)acetyl)hydrazinecarboxamide

To 2-(2-(trityloxy)acetyl)hydrazinecarboxamide (1.0 g, 3.0 mmol) in THF (10 mL) was added octyl isocyanate (0.528 mL, 3.0 mmol, d=0.88). The reaction was allowed to stir at room temperature for 16 hours before it was concentrated to a solid (1.37 g). ¹H NMR (DMSO-d₆): δ 0.85 (t, 3H, J=6.8 Hz), 1.24 (s, 10H), 1.35 (m, 2H), 2.97 (q, 2H, J=6.4 Hz, J=13.2 Hz), 3.48 (s, 2H), 3.62 (t, 1H, J=5.6 Hz), 7.27 (m, 15H), 6.45 (s, 1H), 7.67 (d, 1H, J=1.2 Hz), 9.46 (d, 1H, J=1.6 Hz).

### Preparation of 4-octyl-3-(trityloxymethyl)-1H-1,2,4-triazol-5(4H)-one

To N-octyl-2-(2-(trityloxy)acetyl)hydrazinecarboxamide (1.37 g, 2.8 mmol) in MeOH (30 mL) was added KOH (2.08 g, 40.2 mmol). The reaction was refluxed for 16 hours before HCl (1.2M) was added. The mixture was then subjected to a standard work-up. The organics were filtered and concentrated to an oil. ¹H NMR (DMSO-d₆): δ 0.82 (t, 3H, J=6.8 Hz), 1.13 (brm, 10H), 1.42 (brm, 2H), 3.35 (m, 2H), 3.88 (s, 2H), 7.28 (m, 19H), 11.69 (s, 1H).

### Preparation of 1-(4-methylbenzyl)-4-octyl-3-(trityloxymethyl)-1H-1,2,4-triazol-5(4H)-one

To 4-octyl-3-(trityloxymethyl)-1H-1,2,4-triazol-5(4H)-one (0.465 g, 1.0 mmol), 4-methyl benzyl bromide (0.296 uL, 1.6 mmol) and potassium carbonate (0.787 g, 5.70 mmol) was added DMF (9 mL). The reaction was heated to 50° C. for 48 hours before it was cooled and subjected to a standard work-up. The organics were filtered and concentrated to an oil. ¹H NMR (DMSO-d₆): δ 0.82 (t, 3H, J=7.2 Hz), 1.1 (brm, 10H), 1.44 (m, 2H), 2.26 (s, 3H), 3.39 (m, 2H), 3.90 (s, 2H), 4.82 (s, 2H), 7.30 (m, 23H).

### Preparation of 3-(hydroxymethyl)-1-(4-methylbenzyl)-4-octyl-1H-1,2,4-triazol-5(4H)-one

To 1-(4-methylbenzyl)-4-octyl-3-(trityloxymethyl)-1H-1,2,4-triazol-5(4H)-one (0.569 g, 1.0 mmol) in dichloromethane (10 mL) was added TFA (0.5 mL). The reaction was stirred for 10 min. before it was concentrated and purified over silica gel. The product was eluded with 2:3 hexanes:ethyl acetate and concentrated to an oil (129 mgs). ¹H NMR (DMSO-d₆): δ 0.85 (t, 3H, J=7.2), 1.18 (brs, 10H), 1.62 (brm, 2H), 2.26 (s, 3H), 3.63 (t, 2H, J=7.6 Hz), 4.31 (s, 2H), 4.79 (s, 2H), 7.12 (s, 4H).

### Preparation of sodium 2-methyl-2-(4-(((1-(4-methylbenzyl)-4-octyl-5-oxo-4,5-dihydro-1H-1,2,4-triazol-3-yl)methoxy)carbonyl)phenoxy)propanoate

To 3-(hydroxymethyl)-1-(4-methylbenzyl)-4-octyl-1H-1,2,4-triazol-5(4H)-one (64.5 mgs, 0.197 mmol) and 4-(1-tert-butoxy-2-methyl-1-oxopropan-2-yloxy)benzoic acid (Acid B) (55 mgs, 0.197 mmol) was added EDC (38 mgs, 0.197 mmol), DMAP (24 mgs, 0.197 mmol), and DCM (10 mL). The reaction was allowed to stir for 16 hours before it was subjected to a standard work-up and concentrated to an oil. The residue was dissolved in DCM (6 mL) to which was added TFA (1 mL). The reaction was stirred for 0.5 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (27.7 mgs), which was taken up into a solution with acetonitrile (2 mL) and NaHCO₃ (0.0519 mL, 1N, 0.052 mmol). This mixture was frozen and lyophilized to a white powder. ¹H NMR (DMSO-d₆): δ 0.82 (t, 3H, J=7.0), 1.15 (brm, 10H), 1.39 (s, 6H), 1.57 (brm, 2H), 2.27 (s, 3H), 3.63 (t, 2H, J=7.6 Hz), 4.84 (s, 2H), 5.19 (s, 2H), 6.83 (d, 2H, J=9.2 Hz), 7.14 (s, 4H), 7.73 (d, 2H, J=9.2 Hz).

### Example 6

### Compound 191

### 2-(4-(((4-heptyl-1-(4-methylbenzyl)-5-oxo-4,5-dihydro-1H-1,2,4-triazol-3-yl)methoxy)carbonyl)phenylthio)-2-methylpropanoic acid

### Preparation of methyl 4-(1-tert-butoxy-2-methyl-1-oxopropan-2-ylthio)benzoate

To a solution of methyl 4-mercaptobenzoate (2.51 g, 14.9 mmol) and potassium carbonate (8.23 g, 59.5 mmol) in DMF (40 mL) was added tert-butyl 2-bromo-2-methylpropanoate (8.4 mL, 44.4 mmol, d=1.196). The reaction was stirred at 75° C. for 16 hours. The reaction was cooled and subjected to a standard work-up. The organic layers were concentrated to a colorless oil, which was purified on silica gel; product was eluded with 5:95 EA/Hexanes to provide a colorless oil (4.73 g, 15.3 mmol). ¹H NMR (DMSO-d₆): δ 1.33 (s, 9H), 1.42 (s, 6H), 3.86 (s, 3H), 7.51 (td, 2H, J=2.0 Hz, J=4.4 Hz), 7.84 (td, 2H, J=2.0 Hz, J=4.4 Hz).

### Preparation of 4-(1-tert-butoxy-2-methyl-1-oxopropan-2-ylthio)benzoic acid (Acid A)

To methyl 4-(1-tert-butoxy-2-methyl-1-oxopropan-2-ylthio)benzoate (4.73 g, 15.3 mmol) in MeOH (40 mL) was added KOH (23 mL, 2M, 46 mmol) and THF (10 mL). The reaction was stirred for 16 hours before HCl (1.2 M) was added until a precipitate stopped forming. The solid was filtered and washed with hexane and dried to an off white solid (3.81 g). ¹H NMR (DMSO-d₆): δ 1.34 (s, 9H), 1.42 (s, 6H), 7.55 (d, 2H, J=4.4 Hz), 7.91 (d, 2H, J=4.8 Hz) 13.2 (br, 1H).

### Preparation of 4-heptyl-3-(trityloxymethyl)-1H-1,2,4-triazol-5(4H)-one

To 2-(2-(trityloxy)acetyl)hydrazinecarboxamide (5.0 g, 15.0 mmol) in THF (30 mL) was added hepyl isocyanate (2.43 mL, 15.1 mmol, d=0.876). The reaction was allowed to stir at room temperature for 16 hours before it was concentrated to a solid. To the residue, dissolved in MeOH (50 mL), was added KOH pellets (11.0 g, 196.4 mmol). The reaction mixture was then refluxed 16 hours before HCl (1.2M) was added slowly until precipitate ceased to form. The solid was filtered and the filter cake was rinsed with hexanes before it was dried to a solid (5.64 g, 12.1 mmol). ¹H NMR (DMSO-d₆): δ 0.81 (t, 3H, J=7.2 Hz), 1.1 (m, 8H), 1.41 (m, 2H), 3.34 (t, 2H, J=7.6 Hz), 3.87 (s, 2H), 7.36 (m, 15H), 11.5 (br, 1H).

### Preparation of 4-heptyl-3-(hydroxymethyl)-1-(4-methylbenzyl)-1H-1,2,4-triazol-5(4H)-one

To 4-heptyl-3-(trityloxymethyl)-1H-1,2,4-triazol-5(4H)-one (2.82 g, 6.20 mmol), 4 methyl benzyl bromide (1.836 mL, 9.9 mmol) and potassium carbonate (4.96 g, 35.9 mmol) was added DMF (25 mL). The reaction was heated to 50° C. for 16 hours before it was cooled and DI water was added. The precipitate formed was filtered. The filter cake was taken up into hexanes and stirred vigorously, filtered and dried. The filter cake was dissolved in dichloromethane (10 mL) to which was added TFA (2.5 mL). The reaction was stirred for 75 min. before it was concentrated and purified over silica gel. The product was eluded with 2:3 hexanes:ethyl acetate and concentrated to an oil (801 mgs). ¹H NMR (DMSO-d₆): δ 0.84 (t, 3H, J=6.8 Hz), 1.25 (brs, 8H), 1.62 (brm, 2H), 2.26 (s, 3H), 3.63 (t, 2H, J=7.2 Hz), 4.31 (s, 2H), 4.79 (s, 2H), 7.12 (s, 4H).

### Preparation of 2-(4-(((4-heptyl-1-(4-methylbenzyl)-5-oxo-4,5-dihydro-1H-1,2,4-triazol-3-yl)methoxy)carbonyl)phenylthio)-2-methylpropanoic acid

To 4-heptyl-3-(hydroxymethyl)-1-(4-methylbenzyl)-1H-1,2,4-triazol-5(4H)-one (270 mgs, 0.85 mmol) and 4-(1-tert-butoxy-2-methyl-1-oxopropan-2-ylthio)benzoic acid (252 mgs, 0.85 mmol) was added EDC (162 mgs, 0.85 mmol), DMAP (104 mgs, 0.85 mmol), and DCM (10 mL). The reaction was allowed to stir for 16 hours before it was subjected to a standard work-up and concentrated to an oil. To the residue, which was dissolved in DCM (6 mL), was added TFA (3 mL). The reaction was allowed to stir for 0.5 hour before being concentrated and purified via reverse phase high pressure liquid chromatography 1:9 ACN:water to 8.5:1.5 ACN:water over 20 minutes. The appropriate fractions were pooled, frozen and lyophilized to a white powder (306 mgs). ¹H NMR (DMSO-d₆): δ 0.77 (t, 3H, J=7.2 Hz), 1.13 (m, 8H), 1.42 (s, 6H), 1.57 (brm, 2H), 2.27 (s, 3H), 3.65 (t, 2H, J=7.6 Hz), 4.85 (s, 2H), 5.26 (s, 2H), 7.14 (s, 4H), 7.55 (d, 2H, J=8.8 Hz), 7.91 (d, 2H, J=8.4 Hz).

### Example 7

### Sodium 2-(4-(2-((4-hexyl-1-(4-methylbenzyl)-5-oxo-4,5-dihydro-1H-1,2,4-triazol-3-yl)methoxy)-2-oxoethyl)phenoxy)-2-methylpropanoate

### Preparation of 2-(4-(1-tert-butoxy-2-methyl-1-oxopropan-2-yloxy)phenyl)acetic acid (Acid C)

To a solution of methyl 2-(4-hydroxyphenyl)acetate (2.49 g, 14.98 mmol) and potassium carbonate (3.12 g, 35.5 mmol) in DMF (20 mL) was added tert-butyl 2-bromo-2-methylpropanoate (5.6 mL, 28.7 mmol, d=1.196). The reaction was stirred at 75° C. for 16 hours. The reaction was cooled and subjected to a standard work-up. The organic layer was concentrated to a colorless oil. Purification via silica gel eluding desired product with 1:5 EtOAc/Hexanes. The appropriate fractions were pooled and concentrated. To the residue was added MeOH (6 mL), KOH (7 mL, 1M, 7 mmol) and THF (7 mL). The reaction was allowed to stir for 4 hours before it was washed with EtOH and HCl (1N). The aqueous layer was extracted twice more with EtOH, the combined organic layers were dried over Na₂SO₄, filtered and concentrated in vacuo. ¹H NMR (DMSO-d₆): δ 1.42 (s, 9H), 1.55 (s, 6H), 3.57 (s, 2H), 6.80 (d, 2H, J=8.4 Hz), 7.13 (d, 2H, J=8.4 Hz).

### Preparation of 4-hexyl-3-(trityloxymethyl)-1H-1,2,4-triazol-5(4H)-one

To 2-(2-(trityloxy)acetyl)hydrazinecarboxamide (1.0 g, 3.0 mmol) in THF (10 mL) was added hexyl isocyanate (0.435 mL, 3.0 mmol, d=0.878). The reaction was allowed to stir at room temperature for 16 hours before it was concentrated in vacuo to a solid. To the residue, dissolved in MeOH (30 mL), was added KOH pellets (2.24 g, 40.0 mmol). The reaction mixture was then refluxed 16 hours before HCl (1.2M) was added. The reaction was subjected to a standard work-up, dried with Na₂SO₄, filtered, and concentrated in vacuo to yield an oil (1.1 g). ¹H NMR (DMSO-d₆): δ 0.79 (t, 3H, J=7.2 Hz), 1.08 (m, 6H), 1.41 (m, 2H), 3.34 (t, 2H, J=7.6 Hz), 3.88 (s, 2H), 7.36 (m, 15H), 11.69 (s, 1H).

### Preparation of 4-hexyl-3-(hydroxymethyl)-1-(4-methylbenzyl)-1H-1,2,4-triazol-5(4H)-one

To 4-hexyl-3-(trityloxymethyl)-1H-1,2,4-triazol-5(4H)-one (0.441 g, 1.0 mmol), 4 methyl benzyl bromide (0.296 g, 1.6 mmol) and potassium carbonate (0.787 g, 5.7 mmol) was added DMF (25 mL). The reaction was heated to 50° C. for 16 hours before it was cooled and DI water was added followed by a standard work-up. The residue was dissolved in dichloromethane (10 mL) to which was added TFA (2.5 mL). The reaction was stirred for 10 min. before it was concentrated and purified over silica gel. The product was eluded with 2:3 hexanes:ethyl acetate and concentrated to an oil (0.66 mgs). ¹H NMR (DMSO-d₆): δ 0.85 (t, 3H, J=6.8 Hz), 1.25 (brs, 8H), 1.62 (brm, 2H), 2.26 (s, 3H), 3.63 (t, 2H, J=7.2 Hz), 4.31 (s, 2H), 4.79 (s, 2H), 7.12 (s, 4H).

### Preparation of 2-(4-(2-((4-hexyl-1-(4-methylbenzyl)-5-oxo-4,5-dihydro-1H-1,2,4-triazol-3-yl)methoxy)-2-oxoethyl)phenoxy)-2-methylpropanoic acid

To 4-hexyl-3-(hydroxymethyl)-1-(4-methylbenzyl)-1H-1,2,4-triazol-5(4H)-one (33.2 mgs, 0.11 mmol) and 2-(4-(1-tert-butoxy-2-methyl-1-oxopropan-2-yloxy)phenyl)acetic acid (Acid C) (32 mgs, 0.11 mmol) was added EDC (21 mgs, 0.11 mmol), DMAP (13 mgs, 0.11 mmol), and DCM (10 mL). The reaction was allowed to stir for 16 hours before being subjected to a standard work-up and concentrated. To the residue in DCM (6 mL) was added TFA (3 mL). The reaction was allowed to stir for 0.5 hour before it was concentrated and purified via reverse phase high pressure liquid chromatography with 1:9 ACN:water to 8.5:1.5 ACN:water over 20 minutes. The appropriate fractions were pooled, frozen and lyophilized to a white powder. The solid was dissolved in acetonitrile (3 mL) and 1N NaHCO₃ (22.6 uL) was added, this solution was then frozen and lyophilized to a solid (12 mgs). ¹H NMR (DMSO-d₆): δ 0.85 (t, 3H, J=7.2 Hz), 1.23 (m, 6H), 1.33 (s, 6H), 1.53 (brm, 2H), 2.27 (s, 3H), 3.52 (t, 2H, J=7.6 Hz), 3.57 (s, 2H), 4.82 (s, 2H), 4.98 (s, 2H), 6.71 (d, 2H, J=8.4 Hz), 6.97 (d, 2H, J=8.4 Hz), 7.13 (s, 4H).

### Example 8

### Compound 192

### 2-(4-(((4-heptyl-1-(4-methylbenzyl)-5-oxo-4,5-dihydro-1H-1,2,4-triazol-3-yl)methoxy)carbonyl)phenoxy)-2-methylpropanoic acid

To 4-heptyl-3-(hydroxymethyl)-1-(4-methylbenzyl)-1H-1,2,4-triazol-5(4H)-one (150 mgs, 0.49 mmol) and 4-(1-tert-butoxy-2-methyl-1-oxopropan-2-yloxy)benzoic acid (Acid B) (145 mgs, 0.49 mmol) was added EDC (93 mgs, 0.49 mmol), DMAP (60 mgs, 0.49 mmol), and DMF (10 mL). The reaction was allowed to stir for 16 hours before it was subjected to a standard work-up and concentrated. To the residue, dissolved in DCM (6 mL), was added TFA (3 mL). The reaction was allowed to stir for 1 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (39 mgs). ¹H NMR (DMSO-d₆): δ 0.78 (t, 3H, J=7.0 Hz), 1.13 (m, 8H), 1.42 (s, 8H), 2.27 (s, 3H), 3.63 (t, 2H, J=7.6 Hz), 4.84 (s, 2H), 5.21 (s, 2H), 6.87 (d, 2H, J=8.8 Hz), 7.14 (s, 4H), 7.86 (d, 2H, J=8.8 Hz), 13.3 (brs, 1H).

### Example 9

### Compound 160

### 2-(4-(((4-hexyl-1-(4-methylbenzyl)-5-oxo-4,5-dihydro-1H-1,2,4-triazol-3-yl)methoxy)carbonyl)phenylthio)-2-methylpropanoic acid

To 4-hexyl-3-(hydroxymethyl)-1-(4-methylbenzyl)-1H-1,2,4-triazol-5(4H)-one (102 mgs, 0.34 mmol) and 4-(1-tert-butoxy-2-methyl-1-oxopropan-2-ylthio)benzoic acid (Acid A) (100 mgs, 0.34 mmol) was added EDC (65 mgs, 0.34 mmol), DMAP (42 mgs, 0.34 mmol), and DMF (10 mL). The reaction was allowed to stir for 16 hours before it was subjected to a standard work-up and concentrated. To the residue dissolved in DCM (6 mL) was added TFA (3 mL). The reaction was allowed to stir for 0.5 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder. The solid was dissolved in acetonitrile (3 mL) and 1N NaHCO₃ (22.6 uL) was added, this solution was frozen and lyophilized to a solid (12 mgs). ¹H NMR (DMSO-d₆): δ 0.75 (t, 3H, J=7.2 Hz), 1.12 (brs, 6H), 1.42 (s, 6H), 1.57 (brm, 2H), 2.27 (s, 3H), 3.64 (t, 2H, J=7.2), 4.85 (s, 2H), 5.26 (s, 2H), 7.14 (s, 4H), 7.55 (d, 2H, J=8.8 Hz), 7.90 (d, 2H, J=8.4), 12.8 (brs, 1H).

### Example 10

### Compound 261

### 2-Methyl-2-(4-(((1-(4-methylbenzyl)-5-oxo-4,5-dihydro-1H-1,2,4-triazol-3-yl)methoxy)carbonyl)phenylthio)propanoic acid (ATI-20332)

### Preparation of 1-(4-methylbenzyl)hydrazinecarboxamide hydrochloride

tert-Butyl 2-carbamoyl-2-(4-methylbenzyl)hydrazinecarboxylate (prepared as described in US2004/0102500, Example 122) (6.90 g, 0.0247 mol) and hydrogen chloride in dioxane (4M, 60 mL) are stirred for 4 hours at room temperature. The mixture was evaporated to dryness to give product as a white solid (5.33 g, 99%). MS for C₉H₁₄ClN₃O m/z 216 (M+H)⁺.

### Preparation of 2-(2-(benzyloxy)acetyl)-1-(4-methylbenzyl)hydrazinecarboxamide

Benzyloxyacetyl chloride (3.82 mL, 0.0246 mol) was added dropwise to 1-(4-methylbenzyl)hydrazinecarboxamide hydrochloride (5.30 g, 0.0246 mol) and pyridine (4.97 mL, 0.0615 mol) in ethyl acetate (75 mL) at 0° C. The mixture was allowed to warm to room temperature and stirred for 1 hour. The reaction was washed with water and brine, dried (MgSO₄) and evaporated to give product as a white solid (8.0 g, 99%); MS for C₁₈H₂₁N₃O₃ m/z 328 (M+H)⁺.

### Preparation of 3-(benzyloxymethyl)-1-(4-methylbenzyl)-1H-1,2,4-triazol-5(4H)-one

2-(2-(Benzyloxy)acetyl)-1-(4-methylbenzyl)hydrazinecarboxamide (1.00 g, 3.05 mmol) and methanesulfonic acid (0.4 mL, 6.11 mmol) in dichloroethane (20 mL) were heated at reflux overnight. The mixture was washed with water and brine, dried (MgSO₄) and evaporated. The residue was purified by PTLC (50% ethyl acetate/hexanes) to give product as a white solid (0.71 g, 75%); MS for C₁₈H₁₉N₃O₂ m/z 310 (M+H)⁺.

### Preparation of 3-(hydroxymethyl)-1-(4-methylbenzyl)-1H-1,2,4-triazol-5(4H)-one

3-(Benzyloxymethyl)-1-(4-methylbenzyl)-1H-1,2,4-triazol-5(4H)-one (0.70 g, 2.26 mmol) and 10% palladium on activated carbon (Degussa type E101 NE/W, 0.14 g) in ethanol were hydrogenated (balloon) overnight. The mixture was filtered through celite and evaporated to give product as a white solid (0.5 g, 99%); MS for C₁₁H₁₃N₃O₂ m/z 220 (M+H)⁺.

### Preparation of 2-methyl-2-(4-(((1-(4-methylbenzyl)-5-oxo-4,5-dihydro-1H-1,2,4-triazol-3-yl)methoxy)carbonyl)phenylthio)propanoic acid

4-(1-tert-Butoxy-2-methyl-1-oxopropan-2-ylthio)benzoic acid (1.80 g, 6.08 mmol), 3-(hydroxymethyl)-1-(4-methylbenzyl)-1H-1,2,4-triazol-5(4H)-one (1.33 g, 6.08 mmol), N-(3-dimethylaminopropyl)-N′-ethylcarbodiimide hydrochloride (1.40 g, 7.30 mmol), and 4-(dimethylamino)pyridine (0.73 g, 6.08 mmol) in dimethylformamide (40 mL) were stirred at room temperature overnight. The mixture was diluted with ethyl acetate, washed with 10% aqueous citric acid, saturated aqueous sodium bicarbonate and brine, dried (MgSO₄), and evaporated. The residue was purified using a Biotage SP4 chromatography system (30% ethyl acetate/hexane) to give (1-(4-methylbenzyl)-5-oxo-4,5-dihydro-1H-1,2,4-triazol-3-yl)methyl 4-(1-tert-butoxy-2-methyl-1-oxopropan-2-ylthio)benzoate as a white solid (2.78 g, 92%); MS for C₂₆H₃₁N₃O₅S m/z 498 (M+H)⁺. The ester and 4M hydrogen chloride solution in dioxane (25 mL) were stirred at room temperature overnight and the evaporated under vacuum. The residue was purified using a Biotage SP4 chromatography system (10% methanol/dichloromethane) to give product as a white solid (2.5 g, 99%); HPLC retention time (Phenomenex 150×4.6 mm, 5μ, 1 mL/min, 20 mM ammonium acetate buffer, MeCN:water=15:85 for 2 min, then to 10:90 in 18 minutes)=10.7 min; ¹H-NMR (400 MHz, d₆-DMSO) 12.8 (br s, 1H); 11.9 (br s, 1H); 7.96 (d, J=9.2 Hz, 2H); 7.58 (d, J=9.2 Hz, 2H); 7.14 (s, 4H); 5.12 (s, 2H); 4.80 (s, 2H); 2.28 (s, 3H); 1.44 (s, 6H); MS for C₂₂H₂₃N₃O₅S m/z 442 (M+H)⁺.

### Example 11

### Compound 187

### 2-(4-(((1-(4-tert-butylbenzyl)-4-hexyl-5-oxo-4,5-dihydro-1H-1,2,4-triazol-3-yl)methoxy)carbonyl)phenylthio)-2-methylpropanoic acid

### Preparation of 1-(4-tert-butylbenzyl)-4-hexyl-3-(hydroxymethyl)-1H-1,2,4-triazol-5(4H)-one

To 4-hexyl-3-(trityloxymethyl)-1H-1,2,4-triazol-5(4H)-one (4.41 g, 10.2 mmol), 4 t-butyl benzyl bromide (3.0 mL, 16.3 mmol) and potassium carbonate (8.0 g, 58.0 mmol) was added DMF (75 mL). The reaction was heated to 50° C. for 16 hours before it was cooled and DI water was added followed by a standard work-up. The residue was dissolved in dichloromethane (20 mL) to which was added TFA (10 mL). The reaction was stirred for 2 hr. before it was concentrated and purified over silica gel. The product was eluded with 2:3 hexanes:ethyl acetate and concentrated to an oil (0.838 mgs). ¹H NMR (DMSO-d₆): δ 0.85 (t, 3H, J=6.8 Hz), 1.25 (brs, 15H), 1.62 (brm, 2H), 3.63 (t, 2H, J=7.6 Hz), 4.31 (s, 2H), 4.80 (s, 2H), 7.16 (d, 2H, J=8.4 Hz), 7.33 (d, 2H, J=8.4 Hz).

### Preparation of 2-(4-(((1-(4-tert-butylbenzyl)-4-hexyl-5-oxo-4,5-dihydro-1H-1,2,4-triazol-3-yl)methoxy)carbonyl)phenylthio)-2-methylpropanoic acid

To 1-(4-tert-butylbenzyl)-4-hexyl-3-(hydroxymethyl)-1H-1,2,4-triazol-5(4H)-one (400 mgs, 1.2 mmol) and 4-(1-tert-butoxy-2-methyl-1-oxopropan-2-ylthio)benzoic acid (Acid A) (336 mgs, 1.2 mmol) was added EDC (229 mgs, 1.2 mmol), DMAP (146 mgs, 1.2 mmol), and DMF (10 mL). The reaction was allowed to stir for 16 hours before it was subjected to a standard work-up and concentrated. To the residue, dissolved in DCM (6 mL), was added TFA (3 mL). The reaction was allowed to stir for 0.5 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder. The solid was dissolved in acetonitrile (3 mL) and 1N NaHCO₃ (22.6 uL), this solution was then frozen and lyophilized to a solid (152 mgs). ¹H NMR (DMSO-d₆): δ 0.75 (t, 3H, J=6.8 Hz), 1.12 (brs, 6H), 1.25 (s, 9H), 1.40 (s, 6H), 1.57 (brm, 2H), 3.64 (t, 2H, J=8.0 Hz), 4.86 (s, 2H), 5.26 (s, 2H), 7.18 (d, 4H, J=8.0 Hz), 7.35 (d, 2H, J=8.4 Hz), 7.55 (d, 2H, J=8.0 Hz), 7.8 (brm, 2H).

### Example 12

### Compound 161

### 2-methyl-2-(4-(((1-(4-methylbenzyl)-4-octyl-5-oxo-4,5-dihydro-1H-1,2,4-triazol-3-yl)methoxy)carbonyl)phenylthio)propanoic acid

To 3-(hydroxymethyl)-1-(4-methylbenzyl)-4-octyl-1H-1,2,4-triazol-5(4H)-one (142 mgs, 0.43 mmol) and 4-(1-tert-butoxy-2-methyl-1-oxopropan-2-ylthio)benzoic acid (Acid A) (126 mgs, 0.43 mmol) was added EDC (81 mgs, 0.43 mmol), DMAP (52 mgs, 0.43 mmol), and DMF (10 mL). The reaction was allowed to stir for 16 hours before it was subjected to a standard work-up and concentrated. To the residue, dissolved in DCM (10 mL), was added TFA (1 mL). The reaction was allowed to stir for 0.5 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (27.7 mgs), which was taken up into a solution with acetonitrile (2 mL) and NaHCO₃ (0.0519 mL, 1N, 0.052 mmol). This mixture was frozen and lyophilized to a white powder (167 mgs). ¹H NMR (DMSO-d₆): δ 0.80 (t, 3H, J=6.8 Hz), 1.15 (brm, 10H), 1.42 (s, 6H), 1.57 (brm, 2H), 2.49 (s, 3H), 3.63 (t, 2H, J=7.6 Hz), 4.85 (s, 2H), 5.26 (s, 2H), 7.14 (s, 4H), 7.55 (d, 2H, J=8.4. Hz), 7.90 (d, 2H, J=8.4 Hz), 12.81 (s, 1H).

### Example 13

### Compound 190

### 2-(4-(((1-(4-tert-butylbenzyl)-4-heptyl-5-oxo-4,5-dihydro-1H-1,2,4-triazol-3-yl)methoxy)carbonyl)phenoxy)-2-methylpropanoic acid

### Preparation of 1-(4-tert-butylbenzyl)-4-heptyl-3-(hydroxymethyl)-1H-1,2,4-triazol-5(4H)-one

To 4-heptyl-3-(trityloxymethyl)-1H-1,2,4-triazol-5(4H)-one (2.82 g, 6.20 mmol), 4-t-butyl benzyl bromide (1.82 mL, 9.9 mmol) and potassium carbonate (4.96 g, 35.9 mmol) was added DMF (25 mL). The reaction was heated to 50° C. for 16 hours, cooled, and quenched with DI water. The precipitate was filtered. The filter cake was taken up into hexanes and stirred vigorously, then filtered and dried. The filter cake was dissolved in dichloromethane (10 mL) before TFA (2.5 mL) was added. The reaction was stirred for 75 min. before it was concentrated and purified over silica gel. The product was eluded with 2:3 hexanes:ethyl acetate and concentrated to an oil (550 mgs). ¹H NMR (DMSO-d₆): δ 0.85 (t, 3H, J=6.8 Hz), 1.25 (s, 17H), 1.63 (brm, 2H), 3.63 (t, 2H, J=7.2), 4.31 (s, 2H), 4.79 (s, 2H), 5.54 (br, 1H), 7.15 (d, 2H, J=8.8 Hz), 7.35 (d, 2H, J=8.8 Hz).

### Preparation of 2-(4-(((1-(4-tert-butylbenzyl)-4-heptyl-5-oxo-4,5-dihydro-1H-1,2,4-triazol-3-yl)methoxy)carbonyl)phenoxy)-2-methylpropanoic acid

To 4-heptyl-3-(hydroxymethyl)-1-(4-methylbenzyl)-1H-1,2,4-triazol-5(4H)-one (130 mgs, 0.37 mmol) and 4-(1-tert-butoxy-2-methyl-1-oxopropan-2-yloxy)benzoic acid (Acid B) (104 mgs, 0.37 mmol) was added EDC (71 mgs, 0.37 mmol), DMAP (45 mgs, 0.37 mmol), and DMF (10 mL). The reaction was allowed to stir for 16 hours, subjected to a standard work-up, and concentrated. The residue was dissolved in DCM (6 mL) to which was added TFA (3 mL). The reaction was allowed to stir for 1 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (135 mgs). ¹H NMR (DMSO-d₆): δ 0.78 (t, 3H, J=7.2 Hz), 1.15 (m, 8H), 1.25 (s, 9H), 1.56 (s, 8H), 3.64 (t, 2H, J=7.6 Hz), 4.85 (s, 2H), 5.22 (s, 2H), 6.87 (d, 2H, J=8.8 Hz), 7.17 (s, 2H, J=8.4 Hz), 7.35 (d, 2H, J=8.4 Hz), 7.87 (d, 2H, J=9.2 Hz), 13.3 (brs, 1H).

### Example 14

### Compound 189

### 2-(4-(((1-(4-tert-butylbenzyl)-4-heptyl-5-oxo-4,5-dihydro-1H-1,2,4-triazol-3-yl)methoxy)carbonyl)phenylthio)-2-methylpropanoic acid

To 4-heptyl-3-(hydroxymethyl)-1-(4-methylbenzyl)-1H-1,2,4-triazol-5(4H)-one (130 mgs, 0.37 mmol) and 4-(1-tert-butoxy-2-methyl-1-oxopropan-2-ylthio)benzoic acid (Acid A) (110 mgs, 0.37 mmol) was added EDC (71 mgs, 0.37 mmol), DMAP (45 mgs, 0.37 mmol), and DMF (10 mL). The reaction was allowed to stir for 16 hours before it was subjected to a standard work-up and concentrated. The residue was dissolved in DCM (6 mL) to which was added TFA (3 mL). The reaction was allowed to stir for 1 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (135 mgs). ¹H NMR (DMSO-d₆): δ 0.78 (t, 3H, J=6.8 Hz), 1.08 (m, 8H), 1.25 (s, 9H), 1.42 (s, 6H), 1.57 (m, 2H), 3.64 (t, 2H, J=7.6 Hz), 4.86 (s, 2H), 5.27 (s, 2H), 7.18 (d, 2H, J=8.8 Hz), 7.35 (s, 2H), 7.55 (d, 2H, J=8.8 Hz), 7.91 (d, 2H, J=8.8 Hz), 12.8 (brs, 1H).

### Example 15

### Compound 212

### 2-(4-(((1-(4-tert-butylbenzyl)-4-heptyl-5-oxo-4,5-dihydro-1H-1,2,4-triazol-3-yl)methoxy)carbonyl)-2-methoxyphenoxy)-2-methylpropanoic acid

### Preparation of 4-(1-tert-butoxy-2-methyl-1-oxopropan-2-yloxy)-3-methoxybenzoic acid (Acid D)

To a solution of methyl 4-hydroxy-3-methoxybenzoate (10.0 g, 54.9 mmol) and potassium carbonate (38 g, 275 mmol) in DMF (150 mL) was added tert-butyl 2-bromo-2-methylpropanoate (40 mL, 214 mmol, d=1.196). The reaction was stirred at 75° C. for 16 hours. The reaction was cooled and subjected to a standard work-up. The organic layer was concentrated to a colorless oil. The residue was taken up into MeOH (250 mL) and KOH pellets (20 g, 357 mmol) were added. The reaction was allowed to stir at room temperature for 16 hours before acidification to pH 2 with HCl (6M). Ether (200 mL) was added and the organics were separated. The aqueous layer was extracted twice more with ether (100 mL each). The combined organics were washed with brine, dried over MgSO₄, filtered and concentrated to a white solid (14.5 g). ¹H NMR (DMSO-d₆): δ 1.38 (s, 9H), 1.51 (s, 6H), 3.8 (s, 3H), 6.72 (d, 1H, J=3.2 Hz), 7.47 (m, 3H), 12.71 (br, 1H).

### Preparation of 2-(4-(((1-(4-tert-butylbenzyl)-4-heptyl-5-oxo-4,5-dihydro-1H-1,2,4-triazol-3-yl)methoxy)carbonyl)-2-methoxyphenoxy)-2-methylpropanoic acid

To 4-heptyl-3-(hydroxymethyl)-1-(4-methylbenzyl)-1H-1,2,4-triazol-5(4H)-one (170 mgs, 0.487 mmol) 4-(1-tert-butoxy-2-methyl-1-oxopropan-2-yloxy)-3-methoxybenzoic acid (Acid D) (151 mgs, 0.487 mmol) was added EDC (93 mgs, 0.487 mmol), DMAP (60 mgs, 0.487 mmol), and DMF (10 mL). The reaction was allowed to stir for 16 hours before it was subjected to a standard work-up and concentrated. The residue was dissolved in DCM (6 mL) to which was added TFA (3 mL). The reaction was allowed to stir for 1 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (27 mgs). ¹H NMR (DMSO-d₆): δ 0.78 (t, 3H, J=6.8 Hz), 1.08 (m, 8H), 1.25 (s, 9H), 1.42 (s, 6H), 1.57 (m, 2H), 3.64 (t, 2H, J=7.4 Hz), 3.79 (s, 3H), 4.85 (s, 2H), 5.23 (s, 2H), 6.80 (d, 1H, J=8.4 Hz), 7.80 (d, 2H, J=8.8 Hz), 7.45 (d, 2H, J=8.8 Hz), 7.45 (d, 1H, J=2.0 Hz), 7.51 (dd, 1H, J=2.0 Hz, J=8.4 Hz), 13.2 (brs, 1H).

### Example 16

### Compound 186

### 2-(2-methoxy-4-(((1-(4-methylbenzyl)-4-octyl-5-oxo-4,5-dihydro-1H-1,2,4-triazol-3-yl)methoxy)carbonyl)phenoxy)-2-methylpropanoic acid

To 3-(hydroxymethyl)-1-(4-methylbenzyl)-4-octyl-1H-1,2,4-triazol-5(4H)-one (540 mgs, 1.63 mmol) and 4-(1-tert-butoxy-2-methyl-1-oxopropan-2-yloxy)-3-methoxybenzoic acid (Acid D) (456 mgs, 1.47 mmol) was added EDC (312 mgs, 1.63 mmol), DMAP (199 mgs, 1.63 mmol), and DCM (20 mL). The reaction was allowed to stir for 16 hours, subjected to a standard work-up, and concentrated to an oil. To the residue, dissolved in DCM (6 mL), was added TFA (1 mL). The reaction was allowed to stir for 0.5 hour, concentrated, and purified via reverse phase high pressure liquid chromatography 1:9 ACN:water to 8.5:1.5 ACN:water over 20 minutes. The appropriate fractions were pooled, frozen and lyophilized to a white powder (27.7 mgs). ¹H NMR (DMSO-d₆): δ 0.81 (t, 3H, J=7.2 Hz), 1.25 (brm, 10H), 1.52 (s, 6H), 1.57 (brm, 2H), 2.27 (s, 3H), 3.64 (t, 2H, J=7.6 Hz), 3.79 (s, 3H), 4.84 (s, 2H), 5.23 (s, 2H), 6.80 (d, 1H, J=8.2 Hz), 7.14 (s, 4H), 7.44 (d, 1H, J=2.0 Hz), 7.50 (dd, 1H, J=3.0 Hz, J=8.8 Hz).

### Example 17

### Compound 151

### 2-(4-(((4-hexyl-1-(4-methylbenzyl)-5-oxo-4,5-dihydro-1H-1,2,4-triazol-3-yl)methoxy)carbonyl)phenoxy)-2-methylpropanoic acid

To 4-hexyl-3-(hydroxymethyl)-1-(4-methylbenzyl)-1H-1,2,4-triazol-5(4H)-one (102 mgs, 0.34 mmol) and 4-(1-tert-butoxy-2-methyl-1-oxopropan-2-yloxy)benzoic acid (Acid B) (100 mgs, 0.34 mmol) was added EDC (65 mgs, 0.34 mmol), DMAP (42 mgs, 0.34 mmol), and DMF (10 mL). The reaction was allowed to stir for 16 hours and subjected to a standard work-up. The residue was dissolved in DCM (6 mL) to which was added TFA (3 mL). The reaction was allowed to stir for 1 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (18.5 mgs). ¹H NMR (DMSO-d₆): δ 0.76 (t, 3H, J=6.8 Hz), 1.14 (brs, 6H), 1.56 (s, 8H), 2.27 (s, 3H), 3.64 (t, 2H, J=7.6 Hz), 4.84 (s, 2H), 5.22 (s, 2H), 6.87 (d, 2H J=9.2 Hz), 7.14 (s, 4H), 7.87 (d, 2H, J=8.8 Hz), 13.3 (brs, 1H).

### Example 18

### 2-methyl-2-(4-(2-((1-(4-methylbenzyl)-4-octyl-5-oxo-4,5-dihydro-1H-1,2,4-triazol-3-yl)methoxy)-2-oxoethyl)phenoxy)propanoic acid

To 3-(hydroxymethyl)-1-(4-methylbenzyl)-4-octyl-1H-1,2,4-triazol-5(4H)-one (67.5 mgs, 0.2 mmol) and 2-(4-(1-tert-butoxy-2-methyl-1-oxopropan-2-yloxy)phenyl)acetic acid (Acid C) (59 mgs, 0.2 mmol) was added EDC (38 mgs, 0.2 mmol), DMAP (24 mgs, 0.2 mmol), and DCM (10 mL). The reaction was allowed to stir for 16 hours before it was subjected to a standard work-up and concentrated to an oil. To the residue, dissolved in DCM (6 mL), was added TFA (1 mL). The reaction was allowed to stir for 0.25 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder (27.5 mgs). ¹H NMR (DMSO-d₆): δ 0.85 (t, 3H, J=6.8 Hz), 1.25 (brs, 10H), 1.34 (s, 6H), 1.54 (brm, 2H), 2.27 (s, 3H), 3.53 (t, 2H, J=8.0 Hz), 3.56 (s, 2H), 4.82 (s, 2H), 4.98 (s, 2H), 6.72 (d, 2H, J=8.4 Hz), 6.97 (d, 2H, J=8.4 Hz), 7.13 (s, 4H), 12.8 (brs, 1H).

### Example 19

### Compound 188

### 2-(4-(((1-(4-tert-butylbenzyl)-4-hexyl-5-oxo-4,5-dihydro-1H-1,2,4-triazol-3-yl)methoxy)carbonyl)phenoxy)-2-methylpropanoic acid

To 1-(4-tert-butylbenzyl)-4-hexyl-3-(hydroxymethyl)-1H-1,2,4-triazol-5(4H)-one (156 mgs, 0.45 mmol) and 4-(1-tert-butoxy-2-methyl-1-oxopropan-2-yloxy)benzoic acid (Acid B) (126 mgs, 0.45 mmol) was added EDC (86 mgs, 0.45 mmol), DMAP (55 mgs, 0.45 mmol), and DMF (10 mL). The reaction was allowed to stir for 16 hours before it was subjected to a standard work-up and concentrated to an oil. To the residue, dissolved in DCM (6 mL), was added TFA (3 mL). The reaction was allowed to stir for 0.5 hour before it was concentrated and purified via reverse phase high pressure liquid chromatography 1:9 ACN:water to 8.5:1.5 ACN:water over 20 minutes. The appropriate fractions were pooled, frozen and lyophilized to a white powder. ¹H NMR (DMSO-d₆): δ 0.75 (t, 3H, J=7.2 Hz), 1.16 (brm, 6H), 1.25 (s, 9H), 1.56 (s, 8H), 3.64 (t, 2H, J=8.0 Hz), 4.85 (s, 2H), 5.22 (s, 2H), 6.88 (d, 2H, J=9.2 Hz), 7.18 (d, 2H, J=8.4 Hz), 7.35 (d, 2H, J=8.4 Hz), 7.88 (d, 2H, J=9.6 Hz), 13.3 (brs, 1H).

### Example 20

### Compound 159

### 2-(4-(((1-(4-tert-butylbenzyl)-5-oxo-4-propyl-4,5-dihydro-1H-1,2,4-triazol-3-yl)methoxy)carbonyl)phenylthio)-2-methylpropanoic acid

To 1-(4-tert-butylbenzyl)-3-(hydroxymethyl)-4-propyl-1H-1,2,4-triazol-5(4H)-one (176 mgs, 0.58 mmol) and 4-(1-tert-butoxy-2-methyl-1-oxopropan-2-ylthio)benzoic acid (Acid A) (172 mgs, 0.58 mmol) was added EDC (111 mgs, 0.58 mmol), DMAP (71 mgs, 0.58 mmol), and DMF (10 mL). The reaction was allowed to stir for 16 hours before it was subjected to a standard work-up and concentrated to an oil. To the residue, dissolved in DCM (5 mL), was added TFA (2 mL). The reaction was allowed to stir for 1 hour, concentrated, and purified via reverse phase high pressure liquid chromatography 1:9 ACN:water to 8.5:1.5 ACN:water over 20 minutes. The appropriate fractions were pooled, frozen and lyophilized to a white powder. ¹H NMR (DMSO-d₆): δ 0.82 (t, 3H, J=7.6 Hz), 1.25 (s, 9H), 1.43 (s, 6H), 1.63 (sex, 2H), 3.65 (t, 2H, J=6.8 Hz), 4.87 (s, 2H), 5.26 (s, 2H), 7.18 (d, 2H, J=8.8 Hz), 7.35 (d, 2H, J=8.4 Hz), 7.56 (d, 2H, J=8.8 Hz), 7.91 (d, 2H, J=8.8 Hz), 12.8 (s, 1H).

### Example 21

### Compound 179

### 2-methyl-2-(4-(((5-oxo-4-propyl-1-(4-(trifluoromethylthio)benzyl)-4,5-dihydro-1H-1,2,4-triazol-3-yl)methoxy)carbonyl)phenylthio)propanoic acid

### Preparation of 4-propyl-1-(4-(trifluoromethylthio)benzyl)-3-(trityloxymethyl)-1H-1,2,4-triazol-5(4H)-one

4-Propyl-3-(trityloxymethyl)-1H-1,2,4-triazol-5(4H)-one (0.3 g, 0.751 mmol), 4-(trifluoromethylthio)benzyl bromide (0.20 g, 0.751 mmol), and 1,5,7-triazabicyclo[4.4.0]dec-5-ene bound to polystyrene (loading 2.6 mmol/g, 0.58 g) in dimethylformamide (5 mL) were heated at 65° C. overnight. The mixture was filtered and evaporated to give product as a white solid (0.36 g, 82%); MS for C₃₃H₃₀F₃N₃O₂S m/z 590 (M+H)⁺.

### Preparation of 3-(hydroxymethyl)-4-propyl-1-(4-(trifluoromethylthio)benzyl)-1H-1,2,4-triazol-5(4H)-one

4-Propyl-1-(4-(trifluoromethylthio)benzyl)-3-(trityloxymethyl)-1H-1,2,4-triazol-5(4H)-one (0.30 g, 0.509 mmol) and a solution of 2% trifluoroacetic acid and 5% triethylsilane in dichloromethane (5 mL) were stirred at room temperature for 2 hours. The mixture was evaporated and the residue purified by PTLC (50% ethyl acetate/hexanes) to give product as a white solid (0.17 g, 98%); MS for C₁₄H₁₆F₃N₃O₂S m/z 348 (M+H)⁺.

### Preparation of 2-methyl-2-(4-(((5-oxo-4-propyl-1-(4-(trifluoromethylthio)benzyl)-4,5-dihydro-1H-1,2,4-triazol-3-yl)methoxy)carbonyl)phenylthio)propanoic acid

4-(1-tert-butoxy-2-methyl-1-oxopropan-2-ylthio)benzoic acid (0.077 g, 0.26 mmol), 3-(hydroxymethyl)-4-propyl-1-(4-(trifluoromethylthio)benzyl)-1H-1,2,4-triazol-5(4H)-one (0.090 g, 0.26 mmol), N,N′-dicyclohexylcarbodiimide (0.064 g, 0.31 mmol), and 4-(dimethylamino)pyridine (0.004 mg, 0.031 mmol) in dichloromethane (5 mL) were stirred at room temperature overnight. The mixture was filtered to remove 1,3-dicyclohexylurea and the filtrate evaporated. The residue was purified by PTLC (30% ethyl acetate/hexane) to give (5-oxo-4-propyl-1-(4-(trifluoromethylthio)benzyl)-4,5-dihydro-1H-1,2,4-triazol-3-yl)methyl 4-(1-tert-butoxy-2-methyl-1-oxopropan-2-ylthio)benzoate as a white solid (0.069 g, 42%); MS for C₂₉H₃₄F₃N₃O₅S₂ m/z 626 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.040 g, 79%); HPLC retention time (Phenomenex 150×4.6 mm, 5μ, 1 mL/min, 20 mM ammonium acetate buffer, MeCN:water =15:85 for 2 min, then to 10:90 in 18 minutes)=14.1 min; ¹H-NMR (400 MHz, d₆-DMSO) 12.84 (bs, 1H); 7.91 (d, 2H, J=8 Hz); 7.70 (d, 2H, J=8 Hz); 7.56 (d, 2H, J=8 Hz); 7.39 (d, 2H, J=8 Hz); 5.28 (s, 2H); 5.00 (s, 2H); 3.66 (t, 2H, J=8 Hz); 1.64 (q, 2H, J=7 Hz); 1.42 (s, 6H); 0.83 (t, 3H, J=7 Hz); MS for C₂₅H₂₇F₃N₃O₅S₂ m/z 570 (M+H)⁺.

### Example 22

### Compound 199

### Synthesis of 2-Methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid

Method A Original Medchem Synthesis From Alcohol

### Preparation of methyl 2-(trityloxy)acetate

Trityl chloride (1 eq.) was added to methyl glycolate (1 eq.) and N,N′-diisopropylethylamine (1.5 eq.) in dichloromethane and stirred at room temperature for 48 hours. The mixture was washed with 10% aqueous citric acid and brine, dried (MgSO₄), and evaporated. The residue was purified by flash column chromatography (10% ethyl acetate/hexanes) to give product as a yellow oil (>80% yield).

### Preparation of 2-(trityloxy)acetohydrazide

Methyl 2-(trityloxy)acetate (1 eq.) and hydrazine monohydrate (5 eq.) in methanol were stirred overnight. The reaction was evaporated to give product as a white solid suitable for use directly in the next step (80%-90% yield).

### Preparation of N-Propyl-2-(2-(trityloxy)acetyl)hydrazinecarboxamide

2-(Trityloxy)acetohydrazide (1 eq.) and propyl isocyanate (1 eq.) in tetrahydrofuran were stirred overnight at room temperature. The reaction was evaporated to give product as a white solid suitable for use directly in the next step (>95% yield).

### Preparation of 4-Propyl-3-(trityloxymethyl)-1H-1,2,4-triazol-5(4H)-one

N-Propyl-2-(2-(trityloxy)acetyl)hydrazinecarboxamide (1 eq.) and potassium hydroxide (15 equiv) in methanol were heated at reflux for 16 hours. The reaction was partially evaporated under vacuum, acidified with 10% aqueous citric acid and extracted with dichloromethane. The organic layer was washed with brine, dried (MgSO₄) and evaporated to give product as a white solid suitable for use directly in the next step (70% yield).

### Preparation of 1-(4-Methylbenzyl)-4-propyl-3-(trityloxymethyl)-1H-1,2,4-triazol-5(4H)-one

4-Propyl-3-(trityloxymethyl)-1H-1,2,4-triazol-5(4H)-one (1 eq.), 4-methyl benzyl bromide (1.5 eq.) and potassium carbonate (2 eq.) in dimethylformamide were stirred at 75° C. for 16 hours. The mixture was diluted with water and extracted with dichloromethane. The organic layer was washed with brine, dried (MgSO₄) and evaporated. The residue was purified by flash column chromatography (10% ethyl acetate/hexanes) to give product as a white solid (70%-80% yield).

### Preparation of 3-(Hydroxymethyl)-1-(4-methylbenzyl)-4-propyl-1H-1,2,4-triazol-5(4H)-one

1-(4-Methylbenzyl)-4-propyl-3-(trityloxymethyl)-1H-1,2,4-triazol-5(4H)-one (1 eq.) and a solution containing 5% triethylsilane and 2.5% trifluoroacetic acid in dichloromethane were stirred at room temperature for 1 hour. The reaction was evaporated under vacuum to give product as a white solid suitable for use directly in the next step (80%-90% yield).

### Preparation of 1-(4-Methylbenzyl)-5-oxo-4-propyl-4,5-dihydro-1H-1,2,4-triazole-3-carbaldehyde

3-(Hydroxymethyl)-1-(4-methylbenzyl)-4-propyl-1H-1,2,4-triazol-5(4H)-one (1 eq.) and manganese (IV) oxide (2.2 eq.) in dioxane were heated at reflux for 16 hours. The mixture was filtered through celite and evaporated under vacuum. The residue was purified by flash column chromatography (15% ethyl acetate/hexanes) to give product as an oil (70%-75% yield).

### Preparation of 3-(1-hydroxyethyl)-1-(4-methylbenzyl)-4-propyl-1H-1,2,4-triazol-5(4H)-one

Methyl magnesium chloride (or bromide) in tetrahydrofuran (4 equiv) was added dropwise to 1-(4-methylbenzyl)-5-oxo-4-propyl-4,5-dihydro-1H-1,2,4-triazole-3-carbaldehyde (1 eq.) in tetrahydrofuran at 0° C. The mixture was allowed to warm to room temperature and stirred for 16 hours. The reaction was quenched with saturated aqueous ammonium chloride and extracted with ethyl acetate. The organic layer was washed with brine, dried (MgSO₄) and evaporated. The residue was purified by flash column chromatography (50% ethyl acetate/hexanes) to give product (85% yield).

### Preparation of 2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid

3-(1-hydroxyethyl)-1-(4-methylbenzyl)-4-propyl-1H-1,2,4-triazol-5(4H)-one (1 eq.), 4-(1-tert-butoxy-2-methyl-1-oxopropan-2-ylthio)benzoic acid (1 eq.), 1-ethyl-3-(3-dimethyl aminopropyl)carbodiimide hydrochloride (1.2 eq.), and 4-(dimethylamino)pyridine (1 eq.) in dimethylformamide were stirred at room temperature overnight. The mixture was diluted with ethyl acetate, washed with 10% aqueous citric acid, brine, saturated aqueous sodium bicarbonate and brine, dried (MgSO₄), and evaporated. The residue was purified by flash column chromatography (30% ethyl acetate/hexanes) to give 1-(1-(4-methylbenzyl)-5-oxo-4-propyl-4,5-dihydro-1H-1,2,4-triazol-3-yl)ethyl 4-(1-tert-butoxy-2-methyl-1-oxopropan-2-ylthio)benzoate as a clear oil (>70%). The intermediate t-butyl ester and hydrogen chloride in dioxane (4N) were stirred overnight at room temperature and then evaporated under vacuum. The residue was purified by flash column chromatography (10% methanol/dichloromethane) to give product as a white solid (60%-70% yield).

### Example 23

### Compound 249

### Synthesis of (R)-2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid

Method B Alternatively Compound 199 may be prepared starting from racemic methyl lactate following procedures described below for the preparation of the enantiomers of Compound 199, which are Compounds 248 and 249.

### Preparation of (R)-methyl 2-(benzyloxy)propanoate

Sodium hydride (60% dispersion in mineral oil, 10.6 g, 0.266 mol) was added portionwise to (+)-methyl-D-lactate (25.2 g, 0.242 mol)) and benzyl bromide (41.4 g, 0.242 mol) in dimethylformamide (125 mL) at 0° C. The mixture was stirred at 0° C. for 30 minutes, allowed to warm to room temperature and then stirred overnight. The reaction was cooled, diluted with 10% hydrochloric acid and extracted with ether. The ether extract was washed with water and brine, dried (MgSO₄) and evaporated. The residue was purified by flash column chromatography (5% ethyl acetate/hexanes) to give product as an oil (35.2 g, 75%).

### Preparation of (R)-2-(benzyloxy)propanehydrazide

(R)-methyl 2-(benzyloxy)propanoate (15.0 g, 0.0772 mol) and hydrazine monohydrate (18.7 mL, 0.39 mol) in methanol (100 mL) were stirred at room temperature overnight. The reaction mixture was evaporated under vacuum, the residue diluted with ethyl acetate, washed with water and brine, dried (MgSO₄), and evaporated to give product suitable for use directly in the next step as an oil (14.5 g, 97%).

### Preparation of (R)-2-(2-(benzyloxy)propanoyl)-N-propylhydrazinecarboxamide

Propyl isocyanate (7.09 mL, 0.0747 mol) and (R)-2-(benzyloxy)propanehydrazide (14.5 g, 0.0747 mol) in dichloromethane (100 mL) were stirred at room temperature overnight. The reaction mixture was evaporated and dried under vacuum to give product suitable for use directly in the next step as an oil (20.9 g, 99%).

### Preparation of (R)-3-(1-(benzyloxy)ethyl)-4-propyl-1H-1,2,4-triazol-5(4H)-one

(R)-2-(2-(benzyloxy)propanoyl)-N-propylhydrazinecarboxamide (20.9 g, 0.0747 mol) and potassium hydroxide (54.5 g, 0.971 mol) in ethanol (300 mL) were heated at reflux for 4 days. The reaction mixture was concentrated under vacuum, the residue carefully acidified with 6N hydrochloric acid and extracted with ethyl acetate. The extracts were washed with water and brine, dried (MgSO₄) and evaporated to give product suitable for use directly in the next step as an oil (19 g, 97%).

### Preparation of (R)-3-(1-(benzyloxy)ethyl)-1-(4-methylbenzyl)-4-propyl-1H-1,2,4-triazol-5(4H)-one

(R)-3-(1-(benzyloxy)ethyl)-4-propyl-1H-1,2,4-triazol-5(4H)-one (4.23 g, 0.0162 mol), 4-(methyl)benzyl bromide (3.00 g, 0.0162 mol) and potassium carbonate (3.36 g, 0.0243 mol) in dimethylformamide (20 mL) were heated at 80° C. overnight. The reaction mixture was diluted with water and extracted with ethyl acetate. The extracts were washed with brine, dried (MgSO₄) and evaporated to give residue, which was purified by Biotage flash chromatography system (30% ethyl acetate/hexanes) to give product as an oil (3.35 g, 57%).

### Preparation of (R)-3-(1-hydroxyethyl)-1-(4-methylbenzyl)-4-propyl-1H-1,2,4-triazol-5(4H)-one

(R)-3-(1-(benzyloxy)ethyl)-1-(4-methylbenzyl)-4-propyl-1H-1,2,4-triazol-5(4H)-one (3.35 g, 0.00917 mol), palladium on carbon (10%, wet, Degussa, 0.67 g), and acetic acid (2 mL) in ethanol (18 mL) were hydrogenated at 60 psi on a Parr apparatus overnight. The catalyst was removed by filtration and the filtrate evaporated under vacuum. The residue was dissolved in ethyl acetate, washed with saturated aqueous sodium bicarbonate and brine, dried (MgSO₄), and evaporated to give product suitable for use in the next step as an oil (2.5 g, 99%).

### Preparation of (R)-2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid

N-(3-Dimethylaminopropyl)-N′-ethylcarbodiimide hydrochloride (2.01 g, 0.0105 mol) was added to (R)-3-(1-hydroxyethyl)-1-(4-methylbenzyl)-4-propyl-1H-1,2,4-triazol-5(4H)-one (2.40 g, 0.00872 mol), 4-(1-tert-butoxy-2-methyl-1-oxopropan-2-ylthio)benzoic acid (2.58 g, 0.00872 mol) and 4-di(methylamino)pyridine (1.07 g, 0.00872 mol) in dimethylformamide (20 mL) and stirred at room temperature overnight. The mixture was diluted with ethyl acetate, washed with aqueous 10% citric acid, saturated aqueous sodium bicarbonate and brine. The organic layer was dried (MgSO₄) and evaporated to give (R)-1-(1-(4-methylbenzyl)-5-oxo-4-propyl-4,5-dihydro-1H-1,2,4-triazol-3-yl)ethyl 4-(1-tert-butoxy-2-methyl-1-oxopropan-2-ylthio)benzoate as an oil. The intermediate t-butyl ester and hydrogen chloride in dioxane (4N, 20 mL) were stirred overnight at room temperature and then evaporated to dryness. The residue was purified by Biotage flash chromatography system (5% methanol/dichloromethane) to give product as an oil (3.69 g, 85%).

### Example 24

### Compound 248

### Synthesis of (S)-2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid

### Preparation of (S)-methyl 2-(benzyloxy)propanoate

Sodium hydride (60% dispersion in mineral oil, 10.6 g, 0.264 mol) was added portionwise to (−)-methyl-L-lactate (25 g, 0.240 mol)) and benzyl bromide (28.5 mL, 0.240 mol) in dimethylformamide (125 mL) at 0° C. The mixture was stirred at 0° C. for 30 minutes, allowed to warm to room temperature and then stirred overnight. The reaction was cooled, diluted with 10% hydrochloric acid and extracted with ether. The ether extract was washed with water and brine, dried (MgSO₄) and evaporated. The residue was purified by flash column chromatography (5% ethyl acetate/hexanes) to give product as an oil (33.6 g, 72%).

### Preparation of (S)-2-(benzyloxy)propanehydrazide

(S)-Methyl 2-(benzyloxy)propanoate (15.0 g, 0.0772 mol) and hydrazine monohydrate (18.7 mL, 0.386 mol) in methanol (100 mL) were stirred at room temperature overnight. The reaction mixture was evaporated under vacuum, the residue diluted with ethyl acetate, washed with water and brine, dried (MgSO₄), and evaporated to give product suitable for use directly in the next step as an oil (14.2 g, 95%).

### Preparation of (S)-2-(2-(benzyloxy)propanoyl)-N-propylhydrazinecarboxamide

Propyl isocyanate (6.94 mL, 0.0732 mol) and (S)-2-(benzyloxy)propanehydrazide (14.2 g, 0.0732 mol) in dichloromethane (100 mL) were stirred at room temperature overnight. The reaction mixture was evaporated and dried under vacuum to give product suitable for use directly in the next step as an oil (20.3 g, 99%).

### Preparation of (S)-3-(1-(benzyloxy)ethyl)-4-propyl-1H-1,2,4-triazol-5(4H)-one

(S)-2-(2-(benzyloxy)propanoyl)-N-propylhydrazinecarboxamide (20.3 g, 0.0732 mol) and potassium hydroxide (53.4 g, 0.952 mol) in ethanol (300 mL) were heated at reflux for 4 days. The reaction mixture was concentrated under vacuum, the residue carefully acidified with 6N hydrochloric acid and extracted with ethyl acetate. The extracts were washed with water and brine, dried (MgSO₄) and evaporated to give product suitable for use directly in the next step as an oil.

### Preparation of (S)-3-(1-(benzyloxy)ethyl)-1-(4-methylbenzyl)-4-propyl-1H-1,2,4-triazol-5(4H)-one

(S)-3-(1-(benzyloxy)ethyl)-4-propyl-1H-1,2,4-triazol-5(4H)-one (3.00 g, 0.0115 mol), 4-(methyl)benzyl bromide (2.12 g, 0.0115 mol) and potassium carbonate (2.38 g, 0.0173 mol) in dimethylformamide (50 mL) were heated at 80° C. overnight. The reaction mixture was diluted with water and extracted with ethyl acetate. The extracts were washed with brine, dried (MgSO₄) and evaporated to give a residue, which was purified by Biotage flash chromatography system to give product as an oil (2.73 g, 65%).

### Preparation of (S)-3-(1-hydroxyethyl)-1-(4-methylbenzyl)-4-propyl-1H-1,2,4-triazol-5(4H)-one

(S)-3-(1-(benzyloxy)ethyl)-1-(4-methylbenzyl)-4-propyl-1H-1,2,4-triazol-5(4H)-one (2.70 g, 0.00739 mol), palladium on carbon (10%, wet, Degussa, 0.54 g), and acetic acid (2 mL) in ethanol (18 mL) were hydrogenated at 60 psi on a Parr apparatus overnight. The catalyst was removed by filtration and the filtrate evaporated under vacuum. The residue was dissolved in ethyl acetate, washed with saturated aqueous sodium bicarbonate and brine, dried (MgSO₄), and evaporated to give product suitable for use in the next step as an oil (2.0 g, 99%).

### Preparation of (S)-2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid

N-(3-dimethylaminopropyl)-N′-ethylcarbodiimide hydrochloride (1.67 g, 0.00872 mol) was added to (S)-3-(1-hydroxyethyl)-1-(4-methylbenzyl)-4-propyl-1H-1,2,4-triazol-5(4H)-one (2.00 g, 0.00726 mol), 4-(1-tert-butoxy-2-methyl-1-oxopropan-2-ylthio)benzoic acid (2.15 g, 0.00726 mol) and 4-di(methylamino)pyridine (0.871 g, 0.00726 mol) in dimethylformamide (20 mL) and stirred at room temperature overnight. The mixture was diluted with ethyl acetate, washed with aqueous 10% citric acid, saturated aqueous sodium bicarbonate and brine. The organic layer was dried (MgSO₄) and evaporated to give (S)-1-(1-(4-methylbenzyl)-5-oxo-4-propyl-4,5-dihydro-1H-1,2,4-triazol-3-yl)ethyl 4-(1-tert-butoxy-2-methyl-1-oxopropan-2-ylthio)benzoate as an oil. The intermediate t-butyl ester and hydrogen chloride in dioxane (4N, 20 mL) were stirred overnight at room temperature and then evaporated to dryness. The residue was purified by Biotage flash chromatography system (5% methanol/dichloromethane) to give product as an oil (3.82 g, 88%).

### Example 25

### Compound 252

### 2-(4-((1-(1-(4-tert-butylbenzyl)-5-oxo-4-propyl-4,5-dihydro-1H-1,2,4-triazol-3-yl)-2,2,2-trifluoroethoxy)carbonyl)phenylthio)-2-methylpropanoic acid

### Preparation of methyl 2-(trityloxy)acetate

To a solution of Hunig's base (8.7 ml, 49.9 mmol, 1.5 equiv) in DCM, methyl glycolate (3 g, 33.3 mmol, 1 equiv.) was added followed by trityl chloride (9.28 g, 33.3 mmol, 1 equiv.). The reaction was allowed to stir at room temperature for 48 hours before it was quenched with water. The aqueous layer was extracted 2-3 times with dichloromethane. The combined organic layers were washed with brine, dried (MgSO₄), filtered and concentrated. The desired product was purified using column chromatography (10% ethyl acetate:hexanes) to afford the desired product as a yellow oil (8.85 g, >80% yield).

### Preparation of 2-(trityloxy)acetohydrazide

To a solution of methyl 2-(trityloxy)acetate (8 g, 24.08 mmol, 1 equiv) in MeOH was added hydrazine-monohydrate (5.8 ml, 120.4 mmol, 5 equiv.). The reaction was allowed to stir for 16 hours at room temperature before it was concentrated and dried to a white powder (8.5 g, 80%-90% yield).

### Preparation of N-propyl-2-(2-(trityloxy)acetyl)hydrazinecarboxamide

2-(trityloxy)acetohydrazide (8 g, 24.1 mmol, 1 equiv) was dissolved in THF to which propyl isocyanate (2.3 ml, 24.2 mmol, 1 equiv) was added. The reaction was allowed to stir at room temperature for 16 hours before it was concentrated to a white powder (7.6 g, >95% yield).

### Preparation of 4-Propyl-3-(trityloxymethyl)-1H-1,2,4-triazol-5(4H)-one

To a solution of N-Propyl-2-(2-(trityloxy)acetyl)hydrazinecarboxamide (7 g, 16.78 mmol, 1 equiv) in methanol, was added potassium hydroxide (14.1 g, 252 mmol, 15 equiv). The mixture was refluxed for 16 hours, concentrated in vacuo, and acidified with 10% citric acid. The product was filtered, redissolved in DCM and washed with brine. The organic layer was dried (MgSO₄), filtered, and concentrated. The resulting residue was purified using column chromatography (50% ethyl acetate:hexanes) to yield the desired product as a white solid (4.7 g, 70% yield).

### Preparation of 1-(4-tert-butylbenzyl)-4-propyl-3-(trityloxymethyl)-1H-1,2,4-triazol-5(4H)-one

To a solution of 4-propyl-3-(trityloxymethyl)-1H-1,2,4-triazol-5(4H)-one (4.5 g, 11.2 mmol, 1 equiv) in DMF was added 4-tert-butylbenzyl bromide (3.81 g, 16.8 mmol, 1.5 equiv) and potassium carbonate (3.09 g, 22.4 mmol, 2 equiv). The reaction was heated to 65° C. for 16 hours before water was slowly added. The mixture was allowed to stir for 30 minutes before the solid was filtered and washed with minimal amount of hexanes. The filter cake was dried to a white solid (4.88 g, 70%-80% yield).

### Preparation of 1-(4-tert-butylbenzyl)-3-(hydroxymethyl)-4-propyl-1H-1,2,4-triazol-5(4H)-one

1-(4-tert-butylbenzyl)-4-propyl-3-(trityloxymethyl)-1H-1,2,4-triazol-5(4H)-one (4.5 g, 8.89 mmol, 1 equiv) was dissolved in a mixture of 5% triethylsilane, 2.55 trifluoroacetic acid and 92.5% dichloromethane. The reaction was stirred for 1 hour before it was concentrated and purified using column chromatography (50%-60% ethyl acetate:hexanes) to yield the desired alcohol (2.3 g, 80%-90% yield).

MW=371; [M+H]=371.97.

¹HNMR (DMSO-d₆)-δ 7.46-7.44 (d, 1H, J=7.2 Hz), 7.37-7.34 (m, 2H), 7.17-7.14 (m, 2H), 5.39-5.31 (p, 1H), 4.86 (s, 2H), 3.7-3.63 (m, 2H), 1.7-1.6 (m, 2H), 1.25 (s, 9H), 0.87-0.84 (t, 3H, J=7.2 & 7.6 Hz).

### Preparation of 1-(4-tert-butylbenzyl)-5-oxo-4-propyl-4,5-dihydro-1H-1,2,4-triazole-3-carbaldehyde

3-(Hydroxymethyl)-1-(4-tert-butylbenzyl)-4-propyl-1H-1,2,4-triazol-5(4H)-one (2.3 g, 7.54 mmol, 1 equiv) was taken into dioxane to which manganese (IV) oxide (1.44 g, 16.6 mmol, 2.2 equiv) was added. The reaction was refluxed at 108° C. for 16 hours. The reaction was allowed to cool to room temperature, filtered through celite, and washed the celite thoroughly with DCM. The filtrate was then concentrated and purified using column chromatography (15% ethyl acetate:hexanes) to afford the desired aldehyde (3.5 g, 70%-75% yield) as a clear oil.

### Preparation of 1-(4-tert-butylbenzyl)-4-propyl-3-(2,2,2-trifluoro-1-hydroxyethyl)-1H-1,2,4-triazol-5(4H)-one

A 0.5 M solution of trimethyl(trifluoromethyl)silane in THF (5 ml, 2.5 mmol, 1.5 equiv) was added to a solution of 1-(4-tert-butylbenzyl)-5-oxo-4-propyl-4,5-dihydro-1H-1,2,4-triazole-3-carbaldehyde (500 mg, 1.66 mmol, 1 equiv) and TBAF (83 mL, 0.083 mmol, 0.05 equiv) in THF (10 mL) cooled to 0° C. under argon. The mixture was stirred at 0° C. for 2 h and then at room temperature for 72 h. Trimethyl(trifluoromethyl)silane was added in two portions, spaced by one day. Saturated ammonium chloride was added to the reaction after 3 days and the compound was extracted with ether (2-3 times). The combined organic layers were dried over MgSO₄ and purified using Biotage SP-4 chromatography system (1:1 hexanes:ethyl acetate as the eluent condition) to afford the desired product as a yellow foam (332 mg, 53.9% yield).

MW=371; [M+H]=371.97

¹HNMR (DMSO-d₆)-δ 7.46-7.44 (d, 1H, J=7.2 Hz), 7.37-7.34 (m, 2H), 7.17-7.14 (m, 2H), 5.39-5.31 (p, 1H), 4.86 (s, 2H), 3.7-3.63 (m, 2H), 1.7-1.6 (m, 2H), 1.25 (s, 9H), 0.87-0.84 (t, 3H, J=7.2 & 7.6 Hz).

### Preparation of 1-(1-(4-tert-butylbenzyl)-5-oxo-4-propyl-4,5-dihydro-1H-1,2,4-triazol-3-yl)-2,2,2-trifluoroethyl 4-(1-tert-butoxy-2-methyl-1-oxopropan-2-ylthio)benzoate

1-(4-tert-butylbenzyl)-4-propyl-3-(2,2,2-trifluoro-1-hydroxyethyl)-1H-1,2,4-triazol-5(4H)-one (332 mg, 0.895 mmol, 1 equiv), 4-(1-tert-butoxy-2-methyl-1-oxopropan-2-ylthio)benzoic acid (265 mg, 0.895 mmol, 1 equiv), and dimethylaminopyridine (109.3 mg, 0.895 mmol, 1 equiv) were taken into dimethylformamide (5 ml). 1-(3-dimethylaminopropyl)-3-ethylcarbodiimide hydrochloride (205.13 mg, 1.074 mmol, 1.2 equiv) was added to the reaction mixture and stirred at room temperature for 16 hours. The reaction was worked up with ethyl acetate, 10% citric acid and brine, then dried over MgSO₄. The residue obtained after concentration was purified using Biotage SP-4 chromatography system (4:1 hexanes:ethyl acetate as the eluent condition) to afford the desired product as a syrup (433 mg, 74.5% yield).

MW=649; [M+H]=650.07

¹HNMR (DMSO-d₆)-δ 8.0-7.97 (m, 2H), 7.62-7.60 (m, 2H), 7.30-7.28 (m, 2H), 7.08-7.06 (m, 2H), 6.99-6.95 (q, 1H, J=6.4 Hz), 4.89 (s, 2H), 3.76-3.72 (m, 2H), 1.63-1.58 (m, 2H), 1.44 (bs, 6H), 1.33 (s, 9H), 1.22 (s, 9H), 0.84-0.81 (t, 3H, J=7.2 & 7.6 Hz).

### Preparation of 2-(4-((1-(1-(4-tert-butylbenzyl)-5-oxo-4-propyl-4,5-dihydro-1H-1,2,4-triazol-3-yl)-2,2,2-trifluoroethoxy)carbonyl)phenylthio)-2-methylpropanoic acid

1-(1-(4-tert-butylbenzyl)-5-oxo-4-propyl-4,5-dihydro-1H-1,2,4-triazol-3-yl)-2,2,2-trifluoroethyl 4-(1-tert-butoxy-2-methyl-1-oxopropan-2-ylthio)benzoate (433 mg, 0.667 mmol, 1 equiv) were taken into 4 M HCl/dioxane and the reaction was stirred for 16 hours. It was concentrated and the resulting residue was purified using Biotage SP-4 chromatography system (9:1 mixture of dichloromethane:methanol as the eluent condition) to afford the desired compound as a white powder (320 mg, 80.8% yield).

MW=593; [M+H]=593.99

¹HNMR (DMSO-d₆)-δ 12.88 (bs, 1H), 7.99-7.96 (m, 2H), 7.62-7.59 (m, 2H), 7.31-7.28 (m, 2H), 7.08-7.06 (m, 2H), 6.98-6.94 (q, 1H, J=6.4 Hz), 4.89 (s, 2H), 3.76-3.72 (m, 2H), 1.64-1.59 (m, 2H), 1.45 (bs, 6H), 1.22 (s, 9H), 1.22 (s, 9H), 0.85-0.82 (t, 3H, J=7.2 Hz).

### Example 26

### Compound 78

### 2-(4-(((1-Benzyl-1H-1,2,3-triazol-4-yl)methoxy)carbonyl)phenoxy)-2-methylpropanoic acid

4-(1-tert-Butoxy-2-methyl-1-oxopropan-2-yloxy)benzoic acid (0.44 g, 1.59 mmol), (1-benzyl-1H-1,2,3-triazol-4-yl)methanol (0.30 g, 1.59 mmol), N,N′-dicyclohexylcarbodiimide (0.39 g, 1.91 mmol), and 4-(dimethylamino)pyridine (0.0229 mg, 0.191 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-benzyl-1H-1,2,3-triazol-4-yl)methyl 4-(1-tert-butoxy-2-methyl-1-oxopropan-2-yloxy)benzoate as a white solid (0.65 g, 90%); MS for C₂₅H₂₉N₃O₅ m/z 452 (M+H)⁺. The ester and 4M hydrogen chloride solution in dioxane (4 mL) were stirred at room temperature overnight and the mixture was evaporated under vacuum. The residue was purified by PTLC (10% methanol/dichloromethane) to give product as a white solid (0.46 g, 82%); ¹H NMR (400 MHz, DMSO-d₆) δ 8.28 (s, 1H); 7.82 (m, 2H); 7.35 (m, 4H); 6.86 (m, 2H); 5.60 (s, 2H); 5.33 (s, 2H); 1.51 (s, 6H); MS for C₂₁H₂₁N₃O₅ m/z 396 (M+H)⁺.

### Preparation of (1-Benzyl-1H-1,2,3-triazol-4-yl)methanol

(Azidomethyl)benzene (7.79 g, 58.5 mmol) propargyl alcohol (3.46 mL, 58.5 mmol), copper sulfate (0.73 g, 2.93 mmol), and sodium ascorbate (1.16 g, 5.85 mmol) in water/dimethylformamide (1:4, 80 mL) were stirred at 65° C. overnight. The mixture was diluted with water, filtered, and extracted with ethyl acetate. The extract was washed with brine, dried (MgSO₄), and evaporated to give product as a white solid (9.02 g, 82%); ¹H NMR (400 MHz, DMSO-d₆) δ 8.01 (s, 1H); 7.30-7.39 (m, 5H); 5.57 (s, 2H); 5.16 (t, J=5.6 Hz, 1H); 4.50 (d, J=5.6 Hz, 2H); MS for C₁₀H₁₁N₃O m/z 190 (M+H)⁺.

### Preparation of (Azidomethyl)benzene

Benzyl bromide (10.0 g, 58.4 mmol) and sodium azide (5.70 g, 87.6 mmol) in dimethylformamide (75 mL) were stirred at 65° C. for 4 hours. The mixture was diluted with water and extracted with ether. The extract was washed with brine, dried (MgSO₄), and evaporated to give product as an oil (7.79 g, 99%); MS for C₇H₇N₃ m/z 134 (M+H)⁺.

### Example 27

### Compound 77

### 2-(4-(((1-Benzyl-1H-1,2,3-triazol-4-yl)methoxy)carbonyl)phenylthio)-2-methylpropanoic acid

4-(1-tert-Butoxy-2-methyl-1-oxopropan-2-ylthio)benzoic acid (0.47 g, 1.59 mmol), (1-benzyl-1H-1,2,3-triazol-4-yl)methanol (0.30 g, 1.59 mmol), N,N′-dicyclohexylcarbodiimide (0.39 g, 1.91 mmol), and 4-(dimethylamino)pyridine (0.0229 mg, 0.191 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-benzyl-1H-1,2,3-triazol-4-yl)methyl 4-(1-tert-butoxy-2-methyl-1-oxopropan-2-ylthio)benzoate as a white solid (0.68 g, 92%); MS for C₂₅H₂₉N₃O₄S m/z 468 (M+H)⁺. The ester and 4M hydrogen chloride solution in dioxane (4 mL) were stirred at room temperature overnight and evaporated under vacuum. The residue was purified by PTLC (10% methanol/dichloromethane) to give product as a white solid (0.52 g, 86%); ¹H NMR (400 MHz, DMSO-d₆) δ 8.31 (s, 1H); 7.90 (d, J=7.2 Hz, 2H); 7.54 (d, J=7.2 Hz, 2H); 7.30-7.39 (m, 4H); 5.61 (s, 2H); 5.39 (s, 2H); 1.42 (s, 6H); MS for C₂₁H₂₁N₃O₄S m/z 412 (M+H)⁺.

### Example 28

### Compound 70

### 2-(4-(((1-(4-tert-Butylbenzyl)-1H-1,2,3-triazol-4-yl)methoxy)carbonyl)phenoxy)-2-methylpropanoic acid

4-(1-tert-Butoxy-2-methyl-1-oxopropan-2-yloxy)benzoic acid (0.23 g, 0.815 mmol), (1-(4-tert-butylbenzyl)-1H-1,2,3-triazol-4-yl)methanol (0.20 g, 0.815 mmol), N,N′-dicyclohexylcarbodiimide (0.20 g, 0.978 mmol), and 4-(dimethylamino)pyridine (0.0117 mg, 0.0978 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-tert-butylbenzyl)-1H-1,2,3-triazol-4-yl)methyl 4-(1-tert-butoxy-2-methyl-1-oxopropan-2-yloxy)benzoate as an oil (0.38 g, 91%); MS for C₂₉H₃₇N₃O₅ m/z 508 (M+H)⁺. The ester and 4M hydrogen chloride solution in dioxane (4 mL) were stirred at room temperature overnight and evaporated under vacuum. The residue was purified by PTLC (10% methanol/dichloromethane) to give product as a white solid (0.26 g, 78%); ¹H NMR (400 MHz, DMSO-d₆) δ 8.27 (s, 1H); 7.85 (m, 2H); 7.38 (d, J=8.8 Hz, 2H); 7.26 (d, J=8.8 hz, 2H); 6.87 (m, 2H); 5.55 (s, 2H); 5.33 (s, 2H); 1.55 (s, 6H); 1.25 (s, 9H); MS for C₂₅H₂₉N₃O₅ m/z 452 (M+H)⁺.

### Preparation of (1-(4-tert-Butylbenzyl)-1H-1,2,3-triazol-4-yl)methanol

1-(Azidomethyl)-4-tert-butylbenzene (10.6 g, 56.0 mmol) propargyl alcohol (3.31 mL, 56.0 mmol), copper sulfate (0.70 g, 2.80 mmol), and sodium ascorbate (1.11 g, 5.60 mmol) in water/dimethylformamide (1:4, 80 mL) were stirred at 65° C. overnight. The mixture was diluted with water, filtered, and extracted with ethyl acetate. The extract was washed with brine, dried (MgSO₄), and evaporated to give product as a white solid (11.7 g, 85%); ¹H NMR (400 MHz, DMSO-d₆) δ 7.98 (s, 1H); 7.38 (d, J=8.8 Hz, 2H); 7.24 (d, J=8.8 Hz, 2H); 5.51 (s, 2H); 5.13 (t, J=5.6 Hz, 1H); 4.49 (d, J=5.6 Hz, 2H); 1.25 (s, 9H); MS for C₁₄H₁₉N₃O m/z 246 (M+H)⁺.

### Preparation of 1-(Azidomethyl)-4-tert-butylbenzene

4-(tert-Butyl)benzyl bromide (10.0 mL, 54.4 mmol) and sodium azide (5.31 g, 81.6 mmol) in dimethylformamide (80 mL) were stirred at 65° C. for 4 hours. The mixture was diluted with water and extracted with ether. The extract was washed with brine, dried (MgSO₄), and evaporated to give product as an oil (10.3 g, 99%); MS for C₁₁H₁₅N₃ m/z 190 (M+H)⁺.

### Example 29

### Compound 69

### 2-(4-(((1-(4-tert-Butylbenzyl)-1H-1,2,3-triazol-4-yl)methoxy)carbonyl)phenylthio)-2-methylpropanoic acid

4-(1-tert-Butoxy-2-methyl-1-oxopropan-2-ylthio)benzoic acid (0.24 g, 0.815 mmol), (1-(4-tert-butylbenzyl)-1H-1,2,3-triazol-4-yl)methanol (0.20 g, 0.815 mmol), N,N′-dicyclohexylcarbodiimide (0.20 g, 0.978 mmol), and 4-(dimethylamino)pyridine (0.0117 mg, 0.0978 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-tert-butylbenzyl)-1H-1,2,3-triazol-4-yl)methyl 4-(1-tert-butoxy-2-methyl-1-oxopropan-2-ylthio)benzoate as an oil (0.38 g, 89%); MS for C₂₉H₃₇N₃O₄S m/z 524 (M+H)⁺. The ester and 4M hydrogen chloride solution in dioxane (4 mL) were stirred at room temperature overnight and then evaporated under vacuum. The residue was purified by PTLC (10% methanol/dichloromethane) to give product as a white solid (0.31 g, 92%); ¹H NMR (400 MHz, DMSO-d₆) δ 8.29 (s, 1H); 7.89 (m, 2H); 7.56 (m, 2H); 7.38 (d, J=8.4 Hz, 2H); 7.26 (d, J=8.4 Hz, 2H); 5.56 (s, 2H); 5.38 (s, 2H); 1.41 (s, 6H); 1.25 (s, 9H); MS for C₂₅H₂₉N₃O₄S m/z 468 (M+H)⁺.

### Example 30

### Compound 61

### 2-Methyl-2-(4-(((1-(4-methylbenzyl)-1H-1,2,3-triazol-4-yl)methoxy)carbonyl)phenoxy)propanoic acid

To 4-(1-tert-butoxy-2-methyl-1-oxopropan-2-yloxy)benzoic acid (0.69 g, 2.46 mmol), was added a mixture of (1-(4-methylbenzyl)-1H-1,2,3-triazol-4-yl)methanol and (1-(4-methylbenzyl)-1H-1,2,3-triazol-5-yl)methanol (0.50 g, 2.46 mmol), N,N′-dicyclohexylcarbodiimide (0.61 g, 2.95 mmol), and 4-(dimethylamino)pyridine (0.035 g, 0.295 mmol) in dichloromethane (10 mL) and stirred at room temperature overnight. The mixture was filtered to remove 1,3-dicyclohexylurea and the filtrate evaporated. The residue was purified by PTLC (30% ethyl acetate/hexane) to give the less polar (1-(4-methylbenzyl)-1H-1,2,3-triazol-4-yl)methyl 4-(1-tert-butoxy-2-methyl-1-oxopropan-2-yloxy)benzoate as a white solid (0.60 g); ¹H NMR (400 MHz, DMSO-d₆) δ 8.24 (s, 1H); 7.86 (d, J=9.2 Hz, 2H); 7.20 (dd, J=8 and 24 Hz, 4H); 6.84 (d, J=9.2 Hz, 2H); 5.54 (s, 2H); 5.33 (s, 2H); 2.27 (s, 3H); 1.55 (s, 6H); 1.36 (s, 9H); MS for C₂₆H₃₁N₃O₅ m/z 466 (M+H)⁺ and the more polar (1-(4-methylbenzyl)-1H-1,2,3-triazol-5-yl)methyl 4-(1-tert-butoxy-2-methyl-1-oxopropan-2-yloxy)benzoate (0.43 g); ¹H NMR (400 MHz, DMSO-d₆) δ 7.89 (s, 1H); 7.55 (d, J=9.2 Hz, 2H); 7.06 (dd, J=8 and 16 Hz, 4H); 6.73 (d, J=9.2 Hz, 2H); 5.69 (s, 2H); 5.39 (s, 2H); 2.21 (s, 3H); 1.55 (s, 6H); 1.36 (s, 9H). (1-(4-Methylbenzyl)-1H-1,2,3-triazol-4-yl)methyl 4-(1-tert-butoxy-2-methyl-1-oxopropan-2-yloxy)benzoate and 4M hydrogen chloride solution in dioxane (4 mL) were stirred at room temperature overnight and evaporated under vacuum. The residue was purified by PTLC (10% methanol/dichloromethane) to give product as a white solid (0.46 g, 87%); ¹H NMR (400 MHz, DMSO-d₆) δ 8.24 (s, 1H); 7.86 (d, J=6.4 Hz, 2H); 7.20 (dd, J=8 and 24 Hz, 4H); 6.87 (d, J=8Hz, 2H); 5.54 (s, 2H); 5.33 (s, 2H); 2.27 (s, 3H); 1.56 (s, 6H); MS for C₂₂H₂₃N₃O₅ m/z 410 (M+H)⁺.

### Preparation of regioisomeric mixture of (1-(4-methylbenzyl)-1H-1,2,3-triazol-4-yl)methanol and (1-(4-methylbenzyl)-1H-1,2,3-triazol-5-yl)methanol

1-(Azidomethyl)-4-methylbenzene (3.97 g, 27.0 mmol) and propargyl alcohol (1.57 mL, 27.0 mmol) in toluene (5 mL) were stirred and heated at 90° C. overnight. The mixture was evaporated under vacuum and the residue purified by flash column chromatography (50% ethyl acetate/hexane) to provide a mixture of [1-(4-methyl-benzyl)-1H-[1,2,3]triazol-4-yl]-methanol; EMD Silica Gel 60 250 μM TLC R_{f}(ethyl acetate)=0.3 (59% by ¹H NMR) and (1-(4-methylbenzyl)-1H-1,2,3-triazol-5-yl)methanol; EMD Silica Gel 60 250 μM TLC R_{f} (ethyl acetate)=0.4 (41% by ¹H NMR) used directly in the next step (4.9 g, 90%); MS for C₁₁H₁₃N₃O m/z 204 (M+H)⁺.

### Preparation of (1-(4-methylbenzyl)-1H-1,2,3-triazol-4-yl)methanol

1-(Azidomethyl)-4-methylbenzene (8.30 g, 56.4 mmol) propargyl alcohol (3.30 mL, 56.4 mmol), copper sulfate (0.70 g, 2.82 mmol), and sodium ascorbate (1.12 g, 5.64 mmol) in water/dimethylformamide (1:4, 80 mL) were stirred at 65° C. overnight. The mixture was diluted with water, filtered, and extracted with ethyl acetate. The extract was washed with brine, dried (MgSO₄), and evaporated to give an oil. Titration with ether/hexanes provides product as a white solid (7.2 g, 63%); ¹H NMR (400 MHz, DMSO-d₆) δ 7.96 (s, 1H); 7.19 (dd, J=8.0 and 16 Hz, 4H); 5.50 (s, 2H); 5.13 (t, J=5.6 Hz, 1H); 4.49 (d, J=5.6 Hz, 2H); 2.28 (s, 3H); MS for C₁₁H₁₃N₃O m/z 204 (M+H)⁺.

### Preparation of 1-(Azidomethyl)-4-methylbenzene

4-Methylbenzyl bromide (5.0 g, 27.0 mmol) and sodium azide (1.93 g, 29.7 mmol) in dimethylformamide (20 mL) were stirred at 65° C. for 4 hours. The mixture was diluted with water and extracted with ether. The extract was washed with brine, dried (MgSO₄), and evaporated to give product as an oil (3.9 g, 99%); MS for C₈H₉N₃ m/z 148 (M+H)⁺.

### Example 31

### Compound 63

### 2-Methyl-2-(4-(((1-(4-methylbenzyl)-1H-1,2,3-triazol-5-yl)methoxy)carbonyl)phenoxy)propanoic acid

(1-(4-Methylbenzyl)-1H-1,2,3-triazol-5-yl)methyl 4-(1-tert-butoxy-2-methyl-1-oxopropan-2-yloxy)benzoate (0.43 g, 0.924 mmol) and 4M hydrogen chloride solution in dioxane (4 mL) were stirred at room temperature overnight and the evaporated under vacuum. The residue was purified by PTLC (10% methanol/dichloromethane) to give product as a white solid (0.34 g, 89%); ¹H NMR (400 MHz, DMSO-d₆) δ 7.88 (s, 1H); 7.52 (d, J=8.0 Hz, 2H); 7.07 (dd, J=8 and 20 Hz, 4H); 6.76 (d, J=8.0, 2H); 5.69 (s, 2H); 5.38 (s, 2H); 2.22 (s, 3H); 1.55 (s, 6H); MS for C₂₂H₂₃N₃O₅ m/z 410 (M+H)⁺.

### Example 32

### Compound 60

### 2-Methyl-2-(4-(((1-(4-methylbenzyl)-1H-1,2,3-triazol-4-yl)methoxy)carbonyl)phenylthio)propanoic acid

To 4-(1-tert-Butoxy-2-methyl-1-oxopropan-2-ylthio)benzoic acid (0.728 g, 2.46 mmol) was added a mixture of (1-(4-methylbenzyl)-1H-1,2,3-triazol-4-yl)methanol (0.5 g, 2.46 mmol), N,N′-dicyclohexylcarbodiimide (0.61 g, 2.95 mmol), and 4-(dimethylamino)pyridine (0.035 mg, 0.295 mmol) in dichloromethane (8 mL) and stirred at room temperature overnight. The mixture was filtered to remove 1,3-dicyclohexylurea and the filtrate evaporated. The residue was purified by PTLC (30% ethyl acetate/hexane) to give (1-(4-methylbenzyl)-1H-1,2,3-triazol-4-yl)methyl 4-(1-tert-butoxy-2-methyl-1-oxopropan-2-ylthio)benzoate as a white solid (0.62 g), ¹H NMR (400 MHz, DMSO-d₆) δ 8.27 (s, 1H); 7.91 (d, J=8.4 Hz, 2H); 7.55 (d, J=8.4 Hz, 2H); 7.20 (dd, J=8.0 and 24.4 Hz, 4H); 5.55 (s, 2H); 5.38 (s, 2H); 2.27 (s, 3H); 1.42 (s, 6H); 1.33 (s, 9H); MS for C₂₆H₃₁N₃O₄S m/z 482 (M+H)⁺ and (1-(4-methylbenzyl)-1H-1,2,3-triazol-5-yl)methyl 4-(1-tert-butoxy-2-methyl-1-oxopropan-2-ylthio)benzoate as a white solid (0.25 g); ¹H NMR (400 MHz, DMSO-d₆) δ 7.91 (s, 1H); 7.59 (d, J=8.4 Hz, 2H); 7.44 (d, J=8.4 Hz, 2H); 7.20 (dd, J=8.4 and 12 Hz, 4H); 5.70 (s, 2H); 5.44 (s, 2H); 2.20 (s, 3H); 1.42 (s, 6H); 1.34 (s, 9H); MS for C₂₆H₃₁N₃O₄S m/z 482 (M+H)⁺. (1-(4-Methylbenzyl)-1H-1,2,3-triazol-4-yl)methyl 4-(1-tert-butoxy-2-methyl-1-oxopropan-2-ylthio)benzoate and 4M hydrogen chloride solution in dioxane (4 mL) were stirred at room temperature overnight and the evaporated under vacuum. The residue was purified by PTLC (10% methanol/dichloromethane) to give product as a white solid (0.43 g, 78%); ¹H NMR (400 MHz, DMSO-d₆) δ 8.27 (s, 1H); 7.90 (d, J=6.4 Hz, 2H); 7.54 (d, J=6.4 Hz, 2H); 7.20 (dd, J=8.0 and 24 Hz, 4H); 5.55 (s, 2H); 5.38 (s, 2H); 2.27 (s, 3H); 1.42 (s, 6H); MS for C₂₂H₂₃N₃O₄S m/z 426 (M+H)⁺.

### Example 33

### Compound 62

### 2-Methyl-2-(4-(((1-(4-methylbenzyl)-1H-1,2,3-triazol-5yl)methoxy)carbonyl)phenylthio)propanoic acid

(1-(4-Methylbenzyl)-1H-1,2,3-triazol-5-yl)methyl 4-(1-tert-butoxy-2-methyl-1-oxopropan-2-ylthio)benzoate (0.25 g, 0.519 mmol) and 4M hydrogen chloride solution in dioxane (4 mL) were stirred at room temperature overnight and the evaporated under vacuum. The residue was purified by PTLC (10% methanol/dichloromethane) to give product as a white solid (0.19 g, 85%); ¹H NMR (400 MHz, DMSO-d₆) δ 7.91 (s, 1H); 7.56 (d, J=8.4 Hz, 2H); 7.43 (d, J=8.4 Hz, 2H); 7.05 (dd, J=8.4 and 14.4 Hz, 4H); 5.70 (s, 2H); 5.44 (s, 2H); 2.20 (s, 3H); 1.42 (s, 6H); MS for C₂₂H₂₃N₃O₄S m/z 426 (M+H)⁺.

### Example 34

### Compound 64

### 2-Methyl-2-(4-(((1-(4-(trifluoromethyl)benzyl)-1H-1,2,3-triazol-4-yl)methoxy)carbonyl)phenoxy)propanoic acid

4-(1-tert-Butoxy-2-methyl-1-oxopropan-2-yloxy)benzoic acid (0.22 g, 0.778 mmol), (1-(4-(trifluoromethyl)benzyl)-1H-1,2,3-triazol-4-yl)methanol (0.20 g, 0.778 mmol), N,N′-dicyclohexylcarbodiimide (0.19 g, 0.934 mmol), and 4-(dimethylamino)pyridine (0.0112 mg, 0.0934 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-(trifluoromethyl)benzyl)-1H-1,2,3-triazol-4-yl)methyl 4-(1-tert-butoxy-2-methyl-1-oxopropan-2-yloxy)benzoate as a white solid (0.31 g, 76%); MS for C₂₆H₂₈F₃N₃O₅ m/z 520 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.25 g, 92%); ¹H NMR (400 MHz, DMSO-d₆) δ 8.34 (s, 1H); 7.85 (m, 2H); 7.75 (d, J=8.4 Hz, 2H); 7.51 (d, J=8.4 Hz, 2H); 6.87 (m, 2H); 5.73 (s, 2H); 5.35 (s, 2H); 1.55 (s, 6H); MS for C₂₂H₂₀F₃N₃O₅ m/z 464 (M+H)⁺.

### Preparation of (1-(4-(trifluoromethyl)benzyl)-1H-1,2,3-triazol-4-yl)methanol

1-(Azidomethyl)-4-(trifluoromethyl)benzene (9.54 g, 47.4 mmol) propargyl alcohol (2.80 mL, 47.4 mmol), copper sulfate (0.60 g, 2.37 mmol), and sodium ascorbate (0.94 g, 4.74 mmol) in water/dimethylformamide (1:4, 80 mL) were stirred at 65° C. overnight. The mixture was diluted with water, filtered, and extracted with ethyl acetate. The extract was washed with brine, dried (MgSO₄), and evaporated to give product as a white solid (10.0 g, 82%); ¹H NMR (400 MHz, DMSO-d₆) δ 8.07 (s, 1H); 7.74 (d, J=8.0 Hz, 2H); 7.49 (d, J=8.0 Hz, 2H); 5.70 (s, 2H); 5.17 (t, J=5.6 Hz, 1H); 4.51 (d, J=5.6 Hz, 2H); MS for C₁₁H₁₀F₃N₃O m/z 258 (M+H)⁺.

### Preparation of 1-(Azidomethyl)-4-(trifluoromethyl)benzene

4-(Trifluoromethyl)benzyl bromide (10.4 mL, 43.4 mmol) and sodium azide (3.10 g, 47.7 mmol) in dimethylformamide (80 mL) were stirred at 65° C. for 4 hours. The mixture was diluted with water and extracted with ether. The extract was washed with brine, dried (MgSO₄), and evaporated to give product as an oil (8.7 g, 99%); MS for C₈H₆F₃N₃ m/z 202 (M+H)⁺.

### Example 35

### Compound 102

### 2-Methyl-2-(4-(((1-(4-(trifluoromethyl)benzyl)-1H-1,2,3-triazol-4-yl)methoxy)carbonyl)phenylthio)propanoic acid

4-(1-tert-Butoxy-2-methyl-1-oxopropan-2-ylthio)benzoic acid (0.23 g, 0.778 mmol), (1-(4-(trifluoromethyl)benzyl)-1H-1,2,3-triazol-4-yl)methanol (0.20 g, 0.778 mmol), N,N′-dicyclohexylcarbodiimide (0.19 g, 0.934 mmol), and 4-(dimethylamino)pyridine (0.0112 mg, 0.0934 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-(trifluoromethyl)benzyl)-1H-1,2,3-triazol-4-yl)methyl 4-(1-tert-butoxy-2-methyl-1-oxopropan-2-ylthio)benzoate as a white solid (0.38 g, 90%); MS for C₂₆H₂₈F₃N₃O₄S m/z 536 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.32 g, 95%); ¹H NMR (400 MHz, DMSO-d₆) δ 8.37 (s, 1H); 7.91 (d, J=6.0 Hz, 2H); 7.75 (d, J=8.0 Hz, 2H); 7.55 (d, J=6.0 Hz, 2H); 7.52 (d, J=8.0 Hz, 2H); 5.74 (s, 2H); 5.41 (s, 2H); 1.42 (s, 6H); MS for C₂₂H₂₀F₃N₃O₄S m/z 480 (M+H)⁺.

### Example 36

### Compound 103

### 2-(4-(((1-(4-Methoxybenzyl)-1H-1,2,3-triazol-4-yl)methoxy)carbonyl)phenoxy)-2-methylpropanoic acid

4-(1-tert-Butoxy-2-methyl-1-oxopropan-2-yloxy)benzoic acid (0.26 g, 0.912 mmol), (1-(4-methoxybenzyl)-1H-1,2,3-triazol-4-yl)methanol (0.20 g, 0.912 mmol), N,N′-dicyclohexylcarbodiimide (0.22 g, 1.09 mmol), and 4-(dimethylamino)pyridine (0.013 mg, 0.109 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-methoxybenzyl)-1H-1,2,3-triazol-4-yl)methyl 4-(1-tert-butoxy-2-methyl-1-oxopropan-2-yloxy)benzoate as a white solid (0.38 g, 86%); MS for C₂₆H₃₁N₃O₆ m/z 482 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.26 g, 77%); ¹H NMR (400 MHz, DMSO-d₆) δ 8.22 (s, 1H); 7.84 (m, 2H); 7.30 (d, J=8.4 Hz, 2H); 6.92 (d, J=8.4 Hz, 2H); 6.87 (m, 2H); 5.51 (s, 2H); 5.32 (s, 2H); 3.71 (s, 3H); 1.54 (s, 6H); MS for C₂₂H₂₃N₃O₆ m/z 426 (M+H)⁺.

### Preparation of (1-(4-Methoxybenzyl)-1H-1,2,3-triazol-4-yl)methanol

(Azidomethyl)benzene (12.03 g, 73.7 mmol) propargyl alcohol (4.35 mL, 73.7 mmol), copper sulfate (0.92 g, 3.69 mmol), and sodium ascorbate (1.46 g, 0.737 mmol) in water/dimethylformamide (1:4, 80 mL) were stirred at 65° C. overnight. The mixture was diluted with water, filtered, and extracted with ethyl acetate. The extract was washed with brine, dried (MgSO₄), and evaporated to give product as a white solid (12.6 g, 78%); ¹H NMR (400 MHz, DMSO-d₆) δ 7.94 (s, 1H); 7.29 (d, J=6.8 Hz, 2H); 6.93 (d, J=6.8 Hz, 2H); 5.47 (s, 2H); 5.12 (t, J=5.6 Hz, 1H); 4.48 (d, J=5.6 Hz, 2H); 3.73 (s, 3H); MS for C₁₁H₁₃N₃O₂ m/z 220 (M+H)⁺.

### Preparation of 1-(Azidomethyl)-4-methoxybenzene

4-Methoxybenzyl chloride (10.0 mL, 73.7 mmol) and sodium azide (5.27 g, 81.1 mmol) in dimethylformamide (75 mL) were stirred at 65° C. for 4 hours. The mixture was diluted with water and extracted with ether. The extract was washed with brine, dried (MgSO₄), and evaporated to give product as an oil (12.0 g, 99%); MS for C₈H₈N₃O m/z 164 (M+H)⁺.

### Example 37

### Compound 68

### 2-(4-(((1-(4-Methoxybenzyl)-1H-1,2,3-triazol-4-yl)methoxy)carbonyl)phenylthio)-2-methylpropanoic acid

4-(1-tert-Butoxy-2-methyl-1-oxopropan-2-ylthio) benzoic acid (0.27 g, 0.912 mmol), (1-(4-methoxybenzyl)-1H-1,2,3-triazol-4-yl)methanol (0.20 g, 0.912 mmol), N,N′-dicyclohexylcarbodiimide (0.22 g, 1.09 mmol), and 4-(dimethylamino)pyridine (0.013 mg, 0.109 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-methoxybenzyl)-1H-1,2,3-triazol-4-yl)methyl 4-(1-tert-butoxy-2-methyl-1-oxopropan-2-ylthio)benzoate as a white solid (0.41 g, 91%); MS for C₂₆H₃₁N₃O₅S m/z 498 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.3 g, 82); ¹H NMR (400 MHz, DMSO-d₆) δ 8.25 (s, 1H); 7.89 (d, J=6.4 Hz, 2H); 7.54 (d, J=6.4 Hz, 2H); 7.31 (d, J=8.8 Hz, 2H); 6.92 (d, J=8.8 Hz, 2H); 5.52 (s, 2H); 5.37 (s, 2H); 3.73 (s, 3H); MS for C₂₂H₂₃N₃O₅S m/z 498 (M+H)⁺.

### Example 38

### Compound 74

### 2-Methyl-2-(4-(((1-(4-(trifluoromethoxy)benzyl)-1H-1,2,3-triazol-4-yl)methoxy)carbonyl)phenoxy)propanoic acid

4-(1-tert-Butoxy-2-methyl-1-oxopropan-2-yloxy)benzoic acid (0.31 g, 1.10 mmol), (1-(4-(trifluoromethoxy)benzyl)-1H-1,2,3-triazol-4-yl)methanol (0.30 g, 1.10 mmol), N,N′-dicyclohexylcarbodiimide (0.27 g, 1.32 mmol), and 4-(dimethylamino)pyridine (0.0158 mg, 0.132 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-(trifluoromethoxy)benzyl)-1H-1,2,3-triazol-4-yl)methyl 4-(1-tert-butoxy-2-methyl-1-oxopropan-2-yloxy)benzoate as an oil (0.56 g, 95%); MS for C₂₆H₂₈F₃N₃O₆ m/z 536 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.39 g, 78%); ¹H NMR (400 MHz, DMSO-d₆) δ 8.32 (s, 1H); 7.85 (d, J=6.4 Hz, 2H); 7.45 (d, J=8.8 Hz, 2H); 7.37 (d, J=8.8 hz, 2H); 6.86 (d, J=6.4 Hz, 2H); 5.65 (s, 2H); 5.34 (s, 2H); 1.55 (s, 6H); MS for C₂₂H₂₀F₃N₃O₆ m/z 480 (M+H)⁺.

### Preparation of (1-(4-(Trifluoromethoxy)benzyl)-1H-1,2,3-triazol-4-yl)methanol

1-(Azidomethyl)-4-(trifluoromethoxy)benzene (4.99 g, 23.0 mmol), propargyl alcohol (1.36 g, 23.0 mmol), copper sulfate (0.29 g, 1.15 mmol), and sodium ascorbate (0.46 g, 2.30 mmol) in water/dimethylformamide (1:4, 40 mL) were stirred at 65° C. overnight. The mixture was diluted with water, filtered, and extracted with ethyl acetate. The extract was washed with brine, dried (MgSO₄), and evaporated to give product as a white solid (5.0 g, 80%); ¹H NMR (400 MHz, DMSO-d₆) δ 8.04 (s, 1H); 7.44 (d, J=8.0 Hz, 2H); 7.37 (d, J=8.0 Hz, 2H); 5.62 (s, 2H); 5.16 (d, J=5.6 Hz, 2H); 4.50 (d, J=5.6 Hz, 2H); MS for C₁₁H₁₀F₃N₃O₂ m/z 274 (M+H)⁺.

### Preparation of 1-(Azidomethyl)-4-(trifluoromethoxy)benzene

4-(Trifluoromethoxy)benzyl bromide (5.0 g, 23.0 mmol) and sodium azide (2.25 g, 34.5 mmol) in dimethylformamide (40 mL) were stirred at 65° C. for 4 hours. The mixture was diluted with water and extracted with ether. The extract was washed with brine, dried (MgSO₄), and evaporated to give product as an oil (5.00 g, 99%); MS for C₈H₆F₃N₃O m/z 218 (M+H)⁺.

### Example 39

### Compound 73

### 2-Methyl-2-(4-(((1-(4-(trifluoromethoxy)benzyl)-1H-1,2,3-triazol-4-yl)methoxy)carbonyl)phenylthio)propanoic acid

4-(1-tert-Butoxy-2-methyl-1-oxopropan-2-ylthio)benzoic acid (0.33 g, 1.10 mmol), (1-(4-(trifluoromethoxy)benzyl)-1H-1,2,3-triazol-4-yl)methanol (0.30 g, 1.10 mmol), N,N′-dicyclohexylcarbodiimide (0.27 g, 1.32 mmol), and 4-(dimethylamino)pyridine (0.0158 mg, 0.132 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-(trifluoromethoxy)benzyl)-1H-1,2,3-triazol-4-yl)methyl 4-(1-tert-butoxy-2-methyl-1-oxopropan-2-ylthio)benzoate as a white solid (0.55 g, 90%); MS for C₂₆H₂₈F₃N₃O₅S m/z 552 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.43 g, 87%); ¹H NMR (400 MHz, DMSO-d₆) δ 8.35 (s, 1H); 7.90 (d, J=8.0 Hz, 2H); 7.55 (d, J=8.0 Hz, 2H); 7.46 d, J=8.0 hz, 2H); 7.37 (d, J=8.0 Hz, 2H); 5.66 (s, 2H); 5.40 (s, 2H); 1.42 (s, 6H); MS for C₂₂H₂₀F₃N₃O₅S m/z 496 (M+H)⁺.

### Example 40

### Compound 72

### 2-Methyl-2-(4-(((1-(4-(trifluoromethylthio)benzyl)-1H-1,2,3-triazol-4-yl)methoxy)carbonyl)phenoxy)propanoic acid

4-(1-tert-Butoxy-2-methyl-1-oxopropan-2-yloxy)benzoic acid (0.29 g, 1.04 mmol), (1-(4-(trifluoromethylthio)benzyl)-1H-1,2,3-triazol-4-yl)methanol (0.30 g, 1.04 mmol), N,N′-dicyclohexylcarbodiimide (0.26 g, 1.25 mmol), and 4-(dimethylamino)pyridine (0.015 g, 0.125 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-(trifluoromethylthio)benzyl)-1H-1,2,3-triazol-4-yl)methyl 4-(1-tert-butoxy-2-methyl-1-oxopropan-2-yloxy)benzoate as an oil (0.50 g, 88%); MS for C₂₆H₂₈F₃N₃O₅S m/z 552 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.31 g, 68%); ¹H NMR (400 MHz, DMSO-d₆) δ 8.35 (s, 1H); 7.87 (d, J=7.2 Hz, 2H); 7.72 (d, J=8.0 Hz, 2H); 7.43 (d, J=8.0 hz, 2H); 6.86 (d, J=7.2 Hz, 2H); 5.70 (s, 2H); 5.35 (s, 2H); 1.55 (s, 6H); MS for C₂₂H₂₀F₃N₃O₅S m/z 496 (M+H)⁺.

### Preparation of (1-(4-(trifluoromethylthio)benzyl)-1H-1,2,3-triazol-4-yl)methanol

(4-(Azidomethyl)phenyl)(trifluoromethyl)sulfane (0.86 g, 3.69 mmol) propargyl alcohol (0.22 mL, 3.69 mmol), copper sulfate (0.046 g, 0.185 mmol), and sodium ascorbate (0.073 g, 0.369 mmol) in water/dimethylformamide (1:4, 10 mL) were stirred at 65° C. overnight. The mixture was diluted with water, filtered, and extracted with ethyl acetate. The extract was washed with brine, dried (MgSO₄), and evaporated to give product as a white solid (0.82 g, 77%); ¹H NMR (400 MHz, DMSO-d₆) δ 8.07 (s, 1H); 7.72 (d, J=8.0 Hz, 2H); 7.41 (d, J=8.0 Hz, 2H); 5.67 (s, 2H); 5.17 (d, J=5.6 Hz, 2H); 4.51 (d, J=5.6 Hz, 2H); MS for C₁₁H₁₀F₃N₃OS m/z 290 (M+H)⁺.

### Preparation of (4-(Azidomethyl)phenyl)(trifluoromethyl)sulfane

4-(Trifluoromethylthio)benzyl bromide (1.0 g, 3.69 mmol) and sodium azide (0.36 g, 5.54 mmol) in dimethylformamide (10 mL) were stirred at 65° C. for 4 hours. The mixture was diluted with water and extracted with ether. The extract was washed with brine, dried (MgSO₄), and evaporated to give product as an oil (0.86 g, 99%); MS for C₈H₆F₃N₃S m/z 234 (M+H)⁺.

### Example 41

### Compound 71

### 2-Methyl-2-(4-(((1-(4-(trifluoromethylthio)benzyl)-1H-1,2,3-triazol-4-yl)methoxy)carbonyl)phenylthio)propanoic acid

4-(1-tert-Butoxy-2-methyl-1-oxopropan-2-ylthio)benzoic acid (0.31 g, 1.04 mmol), (1-(4-(trifluoromethylthio)benzyl)-1H-1,2,3-triazol-4-yl)methanol (0.30 g, 1.04 mmol), N,N′-dicyclohexylcarbodiimide (0.26 g, 1.25 mmol), and 4-(dimethylamino)pyridine (0.015 g, 0.125 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-(trifluoromethylthio)benzyl)-1H-1,2,3-triazol-4-yl)methyl 4-(1-tert-butoxy-2-methyl-1-oxopropan-2-ylthio)benzoate as a white solid (0.55 g, 93%); MS for C₂₆H₂₈F₃N₃O₄S₂ m/z 568 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.47g, 96%); ¹H NMR (400 MHz, DMSO-d₆) δ 8.37 (s, 1H); 7.90 (d, J=7.6 Hz, 2H); 7.73 (d, J=8.0 Hz, 2H); 7.55 (d, J=7.6 hz, 2H); 7.44 (d, J=8.0 Hz, 2H); 5.71 (s, 2H); 5.41 (s, 2H); 1.42 (s, 6H); MS for C₂₂H₂₀F₃N₃O₄S₂ m/z 512 (M+H)⁺.

### Example 42

### Compound 76

### 2-(4-(((1-(4-(1,1,1,3,3,3-Hexafluoro-2-methoxypropan-2-yl)benzyl)-1H-1,2,3-triazol-4-yl)methoxy)carbonyl)phenoxy)-2-methylpropanoic acid

4-(1-tert-Butoxy-2-methyl-1-oxopropan-2-yloxy)benzoic acid (0.25 g, 0.886 mmol), (1-(4-(1,1,1,3,3,3-hexafluoro-2-methoxypropan-2-yl)benzyl)-1H-1,2,3-triazol-4-yl)methanol (0.327 g, 0.886 mmol), N,N′-dicyclohexylcarbodiimide (0.22 g, 1.06 mmol), and 4-(dimethylamino)pyridine (0.0127 g, 0.106 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-(1,1,1,3,3,3-hexafluoro-2-methoxypropan-2-yl)benzyl)-1H-1,2,3-triazol-4-yl)methyl 4-(1-tert-butoxy-2-methyl-1-oxopropan-2-yloxy)benzoate as a white solid (0.5 g, 90%); MS for C₂₉H₃₁F₆N₃O₆ m/z 632 (M+H)⁺. The ester and 4M hydrogen chloride solution in dioxane (4 mL) were stirred at room temperature overnight and then evaporated under vacuum. The residue was purified by PTLC (10% methanol/dichloromethane) to give product as a white solid (0.38 g, 85%); ¹H NMR (400 MHz, DMSO-d₆) δ 8.36 (s, 1H); 7.86 (m, 2H); 7.58 (d, J=8 Hz, 2H); 7.49 (d, J=8 Hz, 2H); 6.87 (m, 2H); 5.71 (s, 2H); 5.36 (s, 2H); 3.42 (s, 3H); 1.54 (s, 6H); MS for C₂₅H₂₃F₆N₃O₆ m/z 576 (M+H)⁺.

### Preparation of (1-(4-(1,1,1,3,3,3-Hexafluoro-2-methoxypropan-2-yl)benzyl)-1H-1,2,3-triazol-4-yl)methanol

1-(Azidomethyl)-4-(1,1,1,3,3,3-hexafluoro-2-methoxypropan-2-yl)benzene (4.77 g, 15.2 mmol), propargyl alcohol (0.9 mL, 15.2 mmol), copper sulfate (0.19 g, 0.76 mmol), and sodium ascorbate (1.16 g, 1.52 mmol) in water/dimethylformamide (1:4, 40 mL) were stirred at 65° C. overnight. The mixture was diluted with water, filtered, and extracted with ethyl acetate. The extract was washed with brine, dried (MgSO₄), and evaporated to give product as a white solid (4.30 g, 77%); ¹H NMR (400 MHz, DMSO-d₆) δ 8.08 (s, 1H); 7.59 (d, J=8.4 Hz, 2H); 7.48 (d, J=8.4 Hz, 2H); 5.68 (s, 2H); 5.17 (s, 2H) 2H); 4.52 (d, J=5.6 Hz, 2H); 3.42 (s, 3H); MS for C₁₄H₁₃P₆N₃O₂ m/z 370 (M+H)⁺.

### Preparation of 1-(Azidomethyl)-4-(1,1,1,3,3,3-hexafluoro-2-methoxypropan-2-yl)benzene

1-(Bromomethyl)-4-(1,1,1,3,3,3-hexafluoro-2-methoxypropan-2-yl)benzene (5.0 g, 14.2 mmol) and sodium azide (1.39 g, 21.4 mmol) in dimethylformamide (40 mL) were stirred at 65° C. for 4 hours. The mixture was diluted with water and extracted with ether. The extract was washed with brine, dried (MgSO₄), and evaporated to give product as an oil (4.77 g, 93%); MS for C₁₁H₉F₆N₃O m/z 314 (M+H)⁺.

### Preparation of 1-(Bromomethyl)-4-(1,1,1,3,3,3-hexafluoro-2-methoxypropan-2-yl)benzene

1-(1,1,1,3,3,3-Hexafluoro-2-methoxypropan-2-yl)-4-methylbenzene (7.66 g, 28.1 mmol), N-bromosuccinimide (5.00 g, 28.1 mmol), and benzoyl peroxide (0.34 g, 1.41 mmol) in carbon tetrachloride were stirred and heated at reflux overnight. The mixture was allowed to cool and filtered to remove succinimide. The filtrate was evaporated under vacuum to give nearly pure product as a colorless oil (9.9 g, 99%); MS for C₁₁H₉BrF₆O m/z 314 (M+H)⁺.

### Preparation of 1-(1,1,1,3,3,3-Hexafluoro-2-methoxypropan-2-yl)-4-methylbenzene

Diisopropyl azodicarboxylate (11.4 mL, 58.1 mmol) was added dropwise at room temperature to hexafluoro-2-(p-tolyl)isopropanol (10.0 g, 38.7 mmol) and triphenylphosphine (15.2 g, 58.0 mmol) in dichloromethane (100 mL) and the mixture was stirred overnight. The reaction was evaporated under vacuum and the residue purified by flash column chromatography to give product as a colorless oil (7.66 g, 56%); ¹H NMR (400 MHz, DMSO-d₆) δ 7.42 (dd, J=8 and 20 Hz, 4H); 3.42 (s, 3H); 2.37 (s, 3H); MS for C₁₁H₁₀F₆O m/z 273 (M+H)⁺.

### Example 43

### Compound 75

### 2-(4-(((1-(4-(1,1,1,3,3,3-Hexafluoro-2-methoxypropan-2-yl)benzyl)-1H-1,2,3-triazol-4-yl)methoxy)carbonyl)phenylthio)-2-methylpropanoic acid

4-(1-tert-Butoxy-2-methyl-1-oxopropan-2-ylthio)benzoic acid (0.28 g, 0.937 mmol), (1-(4-(1,1,1,3,3,3-hexafluoro-2-methoxypropan-2-yl)benzyl)-1H-1,2,3-triazol-4-yl)methanol (0.28 g, 0.937 mmol), N,N′-dicyclohexylcarbodiimide (0.23 g, 1.12 mmol), and 4-(dimethylamino)pyridine (0.0134 g, 0.112 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-(1,1,1,3,3,3-hexafluoro-2-methoxypropan-2-yl)benzyl)-1H-1,2,3-triazol-4-yl)methyl 4-(1-tert-butoxy-2-methyl-1-oxopropan-2-ylthio)benzoate as a white solid (0.56 g, 93%); MS for C₂₉H₃₁F₆N₃O₅S m/z 648 (M+H)⁺. The ester and 4M hydrogen chloride solution in dioxane (4 mL) were stirred at room temperature overnight and the evaporated under vacuum. The residue was purified by PTLC (10% methanol/dichloromethane) to give product as a white solid (0.39 g, 75%); ¹H NMR (400 MHz, DMSO-d₆) δ 8.39 (s, 1H); 7.91 (d, J=7.6 Hz, 2H); 7.49-7.60 (m, 6H); 5.72 (s, 2H); 5.41 (s, 2H); 3.42 (s, 3H); 1.42 (s, 6H); MS for C₂₅H₂₃F₆N₃O₅S m/z 592 (M+H)⁺.

### Example 44

### Compound 87

### Synthesis of (R)-2-methyl-2-(4-((1-(1-(4-methylbenzyl)-1H-1,2,3-triazol-4-yl)ethoxy)carbonyl)phenylthio)propanoic acid

### Preparation of 1-(Azidomethyl)-4-methylbenzene

4-Methylbenzyl bromide (1 eq.) and sodium azide (1.2 eq.) in dimethylformamide were stirred at room temperature overnight. The mixture was diluted with water and extracted with ether. The organic extract was washed with brine, dried (MgSO₄), and evaporated to give product (99% yield).

### Preparation of (R)-1-(1-(4-(Methyl)benzyl)-1H-1,2,3-triazol-4-yl)ethanol

1-(Azidomethyl)-4-methylbenzene (1 eq.), R-butyn-2-ol (1 eq.), copper sulfate (0.05 eq.), and sodium ascorbate (0.1 eq.) in water/dimethylformamide (1:4) were stirred at 65° C. overnight. The mixture was diluted with water, filtered, and extracted with ethyl acetate. The organic extract was washed with brine, dried (MgSO₄), and evaporated to give product (75%-80% yield).

### Preparation of (R)-2-methyl-2-(4-((1-(1-(4-methylbenzyl)-1H-1,2,3-triazol-4-yl)ethoxy)carbonyl)phenylthio)propanoic acid

4-(1-tert-Butoxy-2-methyl-1-oxopropan-2-ylthio)benzoic acid (1 equiv.), (R)-1-(1-(4-(methyl)benzyl)-1H-1,2,3-triazol-4-yl)ethanol (1 eq.), 1-ethyl-3-(3-dimethyl aminopropyl)carbodiimide hydrochloride (1.2 eq.), and 4-(dimethylamino)pyridine (1 equiv.) in dimethylformamide were stirred at room temperature overnight. The mixture was diluted with ethyl acetate, washed with 10% aqueous citric acid, brine, saturated aqueous sodium bicarbonate and brine, dried (MgSO₄), and evaporated. The residue was purified by column chromatography (30% ethyl acetate/hexane) to give (R)-1-(1-(4-Methylbenzyl)-1H-1,2,3-triazol-4-yl)ethyl 4-(1-methoxy-2-methyl-1-oxopropan-2-ylthio)benzoate as a white solid (>90. The intermediate t-butyl ester was taken into hydrogen chloride solution in dioxane (4N), stirred at room temperature overnight, and then concentrated under vacuum. The residue was purified by column chromatography (10% methanol/dichloromethane) to give product as a white solid (85%-90% yield).

### Example 45

### Compound 95

### Synthesis of (R)-2-methyl-2-(4-((1-(1-(4-(trifluoromethyl)benzyl)-1H-1,2,3-triazol-4-yl)ethoxy)carbonyl)phenylthio)propanoic acid

### Preparation of 1-(Azidomethyl)-4-(trifluoromethyl)benzene

4-Trifluoromethylbenzyl bromide (1 eq.) and sodium azide (1.2 eq.) in dimethylformamide were stirred at room temperature overnight. The mixture was diluted with water and extracted with ether. The organic extract was washed with brine, dried (MgSO₄), and evaporated to give product (99% yield).

### Preparation of (R)-1-(1-(4-Trifluoromethylbenzyl)-1H-1,2,3-triazol-4-yl)ethanol

1-(Azidomethyl)benzene (1 eq.) R-butyn-2-ol (1 eq.), copper sulfate (0.05 eq.), and sodium ascorbate (0.1 eq.) in water/dimethylformamide (1:4) were stirred at 65° C. overnight. The mixture was diluted with water, filtered, and extracted with ethyl acetate. The organic extract was washed with brine, dried (MgSO₄), and evaporated to give product (75%-80% yield).

### Preparation of (R)-2-Methyl-2-(4-((1-(1-(4-(trifluoromethyl)benzyl)-1H-1,2,3-triazol-4-yl)ethoxy)carbonyl)phenylthio)propanoic acid

4-(1-tert-Butoxy-2-methyl-1-oxopropan-2-ylthio)benzoic acid (1 eq.), (R)-1-(1-(4-trifluoromethylbenzyl)-1H-1,2,3-triazol-4-yl)ethanol (1 eq.), 1-ethyl-3-(3-dimethyl aminopropyl)carbodiimide hydrochloride (1.2 eq.), and 4-(dimethylamino)pyridine (1 eq.) in dimethylformamide were stirred at room temperature overnight. The mixture was diluted with ethyl acetate, washed with 10% citric acid, brine, saturated aqueous sodium bicarbonate and brine, dried (MgSO₄), and evaporated. The residue was purified by column chromatography (30% ethyl acetate/hexane) to give (R)-1-(1-(4-(trifluoromethyl)benzyl)-1H-1,2,3-triazol-4-yl)ethyl 4-(1-methoxy-2-methyl-1-oxopropan-2-ylthio)benzoate as a white solid (>90%). The intermediate t-butyl ester and hydrogen chloride solution in dioxane (4N) were stirred at room temperature overnight and the evaporated under vacuum. The residue was purified by column chromatography (10% methanol/dichloromethane) to give product as a white solid (85%-90% yield).

### Example 46

### Compound 91

### Synthesis of 2-methyl-2-(4-((2-(1-(4-(trifluoromethyl)benzyl)-1H-1,2,3-triazol-4-yl)ethoxy)carbonyl)phenylthio)propanoic acid

### Preparation of 1-(azidomethyl)-4-(trifluoromethyl)benzene

4-Trifluoromethylbenzyl bromide (1 eq) and sodium azide (1.2 eq.) in dimethylformamide were stirred at room temperature overnight. The mixture was diluted with water and extracted with ether. The organic extract was washed with brine, dried (MgSO₄), and evaporated to give product as an oil (99% yield).

### Preparation of 2-(1-(4-(trifluoromethyl)benzyl)-1H-1,2,3-triazol-4-yl)ethanol

1-(Azidomethyl)-4-trifluoromethylbenzene (1 eq.), 1-butynol (1 eq.), copper sulfate (0.05 eq.), and sodium ascorbate (0.1 eq.) in water/dimethylformamide (1:4) were stirred at 65° C. overnight. The mixture was diluted with water, filtered, and extracted with ether. The organic extract was washed with brine, dried (MgSO₄), and evaporated to give product (>80% yield).

### Preparation of 2-methyl-2-(4-((2-(1-(4-(trifluoromethyl)benzyl)-1H-1,2,3-triazol-4-yl)ethoxy)carbonyl)phenylthio)propanoic acid

4-(1-tert-Butoxy-2-methyl-1-oxopropan-2-ylthio)benzoic acid (1 eq.), 2-(1-(4-(trifluoromethyl)benzyl)-1H-1,2,3-triazol-4-yl)ethanol, 1-ethyl-3-(3-dimethyl aminopropyl)carbodiimide hydrochloride (1.2 eq.), and 4-(dimethylamino)pyridine (1 eq.) in dimethylformamide were stirred at room temperature overnight. The mixture was diluted with ethyl acetate, washed with 10% aqueous citric acid, brine, saturated aqueous sodium bicarbonate and brine, dried (MgSO₄), and evaporated. The residue was purified by flash column chromatography (30% ethyl acetate/hexane) to give 2-(1-(4-trifluoromethyl)benzyl)-1H-1,2,3-triazol-4-yl)ethyl 4-(1-methoxy-2-methyl-1-oxopropan-2-ylthio)benzoate as a white solid (76%). The intermediate t-butyl ester and hydrogen chloride in dioxane (4N) were stirred at room temperature overnight and then evaporated. The residue was purified by flash column chromatography (10% methanol/dichloromethane) to give product as a white solid (79% yield).

### Example 47

General Pyrazole Procedure:

Step 1—N-Alkylation of the pyrazole nitrogen

To a solution of ethyl-1H-pyrazole-4-carboxylate (3.567 mmol, 350 mg, 1 equiv) in acetonitrile (10 mL), 1,5,7-triazabicyclo[4,4,0]dec-5-ene bound to polystyrene crosslinked with 2% DVB with a loading of ˜2.6 mmol base/g of resin (3.75 mmol, 2 g, 1.5 equiv) was added followed by the addition of the substituted benzyl bromide (3.567 mmol, 1 equiv). The mixture was heated at 60° C.-65° C. for 16 hours under slow stirring conditions. Upon cooling it was filtered and the resin thoroughly washed with dichloromethane. The filtrate was concentrated to afford the desired substituted ethyl 1-benzyl-1H-pyrazole-4-carboxylate.

Step 2—Reduction of ester to Alcohol

To a cold solution of the substituted pyrazole-4-carboxylate from Step 1 in tetrahydrofuran, 2M lithium aluminium hydride in THF (1 equiv) was added dropwise. The reaction was stirred at 0° C. for 30 minutes and allowed to slowly rise to room temperature. It was stirred for 16 hours, and monitored by TLC for completion in ethyl acetate. In some cases the reaction was left at 0° C. for the length of the reaction time. 10% citric acid was slowly added to the reaction mixture and the compound was extracted with a 9:1 mixture of DCM:MeOH. The organic layer was dried over magnesium sulfate, filtered and concentrated. The residue was purified by preparative TLC in 100% ethyl acetate to afford the desired substituted (1-benzyl-1H-pyrazol-4-yl)methanol.

Step 3—Coupling Reaction

The substituted (pyrazol-4-yl)methanol (1 equiv), 4-(1-tert-butoxy-2-methyl-1-oxopropan-2-yloxy)benzoic acid (1 equiv) from Step 2, dimethylaminopyridine (0.2 equiv) was taken into dicholoromethane. Dicyclohexylcarbodiimide (1.2 equiv) was added to the reaction mixture and the latter was stirred at room temperature for 16 hours. The reaction was filtered to remove any dicyclohexyl urea generated and the filtrate was concentrated in vacuo. The residue obtained was purified by preparative TLC in A 7:3 mixture of hexanes:ethyl acetate to afford the substituted (1-benzyl-1H-pyrazol-4-yl)methyl 4-(1-tert-butoxy-2-methyl-1-oxopropan-2-yloxy)benzoate.

Step 4—Coupling Reaction

The substituted (pyrazol-4-yl)methanol from step 2(1 equiv), 4-(1-tert-butoxy-2-methyl-1-oxopropan-2-ylthio)benzoic acid (1 equiv), dimethylaminopyridine (0.2 equiv) were taken into dicholoromethane. Dicyclohexylcarbodiimide (1.2 equiv) was added to the reaction mixture and the latter was stirred at room temperature for 16 hours. The reaction was filtered to remove any dicyclohexyl urea generated and the filtrate was concentrated in vacuo. The residue obtained was purified by preparative TLC in a 7:3 mixture of hexanes:ethyl acetate to afford the substituted (1-benzyl-1H-pyrazol-4-yl)methyl 4-(1-tert-butoxy-2-methyl-1-oxopropan-2-ylthio)benzoate.

Step 5—Acid Deprotection

The benzoate from step 3 or 4 was taken into 4 M HCl/dioxane (excess) and the reaction was stirred for 16 hours. It was concentrated and the resulting residue was purified by preparative TLC using a 9:1 mixture of dichloromethane:methanol. The extracted compound was lyophilized to a white solid in 30%-40% yields.

Step 6—Coupling Reaction

The substituted (pyrazol-4-yl)methanol from step 2(1 equiv), 2-(4-(1-tert-butoxy-2-methyl-1-oxopropan-2-yloxy)phenyl)acetic acid (1 equiv), Dimethylaminopyridine (0.2 equiv) were taken into dicholoromethane. Dicyclohexylcarbodiimide (1.2 equiv) was added to the reaction mixture and the latter was stirred at room temperature for 16 hours. The reaction was filtered to remove any dicyclohexyl urea generated and the filtrate was concentrated in vacuo. The residue obtained was purified by preparative TLC in a 7:3 mixture of hexanes:ethyl acetate afford the substituted benzoate.

Step 7—Preparation of Pyrazole Ester

To a dry flask sodium hydride (2.7 g, 56 mmol, 1.2 equiv) was added and purged with nitrogen. Ether (50 mL) was then added to the flask and the mixture was cooled to 0° C. This was followed by the addition of ethyl formate (32.8 g, 465 mmol, 10 equiv) using a syringe, and a dropwise addition of ethyl 3,3-diethoxypropionate (8.84 g, 46.5 mmol, 1 equiv) over a period of 1.25 hours. The reaction was allowed to stir overnight at room temperature. It was poured into ice cold water and extracted with three 50 mL portions of ether. The aqueous layer was acidified to pH=3 with 4.5 mL conc. HCl. Extraction with three 50 mL portions of dichloromethane afforded the desired dialdehyde in <10% yield.

The dialdehyde (300 mg, 2.083 mmol, 1 equiv), sodium acetate (283 mg, 4.166 mmol, 2 equiv) and (4-(trifluoromethyl)phenyl)hydrazine (403 mg, 2.2913 mmol, 1.1 equiv) were placed in absolute ethanol (15 mL) and stirred at room temperature for 30 minutes. The reaction was then refluxed till complete by TLC. It was then concentrated and the residue was partitioned between ethyl acetate and water. The organic layer was washed with brine, dried over magnesium sulfate and concentrated to give a brown residue, which was purified by silica gel chromatography using hexanes:ethyl acetate. The compound eluted in 10% ethyl acetate.

Step 8—Coupling

The substituted (pyrazol-4-yl)methanol from step 2 (1 equiv), 4-(1-(benzyloxy)-2-methyl-1-oxopropan-2-yloxy)benzoic acid (1 equiv), dimethylaminopyridine (0.2 equiv) were taken into dicholoromethane. Dicyclohexylcarbodiimide (1.2 equiv) was added to the reaction mixture and the latter was stirred at room temperature for 16 hours. The reaction was filtered to remove any dicyclohexyl urea generated and the filtrate was concentrated in vacuo. The residue obtained was purified by preparative TLC in a 7:3 mixture of hexanes:ethyl acetate afford the substituted benzoate.

Step 9—Hydrogenation

The substituted benzoate was taken into a mixture of ethyl acetate and ethanol and 10% Pd/C (10% by wt.) was added. The reaction was stabilized to a hydrogen atmosphere and stirred overnight under hydrogen. The reaction was filtered through celite, the filtrate was concentrated, and the residue was purified by preparative TLC using 9:1 dichloromethane:methanol mixture.

### Example 48

### Compound 3

### 2-methyl-2-(4-(((1-(4-methylbenzyl)-1H-pyrazol-4-yl)methoxy)carbonyl)phenylthio)propanoic acid

### Preparation of ethyl 1-(4-methylbenzyl)-1H-pyrazole-4-carboxylate

Ethyl 1H-pyrazole-4-carboxylate (0.3 g, 2.14 mmol), 4-methylbenzyl bromide (0.40 g, 2.14 mmol) and 1,5,7-triazabicyclo[4.4.0]dec-5-ene bound to polystyrene (loading 2 6 mmol/g, 1.65 g) in dimethylformamide (5 mL) were heated at 65° C. overnight. The mixture was filtered and evaporated to give product as a white solid (0.47 g, 90%); MS for C₁₄H₁₆N₂O₂ m/z 245 (M+H)⁺.

### Preparation of (1-(4-methylbenzyl)-1H-pyrazol-4-yl)methanol

Lithium aluminum hydride (2.0M in tetrahydrofuran, 0.92 mL, 1.84 mmol) was added dropwise to ethyl 1-(4-methylbenzyl)-1H-pyrazole-4-carboxylate (0.45 g, 1.84 mmol) in tetrahydrofuran (8 mL) at 0° C. The mixture was allowed to warm to room temperature and stirred overnight. The reaction was quenched with 2N aqueous hydrochloric acid and extracted with ethyl acetate. The extracts were washed with water and brine, dried (MgSO₄) and evaporated. The residue was purified by PTLC (50% ethyl acetate/hexanes) to give product as a white solid (0.33 g, 90%); MS for C₁₂H₁₄N₂O m/z 203 (M+H)⁺.

### Preparation of 2-Methyl-2-(4-(((1-(4-methylbenzyl)-1H-pyrazol-4-yl)methoxy)carbonyl)phenylthio)propanoic acid

4-(1-tert-Butoxy-2-methyl-1-oxopropan-2-ylthio)benzoic acid (0.44 g, 1.48 mmol), (1-(4-methylbenzyl)-1H-pyrazol-4-yl)methanol (0.30 g, 1.48 mmol), N,N′-dicyclohexylcarbodiimide (0.37 g, 1.77 mmol), and 4-(dimethylamino)pyridine (0.022 mg, 0.177 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (1-(4-methylbenzyl)-1H-pyrazol-4-yl)methyl 4-(1-tert-butoxy-2-methyl-1-oxopropan-2-ylthio)benzoate as a white solid (0.63 g, 88%); MS for C₂₇H₃₂N₂O₄S m/z 481 (M+H)⁺. The ester and 4M hydrogen chloride solution in dioxane (4 mL) were stirred at room temperature overnight and then evaporated under vacuum. The residue was purified by PTLC (10% methanol/dichloromethane) to give product as a white solid (0.50 g, 89%); HPLC retention time (Phenomenex 150×4.6 mm, 5μ, 1 mL/min, 20 mM ammonium acetate buffer, MeCN:water=15:85 for 2 min, then to 90:10 in 18 minutes)=12.5 min; ¹H-NMR (400 MHz, d₆-DMSO) 7.92 (s, 1H); 7.87 (m, 2H); 7.55 (m, 3H); 7.14 (s, 4H); 5.25 (s, 2H); 5.20 (s, 2H); 2.26 (s, 3H); 1.40 (s, 6H); MS for C₂₃H₂₄N₂O₄S m/z 425 (M+H)⁺.

### Example 49

### Compound 10

### 2-methyl-2-(4-(((1-(4-(trifluoromethoxy)benzyl)-1H-pyrazol-4 yl)methoxy)carbonyl)phenylthio)propanoic acid

The title compound was prepared using the procedures outlined in Steps 2, 4, 5.

MW=494; [M+H]=495.38

¹HNMR (DMSO-d₆)-δ 13.1 (brs, 1H), 8.0 (s, 1H), 7.89-7.88 (d, 2H, J=7.6 Hz), 7.6-7.53 (m, 3H), 7.37-7.32 (m, 4H), 5.36 (s, 2H), 5.22 (s, 2H), 1.4 (s, 6H).

### Preparation of ethyl 1-(4-(trifluoromethoxy)benzyl)-1H-pyrazol-4-carboxylate

The title compound was prepared using the procedure outlined in Step 1.

¹HNMR (DMSO-d₆)-δ 8.5 (d, 1H), 7.88 (d, 1H), 7.40-7.34 (m, 4H), 5.41 (s, 2H), 4.23-4.18 (dd, 2H, J=6.8 Hz), 1.27-1.23 (t, 3H, J=6.8 & 7.6 Hz).

### Example 50

### Compound 31

### 2-methyl-2-(4-(((1-(3-(trifluoromethyl)benzyl)-1H-pyrazol-4-yl)methoxy)carbonyl)phenoxy)propanoic acid

The title compound was prepared using the procedures outlined in Steps 3 and 5.

MW=462; [M+H]=463.34

¹HNMR (DMSO-d₆)-δ 13.1 (brs, 1H), 8.0 (s, 1H), 7.84 (brs, 2H), 7.66-7.65 (d, 1H, J=8.8 Hz), 7.6-7.56 (m, 3H), 7.53-7.51 (d, 1H, J=7.6 Hz), 6.86 (brs, 2H), 5.43 (s, 2H), 5.18 (s, 2H), 1.54 (s, 6H).

### Preparation of (1-(3-(trifluoromethyl)benzyl)-1H-pyrazol-4-yl)methanol

The title compound was prepared using the procedure outlined in Step 2 in 50% yield.

MW=256; [M+H]=256.99

¹HNMR (DMSO-d₆)-δ 7.76 (d, 1H), 7.66-7.65 (d, 1H, J=7.6 Hz), 7.6-7.56 (m, 2H), 7.52-7.5 (d, 1H, J=7.6 Hz), 7.41 (s, 1H), 5.4 (s, 2H), 4.86-4.83 (t, 1H, J=5.6 Hz), 4.35-4.34 (d, 2H, J=5.2 Hz).

### Preparation of ethyl 1-(3-(trifluoromethyl)benzyl)-1H-pyrazole-4-carboxylate

The title compound was prepared using the procedure outlined in Step 1 in a 91% yield.

MW=298; [M+H]=299.52

¹HNMR (DMSO-d₆)-δ 8.54 (d, 1H, J=2.4 Hz), 7.89 (s, 1H), 7.67-7.56 (m, 4H), 5.48 (s, 2H), 4.23-4.18 (dd, 1H, J=7.6 & 7.2 Hz), 1.27-1.24 (t, 2H, J=7.2 & 6.8 Hz).

### Example 51

### Compound 30

### 2-methyl-2-(4-(((1-(3-(trifluoromethyl)benzyl)-1H-pyrazol-4-yl)methoxy)carbonyl)phenylthio)propanoic acid

The title compound was prepared using the procedures outlined in Step 5 in 23% yield.

MW=478; [M+H]=479.34

¹HNMR (DMSO-d₆)-δ 12.9 (brs, 1H), 8.04 (s, 1H), 7.9-7.88 (d, 2H, J=6.8 Hz), 7.66-7.52 (m, 7H), 5.43 (brs, 2H), 5.23 (s, 2H), 1.41 (s, 6H).

### Preparation of (1-(3-(trifluoromethyl)benzyl)-1H-pyrazol-4-yl)methyl 4-(1-tert-butoxy-2-methyl-1-oxopropan-2-ylthio)benzoate

The title compound was prepared using the procedure outlined in Step 4 in 69% yield.

MW=534; [M+H]−Bu^{t}=478.86

¹HNMR (DMSO-d₆)-δ 8.05 (s, 1H), 7.92-7.90 (d, 2H, J=8.4 Hz), 7.62-7.54 (m, 7H), 5.44 (brs, 2H), 5.23 (s, 2H), 1.41 (s, 6H), 1.33 (s, 9H).

### Preparation of (1-(3-(trifluoromethyl)benzyl)-1H-pyrazol-4-yl)methanol

The title compound was prepared using the procedure outlined in Step 2 in 50% yield.

MW=256; [M+H]=256.99

¹HNMR (DMSO-d₆)-δ 7.76 (d, 1H), 7.66-7.65 (d, 1H, J=7.6 Hz), 7.6-7.56 (m, 2H), 7.52-7.5 (d, 1H, J=7.6 Hz), 7.41 (s, 1H), 5.4 (s, 2H), 4.86-4.83 (t, 1H, J=5.6 Hz), 4.35-4.34 (d, 2H, J=5.2 Hz).

### Preparation of ethyl 1-(3-(trifluoromethyl)benzyl)-1H-pyrazole-4-carboxylate

The title compound was prepared using the procedure outlined in Step 1 in a 91% yield.

MW=298; [M+H]=299.52

¹HNMR (DMSO-d₆)-δ 8.54 (d, 1H, J=2.4 Hz), 7.89 (s, 1H), 7.67-7.56 (m, 4H), 5.48 (s, 2H), 4.23-4.18 (dd, 1H, J=7.6 & 7.2 Hz), 1.27-1.24 (t, 2H, J=7.2 & 6.8 Hz).

### Example 52

### Compound 12

### 2-methyl-2-(4-(((3-propyl-1-(4-(trifluoromethyl)benzyl)-1H-pyrazol-4-yl)methoxy)carbonyl)phenylthio)propanoic acid

### Preparation of ethyl 3-propyl-1H-pyrazole-4-carboxylate

Ethyl 2-((dimethylamino)methylene)-3-oxohexanoate [prepared according to the method described in J. Heterocycl. Chem. 1987, 24, 1669-1676] (15 g, 0.0703 mol) and t-butyl carbazate (9.29 g, 0.0703 mol) in ethanol (100 mL) were heated at reflux overnight. The mixture was evaporated under vacuum and the residue purified by flash column chromatography (30% ethyl acetate/hexanes) to give product as a yellow oil (11.2 g, 88%).

### Preparation of ethyl 3-propyl-1-(4-(trifluoromethyl)benzyl)-1H-pyrazole-4-carboxylate

Ethyl 3-propyl-1H-pyrazole-4-carboxylate (1.00 g, 5.49 mmol), 4-trifluoromethylbenzyl bromide (1.31 g, 5.49 mmol), and potassium carbonate (1.14 g, 8.24 mmol) in dimethylformamide (15 mL) were heated at 65° C. overnight. The mixture was diluted with water and extracted with ethyl acetate. The organic layer was washed with brine, dried (MgSO₄), and evaporated. The residue was purified by flash column chromatography (30% ethyl acetate/hexanes) to give an inseparable mixture of the desired product and the regioisomeric ethyl 5-propyl-1-(4-(trifluoromethyl)benzyl)-1H-pyrazole-4-carboxylate (approx. 70% desired isomer by ¹H NMR, 1.69 g, 90%); MS for C₁₇H₁₉F₃N₂O₂ m/z 341 (M+H)⁺.

### Preparation of (3-propyl-1-(4-(trifluoromethyl)benzyl)-1H-pyrazol-4-yl)methanol

Lithium aluminum hydride (2.0M in tetrahydrofuran, 2.49 mL, 4.97 mmol) was added dropwise to the above mixture of ethyl 3-propyl-1-(4-(trifluoromethyl)benzyl)-1H-pyrazole-4-carboxylate and ethyl 5-propyl-1-(4-(trifluoromethyl)benzyl)-1H-pyrazole-4-carboxylate (1.69 g, 4.97 mmol) in tetrahydrofuran (25 mL) at 0° C. The mixture was allowed to warm to room temperature and stirred overnight. The reaction was quenched with 2N aqueous hydrochloric acid and extracted with ethyl acetate. The extracts were washed with water and brine, dried (MgSO₄), and evaporated. The residue was purified by PTLC (50% ethyl acetate/hexanes) to give (3-propyl-1-(4-(trifluoromethyl)benzyl)-1H-pyrazol-4-yl)methanol (0.60 g); MS for C₁₅H₁₇F₃N₂O m/z 299 (M+H)⁺ and the more polar (5-propyl-1-(4-(trifluoromethyl)benzyl)-1H-pyrazol-4-yl)methanol (0.3 g) as oils.

### Preparation of 2-methyl-2-(4-(((3-propyl-1-(4-(trifluoromethyl)benzyl)-1H-pyrazol-4-yl)methoxy)carbonyl)phenylthio)propanoic acid

4-(1-tert-Butoxy-2-methyl-1-oxopropan-2-ylthio)benzoic acid (0.44 g, 1.47 mmol), (3-propyl-1-(4-(trifluoromethyl)benzyl)-1H-pyrazol-4-yl)methanol (0.44 g, 1.47 mmol), N,N′-dicyclohexylcarbodiimide (0.36 g, 1.76 mmol), and 4-(dimethylamino)pyridine (0.021 mg, 0.176 mmol) in dichloromethane (8 mL) were stirred at room temperature overnight. The mixture was filtered to remove 1,3-dicyclohexylurea and the filtrate evaporated. The residue was purified by PTLC (30% ethyl acetate/hexane) to give (3-propyl-1-(4-(trifluoromethyl)benzyl)-1H-pyrazol-4-yl)methyl 4-(1-tert-butoxy-2-methyl-1-oxopropan-2-ylthio)benzoate as a white solid (0.73 g, 86%); MS for C_{3O}H₃₅ F₃N₂O₄S m/z 577 (M+H)⁺. The ester and 4M hydrogen chloride in dioxane (4 mL) were stirred at room temperature overnight then evaporated under vacuum. The residue was purified by PTLC (10% methanol/dichloromethane) to give product as a white solid (0.48 g, 73%); HPLC retention time (Phenomenex 150×4.6 mm, 5μ, 1 mL/min, 20 mM ammonium acetate buffer, MeCN:water=15:85 for 2 min, then to 10:90 in 18 minutes)=14.8 min; ¹H-NMR (400 MHz, d₆-DMSO) 7.92 (s, 1H); 7.88 (d, J=8 Hz, 2H); 7.71 (d, J=8 Hz); 7.54 (d, J=8 Hz, 2H); 7.40 (d, J=8 Hz, 2H); 5.37 (s, 2H); 5.20 (s, 2H); 2.56 (t, J=7.2 Hz, 2H); 1.58 (m, 2H); 1.42 (s, 6H); 0.87 (t, J=7.2 Hz, 3H); MS for C₂₆H₂₇F₃N₂O₄S m/z 521 (M+H)⁺.

### Example 53

### Compound 54

### 2-methyl-2-(4-(((3-(4-methylbenzyl)-1-propyl-1H-pyrazol-5 yl)methoxy)carbonyl)phenoxy)propanoic acid (ATI-20346)

### Preparation of ethyl 3-(4-methylbenzyl)-1H-pyrazole-5-carboxylate

The ester was prepared as outlined in the following reference: J. Med. Chem. 2003, 46, 3945-3951.

### Preparation of ethyl 3-(4-methylbenzyl)-1-propyl-1H-pyrazole-5-carboxylate

Ethyl 3-(4-methylbenzyl)-1H-pyrazole-5-carboxylate (1.15 g, 4.71 mmol, 1 equiv) was dissolved in DMF (10 mL) and potassium carbonate (1.953 g, 14.13 mmol, 3 equiv) and 1-iodopropane (459.62 μl, 4.71 mmol, 1.1 equiv) were sequentially added. The reaction was stirred at 65° C. for 16 hours and quenched with water. The product was extracted with ethyl acetate. The organic layer was washed with brine, dried over MgSO₄ and concentrated. The resulting residue was purified using Biotage SP-4 chromatography system (7:3 hexanes:ethyl acetate as the eluent condition) to afford the desired product as a clear oil (0.773 g, 57.3%).

MW=286; [M+H]=287.3

¹HNMR (DMSO-d₆)-δ 7.12-7.07 (m, 4H), 6.55 (s, 1H), 4.39-4.35 (t, 2H, J=6.8 & 7.2 Hz), 4.27-4.22 (q, 2H, J=6.8 Hz), 3.84 (s, 2H), 2.25 (s, 3H), 1.75-1.7 (p, 2H), 1.27-1.24 (t, 3H, J=6.8 & 7.2 Hz), 0.82-0.79 (t, 3H, J=7.6 & 7.2 Hz).

### Preparation of (3-(4-methylbutylbenzyl)-1-propyl-1H-pyrazol-5-yl)methanol

Ethyl 3-(4-methylbenzyl)-1-propyl-1H-pyrazole-5-carboxylate (0.73 g, 2.7 mmol, 1 equiv) was dissolved in THF (5 mL) and cooled to 0° C. 2M Lithium aluminium hydride in THF (1.35 mL, 2.7 mmol, 1 equiv) was added to the stirring mixture. The reaction was allowed to warm to room temperature slowly and stirred as such for 16 hours. After removal of the THF the residue was taken into 5% methanol/dicholormethane and washed with 10% HCl and brine. The organic layer was dried over MgSO₄ and concentrated to afford the desired alcohol as a clear oil (0.496 g, 75% yield).

¹HNMR (DMSO-d₆)-δ 7.12-7.07 (m, 4H), 4.40 (s, 1H), 3.96-3.92 (t, 2H, J=7.6 & 6.8 Hz), 3.75 (s, 2H), 2.24 (s, 3H), 1.76-1.7 (p, 2H), 0.85-0.81 (t, 3H, J=7.2 & 7.6 Hz).

### Preparation of (3-(4-methylbenzyl)-1-propyl-1H-pyrazol-5-yl)methyl 4-(1-tert-butoxy-2-methyl-1-oxopropan-2-yloxy)benzoate

(3-(4-methylbutylbenzyl)-1-propyl-1H-pyrazol-5-yl)methanol (0.248 g, 1.012 mmol, 1 equiv), 4-(1-tert-butoxy-2-methyl-1-oxopropan-2-yloxy)benzoic acid (0.283 g, 1.012 mmol, 1 equiv), dimethylaminopyridine (0.124 g, 1.012 mmol, 1 equiv) were taken into dimethylformamide (5 mL). 1-(3-dimethylaminopropyl)-3-ethylcarbodiimide hydrochloride (0.232 g, 1.214 mmol, 1.2 equiv) was added to the reaction mixture and the latter was stirred at room temperature for 16 hours. The reaction was worked up using ethyl acetate, 10% citric acid and brine, dried over MgSO₄. The residue obtained upon concentration of the organic layer afforded the product (0.477 g, 93%) in high purity and was used as such.

MW=506; [M+H]=507.07

¹HNMR (DMSO-d₆)-δ 7.87-7.83 (m, 2H), 7.12-7.06 (m, 4H), 6.86-6.82 (m, 2H), 6.08 (s, 1H), 5.29 (s, 2H), 4.04-4.01 (t, 2H, J=7.2 Hz), 3.79 (s, 2H), 2.24 (s, 3H), 1.75-1.74 (m, 2H), 1.55 (bs, 6H), 1.35 (s, 9H), 0.83-0.79 (t, 3H, J=7.6 & 7.2 Hz).

### Preparation of 2-methyl-2-(4-(((3-(4-methylbenzyl)-1-propyl-1H-pyrazol-5-yl)methoxy)carbonyl)phenoxy)propanoic acid

(3-(4-methylbenzyl)-1-propyl-1H-pyrazol-5-yl)methyl 4-(1-tert-butoxy-2-methyl-1-oxopropan-2-yloxy)benzoate (2.38 g, 4.32 mmol, 1 equiv) was taken into 4 M HCl/dioxane and the reaction was stirred for 16 hours. It was concentrated and the resulting residue was purified using Biotage SP-4 chromatography system (9:1 mixture of dichloromethane:methanol as the eluent condition) to afford the desired compound as a crystalline solid (2.04 g, 95% yield).

MW=450; [M+H]=451

¹HNMR (DMSO-d₆)-δ 12.82 (s, 1H), 7.85-7.83 (m, 2H), 7.12-7.06 (m, 4H), 6.88-6.85 (m, 2H), 6.08 (s, 1H), 5.29 (s, 2H), 4.05-4.01 (t, 2H, J=7.2 Hz), 3.79 (s, 2H), 2.24 (s, 3H), 1.78-1.72 (m, 2H), 1.55 (bs, 6H), 0.84-0.80 (t, 3H, J=7.2 & 7.6 Hz).

### Example 54

### Compound 55

### 2-methyl-2-(4-(((3-(4-methylbenzyl)-1-propyl-1H-pyrazol-5-yl)methoxy)carbonyl)phenylthio)propanoic acid (ATI-20347)

### Preparation of ethyl 3-(4-methylbenzyl)-1H-pyrazole-5-carboxylate

The ester was prepared as outlined in the following reference: J. Med. Chem. 2003, 46, 3945-3951.

### Preparation of ethyl 3-(4-methylbenzyl)-1-propyl-1H-pyrazole-5-carboxylate

Ethyl 3-(4-methylbenzyl)-1H-pyrazole-5-carboxylate (1.15 g, 4.71 mmol, 1 equiv) was dissolved in DMF (10 mL) and potassium carbonate (1.953 g, 14.13 mmol, 3 equiv) and 1-iodopropane (459.62 μl, 4.71 mmol, 1.1 equiv) were sequentially added. The reaction was stirred at 65° C. for 16 hours and quenched with water. The product was extracted with ethyl acetate. The organic layer was washed with brine, dried over MgSO₄ and concentrated. The resulting residue was purified using Biotage SP-4 chromatography system (7:3 hexanes:ethyl acetate as the eluent condition) to afford the desired product as a clear oil (0.773 g, 57.3%).

MW=286; [M+H]=287.3

¹HNMR (DMSO-d₆)-δ 7.12-7.07 (m, 4H), 6.55 (s, 1H), 4.39-4.35 (t, 2H, J=6.8 & 7.2 Hz), 4.27-4.22 (q, 2H, J=6.8 Hz), 3.84 (s, 2H), 2.25 (s, 3H), 1.75-1.7 (p, 2H), 1.27-1.24 (t, 3H, J=6.8 & 7.2 Hz), 0.82-0.79 (t, 3H, J=7.6 & 7.2 Hz).

### Preparation of (3-(4-methylbutylbenzyl)-1-propyl-1H-pyrazol-5-yl)methanol

Ethyl 3-(4-methylbenzyl)-1-propyl-1H-pyrazole-5-carboxylate (0.73 g, 2.7 mmol, 1 equiv) was dissolved in THF (5 mL) and cooled to 0° C. 2M Lithium aluminium hydride in THF (1.35 mL, 2.7 mmol, 1 equiv) was added to the stirring mixture. The reaction was allowed to warm to room temperature slowly and stirred as such for 16 hours. After removing most of the THF the residue was taken into 5% methanol/dicholormethane and washed with 10% HCl and brine. The organic layer was dried over MgSO₄ and concentrated to afford the desired alcohol as a clear oil (0.496 g, 75% yield).

¹HNMR (DMSO-d₆)-δ 7.12-7.07 (m, 4H), 4.40 (s, 1H), 3.96-3.92 (t, 2H, J=7.6 & 6.8 Hz), 3.75 (s, 2H), 2.24 (s, 3H), 1.76-1.7 (p, 2H), 0.85-0.81 (t, 3H, J=7.2 & 7.6 Hz).

### Preparation of (3-(4-methylbenzyl)-1-propyl-1H-pyrazol-5-yl)methyl 4-(1-tert-butoxy-2-methyl-1-oxopropan-2-ylthio)benzoate

(3-(4-methylbutylbenzyl)-1-propyl-1H-pyrazol-5-yl)methanol (0.248 g, 1.012 mmol, 1 equiv), 4-(1-tert-butoxy-2-methyl-1-oxopropan-2-ylthio)benzoic acid (0.300 g, 1.012 mmol, 1 equiv), dimethylaminopyridine (0.124 g, 1.012 mmol, 1 equiv) were taken into dimethylformamide (5 mL). 1-(3-Dimethylaminopropyl)-3-ethylcarbodiimide hydrochloride (0.232 g, 1.214 mmol, 1.2 equiv) was added to the reaction mixture and the latter was stirred at room temperature for 16 hours. The reaction was worked up using ethyl acetate, 10% citric acid and brine, dried over MgSO₄. The residue obtained upon concentration of the organic layer afforded the product (0.494 g, 93%) in high purity and was used as such.

MW=522; [M+H]=523.06

¹HNMR (DMSO-d₆)-δ 7.91-7.89 (m, 2H), 7.57-7.55 (m, 2H), 7.12-7.06 (m, 4H), 6.10 (s, 1H), 5.34 (s, 2H), 4.06-4.02 (t, 2H, J=7.2 Hz), 3.79 (s, 2H), 2.24 (s, 3H), 1.78-1.72 (m, 2H), 1.42 (bs, 6H), 1.32 (s, 9H), 0.83-0.79 (t, 3H, J=7.6 Hz).

### Preparation of 2-methyl-2-(4-(((3-(4-methylbenzyl)-1-propyl-1H-pyrazol-5-yl)methoxy)carbonyl)phenylthio)propanoic acid (ATI-20347)

(3-(4-methylbenzyl)-1-propyl-1H-pyrazol-5-yl)methyl 4-(1-tert-butoxy-2-methyl-1-oxopropan-2-ylthio)benzoate (0.494 g, 0.945 mmol, 1 equiv) was taken into 4 M HO/dioxane and the reaction was stirred for 16 hours. It was concentrated and the resulting residue was purified using Biotage SP-4 chromatography system (9:1 mixture of dichloromethane:methanol as the eluent condition) to afford the desired compound as a crystalline solid (0.338 g, 77% yield).

MW=466; [M+H]=466.99

¹HNMR (DMSO-d₆)-δ 12.81 (s, 1H), 7.89-7.87 (m, 2H), 7.55-7.53 (m, 2H), 7.12-7.06 (m, 4H), 6.10 (s, 1H), 5.34 (s, 2H), 4.06-4.03 (t, 2H, J=6.8 & 7.2 Hz), 3.79 (s, 2H), 2.24 (s, 3H), 1.78-1.73 (m, 2H), 1.42 (bs, 6H), 0.84-0.80 (t, 3H, J=7.2 & 7.6 Hz).

### Example 55

### Compound 21

2-methyl-2-(4-(((1-(4-(methylthio)benzyl)-1H-pyrazol-4 yl)methoxy)carbonyl)phenoxy)propanoic acid

The title compound was prepared using the procedure outlined in Step 5 in 36% yield.

MW=440; [M−H]=438.88

¹HNMR (DMSO-d₆)-δ 13.4 (brs, 1H), 7.92 (s, 1H), 7.85-7.83 (d, 2H, J=8.4 Hz), 7.54 (s, 1H), 7.24-7.18 (m, 4H), 6.86-6.84 (d, 2H, J=8.4 Hz), 5.25 (brs, 2H), 5.16 (s, 2H), 2.44 (s, 3H), 1.54 (s, 6H).

### Preparation of (1-(4-(methylthio)benzyl)-1H-pyrazol-4-yl)methyl 4-(1-tert-butoxy-2-methyl-1-oxopropan-2-yloxy)benzoate

The title compound was prepared using the procedure outlined in Step 3 in 82% yield.

MW=496; [M+H]=496.97

¹HNMR (DMSO-d₆)-δ 7.92 (s, 1H), 7.87-7.85 (d, 2H, J=2.4 Hz), 7.54 (d, 1H), 7.24-7.21 (m, 4H), 6.84-6.82 (m, 2H), 5.25 (s, 2H), 5.13 (s, 2H), 2.44 (s, 3H), 1.55 (s, 6H), 1.36 (s, 9H).

### Preparation of (1-(4-(methylthio)benzyl)-1H-pyrazol-4-yl)methanol

The title compound was prepared using the procedure outlined in Step 2 in 51% yield.

¹HNMR (DMSO-d₆)-δ 7.66 (d, 1H), 7.36 (s, 1H), 7.24-7.17 (m, 4H), 5.22 (s, 2H), 4.86-4.82-4.80 (t, 1H, J=5.2 & 5.6 Hz), 4.33-4.32 (d, 2H, J=5.2 Hz).

### Preparation of ethyl 1-(4-(methylthio)benzyl)-1H-pyrazole-4-carboxylate

The title compound was prepared using the procedure outlined in Step 1 in 49% yield.

MW=276

¹HNMR (DMSO-d₆)-δ 8.44 (d, 1H, J=2.4 Hz), 7.86 (d, 1H), 7.23-7.17 (m, 4H), 5.31 (s, 2H), 4.22-4.17 (dd, 1H, J=7.6 & 6.8 Hz), 2.44 (s, 3H), 1.27-1.23 (t, 2H, J=7.2 Hz).

### Example 56

### Compound 20

### 2-methyl-2-(4-(((1-(4-(methylthio)benzyl)-1H-pyrazol-4-yl)methoxy)carbonyl)phenylthio)propanoic acid

The title compound was prepared using the procedure outlined in Step 5 in 53% yield.

MW=456; [M−H]=454.74

¹HNMR (DMSO-d₆)-δ 12.9 (brs, 1H), 7.94 (s, 1H), 7.89-7.87 (d, 2H, J=8 Hz), 7.56-7.53 (m, 3H), 7.24-7.18 (m, 4H), 5.26 (s, 2H), 5.21 (s, 2H), 2.44 (s, 3H), 1.41 (s, 6H).

### Preparation of (1-(4-methylthio)benzyl)-1H-pyrazol-4-yl)methyl 4-(1-tert-butoxy-2-methyl-1-oxopropan-2-ylthio)benzoate

The title compound was prepared using the procedure outlined in Step 4 in 82% yield.

MW=512; [M+H]=513.02

¹HNMR (DMSO-d₆)-δ 7.95-7.9 (m, 3H), 7.57-7.54 (m, 3H), 7.21-7.21 (d, 4H, J=2.4 Hz), 5.26 (s, 2H), 5.21 (s, 2H), 2.44 (s, 3H), 1.41 (s, 6H), 1.33 (s, 9H).

### Preparation of (1-(4-(methylthio)benzyl)-1H-pyrazol-4-yl)methanol

The title compound was prepared using the procedure outlined in Step 2 in 51% yield.

¹HNMR (DMSO-d₆)-δ 7.66 (d, 1H), 7.36 (s, 1H), 7.24-7.17 (m, 4H), 5.22 (s, 2H), 4.82-4.80 (t, 1H, J=5.2 & 5.6 Hz), 4.33-4.32 (d, 2H, J=5.2 Hz).

### Preparation of ethyl 1-(4-(methylthio)benzyl)-1H-pyrazole-4-carboxylate

The title compound was prepared using the procedure outlined in Step 1 in 49% yield.

MW=276

¹HNMR (DMSO-d₆)-δ 8.44 (d, 1H, J=2.4 Hz), 7.86 (d, 1H), 7.23-7.17 (m, 4H), 5.31 (s, 2H), 4.22-4.17 (dd, 1H, J=7.6 & 6.8 Hz), 2.44 (s, 3H), 1.27-1.23 (t, 2H, J=7.2 Hz).

### Example 57

### Compound 23

### 2-(4-(((1-(biphenyl-4-ylmethyl)-1H-pyrazol-4-yl)methoxy)carbonyl)phenoxy)-2-methylpropanoic acid

The title compound was prepared using the procedure outlined in Step 5 in 63% yield.

MW=470; [M−H]=470.99

¹HNMR (DMSO-d₆)-δ 13.1 (brs, 1H), 7.98 (s, 1H), 7.82 (brs, 2H), 7.64-7.62 (m, 4H), 7.57 (s, 1H), 7.46-7.43 (m, 2H), 7.37-7.32 (m, 3H), 6.86 (brs, 2H), 5.35 (s, 2H), 5.17 (s, 2H), 1.50 (s, 6H).

### Preparation of (1-(biphenyl-4-ylmethyl)-1H-pyrazol-4-yl)methyl 4-(1-tert-butoxy-2-methyl-1-oxopropan-2-yloxy)benzoate

The title compound was prepared using the procedure outlined in Step 3 in 73% yield.

MW=526; [M+H]=527.04

¹HNMR (DMSO-d₆)-δ 7.99 (s, 1H), 7.88-7.86 (dd, 2H, J=2.4 Hz), 7.65-7.62 (m, 4H), 7.57 (d, 1H), 7.47-7.43 (m, 2H), 7.36-7.33 (m, 3H), 5.36 (s, 2H), 5.18 (s, 2H), 1.55 (s, 6H), 1.35 (s, 9H).

### Preparation of (1-(biphenyl-4-ylmethyl)-1H-pyrazol-4-yl)methanol

The title compound was prepared using the procedure outlined in Step 2 in 41% yield. ¹HNMR (DMSO-d₆)-δ 7.72 (d, 1H), 7.65-7.61 (m, 4H), 7.47-7.44 (m, 2H), 7.39-7.3 (m, 4H), 5.32 (s, 2H), 4.84-4.82 (t, 1H, J=5.2 & 5.6 Hz), 4.35-4.34 (d, 2H, J=5.6 Hz).

### Preparation of ethyl 1-(biphenyl-4-ylmethyl)-1H-pyrazole-4-carboxylate

The title compound was prepared using the procedure outlined in Step 1 in quantitative yield.

¹HNMR (DMSO-d₆)-δ 8.51 (s, 1H), 7.89 (d, 1H), 7.65-7.63 (m, 4H), 7.47-7.43 (m, 2H), 7.38-7.35 (m, 3H), 5.41 (s, 2H), 4.23-4.18 (dd, 1H, J=7.2 Hz), 1.27-1.24 (t, 2H, J=6.8 & 7.2 Hz).

### Example 58

### Compound 22

### 2-(4-(((1-(biphenyl-4-ylmethyl)-1H-pyrazol-4-yl)methoxy)carbonyl)phenylthio)-2-methylpropanoic acid

The title compound was prepared using the procedure outlined in Step 5 in 37% yield.

MW=486; [M−H]=484.88

¹HNMR (DMSO-d₆)-δ 12.9 (brs, 1H), 8.01 (s, 1H), 7.90-7.88 (d, 2H), 7.64-7.62 (m, 4H), 7.59 (s, 1H), 7.55-7.53 (m, 2H), 7.47-7.43 (m, 2H), 7.37-7.33 (m, 3H), 5.36 (s, 2H), 5.23 (s, 2H), 1.41 (s, 6H).

### Preparation of (1-(biphenyl-4-ylmethyl)-1H-pyrazol-4-yl)methyl 4-(1-tert-butoxy-2-methyl-1-oxopropan-2-ylthio)benzoate

The title compound was prepared using the procedure outlined in Step 4 in 76% yield.

MW=542; [M+H]=543.05

¹HNMR (DMSO-d₆)-δ 8.01 (s, 1H), 7.93-7.91 (dd, 2H, J=2.4 Hz), 7.64-7.62 (m, 4H), 7.60 (s, 1H), 7.57-7.53 (m, 2H), 7.47-7.43 (m, 2H), 7.38-7.32 (m, 3H), 5.36 (s, 2H), 5.23 (s, 2H), 1.41 (s, 6H), 1.32 (s, 9H).

### Preparation of (1-(biphenyl-4-ylmethyl)-1H-pyrazol-4-yl)methanol

The title compound was prepared using the procedure outlined in Step 2 in 41% yield.

¹HNMR (DMSO-d₆)-δ 7.72 (d, 1H), 7.65-7.61 (m, 4H), 7.47-7.44 (m, 2H), 7.39-7.3 (m, 4H), 5.32 (s, 2H), 4.84-4.82 (t, 1H, J=5.2 & 5.6 Hz), 4.35-4.34 (d, 2H, J=5.6 Hz).

### Preparation of ethyl 1-(biphenyl-4-ylmethyl)-1H-pyrazole-4-carboxylate

The title compound was prepared using the procedure outlined in Step 1 in quantitative yield.

¹HNMR (DMSO-d₆)-δ 8.51 (s, 1H), 7.89 (d, 1H), 7.65-7.63 (m, 4H), 7.47-7.43 (m, 2H), 7.38-7.35 (m, 3H), 5.41 (s, 2H), 4.23-4.18 (dd, 1H, J=7.2 Hz), 1.27-1.24 (t, 2H, J=6.8 & 7.2 Hz).

### Example 59

### Compound 25

### 2-(4-(((1-(3,4-difluorobenzyl)-1H-pyrazol-4-yl)methoxy)carbonyl)phenoxy)-2-methylpropanoic acid

The title compound was prepared using the procedure outlined in Step 5 in 45% yield.

MW=430; [M−H]=430.96

¹HNMR (DMSO-d₆)-δ 13.4 (brs, 1H), 7.97 (s, 1H), 7.82 (brs, 2H), 7.57 (s, 1H), 7.44-7.3 (m, 2H), 7.09 (brs, 1H), 6.86 (brs, 2H), 5.3 (s, 2H), 5.16 (s, 2H), 1.52 (s, 6H).

### Preparation of (1-(3,4-difluorobenzyl)-1H-pyrazol-4-yl)methyl 4-(1-tert-butoxy-2-methyl-1-oxopropan-2-yloxy)benzoate

The title compound was prepared using the procedure outlined in Step 3 in 81% yield.

MW=486; [M+H]=486.99

¹HNMR (DMSO-d₆)-δ 7.98 (s, 1H), 7.88-7.85 (dd, 2H, J=2.4 Hz), 7.58 (d, 4H), 7.42-7.37 (m, 2H), 7.12-7.06 (m, 1H), 6.85-6.83 (m, 1H), 5.31 (s, 2H), 5.17 (s, 2H), 1.55 (s, 6H), 1.36 (s, 9H).

### Preparation of (1-(3,4-difluorobenzyl)-1H-pyrazol-4-yl)methanol

The title compound was prepared using the procedure outlined in Step 2 in 24% yield.

¹HNMR (DMSO-d₆)-δ 7.72 (d, 1H), 7.44-7.37 (m, 2H), 7.32-7.26 (m, 1H), 7.09-7.06 (m, 1H), 5.28 (s, 2H), 4.85-4.82 (t, 1H, J=5.2 & 5.6 Hz), 4.35-4.33 (d, 2H, J=5.6 Hz).

### Preparation of ethyl 1-(3,4-difluorobenzyl)-1H-pyrazole-4-carboxylate

The title compound was prepared using the procedure outlined in Step 1 in 90% yield.

MW=266; [M+H]=267.51

¹HNMR (DMSO-d₆)-δ 8.49 (d, 1H), 7.88 (d, 1H), 7.46-7.36 (m, 2H), 7.14-7.11 (m, 1H), 5.36 (s, 2H), 4.23-4.18 (dd, 1H, J=6.8 Hz), 1.27-1.23 (t, 2H, J=7.2 & 6.8 Hz).

### Example 60

### Compound 24

### 2-(4-(((1-(3,4-Difluorobenzyl)-1H-pyrazol-4-yl)methoxy)carbonyl)phenylthio)-2-methylpropanoic acid

The title compound was prepared using the procedure outlined in Step 5 in 26% yield.

MW=446; [M+H]=446.86

¹HNMR (DMSO-d₆)-δ 12.9 (brs, 1H), 7.99 (s, 1H), 7.88-7.87 (d, 2H), 7.59 (s, 1H), 7.55-7.53 (d, 2H, J=8.0 Hz), 7.44-7.3 (m, 2H), 7.10 (brs, 1H), 6.86 (brs, 1H), 5.31 (s, 2H), 5.21 (s, 2H), 1.4 (s, 6H).

### Preparation of (1-(3,4-difluorobenzyl)-1H-pyrazol-4-yl)methyl 4-(1-tert-butoxy-2-methyl-1-oxopropan-2-ylthio)benzoate

The title compound was prepared using the procedure outlined in Step 4 in 88% yield.

MW=502; [M+Na]=525

¹HNMR (DMSO-d₆)-δ 8 (s, 1H), 7.93-7.91 (d, 2H, J=8.4 Hz), 7.59 (s, 1H), 7.57-7.54 (m, 2H), 7.42-7.30 (m, 2H), 7.11-7.08 (m, 1H), 5.31 (s, 2H), 5.22 (s, 2H), 1.41 (s, 6H), 1.33 (s, 9H).

### Preparation of (1-(3,4-difluorobenzyl)-1H-pyrazol-4-yl)methanol

The title compound was prepared using the procedure outlined in Step 2 in 24% yield.

¹HNMR (DMSO-d₆)-δ 7.72 (d, 1H), 7.44-7.37 (m, 2H), 7.32-7.26 (m, 1H), 7.09-7.06 (m, 1H), 5.28 (s, 2H), 4.85-4.82 (t, 1H, J=5.2 & 5.6 Hz), 4.35-4.33 (d, 2H, J=5.6 Hz).

### Preparation of ethyl 1-(3,4-difluorobenzyl)-1H-pyrazole-4-carboxylate

The title compound was prepared using the procedure outlined in Step 1 in 90% yield.

[MW]=266; [M+H]=267.51

¹HNMR (DMSO-d₆)-δ 8.49 (d, 1H), 7.88 (d, 1H), 7.46-7.36 (m, 2H), 7.14-7.11 (m, 1H), 5.36 (s, 2H), 4.23-4.18 (dd, 1H, J=6.8 Hz), 1.27-1.23 (t, 2H, J=7.2 & 6.8 Hz).

### Example 61

### Compound 27

2-methyl-2-(4-(((1-(naphthalen-2-ylmethyl)-1H-pyrazol-4-yl)methoxy)carbonyl)phenoxy)propanoic acid

The title compound was prepared using the procedure outlined in Step 5 in 34% yield.

MW=444; [M+H]=444.99

¹HNMR (DMSO-d₆)-δ 13.4 (brs, 1H), 7.99 (s, 1H), 7.89-7.85 (m, 4H), 7.77 (s, 1H), 7.57 (s, 1H), 7.52-7.5 (m, 2H), 7.41-7.39 (m, 1H), 6.86-6.84 (d, 2H, J=8 Hz), 5.47 (s, 2H), 5.17 (s, 2H), 1.53 (s, 6H).

### Preparation of (1-(naphthalen-2-ylmethyl)-1H-pyrazol-4-yl)methyl 4-(1-tert-butoxy-2-methyl-1-oxopropan-2-yloxy)benzoate

The title compound was prepared using the procedure outlined in Step 3 in 78% yield.

MW=500; [M+Na]=501.03

¹HNMR (DMSO-d₆)-δ 8 (s, 1H), 7.9-7.85 (m, 5H), 7.77 (s, 1H), 7.58 (s, 1H), 7.52-7.50 (m, 2H), 7.41-7.39 (dd, 1H, J=1.6 & 2.0 Hz), 5.48 (s, 2H), 5.18 (s, 2H), 1.55 (s, 6H), 1.31 (s, 9H).

### Preparation of (1-(naphthalen-2-ylmethyl)-1H-pyrazol-4-yl)methanol

The title compound was prepared using the procedure outlined in Step 2 in 50% yield.

¹HNMR (DMSO-d₆)-δ 7.89-7.87 (m, 3H), 7.76 (s, 1H), 7.74 (d, 1H), 7.52-7.50 (m, 2H), 7.39 (s, 1H), 7.37 (d, 1H), 5.44 (s, 2H), 4.84-4.81 (t, 1H, J=5.6 & 5.2 Hz), 4.35-4.34 (d, 2H, J=5.2 Hz).

### Preparation of ethyl 1-(naphthalen-2-ylmethyl)-1H-pyrazole-4-carboxylate

The title compound was prepared using the procedure outlined in Step 1 in 78% yield.

MW=280; [M+H]=280.55

¹HNMR (DMSO-d₆)-δ 8.53 (d, 1H), 7.91-7.89 (m, 4H), 7.8 (s, 1H), 7.53-7.5 (m, 2H), 7.43-7.41 (dd, 1H, J=2.0 & 1.6 Hz), 5.54 (s, 2H), 4.23-4.18 (dd, 1H, J=7.2 & 6.8 Hz), 1.28-1.23 (m, 2H).

### Example 62

### Compound 26

### 2-methyl-2-(4-(((1-(naphthalen-2-ylmethyl)-1H-pyrazol-4-yl)methoxy)carbonyl)phenylthio)propanoic acid

The title compound was prepared using the procedure outlined in Step 5 in 34% yield.

MW=460; [M+H]=460.92

¹HNMR (DMSO-d₆)-δ 12.9 (brs, 1H), 8.02 (s, 1H), 7.9-7.85 (m, 5H), 7.77 (s, 1H), 7.60 (s, 1H), 7.52-7.5 (m, 4H), 7.41-7.39 (dd, 1H, J=2.0 & 1.6 Hz), 5.48 (s, 2H), 5.22 (s, 2H), 1.41 (s, 6H).

### Preparation of (1-(naphthalen-2-ylmethyl)-1H-pyrazol-4-yl)methyl 4-(1-tert-butoxy-2-methyl-1-oxopropan-2-ylthio)benzoate

The title compound was prepared using the procedure outlined in Step 4 in 78% yield.

MW=516; [M+Na]=517.03

¹HNMR (DMSO-d₆)-δ 8.03 (s, 1H), 7.92-7.87 (m, 5H), 7.77 (s, 1H), 7.60 (s, 1H), 7.55-7.49 (m, 4H), 7.41-7.39 (dd, 1H, J=2.0 & 1.6 Hz), 5.48 (s, 2H), 5.23 (s, 2H), 1.41 (s, 6H), 1.32 (s, 9H).

### Preparation of (1-(naphthalen-2-ylmethyl)-1H-pyrazol-4-yl)methanol

The title compound was prepared using the procedure outlined in Step 2 in 50% yield.

¹HNMR (DMSO-d₆)-δ 7.89-7.87 (m, 3H), 7.76 (s, 1H), 7.74 (d, 1H), 7.52-7.50 (m, 2H), 7.39 (s, 1H), 7.37 (d, 1H), 5.44 (s, 2H), 4.84-4.81 (t, 1H, J=5.6 & 5.2 Hz), 4.35-4.34 (d, 2H, J=5.2 Hz).

### Preparation of ethyl 1-(naphthalen-2-ylmethyl)-1H-pyrazole-4-carboxylate

The title compound was prepared using the procedure outlined in Step 1 in 78% yield.

[MW]=280; [M+H]=280.55

¹HNMR (DMSO-d₆)-δ 8.53 (d, 1H), 7.91-7.89 (m, 4H), 7.8 (s, 1H), 7.53-7.5 (m, 2H), 7.43-7.41 (dd, 1H, J=2.0 & 1.6 Hz), 5.54 (s, 2H), 4.23-4.18 (dd, 1H, J=7.2 & 6.8 Hz), 1.28-1.23 (m, 2H).

### Example 63

### Compound 28

### 2-methyl-2-(4-(((1-((5,5,8,8-tetramethyl-5,6,7,8-tetrahydronaphthalen-2-yl)methyl)-1H-pyrazol-4-yl)methoxy)carbonyl)phenoxy)propanoic acid

The title compound was prepared using the procedure outlined in Step 5 in 42% yield.

MW=504; [M+H]=505.09

¹HNMR (DMSO-d₆)-δ 13.4 (brs, 1H), 7.93 (s, 1H), 7.82-7.81 (d, 2H, J=4.8 Hz), 7.54 (s, 1H), 7.26-7.24 (d, 1H, J=8.0 Hz), 7.16 (s, 4H), 6.95-6.92 (dd, 1H, J=1.2 & 1.6 Hz), 6.86-6.84 (d, 2H), 5.22 (s, 2H), 5.16 (s, 2H), 1.6-1.52 (m, 12H), 1.23-1.16 (m, 14H).

### Preparation of (1-((5,5,8,8-tetramethyl-5,6,7,8-tetrahydronaphthalen-2-yl)methyl)-1H-pyrazol-4-yl)methyl 4-(1-tert-butoxy-2-methyl-1-oxopropan-2-yloxy)benzoate

The title compound was prepared using the procedure outlined in Step 3 in quantitative yield.

MW=560; [M+H]=560.99

¹HNMR (DMSO-d₆)-δ 7.94 (s, 1H), 7.87-7.83 (m, 2H), 7.55 (d, 1H), 7.26-7.24 (d, 1H, J=8.4 Hz), 7.15-7.14 (d, 1H, J=1.6 Hz), 6.96-6.93 (dd, 1H, J=1.6 & 2 Hz), 6.86-6.84 (m, 2H), 5.23 (s, 2H), 5.17 (s, 2H), 1.55 (s, 10H), 1.36 (s, 9H), 1.2 (s, 7H), 1.15 (s, 6H).

### Preparation of (1-((5,5,8,8-tetramethyl-5,6,7,8-tetrahydronaphthalen-2-yl)methyl)-1H-pyrazol-4-yl)methanol

The title compound was prepared using the procedure outlined in Step 2 in 45% yield.

¹HNMR (DMSO-d₆)-δ 7.65 (d, 1H), 7.35 (s, 1H), 7.26 (s, 1H), 7.24 (s, 1H), 6.93-6.91 (dd, 1H, J=2 Hz), 5.18 (s, 2H), 4.82-4.79 (t, 1H, J=5.6 Hz), 4.33-4.32 (d, 2H, J=5.6 Hz), 1.61 (s, 4H), 1.21-1.2 (d, 12H, J=2.8 Hz).

### Preparation of ethyl 1-((5,5,8,8-tetramethyl-5,6,7,8-tetrahydronaphthalen-2-yl)methyl)-1H-pyrazole-4-carboxylate

The title compound was prepared using the procedure outlined in Step 1 in 87% yield.

MW=340; [M+H]=341.68

¹HNMR (DMSO-d₆)-δ 8.45 (d, 1H), 7.85 (d, 1H), 7.28-7.26 (m, 2H), 6.97-6.95 (dd, 1H, J=2 Hz), 5.27 (s, 2H), 4.23-4.17 (m, 2H), 1.61-1.6 (d, 4H, J=3.6 Hz), 1.27-1.25 (t, 3H, J=3.2 & 4 Hz), 1.2-1.19 (d, 12H, J=2.4 Hz).

### Example 64

### Compound 29

### 2-methyl-2-(4-(((1-((5,5,8,8-tetramethyl-5,6,7,8-tetrahydronaphthalen-2-yl)methyl)-1H-pyrazol-4-yl)methoxy)carbonyl)phenylthio)propanoic acid

The title compound was prepared using the procedure outlined in Step 5 in 28% yield.

MW=520; [M+H]=521.06

¹HNMR (DMSO-d₆)-δ 12.9 (brs, 1H), 7.89-7.87 (d, 1H, J=7.6 Hz), 7.56 (s, 1H), 7.55 (s, 1H), 7.53 (s, 1H), 7.26-7.24 (d, 1H, J=8.0 Hz), 7.14 (d, 1H, J=1.6 Hz), 6.96-6.93 (dd, 1H, J=2 Hz), 5.23 (s, 2H), 5.22 (s, 2H), 1.6 (s, 4H), 1.41 (s, 6H), 1.19 (s, 6H), 1.15 (s, 6H).

### Preparation of (1-((5,5,8,8-tetramethyl-5,6,7,8-tetrahydronaphthalen-2-yl)methyl)-1H-pyrazol-4-yl)methyl 4-(1-tert-butoxy-2-methyl-1-oxopropan-2-ylthio)benzoate

The title compound was prepared using the procedure outlined in Step 4 in quantitative yield.

MW=576; [M+H]=577.05

¹HNMR (DMSO-d₆)-δ 7.97 (s, 1H), 7.91-7.89 (m, 2H), 7.57 (s, 1H), 7.55-7.53 (m, 2H), 7.26-7.24 (d, 1H), 7.14-7.13 (d, 1H, J=1.6 Hz), 6.96-6.93 (dd, 1H, J=2 Hz), 5.23 (s, 2H), 5.23 (s, 2H), 5.22 (s, 2H), 1.59 (s, 4H), 1.41 (s, 6H), 1.34 (s, 9H), 1.19 (s, 6H), 1.14 (s, 6H).

### Preparation of (1-((5,5,8,8-tetramethyl-5,6,7,8-tetrahydronaphthalen-2-yl)methyl)-1H-pyrazol-4-yl)methanol

The title compound was prepared using the procedure outlined in Step 2 in 45% yield.

¹HNMR (DMSO-d₆)-δ 7.65 (d, 1H), 7.35 (s, 1H), 7.26 (s, 1H), 7.24 (s, 1H), 6.93-6.91 (dd, 1H, J=2 Hz), 5.18 (s, 2H), 4.82-4.79 (t, 1H, J=5.6 Hz), 4.33-4.32 (d, 2H, J=5.6 Hz), 1.61 (s, 4H), 1.21-1.2 (d, 12H, J=2.8 Hz).

### Preparation of ethyl 1-((5,5,8,8-tetramethyl-5,6,7,8-tetrahydronaphthalen-2-yl)methyl)-1H-pyrazole-4-carboxylate

The title compound was prepared using the procedure outlined in Step 1 in 87% yield.

MW=340; [M+H]=341.68

¹HNMR (DMSO-d₆)-δ 8.45 (d, 1H), 7.85 (d, 1H), 7.28-7.26 (m, 2H), 6.97-6.95 (dd, 1H, J=2 Hz), 5.27 (s, 2H), 4.23-4.17 (m, 2H), 1.61-1.6 (d, 4H, J=3.6 Hz), 1.27-1.25 (t, 3H, J=3.2 & 4 Hz), 1.2-1.19 (d, 12H, J=2.4 Hz).

### Example 65

### Compound 19

### 2-methyl-2-(4-(((1-(4-(trifluoromethyl)phenyl)-1H-pyrazol-4-yl)methoxy)carbonyl)phenoxy)propanoic acid

The title compound was prepared using the procedure outlined in Step 5 in 29% yield.

MW=448; [M+Na]=471.65

¹HNMR (DMSO-d₆)-δ 8.78 (s, 1H), 8.09-8.07 (d, 2H, J=8 Hz), 7.93 (s, 1H), 7.88 (s, 1H), 7.86 (s, 1H), 7.82 (brs, 2H), 6.85 (s, 2H), 5.26 (s, 2H), 1.39 (s, 6H).

### Preparation of (1-(4-(trifluoromethyl)phenyl)-1H-pyrazol-4-yl)methyl 4-(1-tert-butoxy-2-methyl-1-oxopropan-2-yloxy)benzoate

The title compound was prepared using the procedure outline in Step 3 in 77% yield.

MW=504; [M+Na]=505.54

¹HNMR (DMSO-d₆)-δ 8.79 (s, 1H), 8.09-8.07 (d, 2H, J=8.8 Hz), 7.93-7.92 (d, 1H, J=2 Hz), 7.91-7.90 (d, 1H, J=2.4 Hz), 7.88 (s, 1H), 7.86 (brs, 2H), 6.86-6.84 (dd, 2H, J=2 Hz), 5.27 (s, 2H), 1.55 (s, 6H), 1.36 (s, 9H).

### Preparation of (1-(4-(trifluoromethyl)phenyl)-1H-pyrazol-4-yl)methanol

The title compound was prepared using the procedure outlined in Step 2 in 60% yield.

MW=242; [M+H]=243.41

¹HNMR (DMSO-d₆)-δ 8.53 (d, 1H), 8.06-8.04 (d, 2H, J=8.4 Hz), 7.86-7.84 (d, 1H, J=8.4 Hz), 7.76 (s, 1H), 5.07-5.05 (t, 1H, J=5.6 & 5.2 Hz), 4.45-4.44 (d, 2H, J=4.8 Hz).

### Preparation of ethyl 1-(4-(trifluoromethyl)phenyl)-1H-pyrazole-4-carboxylate

The title compound was prepared using the procedure outlined in Step 7.

MW=284; [M+H]=285.36

¹HNMR (DMSO-d₆)-δ 9.27 (d, 1H), 8.21-8.18 (m, 3H), 7.92-7.9 (d, 2H, J=8.4 Hz), 4.31-4.26 (dd, 1H, J=7.6 & 7.2 Hz), 1.33-1.29 (t, 2H, J=7.2 & 6.8 Hz).

### Example 66

### Compound 18

### 2-methyl-2-(4-(((1-(4-(trifluoromethyl)phenyl)-1H-pyrazol-4-yl)methoxy)carbonyl)phenylthio)propanoic acid

The title compound was prepared using the procedure outlined in Step 5 in 52% yield.

MW=464; [M+Na]=465.47

¹HNMR (DMSO-d₆)-δ 12.9 (brs, 1H), 8.81 (s, 1H), 8.09-8.07 (d, 2H, J=8.4 Hz), 7.95 (s, 1H), 7.93 (s, 1H), 7.91 (s, 1H), 7.89 (s, 2H), 7.87 (s, 1H), 7.57-7.55 (d, 2H, J=8.0 Hz), 5.32 (s, 2H), 1.39 (s, 6H).

### Preparation of (1-(4-(trifluoromethyl)phenyl)-1H-pyrazol-4-yl)methyl 4-(1-tert-butoxy-2-methyl-1-oxopropan-2-ylthio)benzoate

The title compound was prepared using the procedure outline in Step 4 in 88% yield.

MW=520; [M+H]−Bu^{t}=465.39

¹HNMR (DMSO-d₆)-δ 8.82 (s, 1H), 8.09-8.07 (d, 2H, J=8.4 Hz), 7.98-7.96 (m, 3H), 7.89-7.87 (d, 2H, J=8.4 Hz), 6.86-6.84 (dd, 2H, J=2 Hz), 5.33 (s, 2H), 1.41 (s, 6H), 1.29 (s, 9H).

### Preparation of (1-(4-(trifluoromethyl)phenyl)-1H-pyrazol-4-yl)methanol

The title compound was prepared using the procedure outlined in Step 2 in 60% yield.

MW=242; [M+H]=243.41

¹HNMR (DMSO-d₆)-δ 8.53 (d, 1H), 8.06-8.04 (d, 2H, J=8.4 Hz), 7.86-7.84 (d, 1H, J=8.4 Hz), 7.76 (s, 1H), 5.07-5.05 (t, 1H, J=5.6 & 5.2 Hz), 4.45-4.44 (d, 2H, J=4.8 Hz).

### Preparation of ethyl 1-(4-(trifluoromethyl)phenyl)-1H-pyrazole-4-carboxylate

The title compound was prepared using the procedure outlined in Step 7.

MW=284; [M+H]=285.36

¹HNMR (DMSO-d₆)-δ 9.27 (d, 1H), 8.21-8.18 (m, 3H), 7.92-7.9 (d, 2H, J=8.4 Hz), 4.31-4.26 (dd, 1H, J=7.6 & 7.2 Hz), 1.33-1.29 (t, 2H, J=7.2 & 6.8 Hz).

### Example 67

### Compound 35

### 2-methyl-2-(4-(2-oxo-2-((1-(4-(trifluoromethyl)phenyl)-1H-pyrazol-4-yl)methoxy)ethyl)phenoxy)propanoic acid

The title compound was prepared using the procedure outlined in Step 5 in 36% yield.

MW=462; [M+H]=463.4

¹HNMR (DMSO-d₆)-δ 8.67 (s, 1H), 8.05-8.03 (d, 2H, J=8.4 Hz), 7.88-7.86 (d, 2H, J=8.4 Hz), 7.83 (s, 1H), 7.06 (brs, 2H), 6.78 (brs, 2H), 5.06 (s, 2H), 3.58 (s, 2H), 1.33 (s, 6H).

### Preparation of tert-butyl 2-methyl-2-(4-(2-oxo-2-((1-(4-(trifluoromethyl)phenyl)-1H-pyrazol-4-yl)methoxy)ethyl)phenoxy)propanoate

The title compound was prepared using the procedure outline in Step 6 in 89% yield.

MW=518; [M+Na]=541.42

¹HNMR (DMSO-d₆)-δ 8.67 (s, 1H), 8.05-8.03 (d, 2H, J=8.8 Hz), 7.88-7.86 (d, 2H, J=8.4 Hz), 7.81 (s, 1H), 7.16-7.14 (d, 2H, J=8.4 Hz), 6.74-6.72 (dd, 2H, J=2 Hz), 5.07 (s, 2H), 3.63 (s, 2H), 1.46 (s, 6H), 1.36 (s, 9H).

### Preparation of (1-(4-(trifluoromethyl)phenyl)-1H-pyrazol-4-yl)methanol

The title compound was prepared using the procedure outlined in Step 2 in 60% yield.

MW=242; [M+H]=243.41

¹HNMR (DMSO-d₆)-δ 8.53 (d, 1H), 8.06-8.04 (d, 2H, J=8.4 Hz), 7.86-7.84 (d, 1H, J=8.4 Hz), 7.76 (s, 1H), 5.07-5.05 (t, 1H, J=5.6 & 5.2 Hz), 4.45-4.44 (d, 2H, J=4.8 Hz).

### Preparation of ethyl 1-(4-(trifluoromethyl)phenyl)-1H-pyrazole-4-carboxylate

The title compound was prepared using the procedure outlined in Step 7.

MW=284; [M+H]=285.36

¹HNMR (DMSO-d₆)-δ 9.27 (d, 1H), 8.21-8.18 (m, 3H), 7.92-7.9 (d, 2H, J=8.4 Hz), 4.31-4.26 (dd, 1H, J=7.6 & 7.2 Hz), 1.33-1.29 (t, 2H, J=7.2 & 6.8 Hz).

### Example 68

### Compound 17

### 2-methyl-2-(4-(((3-phenyl-1-p-tolyl-1H-pyrazol-4-yl)methoxy)carbonyl)phenoxy)propanoic acid

The title compound was prepared using the procedure outlined in Step 9 in 83% yield.

MW=470; [M+H]=471.59

¹HNMR (DMSO-d₆)-δ 12.9 (brs, 1H), 8.69 (s, 1H), 7.84-7.79 (m, 6H), 7.51-7.32 (m, 5H), 6.85 (brs, 1H), 5.36 (s, 2H), 2.35 (s, 3H), 1.52 (s, 6H).

### Preparation of (3-phenyl-1-p-tolyl-1H-pyrazol-4-yl)methyl 4-(1-(benzyloxy)-2-methyl-1-oxopropan-2-yloxy)benzoate

The title compound was prepared using the procedure outlined in Step 8 in 79% yield.

MW=560; [M+H]=561.55

¹HNMR (DMSO-d₆)-δ 8.69 (s, 1H), 7.82-7.79 (m, 6H), 7.5-7.24 (m, 10H), 6.79-6.77 (d, 2H, J=8.8 Hz), 5.38 (s, 2H), 5.16 (s, 2H), 2.35 (s, 3H), 1.59 (s, 6H).

### Example 69

Compound Potency

Construction of recombinant plasmids. Plasmids pGal4-hPPARα, pGal4-hPPARγ, pGal4-hPPARδ and pG5-TK-pGL3 were constructed as previously described by Staels, Bart, et al., Nature 1998 Jun. 25; 393 (6687): 790-793, by Staels (Institut Pasteur de Lille, FR).

Transient transfection assays. Cos cells were seeded in 60-mm dishes at a density of 5.5×10⁵ cells/dish in Dulbecco's modified Eagle's medium (DMEM) supplemented with 10% fetal calf serum (FCS) and incubated at 37° C. for 24 hours before transfection. Cells were transfected in Opti-MEM (Gibco; Invitrogen, Carlsbad, Calif.) without FCS for 3 hours at 37° C., using polyethylenimine, with reporter and expression plasmids. The plasmid pBluescript (Stratagene, La Jolla, Calif.) was used as carrier DNA to set the final amount of DNA to 5.5 μg/dish. The pCMV-b-galactosidase expression plasmid was cotransfected as a control for transfection efficiency. Transfection was stopped by the addition of DMEM supplemented with 10% FCS, and cells were then incubated at 37° C. After 16 hours, cells were trypsinized and seeded in 96-well plates at the density of 2×10⁴ cells/well and incubated for 6 hours in 10% FCS-containing DMEM. Cells were then incubated for 24 hours in DMEM containing 0.2% FCS and increasing concentrations of the compound tested or vehicle (DMSO). At the end of the experiment, cells were washed once with ice-cold PBS, lysed and the luciferase and b-galactosidase assays were performed in a luminometer and spectrophotomer. Transfection efficiencies were normalized with the b-galactosidase activity assay. Half-maximal effective concentration (EC₅₀) was estimated using Prism software (GraphPad). All transfection experiments were performed at least three times.

In the following tables, proprietary compound EC₅₀ values for hPPAR α, β, δ and γ are shown. The EC₅₀ value is the concentration of a drug that induces a response halfway between the baseline and maximum, and is commonly used as a measure of drug potency. EC₅₀ values are reported on a scale of 1 through 5, wherein the value scale is defined for each receptor as follows:

Receptor Activity Scale

| | EC₅₀ value | | | | |
|---|---|---|---|---|---|
| | <1 nM | 1-50 nM | 51-200 nM | 201-999 nM | >1 uM |
| Scaled | 1 | 2 | 3 | 4 | 5 |
| number | | | | | |

| | | | | Full Length | | |
|---|---|---|---|---|---|---|
| | Gal-4 hPPAR | | | hPPAR | | |
| Compound | α | γ | β/δ | α | γ | β/δ |
| 150 | 2 | 5 | 5 | 2 | | |
| 152 | 3 | 5 | 5 | 1 | | |
| 151 | 2 | 5 | 5 | 1 | | |
| 168 | 4 | 5 | 5 | 3 | 5 | |
| 180 | 4 | 5 | 5 | 1 | | |
| 185 | 2 | 5 | 5 | 1 | | |
| 188 | 2 | 5 | 5 | 1 | | |
| 159 | 2 | 5 | 5 | 2 | | |
| 192 | 2 | 5 | 5 | 1 | | |
| 191 | 2 | 3 | 5 | 1 | 3 | 3 |
| 190 | 2 | 5 | 5 | 1 | | |
| 189 | 2 | 2 | 5 | 1 | 3 | |
| 212 | 1 | 3 | 5 | 1 | 3 | |
| 160 | 2 | 3 | 5 | 1 | 4 | 4 |
| 187 | 2 | 3 | 5 | 1 | 3 | |
| 161 | 2 | 3 | 3 | 1 | 4 | 4 |
| 186 | 2 | 3 | 5 | 3 | 3 | |
| 56 | 3 | 5 | 5 | 2 | 4 | |
| 60 | 2 | 5 | 5 | 2 | | |
| 62 | 3 | 5 | 5 | 3 | | |
| 1 | 2 | 5 | 5 | 1 | 3 | 5 |
| 2 | 4 | 5 | 5 | 2 | | |
| 81 | 2 | 5 | 5 | 1 | | |
| 67 | 2 | 5 | 5 | 1 | | |
| 3 | 3 | 5 | 5 | 1 | | |
| 89 | 3 | 5 | 5 | 2 | | |
| 68 | 3 | 5 | 5 | 3 | | |
| 69 | 2 | 5 | 5 | | | |
| 71 | 3 | 5 | 5 | | | |
| 73 | 3 | 5 | 5 | | | |
| 6 | 2 | 4 | 5 | | | |
| 8 | 3 | 5 | 5 | | | |
| 10 | 2 | 4 | 5 | | | |
| 75 | 3 | 5 | 5 | | | |
| 12 | 3 | 5 | 5 | | | |
| 17 | 3 | 4 | 2 | | | |
| 18 | 2 | 4 | 4 | | | |
| 32 | 2 | 5 | 5 | | | |
| 87 | 2 | 5 | 5 | | | |
| 85 | 4 | 5 | 5 | | | |
| 176 | 3 | 5 | 5 | | | |
| 218 | 3 | 5 | 5 | | | |
| 165 | 3 | 5 | 5 | | | |
| 179 | 3 | 5 | 5 | | | |
| 164 | 2 | 5 | 5 | | | |
| 177 | 2 | 4 | 5 | | | |
| 163 | 2 | 5 | 5 | | | |
| 28 | 4 | 5 | 5 | | | |
| 20 | 2 | 5 | 5 | | | |
| 22 | 3 | 5 | 5 | | | |
| 24 | 3 | 5 | 5 | | | |
| 26 | 2 | 5 | 5 | | | |
| 29 | 2 | 5 | 5 | | | |
| 30 | 2 | 5 | 5 | | | |
| 95 | 2 | 5 | 5 | | | |
| 97 | 4 | 5 | 5 | | | |
| 201 | 2 | 5 | 5 | | | |
| 203 | 2 | 5 | 5 | | | |
| 206 | 4 | 5 | 5 | | | |
| 210 | 4 | 5 | 5 | | | |
| 102 | 3 | 5 | 5 | | | |
| 204 | 3 | 5 | 5 | | | |
| 202 | 2 | 5 | 5 | | | |
| 196 | 3 | 5 | 5 | | | |
| 199 | 2 | 5 | 5 | | | |
| 193 | 3 | 5 | 5 | | | |
| 195 | 2 | 5 | 5 | | | |
| 197 | 2 | 5 | 5 | | | |
| 266 | 4 | 5 | 5 | | | |
| 144 | 3 | 5 | 5 | | | |
| 145 | 4 | 5 | 5 | | | |
| 264 | 1 | 3 | 5 | | | |
| 136 | 2 | 3 | 5 | | | |
| 137 | 3 | 5 | 5 | | | |
| 138 | 2 | 5 | 5 | | | |
| 139 | 2 | 5 | 5 | | | |
| 140 | 2 | 5 | 5 | | | |
| 261 | 3 | 5 | 5 | | | |
| 133 | 2 | 5 | 5 | | | |
| 248 | 3 | 5 | 5 | | | |
| 249 | 2 | 5 | 5 | | | |
| 91 | 4 | 5 | 5 | | | |

### Example 70

### Metabolism Studies Using Human Liver Microsomal Preparations

Compounds were incubated in pooled human liver microsomes (HLMs) in the presence or absence of an NADPH-generating system. NADPH is required for the activity of cytochrome P450 (CYP) and other oxidative enzymes present in HLMs, but not for esterase activity. An additional incubation was carried out in the absence of HLM to assess compound stability. The disappearance of parent and the appearance of the corresponding acid metabolite were monitored over the time course of the incubations using LC-MS/MS.

A stock solution (20 mM) of each proprietary compound in DMSO was prepared and store at −20° C. until needed. A working stock solution (0.2 mM) was prepared for each proprietary compound by adding an aliquot of the stock solution (10 μL) to acetonitrile (990 μL).

An aliquot of the HLM solution was removed from the −80° C. freezer and placed on ice. Tris Buffer (50 mM, pH 7.4) containing MgCl2 (hexahydrate, 5 mM) was pre-incubated in a 37° C. water bath. A set of Eppendorf microfuge tubes (1.5 mL) was appropriately labeled. To each tube was added Tris buffer with or without the NADPH generating system. An aliquot of pooled HLMs was added to all the incubations except those that were the buffer control.

Tubes were preincubated for 5 min in a shaking incubator bath (37° C., 900 rpm) and the reaction was initiated by adding an aliquot of the proprietary working solution (5 μL). The additions were timed carefully in order to coordinate the sample collections below. A small aliquot (50 μL) was removed at 0, 5, 15, 30, 60 and 90 mM and delivered to the respective tubes containing dextrorphan (100 μL of 0.5 μg/mL solution) in dissolved in methanol or acetonitrile. The samples were centrifuged at 14000 g for 15 minutes at 4° C. The supernatant was transferred to clean labeled HPLC injection vials and stored at −80° C. until analyzed. An aliquot of the supernatant (25 μ) was added to water (75 μL), mixed and injected onto the LC-MS/MS system for analysis. Results for some compounds of the invention can be found in the table that follows.

“HLM t_{1/2}” represents the observed half life of the compound under conditions that included NADPH, i.e., the CYP system was active. Comparison to the half life of the test article under (−)NADPH conditions, the enzyme predominant ly responsible for the metabolism of the test article. For example, if the observed half life of the (−)NADPH reaction is 45 minutes, and the observed half life of the (+)NADPH system is 40 minutes, the enzyme active in the absence of NADPH (esterase) is responsible for the bulk of the metabolic activity. If the test article is stable in the absence of NADPH and significantly metabolized in the presence of NADPH, the CYP system is responsible for the bulk or all of the metabolic activity. Where roughly equal metabolism is seen by each enzyme system, “mixed” is noted in the “Predominant enzyme” column.

### Example 71

### Metabolism Studies Using Pig Liver Esterase.

The esterase-mediated metabolism of these compounds was studied using commercially available pig liver esterase in the presence and absence of human plasma proteins. The disappearance of parent was monitored over the time course of the incubations using a LC-UV detection system.

A stock solution and working stock solution was prepared for each proprietary compound as described for the human liver microsomal incubations. Working solutions of pig liver esterase in potassium phosphate buffer (1s0 mM, pH 7.4) and pig liver esterase in human plasma protein were prepared by dissolving pig liver esterase (2.51 mg) in the phosphate buffer or plasma protein solutions, respectively.

A set of three Eppendorf microfuge tubes (1.5 mL) was appropriately labeled. To each tube was added 1.5 mL of phosphate buffer, pig liver esterase in buffer, or pig liver esterase in human plasma protein solution. These solutions were pre-incubated (37° C., 900 rpm) and the reactions were initiated by adding an aliquot (37.5 μL) of the working solution of proprietary compound to each tube. An aliquot (100 μL) from each tube was removed at time 0, 5, 30, 60, and 120 min and added to a tube containing acetonitrile (200 μL) to stop the reaction.

The samples were centrifuged at 14000 g for 15 minutes at 4° C. The supernatant was transferred to clean labeled HPLC injection vials and stored at −80° C. until analyzed. An aliquot of the supernatant (25 μL) was injected onto the LC-UV system for analysis. Results for some compounds of the invention are presented in the table below.

Half lives of a sample of test articles is noted in the following table. Half life was determined in the presence or absence of plasma.

| | | | | Pig Liver | |
|---|---|---|---|---|---|
| | | | | Esterase | |
| | | HLMs | | (t_{1/2} min.) | |
| | | t_{1/2} | Predominant | (+) | (−) |
| | Compound | (min) | Enzyme | Plasma | Plasma |
| 60 | 2-methyl-2-(4-(((1-(4-methylbenzyl)-1H- | 90 | Esterase | 89 | 31 |
| | 1,2,3-triazol-4- | | | | |
| | yl)methoxy)carbonyl)phenylthio)propanoic | | | | |
| | acid | | | | |
| 59 | 2-(4-(((1-(4-isopropylphenyl)-1H-1,2,3- | 30 | Esterase | 44 | 15 |
| | triazol-5-yl)methoxy)carbonyl)phenylthio)- | | | | |
| | 2-methylpropanoic acid | | | | |
| 63 | 2-methyl-2-(4-(((1-(4-methylbenzyl)-1H- | Stable | Stable | Stable | 120 |
| | 1,2,3-triazol-5- | | | | |
| | yl)methoxy)carbonyl)phenoxy)propanoic | | | | |
| | acid | | | | |
| 152 | sodium 2-methyl-2-(4-(((1-(4- | 180 | Esterase | Stable | 30 |
| | methylbenzyl)-4-octyl-5-oxo-4,5-dihydro- | | | | |
| | 1H-1,2,4-triazol-3- | | | | |
| | yl)methoxy)carbonyl)phenoxy)propanoate | | | | |
| 151 | sodium 2-(4-(((4-hexyl-1-(4-methylbenzyl)- | Stable | Stable | Stable | 31 |
| | 5-oxo-4,5-dihydro-1H-1,2,4-triazol-3- | | | | |
| | yl)methoxy)carbonyl)phenoxy)-2- | | | | |
| | methylpropanoate | | | | |
| 014 | 2-(4-(2-((4-hexyl-1-(4-methylbenzyl)-5- | Stable | — | Stable | 19 |
| | oxo-4,5-dihydro-1H-1,2,4-triazol-3- | | | | |
| | yl)methoxy)-2-oxoethyl)phenoxy)-2- | | | | |
| | methylpropanoic acid | | | | |
| 174 | sodium (R)-2-methyl-2-(4-(((5-oxo-1-(4- | 10 | Esterase | 17 | <5 |
| | ((3,3,4,4,4-pentafluorobutan-2- | | | | |
| | yloxy)carbonyl)benzyl)-4-propyl-4,5- | | | | |
| | dihydro-1H-1,2,4-triazol-3- | | | | |
| | yl)methoxy)carbonyl)phenoxy)propanoate | | | | |
| 188 | 2-(4-(((1-(4-tert-butylbenzyl)-4-hexyl-5- | >90 | CYP | Stable | 9 |
| | oxo-4,5-dihydro-1H-1,2,4-triazol-3- | | | | |
| | yl)methoxy)carbonyl)phenoxy)-2- | | | | |
| | methylpropanoic acid | | | | |
| 159 | 2-(4-(((1-(4-tert-butylbenzyl)-5-oxo-4- | Stable | — | 97 | 3 |
| | propyl-4,5-dihydro-1H-1,2,4-triazol-3- | | | | |
| | yl)methoxy)carbonyl)phenylthio)-2- | | | | |
| | methylpropanoic acid | | | | |
| 187 | 2-(4-(((1-(4-tert-butylbenzyl)-4-hexyl-5- | 38 | Mixed | 64 | 26 |
| | oxo-4,5-dihydro-1H-1,2,4-triazol-3- | | | | |
| | yl)methoxy)carbonyl)phenylthio)-2- | | | | |
| | methylpropanoic acid | | | | |
| 191 | 2-(4-(((4-heptyl-1-(4-methylbenzyl)-5-oxo- | 15 | Esterase | Stable | <5 |
| | 4,5-dihydro-1H-1,2,4-triazol-3- | | | | |
| | yl)methoxy)carbonyl)phenylthio)-2- | | | | |
| | methylpropanoic acid | | | | |
| 150 | 2-(4-(((1-(4-tert-butylbenzyl)-5-oxo-4- | Stable | — | Stable | 31 |
| | propyl-4,5-dihydro-1H-1,2,4-triazol-3- | | | | |
| | yl)methoxy)carbonyl)phenoxy)-2- | | | | |
| | methylpropanoic acid | | | | |
| 012 | 2-methyl-2-(4-(2-((1-(4-methylbenzyl)-4- | Stable | — | Stable | 12 |
| | octyl-5-oxo-4,5-dihydro-1H-1,2,4-triazol-3- | | | | |
| | yl)methoxy)-2-oxoethyl)phenoxy)propanoic | | | | |
| | acid | | | | |
| 192 | 2-(4-(((4-heptyl-1-(4-methylbenzyl)-5-oxo- | 33 | Mixed | Stable | 27 |
| | 4,5-dihydro-1H-1,2,4-triazol-3- | | | | |
| | yl)methoxy)carbonyl)phenoxy)-2- | | | | |
| | methylpropanoic acid | | | | |
| 190 | 2-(4-(((1-(4-tert-butylbenzyl)-4-heptyl-5- | 36 | CYP | Stable | 8 |
| | oxo-4,5-dihydro-1H-1,2,4-triazol-3- | | | | |
| | yl)methoxy)carbonyl)phenoxy)-2- | | | | |
| | methylpropanoic acid | | | | |
| 189 | 2-(4-(((1-(4-tert-butylbenzyl)-4-heptyl-5- | 90 | CYP | 105 | <5 |
| | oxo-4,5-dihydro-1H-1,2,4-triazol-3- | | | | |
| | yl)methoxy)carbonyl)phenylthio)-2- | | | | |
| | methylpropanoic acid | | | | |
| 160 | 2-(4-(((4-hexyl-1-(4-methylbenzyl)-5-oxo- | Stable | — | >120 | <5 |
| | 4,5-dihydro-1H-1,2,4-triazol-3- | | | | |
| | yl)methoxy)carbonyl)phenylthio)-2- | | | | |
| | methylpropanoic acid | | | | |
| 161 | 2-methyl-2-(4-(((1-(4-methylbenzyl)-4- | 12 | Esterase | >120 | <5 |
| | octyl-5-oxo-4,5-dihydro-1H-1,2,4-triazol-3- | | | | |
| | yl)methoxy)carbonyl)phenylthio)propanoic | | | | |
| | acid | | | | |
| 186 | 2-(2-methoxy-4-(((1-(4-methylbenzyl)-4- | 42 | CYP | Stable | 45 |
| | octyl-5-oxo-4,5-dihydro-1H-1,2,4-triazol-3- | | | | |
| | yl)methoxy)carbonyl)phenoxy)-2- | | | | |
| | methylpropanoic acid | | | | |
| 67 | 2-methyl-2-(4-(((1-(4- | Stable | — | 121 | 21 |
| | (trifluoromethyl)benzyl)-1H-1,2,3-triazol-4- | | | | |
| | yl)methoxy)carbonyl)phenylthio)propanoic | | | | |
| | acid | | | | |
| 3 | 2-methyl-2-(4-(((1-(4-methylbenzyl)-1H- | Stable | — | >120 | 19 |
| | pyrazol-4- | | | | |
| | yl)methoxy)carbonyl)phenylthio)propanoic | | | | |
| | acid | | | | |
| 69 | 2-(4-(((1-(4-tert-butylbenzyl)-1H-1,2,3- | 60 | Esterase | 126 | 14 |
| | triazol-4-yl)methoxy)carbonyl)phenylthio)- | | | | |
| | 2-methylpropanoic acid | | | | |
| 75 | 2-(4-(((1-(4-(1,1,1,3,3,3-hexafluoro-2- | 25 | Esterase | 160 | 29 |
| | methoxypropan-2-yl)benzyl)-1H-1,2,3- | | | | |
| | triazol-4-yl)methoxy)carbonyl)phenylthio)- | | | | |
| | 2-methylpropanoic acid | | | | |
| 12 | 2-methyl-2-(4-(((3-propyl-1-(4- | 60 | Esterase | 61 | <5 |
| | (trifluoromethyl)benzyl)-1H-pyrazol-4- | | | | |
| | yl)methoxy)carbonyl)phenylthio)propanoic | | | | |
| | acid | | | | |
| 91 | 2-methyl-2-(4-((2-(1-(4- | 55 | Mixed | >120 | 64 |
| | (trifluoromethyl)benzyl)-1H-1,2,3-triazol-4- | | | | |
| | yl)ethoxy)carbonyl)phenylthio)propanoic | | | | |
| | acid | | | | |
| 87 | (R)-2-methyl-2-(4-((1-(1-(4-methylbenzyl)- | 128 | Esterase | 83 | 19 |
| | 1H-1,2,3-triazol-4- | | | | |
| | yl)ethoxy)carbonyl)phenylthio)propanoic | | | | |
| | acid | | | | |
| 95 | (R)-2-methyl-2-(4-((1-(1-(4- | Stable | — | >120 | 68 |
| | (trifluoromethyl)benzyl)-1H-1,2,3-triazol-4- | | | | |
| | yl)ethoxy)carbonyl)phenylthio)propanoic | | | | |
| | acid | | | | |
| 199 | 2-methyl-2-(4-((1-(1-(4-methylbenzyl)-5- | Stable | — | >120 | >120 |
| | oxo-4-propyl-4,5-dihydro-1H-1,2,4-triazol- | | | | |
| | 3-yl)ethoxy)carbonyl)phenylthio)propanoic | | | | |
| | acid | | | | |
| 248 | 4-(1-Carboxy-1-methyl-ethylsulfanyl)- | Stable | — | >120 | >120 |
| | benzoic acid (S)-1-[1-(4-methyl-benzyl)-5- | | | | |
| | oxo-4-propyl-4,5-dihydro-1H-1,2,4-triazol- | | | | |
| | 3-yl]-ethyl ester | | | | |
| 249 | 4-(1-Carboxy-1-methyl-ethylsulfanyl)- | Stable | — | >120 | >120 |
| | benzoic acid (R)-1-[1-(4-methyl-benzyl)-5- | | | | |
| | oxo-4-propyl-4,5-dihydro-1H-1,2,4-triazol- | | | | |
| | 3-yl]-ethyl ester | | | | |
| 54 | 4-(1-Carboxy-1-methyl-ethoxy)-benzoic | 30 | Esterase | >120 | 3 |
| | acid 5-(4-methyl-benzyl)-2-propyl-2H- | | | | |
| | pyrazol-3-ylmethyl ester | | | | |
| 55 | 4-(1-Carboxy-1-methyl-ethylsulfanyl)- | 5 | Esterase | 16 | <1 |
| | benzoic acid 5-(4-methyl-benzyl)-2-propyl- | | | | |
| | 2H-pyrazol-3-ylmethyl ester | | | | |
| 133 | 4-(1-Carboxy-1-methyl-ethylsulfanyl)- | 29 | Mixed | | |
| | benzoic acid (S)-2-[1-(4-tert-butyl-benzyl)- | | | | |
| | 1H-1,2,3-triazol-4-yl]-1-methyl-ethyl ester | | | | |
| 147 | 4-(1-Carboxy-1-methyl-ethylsulfanyl)- | 73 | Mixed | Stable | 77 |
| | benzoic acid (S)-1-methyl-2-[1-(4- | | | | |
| | trifluoromethyl-benzyl)-1H-1,2,3-triazol-4- | | | | |
| | yl]-ethyl ester | | | | |

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. Compounds of the formula: 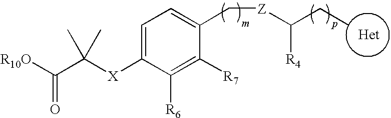
or pharmaceutically acceptable salts thereof, wherein
m is 0, 1, or 2;
p is 0, 1, or 2;
R₄ is H; C₁-C₈ alkyl or C₁-C₄ haloalkyl;
R₆ is H or C₁-C₄ alkoxy;
R₂ is H or C₁-C₄ alkoxy;
R₁₀ is H, C₁-C₆ alkyl, allyl, or —C₁-C₂ alkyl-phenyl;
X is 0, S, SO, or SO₂;
Z is —C(O)O—; and wherein 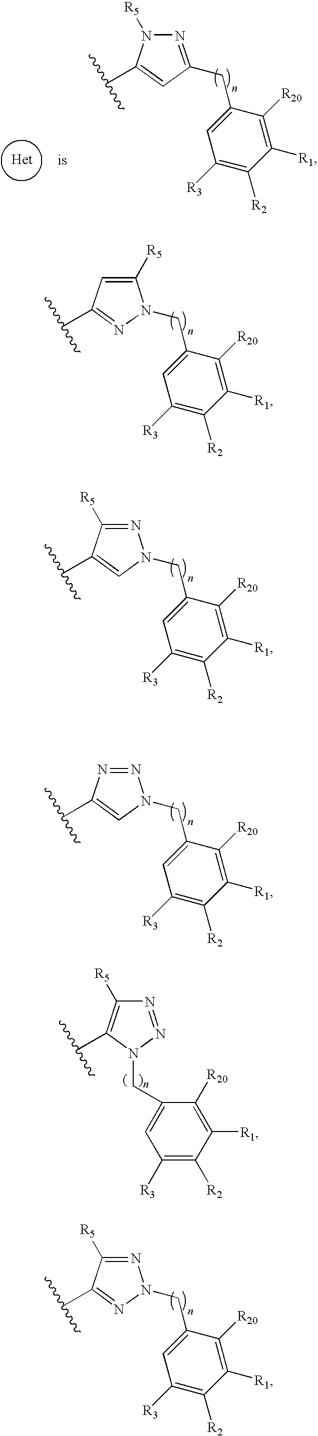 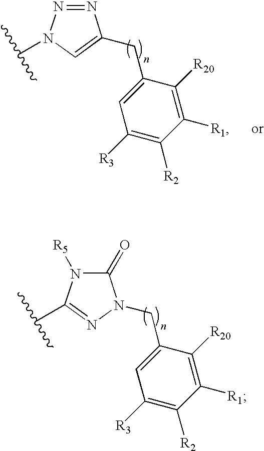
where
n is 0, 1, or 2;
R₁ is H, halogen, C₁-C₆ alkyl, C₁-C₄ haloalkyl;
R₂ is H, C₁-C₆ alkyl optionally substituted with 1, 2, or 3 groups that are independently halo, C₁-C₄ alkoxy, C₁-C₄ haloalkyl, or C₁-C₄ haloalkoxy; C₁-C₆ alkoxy; C₁-C₆ thioalkoxy;
halogen; C₁-C₄ haloalkyl; C₁-C₄ haloalkoxy; C₁-C₄ thiohaloalkoxy; —CO₂-C₁-C₆ alkyl where the alkyl group is optionally substituted with one or more halogens;
R₃ is H, halogen, C₁-C₆ alkyl, C₁-C₄ haloalkyl;
or R₂ and R₃ and the carbons to which they are attached form a phenyl ring or a cyclohexyl ring, each of which is optionally substituted with up to four groups that are independently halogen, C₁-C₄ alkyl, or C₁-C₄ alkoxy;
R₅ is H, C₁-C₁₀ alkyl, C₁-C₄ haloalkyl, C₃-C₆ cycloalkyl, aryl, aryl C₁-C₄ alkyl, wherein the aryl portions of the aryl and arylalkyl groups are unsubstituted or substituted with 1, 2, or 3 groups that are independently C₁-C₄ alkyl, C₁-C₄ alkoxy, halogen, C₁-C₄ haloalkyl, or C₁-C₄ haloalkoxy;
and
R₂₀ is H, halogen, C₁-C₆ alkyl, or C₁-C₆ alkoxy;
with the proviso that when 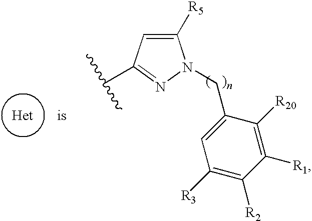
R₅ is not methyl.

2. Compounds or salts according to claim 1, of the formula:

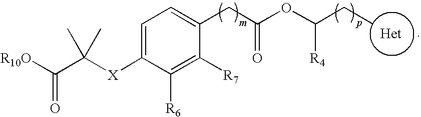

3. Compounds or salts according to claim 2, wherein
R₆ is H, methyl, ethyl, methoxy, or ethoxy; and
R₇ is H, methyl, ethyl, methoxy, or ethoxy.

4. Compounds or salts according to claim 3, wherein
R₁₀ is H, methyl, allyl or benzyl; and
m is 0 or 1.

5. Compounds or salts according to claim 4, wherein
R₄ is H, C₁-C₆ alkyl, or C₁-C₂ haloalkyl; and
p is 0 or 1.

6. Compounds or salts according to claim 5, wherein
R₁ is H, F, tert-butyl, CF₃, methyl;
R₂ is H, tert-butyl, methoxy, CF₃, phenyl, thiomethoxy, F, Cl, Br, OCF₃, methyl, isopropyl, —SCF₃, —OCF₃, —C(CF₃)₂OCH₃, phenyl, —CO₂Me, —CO₂Et, —CO₂CH₂CH₂CF₃, —CO₂C(CF₃)₂CH₃, 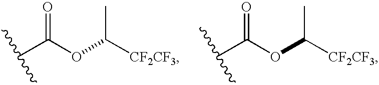
or —C(CF₃)₂F;
R₃ is H, F, tert-butyl, CF₃, methyl;
R₄ is H, methyl, CF₃, or hexyl;
and
R₂₀ is H or F.

7. Compounds or salts according to claim 6, wherein
X is O; and
R₅ is H, methyl, CF₃, propyl, phenyl, hexyl, octyl, cyclopentyl, 2-fluorobenzyl, benzyl, heptyl, or ethyl.

8. Compounds or salts according to claim 6, wherein
X is S; and
R₅ is H, CF₃, propyl, phenyl, hexyl, octyl, cyclopentyl, 2-fluorobenzyl, benzyl, heptyl, or ethyl.

9. Compounds according to claim 1 that are
2-methyl-2-(4-(((1-methyl-3-phenyl-1H-pyrazol-5-yl)methoxy)carbonyl)phenylthio)propanoic acid;
2-methyl-2-(4-(((1-methyl-3-phenyl-1H-pyrazol-5-yl)methoxy)carbonyl)phenoxy)propanoic acid;
2-methyl-2-(4-(((1-(4-methylbenzyl)-1H-pyrazol-4-yl)methoxy)carbonyl)phenylthio)propanoic acid;
2-methyl-2-(4-(((1-(4-methylbenzyl)-3-(trifluoromethyl)-1H-pyrazol-4-yl)methoxy)carbonyl)phenoxy)propanoic acid;
2-methyl-2-(4-(((1-(4-methylbenzyl)-3-(trifluoromethyl)-1H-pyrazol-4-yl)methoxy)carbonyl)phenylthio)propanoic acid;
2-(4-(((1-(4-tert-butylbenzyl)-1H-pyrazol-4-yl)methoxy)carbonyl)phenylthio)-2-methylpropanoic acid;
2-(4-(((1-(4-tert-butylbenzyl)-1H-pyrazol-4-yl)methoxy)carbonyl)phenoxy)-2-methylpropanoic acid;
2-(4-(((1-(4-methoxybenzyl)-1H-pyrazol-4-yl)methoxy)carbonyl)phenylthio)-2-methylpropanoic acid;
2-(4-(((1-(4-methoxybenzyl)-1H-pyrazol-4-yl)methoxy)carbonyl)phenoxy)-2-methylpropanoic acid;
2-methyl-2-(4-(((1-(4-(trifluoromethyl)benzyl)-1H-pyrazol-4-yl)methoxy)carbonyl)phenylthio)propanoic acid;
2-methyl-2-(4-(((1-(4-(trifluoromethyl)benzyl)-1H-pyrazol-4-yl)methoxy)carbonyl)phenoxy)propanoic acid;
2-methyl-2-(4-(((3-propyl-1-(4-(trifluoromethyl)benzyl)-1H-pyrazol-4-yl)methoxy)carbonyl)phenylthio)propanoic acid;
2-methyl-2-(4-(((3-propyl-1-(4-(trifluoromethyl)benzyl)-1H-pyrazol-4-yl)methoxy)carbonyl)phenoxy)propanoic acid;
2-methyl-2-(4-(((1-propyl-3-p-tolyl-1H-pyrazol-5-yl)methoxy)carbonyl)phenylthio)propanoic acid;
2-methyl-2-(4-(((1-propyl-3-p-tolyl-1H-pyrazol-5-yl)methoxy)carbonyl)phenoxy)propanoic acid;
2-methyl-2-(4-(((3-phenyl-1-propyl-1H-pyrazol-5-yl)methoxy)carbonyl)phenylthio)propanoic acid;
2-methyl-2-(4-(((3-phenyl-1-p-tolyl-1H-pyrazol-4-yl)methoxy)carbonyl)phenoxy)propanoic acid;
2-methyl-2-(4-(((1-(4-(trifluoromethyl)phenyl)-1H-pyrazol-4-yl)methoxy)carbonyl)phenylthio)propanoic acid;
2-methyl-2-(4-(((1-(4-(trifluoromethyl)phenyl)-1H-pyrazol-4-yl)methoxy)carbonyl)phenoxy)propanoic acid;
2-methyl-2-(4-(((1-(4-(methylthio)benzyl)-1H-pyrazol-4-yl)methoxy)carbonyl)phenylthio)propanoic acid;
2-methyl-2-(4-(((1-(4-(methylthio)benzyl)-1H-pyrazol-4-yl)methoxy)carbonyl)phenoxy)propanoic acid;
2-(4-(((1-(biphenyl-4-ylmethyl)-1H-pyrazol-4-yl)methoxy)carbonyl)phenylthio)-2-methylpropanoic acid;
2-(4-(((1-(biphenyl-4-ylmethyl)-1H-pyrazol-4-yl)methoxy)carbonyl)phenoxy)-2-methylpropanoic acid;
2-(4-(((1-(3,4-difluorobenzyl)-1H-pyrazol-4-yl)methoxy)carbonyl)phenylthio)-2-methylpropanoic acid;
2-(4-(((1-(3,4-difluorobenzyl)-1H-pyrazol-4-yl)methoxy)carbonyl)phenoxy)-2-methylpropanoic acid;
2-methyl-2-(4-(((1-(naphthalen-2-ylmethyl)-1H-pyrazol-4-yl)methoxy)carbonyl)phenylthio)propanoic acid;
2-methyl-2-(4-(((1-(naphthalen-2-ylmethyl)-1H-pyrazol-4-yl)methoxy)carbonyl)phenoxy)propanoic acid;
2-(4-(((1-(3,4-di-tert-butylbenzyl)-1H-pyrazol-4-yl)methoxy)carbonyl)phenoxy)-2-methylpropanoic acid;
2-methyl-2-(4-(((1-((5,5,8,8-tetramethyl-5,6,7,8-tetrahydronaphthalen-2-yl)methyl)-1H-pyrazol-4-yl)methoxy)carbonyl)phenylthio)propanoic acid;
2-methyl-2-(4-(((1-(3-(trifluoromethyl)benzyl)-1H-pyrazol-4-yl)methoxy)carbonyl)phenylthio)propanoic acid;
2-methyl-2-(4-(((1-(3-(trifluoromethyl)benzyl)-1H-pyrazol-4-yl)methoxy)carbonyl)phenoxy)propanoic acid;
2-methyl-2-(4-(((1-(4-(trifluoromethoxy)benzyl)-1H-pyrazol-4-yl)methoxy)carbonyl)phenylthio)propanoic acid;
2-methyl-2-(4-(2-oxo-2-((1-(4-(trifluoromethyl)phenyl)-1H-pyrazol-4-yl)methoxy)ethyl)phenoxy)propanoic acid;
(R)-2-methyl-2-(4-((1-(3-(4-methylbenzyl)-1-propyl-1H-pyrazol-5-yl)ethoxy)carbonyl)phenoxy)propanoic acid;
(S)-2-methyl-2-(4-((1-(3-(4-methylbenzyl)-1-propyl-1H-pyrazol-5-yl)ethoxy)carbonyl)phenoxy)propanoic acid;
(R)-2-methyl-2-(4-((2,2,2-trifluoro-1-(3-(4-methylbenzyl)-1-propyl-1H-pyrazol-5-yl)ethoxy)carbonyl)phenoxy)propanoic acid;
(S)-2-methyl-2-(4-((2,2,2-trifluoro-1-(3-(4-methylbenzyl)-1-propyl-1H-pyrazol-5-yl)ethoxy)carbonyl)phenoxy)propanoic acid;
(R)-2-methyl-2-(4-((1-(3-(4-methylbenzyl)-1-propyl-1H-pyrazol-5-yl)ethoxy)carbonyl)phenylthio)propanoic acid;
(S)-2-methyl-2-(4-((1-(3-(4-methylbenzyl)-1-propyl-1H-pyrazol-5-yl)ethoxy)carbonyl)phenylthio)propanoic acid;
(R)-2-methyl-2-(4-((2,2,2-trifluoro-1-(3-(4-methylbenzyl)-1-propyl-1H-pyrazol-5-yl)ethoxy)carbonyl)phenylthio)propanoic acid;
(S)-2-methyl-2-(4-((2,2,2-trifluoro-1-(3-(4-methylbenzyl)-1-propyl-1H-pyrazol-5-yl)ethoxy)carbonyl)phenylthio)propanoic acid;
4-(1-Carboxy-1-methyl-ethoxy)-benzoic acid 5-(4-methyl-benzyl)-2-propyl-2H-pyrazol-3-ylmethyl ester;
4-(1-Carboxy-1-methyl-ethylsulfanyl)-benzoic acid 5-(4-methyl-benzyl)-2-propyl-2H-pyrazol-3-ylmethyl ester;
2-(4-(((1-(4-isopropylphenyl)-1H-1,2,3-triazol-4-yl)methoxy)carbonyl)phenylthio)-2-ethylpropanoic acid;
2-methyl-2-(4-(((1-(4-(trifluoromethyl)phenyl)-1H-1,2,3-triazol-4-yl)methoxy)carbonyl)phenylthio)propanoic acid;
2-methyl-2-(4-(((1-(4-(trifluoromethyl)phenyl)-1H-1,2,3-triazol-4-yl)methoxy)carbonyl)phenoxy)propanoic acid;
2-(4-(((1-(4-isopropylphenyl)-1H-1,2,3-triazol-5-yl)methoxy)carbonyl)phenylthio)-2-methylpropanoic acid;
2-methyl-2-(4-(((1-(4-methylbenzyl)-1H-1,2,3-triazol-4-yl)methoxy)carbonyl)phenylthio)propanoic acid;
2-methyl-2-(4-(((1-(4-methylbenzyl)-1H-1,2,3-triazol-4-yl)methoxy)carbonyl)phenoxy)propanoic acid;
2-methyl-2-(4-(((1-(4-methylbenzyl)-1H-1,2,3-triazol-5-yl)methoxy)carbonyl)phenylthio)propanoic acid;
2-methyl-2-(4-(((1-(4-methylbenzyl)-1H-1,2,3-triazol-5-yl)methoxy)carbonyl)phenoxy)propanoic acid;
2-methyl-2-(4-(((1-(4-(trifluoromethyl)benzyl)-1H-1,2,3-triazol-4-yl)methoxy)carbonyl)phenoxy)propanoic acid;
2-methyl-2-(4-(((5-methyl-2-phenyl-2H-1,2,3-triazol-4-yl)methoxy)carbonyl)phenylthio)propanoic acid;
2-methyl-2-(4-(((5-methyl-2-phenyl-2H-1,2,3-triazol-4-yl)methoxy)carbonyl)phenoxy)propanoic acid;
2-methyl-2-(4-(((1-(4-(trifluoromethyl)benzyl)-1H-1,2,3-triazol-4-yl)methoxy)carbonyl)phenylthio)propanoic acid;
2-(4-(((1-(4-methoxybenzyl)-1H-1,2,3-triazol-4-yl)methoxy)carbonyl)phenylthio)-2-methylpropanoic acid;
2-(4-(((1-(4-tert-butylbenzyl)-1H-1,2,3-triazol-4-yl)methoxy)carbonyl)phenylthio)-2-methylpropanoic acid;
2-(4-(((1-(4-tert-butylbenzyl)-1H-1,2,3-triazol-4-yl)methoxy)carbonyl)phenoxy)-2-methylpropanoic acid;
2-methyl-2-(4-(((1-(4-(trifluoromethylthio)benzyl)-1H-1,2,3-triazol-4-yl)methoxy)carbonyl)phenylthio)propanoic acid;
2-methyl-2-(4-(((1-(4-(trifluoromethylthio)benzyl)-1H-1,2,3-triazol-4-yl)methoxy)carbonyl)phenoxy)propanoic acid;
2-methyl-2-(4-(((1-(4-(trifluoromethoxy)benzyl)-1H-1,2,3-triazol-4-yl)methoxy)carbonyl)phenylthio)propanoic acid;
2-methyl-2-(4-(((1-(4-(trifluoromethoxy)benzyl)-1H-1,2,3-triazol-4-yl)methoxy)carbonyl)phenoxy)propanoic acid;
2-(4-(((1-(4-(1,1,1,3,3,3-hexafluoro-2-methoxypropan-2-yl)benzyl)-1H-1,2,3-triazol-4-yl)methoxy)carbonyl)phenylthio)-2-methylpropanoic acid;
2-(4-(((1-(4-(1,1,1,3,3,3-hexafluoro-2-methoxypropan-2-yl)benzyl)-1H-1,2,3-triazol-4-yl)methoxy)carbonyl)phenoxy)-2-methylpropanoic acid;
2-(4-(((1-benzyl-1H-1,2,3-triazol-4-yl)methoxy)carbonyl)phenylthio)-2-methylpropanoic acid;
2-(4-(((1-benzyl-1H-1,2,3-triazol-4-yl)methoxy)carbonyl)phenoxy)-2-methylpropanoic acid;
2-(3-methoxy-4-(((1-(4-(trifluoromethyl)benzyl)-1H-1,2,3-triazol-4-yl)methoxy)carbonyl)phenoxy)-2-methylpropanoic acid;
2-methyl-2-(3-methyl-4-(((1-(4-(trifluoromethyl)benzyl)-1H-1,2,3-triazol-4-yl)methoxy)carbonyl)phenoxy)propanoic acid;
2-methyl-2-(4-((1-(1-(4-methylbenzyl)-1H-1,2,3-triazol-4-yl)ethoxy)carbonyl)phenylthio)propanoic acid;
2-methyl-2-(4-((1-(1-(4-methylbenzyl)-1H-1,2,3-triazol-5-yl)ethoxy)carbonyl)phenylthio)propanoic acid;
2-methyl-2-(4-((1-(1-(4-methylbenzyl)-1H-1,2,3-triazol-4-yl)heptyloxy)carbonyl)phenylthio)propanoic acid;
2-methyl-2-(4-((1-(1-(4-methylbenzyl)-1H-1,2,3-triazol-4-yl)heptyloxy)carbonyl)phenoxy)propanoic acid;
(S)-2-methyl-2-(4-((1-(1-(4-methylbenzyl)-1H-1,2,3-triazol-4-yl)ethoxy)carbonyl)phenylthio)propanoic acid;
(S)-2-methyl-2-(4-((1-(1-(4-methylbenzyl)-1H-1,2,3-triazol-4-yl)ethoxy)carbonyl)phenoxy)propanoic acid;
(R)-2-methyl-2-(4-((1-(1-(4-methylbenzyl)-1H-1,2,3-triazol-4-yl)ethoxy)carbonyl)phenylthio)propanoic acid;
(R)-2-methyl-2-(4-((1-(1-(4-methylbenzyl)-1H-1,2,3-triazol-4-yl)ethoxy)carbonyl)phenoxy)propanoic acid;
2-(4-((2-(4-(4-tert-butylphenyl)-1H-1,2,3-triazol-1-yl)ethoxy)carbonyl)phenylthio)-2-methylpropanoic acid;
2-(4-((2-(4-(4-tert-butylphenyl)-1H-1,2,3-triazol-1-yl)ethoxy)carbonyl)phenoxy)-2-methylpropanoic acid;
2-methyl-2-(4-((2-(1-(4-(trifluoromethyl)benzyl)-1H-1,2,3-triazol-4-yl)ethoxy)carbonyl)phenylthio)propanoic acid;
2-methyl-2-(4-((2-(1-(4-(trifluoromethyl)benzyl)-1H-1,2,3-triazol-4-yl)ethoxy)carbonyl)phenoxy)propanoic acid;
2-methyl-2-(4-(((1-(4-methylphenethyl)-1H-1,2,3-triazol-4-yl)methoxy)carbonyl)phenylthio)propanoic acid;
2-methyl-2-(4-(((1-(4-methylphenethyl)-1H-1,2,3-triazol-4-yl)methoxy)carbonyl)phenoxy)propanoic acid;
(R)-2-methyl-2-(4-((1-(1-(4-(trifluoromethyl)benzyl)-1H-1,2,3-triazol-4-yl)ethoxy)carbonyl)phenylthio)propanoic acid;
(R)-2-methyl-2-(4-((1-(1-(4-(trifluoromethyl)benzyl)-1H-1,2,3-triazol-4-yl)ethoxy)carbonyl)phenoxy)propanoic acid;
(S)-2-methyl-2-(4-((1-(1-(4-(trifluoromethyl)benzyl)-1H-1,2,3-triazol-4-yl)ethoxy)carbonyl)phenylthio)propanoic acid;
(S)-2-methyl-2-(4-((1-(1-(4-(trifluoromethyl)benzyl)-1H-1,2,3-triazol-4-yl)ethoxy)carbonyl)phenoxy)propanoic acid;
2-(2-methoxy-4-(((1-(4-methylbenzyl)-1H-1,2,3-triazol-4-yl)methoxy)carbonyl)phenoxy)-2-methylpropanoic acid;
2-(4-(((1-(4-tert-butylbenzyl)-1H-1,2,3-triazol-4-yl)methoxy)carbonyl)-2-methoxyphenoxy)-2-methylpropanoic acid;
2-(2-methoxy-4-(((1-(4-(trifluoromethyl)benzyl)-1H-1,2,3-triazol-4-yl)methoxy)carbonyl)phenoxy)-2-methylpropanoic acid;
2-methyl-2-(4-(((1-(4-(trifluoromethyl)benzyl)-1H-1,2,3-triazol-4-yl)methoxy)carbonyl)phenylthio)propanoic acid;
2-(4-(((1-(4-methoxybenzyl)-1H-1,2,3-triazol-4-yl)methoxy)carbonyl)phenoxy)-2-methylpropanoic acid;
2-(3-methoxy-4-(((1-(4-(trifluoromethyl)benzyl)-1H-1,2,3-triazol-4-yl)methoxy)carbonyl)phenoxy)-2-methylpropanoic acid;
(R)-2-(4-((1-(1-(4-tert-butylbenzyl)-1H-1,2,3-triazol-4-yl)propan-2-yloxy)carbonyl)phenylthio)-2-methylpropanoic acid;
(S)-2-(4-((3-(1-(4-tert-butylbenzyl)-1H-1,2,3-triazol-4-yl)-1,1,1-trifluoropropan-2-yloxy)carbonyl)phenylthio)-2-methylpropanoic acid;
(R)-2-(4-((3-(1-(4-tert-butylbenzyl)-1H-1,2,3-triazol-4-yl)-1,1,1-trifluoropropan-2-yloxy)carbonyl)phenylthio)-2-methylpropanoic acid;
(R)-2-methyl-2-(4-((1-(1-(4-(trifluoromethyl)benzyl)-1H-1,2,3-triazol-4-yl)propan-2-yloxy)carbonyl)phenylthio)propanoic acid;
(S)-2-methyl-2-(4-((1-(1-(4-(trifluoromethyl)benzyl)-1H-1,2,3-triazol-4-yl)propan-2-yloxy)carbonyl)phenylthio)propanoic acid;
(R)-2-methyl-2-(4-((1,1,1-trifluoro-3-(1-(4-(trifluoromethyl)benzyl)-1H-1,2,3-triazol-4-yl)propan-2-yloxy)carbonyl)phenylthio)propanoic acid;
(S)-2-methyl-2-(4-((1,1,1-trifluoro-3-(1-(4-(trifluoromethyl)benzyl)-1H-1,2,3-triazol-4-yl)propan-2-yloxy)carbonyl)phenylthio)propanoic acid;
(S)-2-methyl-2-(4-((2,2,2-trifluoro-1-(1-(4-methylbenzyl)-1H-1,2,3-triazol-4-yl)ethoxy)carbonyl)phenylthio)propanoic acid;
(R)-2-methyl-2-(4-((2,2,2-trifluoro-1-(1-(4-methylbenzyl)-1H-1,2,3-triazol-4-yl)ethoxy)carbonyl)phenylthio)propanoic acid;
(R)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid;
(S)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid;
4-(1-Carboxy-1-methyl-ethylsulfanyl)-benzoic acid (S)-2-[1-(4-tert-butyl-benzyl)-1H-1,2,3-triazol-4-yl]-1-methyl-ethyl ester;
4-(1-Carboxy-1-methyl-ethylsulfanyl)-benzoic acid 1-methyl-2-[1-(4-trifluoromethyl-benzyl)-1H-1,2,3-triazol-4-yl]-ethyl ester;
4-(1-Carboxy-1-methyl-ethylsulfanyl)-benzoic acid (R)-1-[1-(4-tert-butyl-benzyl)-1H-1,2,3-triazol-4-yl]-ethyl ester;
4-(1-Carboxy-1-methyl-ethylsulfanyl)-benzoic acid (R)-1-[1-(4-trifluoromethylsulfanyl-benzyl)-1H-1,2,3-triazol-4-yl]-ethyl ester;
4-(1-Carboxy-1-methyl-ethylsulfanyl)-benzoic acid (R)-1-[1-(4-trifluoromethoxy-benzyl)-1H-1,2,3-triazol-4-yl]-ethyl ester;
4-(1-Carboxy-1-methyl-ethylsulfanyl)-benzoic acid (R)-2-[1-(4-tert-butyl-benzyl)-1H-1,2,3-triazol-4-yl]-1-methyl-ethyl ester;
4-(1-Carboxy-1-methyl-ethylsulfanyl)-benzoic acid (R)-1-[1-(3-trifluoromethyl-benzyl)-1H-1,2,3-triazol-4-yl]-ethyl ester;
4-(1-Carboxy-1-methyl-ethylsulfanyl)-benzoic acid (R)-1-[1-(3,5-bis-trifluoromethyl-benzyl)-1H-1,2,3-triazol-4-yl]-ethyl ester;
4-(1-Carboxy-1-methyl-ethylsulfanyl)-benzoic acid (R)-1-[1-(3,5-difluoro-benzyl)-1H-1,2,3-triazol-4-yl]-ethyl ester;
4-(1-Carboxy-1-methyl-ethoxy)-3-methyl-benzoic acid 1-(4-trifluoromethyl-benzyl)-1H-1,2,3-triazol-4-ylmethyl ester;
2-methyl-2-(4-(2-oxo-2-((1-(4-(trifluoromethyl)benzyl)-1H-1,2,3-triazol-4-yl)methoxy)ethyl)phenoxy)propanoic acid;
4-(1-Carboxy-1-methyl-ethylsulfanyl)-benzoic acid 2-[1-(4-tert-butyl-benzyl)-1H-1,2,3-triazol-4-yl]-ethyl ester;
4-(1-Carboxy-1-methyl-ethylsulfanyl)-benzoic acid 2-[1-(4-methyl-benzyl)-1H-1,2,3-triazol-4-yl]-ethyl ester;
4-(1-Carboxy-1-methyl-ethylsulfanyl)-benzoic acid (R)-1-methyl-2-[1-(4-trifluoromethyl-benzyl)-1H-1,2,3-triazol-4-yl]-ethyl ester;
4-(1-Carboxy-1-methyl-ethylsulfanyl)-benzoic acid (S)-1-methyl-2-[1-(4-trifluoromethyl-benzyl)-1H-1,2,3-triazol-4-yl]-ethyl ester;
2-methyl-2-(4-(((1-(4-methylbenzyl)-5-oxo-4-propyl-4,5-dihydro-1H-1,2,4-triazol-3-yl)methoxy)carbonyl)phenylthio)propanoic acid;
2-methyl-2-(4-(((1-(4-methylbenzyl)-5-oxo-4-propyl-4,5-dihydro-1H-1,2,4-triazol-3-yl)methoxy)carbonyl)phenoxy)propanoic acid;
2-(4-(((1-(4-tert-butylbenzyl)-5-oxo-4-propyl-4,5-dihydro-1H-1,2,4-triazol-3-yl)methoxy)carbonyl)phenoxy)-2-methylpropanoic acid;
2-(4-(((4-hexyl-1-(4-methylbenzyl)-5-oxo-4,5-dihydro-1H-1,2,4-triazol-3-yl)methoxy)carbonyl)phenoxy)-2-methylpropanoic acid;
2-methyl-2-(4-(((1-(4-methylbenzyl)-4-octyl-5-oxo-4,5-dihydro-1H-1,2,4-triazol-3-yl)methoxy)carbonyl)phenoxy)propanoic acid;
2-(4-(((4-cyclopentyl-1-(4-methylbenzyl)-5-oxo-4,5-dihydro-1H-1,2,4-triazol-3-yl)methoxy)carbonyl)phenoxy)-2-methylpropanoic acid;
2-(4-(((4-benzyl-1-(4-methylbenzyl)-5-oxo-4,5-dihydro-1H-1,2,4-triazol-3-yl)methoxy)carbonyl)phenoxy)-2-methylpropanoic acid;
2-(4-(((4-(2-fluorobenzyl)-1-(4-methylbenzyl)-5-oxo-4,5-dihydro-1H-1,2,4-triazol-3-yl)methoxy)carbonyl)phenoxy)-2-methylpropanoic acid;
2-methyl-2-(4-(((5-oxo-1-phenethyl-4-propyl-4,5-dihydro-1H-1,2,4-triazol-3-yl)methoxy)carbonyl)phenoxy)propanoic acid;
2-(4-(((1-(4-fluorobenzyl)-5-oxo-4-propyl-4,5-dihydro-1H-1,2,4-triazol-3-yl)methoxy)carbonyl)phenoxy)-2-methylpropanoic acid;
2-(4-(((1-(2-fluorobenzyl)-5-oxo-4-propyl-4,5-dihydro-1H-1,2,4-triazol-3-yl)methoxy)carbonyl)phenoxy)-2-methylpropanoic acid;
2-(4-(((1-(4-tert-butylbenzyl)-5-oxo-4-propyl-4,5-dihydro-1H-1,2,4-triazol-3-yl)methoxy)carbonyl)phenylthio)-2-methylpropanoic acid;
2-(4-(((4-hexyl-1-(4-methylbenzyl)-5-oxo-4,5-dihydro-1H-1,2,4-triazol-3-yl)methoxy)carbonyl)phenylthio)-2-methylpropanoic acid;
2-methyl-2-(4-(((1-(4-methylbenzyl)-4-octyl-5-oxo-4,5-dihydro-1H-1,2,4-triazol-3-yl)methoxy)carbonyl)phenylthio)propanoic acid;
2-(4-(((1-(3,4-dimethylbenzyl)-5-oxo-4-propyl-4,5-dihydro-1H-1,2,4-triazol-3-yl)methoxy)carbonyl)phenylthio)-2-methylpropanoic acid;
2-(4-(((1-(biphenyl-4-ylmethyl)-5-oxo-4-propyl-4,5-dihydro-1H-1,2,4-triazol-3-yl)methoxy)carbonyl)phenylthio)-2-methylpropanoic acid;
2-(4-(((1-(4-bromobenzyl)-5-oxo-4-propyl-4,5-dihydro-1H-1,2,4-triazol-3-yl)methoxy)carbonyl)phenylthio)-2-methylpropanoic acid;
2-(4-(((1-(4-fluorobenzyl)-5-oxo-4-propyl-4,5-dihydro-1H-1,2,4-triazol-3-yl)methoxy)carbonyl)phenylthio)-2-methylpropanoic acid;
2-(4-(((1-(2,4-difluorobenzyl)-5-oxo-4-propyl-4,5-dihydro-1H-1,2,4-triazol-3-yl)methoxy)carbonyl)phenoxy)-2-methylpropanoic acid;
2-methyl-2-(4-(((5-oxo-4-propyl-1-(4-(trifluoromethyl)benzyl)-4,5-dihydro-1H-1,2,4-triazol-3-yl)methoxy)carbonyl)phenoxy)propanoic acid;
2-methyl-2-(4-(((5-oxo-4-propyl-1-(4-(trifluoromethoxy)benzyl)-4,5-dihydro-1H-1,2,4-triazol-3-yl)methoxy)carbonyl)phenoxy)propanoic acid;
2-(4-(((1-(4-(methoxycarbonyl)benzyl)-5-oxo-4-propyl-4,5-dihydro-1H-1,2,4-triazol-3-yl)methoxy)carbonyl)phenoxy)-2-methylpropanoic acid;
2-(4-(((1-(4-(ethoxycarbonyl)benzyl)-5-oxo-4-propyl-4,5-dihydro-1H-1,2,4-triazol-3-yl)methoxy)carbonyl)phenoxy)-2-methylpropanoic acid;
2-methyl-2-(4-(((5-oxo-4-propyl-1-(4-((3,3,3-trifluoropropoxy)carbonyl)benzyl)-4,5-dihydro-1H-1,2,4-triazol-3-yl)methoxy)carbonyl)phenoxy)propanoic acid;
2-(4-(((1-(4-((1,1,1,3,3,3-hexafluoro-2-methylpropan-2-yloxy)carbonyl)benzyl)-5-oxo-4-propyl-4,5-dihydro-1H-1,2,4-triazol-3-yl)methoxy)carbonyl)phenoxy)-2-methylpropanoic acid;
(S)-2-methyl-2-(4-(((5-oxo-1-(4-((3,3,4,4,4-pentafluorobutan-2-yloxy)carbonyl)benzyl)-4-propyl-4,5-dihydro-1H-1,2,4-triazol-3-yl)methoxy)carbonyl)phenoxy)propanoic acid;
(R)-2-methyl-2-(4-(((5-oxo-1-(4-(3,3,4,4,4-pentafluorobutan-2-yloxy)carbonyl)benzyl)-4-propyl-4,5-dihydro-1H-1,2,4-triazol-3-yl)methoxy)carbonyl)phenoxy)propanoic acid;
2-methyl-2-(4-(((5-oxo-4-propyl-1-(4-(trifluoromethyl)benzyl)-4,5-dihydro-1H-1,2,4-triazol-3-yl)methoxy)carbonyl)phenylthio)propanoic acid;
2-methyl-2-(4-(((5-oxo-4-propyl-1-(3-(trifluoromethyl)benzyl)-4,5-dihydro-1H-1,2,4-triazol-3-yl)methoxy)carbonyl)phenylthio)propanoic acid;
2-(4-(((1-(3,5-bis(trifluoromethyl)benzyl)-5-oxo-4-propyl-4,5-dihydro-1H-1,2,4-triazol-3-yl)methoxy)carbonyl)phenylthio)-2-methylpropanoic acid;
2-methyl-2-(4-(((5-oxo-4-propyl-1-(4-(trifluoromethoxy)benzyl)-4,5-dihydro-1H-1,2,4-triazol-3-yl)methoxy)carbonyl)phenylthio)propanoic acid;
2-methyl-2-(4-(((5-oxo-4-propyl-1-(4-(trifluoromethylthio)benzyl)-4,5-dihydro-1H-1,2,4-triazol-3-yl)methoxy)carbonyl)phenylthio)propanoic acid;
2-(4-(((1-(4-tert-butylbenzyl)-5-oxo-4-propyl-4,5-dihydro-1H-1,2,4-triazol-3-yl)methoxy)carbonyl)-2-methoxyphenoxy)-2-methylpropanoic acid;
2-(2-methoxy-4-(((1-(4-methylbenzyl)-5-oxo-4-propyl-4,5-dihydro-1H-1,2,4-triazol-3-yl)methoxy)carbonyl)phenoxy)-2-methylpropanoic acid;
2-(3-methoxy-4-(((1-(4-methylbenzyl)-5-oxo-4-propyl-4,5-dihydro-1H-1,2,4-triazol-3-yl)methoxy)carbonyl)phenoxy)-2-methylpropanoic acid;
2-methyl-2-(3-methyl-4-(((1-(4-methylbenzyl)-5-oxo-4-propyl-4,5-dihydro-1H-1,2,4-triazol-3-yl)methoxy)carbonyl)phenoxy)propanoic acid;
2-(4-(((4-hexyl-1-(4-methylbenzyl)-5-oxo-4,5-dihydro-1H-1,2,4-triazol-3-yl)methoxy)carbonyl)-2-methoxyphenoxy)-2-methylpropanoic acid;
2-(4-(((1-(4-tert-butylbenzyl)-4-hexyl-5-oxo-4,5-dihydro-1H-1,2,4-triazol-3-yl)methoxy)carbonyl)-2-methoxyphenoxy)-2-methylpropanoic acid;
2-(2-methoxy-4-(((1-(4-methylbenzyl)-4-octyl-5-oxo-4,5-dihydro-1H-1,2,4-triazol-3-yl)methoxy)carbonyl)phenoxy)-2-methylpropanoic acid;
2-(4-(((1-(4-tert-butylbenzyl)-4-hexyl-5-oxo-4,5-dihydro-1H-1,2,4-triazol-3-yl)methoxy)carbonyl)phenylthio)-2-methylpropanoic acid;
2-(4-(((1-(4-tert-butylbenzyl)-4-hexyl-5-oxo-4,5-dihydro-1H-1,2,4-triazol-3-yl)methoxy)carbonyl)phenoxy)-2-methylpropanoic acid;
2-(4-(((1-(4-tert-butylbenzyl)-4-heptyl-5-oxo-4,5-dihydro-1H-1,2,4-triazol-3-yl)methoxy)carbonyl)phenylthio)-2-methylpropanoic acid;
2-(4-(((1-(4-tert-butylbenzyl)-4-heptyl-5-oxo-4,5-dihydro-1H-1,2,4-triazol-3-yl)methoxy)carbonyl)phenoxy)-2-methylpropanoic acid;
2-(4-(((4-heptyl-1-(4-methylbenzyl)-5-oxo-4,5-dihydro-1H-1,2,4-triazol-3-yl)methoxy)carbonyl)phenylthio)-2-methylpropanoic acid;
2-(4-(((4-heptyl-1-(4-methylbenzyl)-5-oxo-4,5-dihydro-1H-1,2,4-triazol-3-yl)methoxy)carbonyl)phenoxy)-2-methylpropanoic acid;
2-(4-(((4-ethyl-1-(4-methylbenzyl)-5-oxo-4,5-dihydro-1H-1,2,4-triazol-3-yl)methoxy)carbonyl)phenylthio)-2-methylpropanoic acid;
2-(4-(((4-ethyl-1-(4-methylbenzyl)-5-oxo-4,5-dihydro-1H-1,2,4-triazol-3-yl)methoxy)carbonyl)phenoxy)-2-methylpropanoic acid;
2-(4-(((1-(4-tert-butylbenzyl)-4-ethyl-5-oxo-4,5-dihydro-1H-1,2,4-triazol-3-yl)methoxy)carbonyl)phenylthio)-2-methylpropanoic acid;
2-(4-(((1-(4-tert-butylbenzyl)-4-ethyl-5-oxo-4,5-dihydro-1H-1,2,4-triazol-3-yl)methoxy)carbonyl)phenoxy)-2-methylpropanoic acid;
2-(4-(((4-ethyl-5-oxo-1-(4-(trifluoromethyl)benzyl)-4,5-dihydro-1H-1,2,4-triazol-3-yl)methoxy)carbonyl)phenylthio)-2-methylpropanoic acid;
2-(4-(((4-ethyl-5-oxo-1-(4-(trifluoromethyl)benzyl)-4,5-dihydro-1H-1,2,4-triazol-3-yl)methoxy)carbonyl)phenoxy)-2-methylpropanoic acid;
2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid;
2-methyl-2-(4-((1-(1-(4-methylbenzyl)-5-oxo-4-propyl-4,5-dihydro-1H-1,2,4-triazol-3-yl)ethoxy)carbonyl)phenoxy)propanoic acid;
2-(4-(((1-(4-(1,1,1,3,3,3-hexafluoro-2-methoxypropan-2-yl)benzyl)-4-hexyl-5-oxo-4,5-dihydro-1H-1,2,4-triazol-3-yl)methoxy)carbonyl)phenylthio)-2-methylpropanoic acid;
2-(4-(((1-(4-(1,1,1,3,3,3-hexafluoro-2-methoxypropan-2-yl)benzyl)-4-hexyl-5-oxo-4,5-dihydro-1H-1,2,4-triazol-3-yl)methoxy)carbonyl)phenoxy)-2-methylpropanoic acid;
2-(4-(((4-hexyl-5-oxo-1-(4-(perfluoropropan-2-yl)benzyl)-4,5-dihydro-1H-1,2,4-triazol-3-yl)methoxy)carbonyl)phenylthio)-2-methylpropanoic acid;
2-(4-(((4-hexyl-5-oxo-1-(4-(perfluoropropan-2-yl)benzyl)-4,5-dihydro-1H-1,2,4-triazol-3-yl)methoxy)carbonyl)phenoxy)-2-methylpropanoic acid;
2-methyl-2-(4-(((5-oxo-4-propyl-1-(3-(trifluoromethyl)benzyl)-4,5-dihydro-1H-1,2,4-triazol-3-yl)methoxy)carbonyl)phenoxy)propanoic acid;
2-(4-(((1-(3,5-bis(trifluoromethyl)benzyl)-5-oxo-4-propyl-4,5-dihydro-1H-1,2,4-triazol-3-yl)methoxy)carbonyl)phenoxy)-2-methylpropanoic acid;
2-(4-(((1-(3,5-difluorobenzyl)-5-oxo-4-propyl-4,5-dihydro-1H-1,2,4-triazol-3-yl)methoxy)carbonyl)phenoxy)-2-methylpropanoic acid;
2-(4-(((1-(biphenyl-4-ylmethyl)-5-oxo-4-propyl-4,5-dihydro-1H-1,2,4-triazol-3-yl)methoxy)carbonyl)phenoxy)-2-methylpropanoic acid;
2-(4-(((1-(3,4-dimethylbenzyl)-5-oxo-4-propyl-4,5-dihydro-1H-1,2,4-triazol-3-yl)methoxy)carbonyl)phenoxy)-2-methylpropanoic acid;
2-methyl-2-(4-(((5-oxo-4-propyl-1-(4-(trifluoromethylthio)benzyl)-4,5-dihydro-1H-1,2,4-triazol-3-yl)methoxy)carbonyl)phenoxy)propanoic acid;
2-(4-(((1-(4-bromobenzyl)-5-oxo-4-propyl-4,5-dihydro-1H-1,2,4-triazol-3-yl)methoxy)carbonyl)phenoxy)-2-methylpropanoic acid;
2-(4-(((1-(4-tert-butylbenzyl)-4-heptyl-5-oxo-4,5-dihydro-1H-1,2,4-triazol-3-yl)methoxy)carbonyl)-2-methoxyphenoxy)-2-methylpropanoic acid;
2-methyl-2-(4-(((1-(4-methylbenzyl)-5-oxo-4,5-dihydro-1H-1,2,4-triazol-3-yl)methoxy)carbonyl)phenoxy)propanoic acid;
2-(4-(((1-(4-tert-butylbenzyl)-5-oxo-4,5-dihydro-1H-1,2,4-triazol-3-yl)methoxy)carbonyl)phenoxy)-2-methylpropanoic acid;
2-methyl-2-(4-(((1-(4-methylbenzyl)-5-oxo-4,5-dihydro-1H-1,2,4-triazol-3-yl)methoxy)carbonyl)phenylthio)propanoic acid;
2-(4-(((1-(4-tert-butylbenzyl)-5-oxo-4,5-dihydro-1H-1,2,4-triazol-3-yl)methoxy)carbonyl)phenylthio)-2-methylpropanoic acid;
(4-heptyl-1-(4-methylbenzyl)-5-oxo-4,5-dihydro-1H-1,2,4-triazol-3-yl)methyl 4-(1-ethoxy-2-methyl-1-oxopropan-2-ylthio)benzoate;
2-(4-(((1-(3,5-difluorobenzyl)-5-oxo-4-propyl-4,5-dihydro-1H-1,2,4-triazol-3-yl)methoxy)carbonyl)phenylthio)-2-methylpropanoic acid;
2-methyl-2-(4-(((1-(4-methylbenzyl)-5-oxo-4,5-dihydro-1H-1,2,4-triazol-3-yl)methoxy)carbonyl)phenoxy)propanoic acid;
2-(4-(((1-(4-tert-butylbenzyl)-5-oxo-4,5-dihydro-1H-1,2,4-triazol-3-yl)methoxy)carbonyl)phenoxy)-2-methylpropanoic acid;
2-methyl-2-(4-(((4-methyl-1-(4-methylbenzyl)-5-oxo-4,5-dihydro-1H-1,2,4-triazol-3-yl)methoxy)carbonyl)phenoxy)propanoic acid;
2-(4-(((1-(4-tert-butylbenzyl)-4-methyl-5-oxo-4,5-dihydro-1H-1,2,4-triazol-3-yl)methoxy)carbonyl)phenoxy)-2-methylpropanoic acid;
2-methyl-2-(4-(((1-(4-methylbenzyl)-5-oxo-4,5-dihydro-1H-1,2,4-triazol-3-yl)methoxy)carbonyl)phenylthio)propanoic acid;
2-(4-(((1-(4-tert-butylbenzyl)-5-oxo-4,5-dihydro-1H-1,2,4-triazol-3-yl)methoxy)carbonyl)phenylthio)-2-methylpropanoic acid;
2-methyl-2-(4-(((4-methyl-1-(4-methylbenzyl)-5-oxo-4,5-dihydro-1H-1,2,4-triazol-3-yl)methoxy)carbonyl)phenylthio)propanoic acid;
2-(4-(((1-(4-tert-butylbenzyl)-4-methyl-5-oxo-4,5-dihydro-1H-1,2,4-triazol-3-yl)methoxy)carbonyl)phenylthio)-2-methylpropanoic acid;
2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid;
2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid;
(R)-2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid;
(S)-2-methyl-2-(4-((1-(1-(4-methylbenzyl)-5-oxo-4,5-dihydro-1H-1,2,4-triazol-3-yl)ethoxy)carbonyl)phenylthio)propanoic acid;
(R)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid;
(S)-2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid;
(S)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid;
(R)-2-methyl-2-(4-((1-(1-(4-methylbenzyl)-5-oxo-4,5-dihydro-1H-1,2,4-triazol-3-yl)ethoxy)carbonyl)phenylthio)propanoic acid;
4-(1-Carboxy-1-methyl-ethylsulfanyl)-benzoic acid (S)-1-[1-(4-methyl-benzyl)-5-oxo-4-propyl-4,5-dihydro-1H-1,2,4-triazol-3-yl]-ethyl ester;
4-(1-Carboxy-1-methyl-ethylsulfanyl)-benzoic acid (R)-1-[1-(4-methyl-benzyl)-5-oxo-4-propyl-4,5-dihydro-1H-1,2,4-triazol-3-yl]-ethyl ester;
4-(1-Carboxy-1-methyl-ethoxy)-benzoic acid (S)-1-[1-(4-methyl-benzyl)-5-oxo-4-propyl-4,5-dihydro-1H-1,2,4-triazol-3-yl]-ethyl ester;
4-(1-Carboxy-1-methyl-ethoxy)-benzoic acid (R)-1-[1-(4-methyl-benzyl)-5-oxo-4-propyl-4,5-dihydro-1H-1,2,4-triazol-3-yl]-ethyl ester;
4-(1-Carboxy-1-methyl-ethylsulfanyl)-benzoic acid 1-[1-(4-tert-butyl-benzyl)-5-oxo-4-propyl-4,5-dihydro-1H-1,2,4-triazol-3-yl]-2,2,2-trifluoro-ethyl ester;
4-(1-Carboxy-1-methyl-ethylsulfanyl)-benzoic acid (S)-1-[1-(4-tert-butyl-benzyl)-5-oxo-4-propyl-4,5-dihydro-1H-1,2,4-triazol-3-yl]-ethyl ester;
4-(1-Carboxy-1-methyl-ethylsulfanyl)-benzoic acid (R)-1-[5-oxo-4-propyl-1-(4-trifluoromethyl-benzyl)-4,5-dihydro-1H-1,2,4-triazol-3-yl]-ethyl ester;
4-(1-Carboxy-1-methyl-ethylsulfanyl)-benzoic acid (R)-1-[1-(4-tert-butyl-benzyl)-5-oxo-4-propyl-4,5-dihydro-1H-1,2,4-triazol-3-yl]-ethyl ester;
4-(1-Carboxy-1-methyl-ethoxy)-benzoic acid (S)-1-[5-oxo-4-propyl-1-(4-trifluoromethyl-benzyl)-4,5-dihydro-1H-1,2,4-triazol-3-yl]-ethyl ester;
4-(1-Carboxy-1-methyl-ethoxy)-benzoic acid (S)-1-[1-(4-tert-butyl-benzyl)-5-oxo-4-propyl-4,5-dihydro-1H-1,2,4-triazol-3-yl]-ethyl ester;
4-(1-Carboxy-1-methyl-ethoxy)-benzoic acid (R)-1-[5-oxo-4-propyl-1-(4-trifluoromethyl-benzyl)-4,5-dihydro-1H-1,2,4-triazol-3-yl]-ethyl ester;
4-(1-Carboxy-1-methyl-ethoxy)-benzoic acid (R)-1-[1-(4-tert-butyl-benzyl)-5-oxo-4-propyl-4,5-dihydro-1H-1,2,4-triazol-3-yl]-ethyl ester;
4-(1-Carboxy-1-methyl-ethylsulfanyl)-benzoic acid (S)-1-[5-oxo-4-propyl-1-(4-trifluoromethyl-benzyl)-4,5-dihydro-1H-1,2,4-triazol-3-yl]-ethyl ester;
4-(1-Carboxy-1-methyl-ethylsulfanyl)-benzoic acid 1-(4-methyl-benzyl)-5-oxo-4,5-dihydro-1H-1,2,4-triazol-3-ylmethyl ester;
4-Carboxymethylsulfanyl-benzoic acid 1-(4-tert-butyl-benzyl)-4-hexyl-5-oxo-4,5-dihydro-1H-1,2,4-triazol-3-ylmethyl ester;
4-(1-Carboxy-1-methyl-ethoxy)-benzoic acid 1-[4-heptyl-1-(4-methyl-benzyl)-5-oxo-4,5-dihydro-1H-1,2,4-triazol-3-yl]-ethyl ester;
4-(1-Carboxy-1-methyl-ethylsulfanyl)-benzoic acid 1-[4-heptyl-1-(4-methyl-benzyl)-5-oxo-4,5-dihydro-1H-1,2,4-triazol-3-yl]-ethyl ester;
4-(1-Carboxy-1-methyl-ethoxy)-3-methyl-benzoic acid 1-(4-methyl-benzyl)-5-oxo-4-propyl-4,5-dihydro-1H-1,2,4-triazol-3-ylmethyl ester;
4-(1-Carboxy-1-methyl-ethoxy)-3-methyl-benzoic acid 1-(4-tert-butyl-benzyl)-5-oxo-4-propyl-4,5-dihydro-1H-1,2,4-triazol-3-ylmethyl ester; or pharmaceutically acceptable salts thereof

10. Compounds according to claim 9 that are
2-methyl-2-(4-(((1-(4-methylbenzyl)-1H-pyrazol-4-yl)methoxy)carbonyl)phenylthio)propanoic acid
2-methyl-2-(4-(((3-propyl-1-(4-(trifluoromethyl)benzyl)-1H-pyrazol-4-yl)methoxy)carbonyl)phenylthio)propanoic acid
2-methyl-2-(4-(((1-propyl-3-p-tolyl-1H-pyrazol-5-yl)methoxy)carbonyl)phenylthio)propanoic acid
2-methyl-2-(4-(((1-propyl-3-p-tolyl-1H-pyrazol-5-yl)methoxy)carbonyl)phenoxy)propanoic acid
2-methyl-2-(4-(((3-phenyl-1-propyl-1H-pyrazol-5-yl)methoxy)carbonyl)phenylthio)propanoic acid
2-methyl-2-(4-(((1-(4-(methylthio)benzyl)-1H-pyrazol-4-yl)methoxy)carbonyl)phenylthio)propanoic acid
2-(4-(((1-(3,4-difluorobenzyl)-1H-pyrazol-4-yl)methoxy)carbonyl)phenylthio)-2-methylpropanoic acid
(R)-2-methyl-2-(4-((1-(3-(4-methylbenzyl)-1-propyl-1H-pyrazol-5-yl)ethoxy)carbonyl)phenoxy)propanoic acid
(S)-2-methyl-2-(4-((1-(3-(4-methylbenzyl)-1-propyl-1H-pyrazol-5-yl)ethoxy)carbonyl)phenoxy)propanoic acid
(R)-2-methyl-2-(4-((2,2,2-trifluoro-1-(3-(4-methylbenzyl)-1-propyl-1H-pyrazol-5-yl)ethoxy)carbonyl)phenoxy)propanoic acid
(S)-2-methyl-2-(4-((2,2,2-trifluoro-1-(3-(4-methylbenzyl)-1-propyl-1H-pyrazol-5-yl)ethoxy)carbonyl)phenoxy)propanoic acid
(R)-2-methyl-2-(4-((1-(3-(4-methylbenzyl)-1-propyl-1H-pyrazol-5-yl)ethoxy)carbonyl)phenylthio)propanoic acid
(S)-2-methyl-2-(4-((1-(3-(4-methylbenzyl)-1-propyl-1H-pyrazol-5-yl)ethoxy)carbonyl)phenylthio)propanoic acid
(R)-2-methyl-2-(4-((2,2,2-trifluoro-1-(3-(4-methylbenzyl)-1-propyl-1H-pyrazol-5-yl)ethoxy)carbonyl)phenylthio)propanoic acid
(S)-2-methyl-2-(4-((2,2,2-trifluoro-1-(3-(4-methylbenzyl)-1-propyl-1H-pyrazol-5-yl)ethoxy)carbonyl)phenylthio)propanoic acid
4-(1-Carboxy-1-methyl-ethoxy)-benzoic acid 5-(4-methyl-benzyl)-2-propyl-2H-pyrazol-3-ylmethyl ester
4-(1-Carboxy-1-methyl-ethylsulfanyl)-benzoic acid 5-(4-methyl-benzyl)-2-propyl-2H-pyrazol-3-ylmethyl ester
2-methyl-2-(4-(((1-(4-(trifluoromethyl)benzyl)-1H-1,2,3-triazol-4-yl)methoxy)carbonyl)phenylthio)propanoic acid
2-(4-(((1-(4-tert-butylbenzyl)-1H-1,2,3-triazol-4-yl)methoxy)carbonyl)phenylthio)-2-methylpropanoic acid
2-methyl-2-(4-(((1-(4-(trifluoromethylthio)benzyl)-1H-1,2,3-triazol-4-yl)methoxy)carbonyl)phenylthio)propanoic acid
2-methyl-2-(4-(((1-(4-(trifluoromethoxy)benzyl)-1H-1,2,3-triazol-4-yl)methoxy)carbonyl)phenylthio)propanoic acid
2-(4-(((1-(4-(1,1,1,3,3,3-hexafluoro-2-methoxypropan-2-yl)benzyl)-1H-1,2,3-triazol-4-yl)methoxy)carbonyl)phenylthio)-2-methylpropanoic acid
2-methyl-2-(4-((1-(1-(4-methylbenzyl)-1H-1,2,3-triazol-4-yl)ethoxy)carbonyl)phenylthio)propanoic acid
(R)-2-methyl-2-(4-((1-(1-(4-methylbenzyl)-1H-1,2,3-triazol-4-yl)ethoxy)carbonyl)phenylthio)propanoic acid
2-methyl-2-(4-((2-(1-(4-(trifluoromethyl)benzyl)-1H-1,2,3-triazol-4-yl)ethoxy)carbonyl)phenylthio)propanoic acid
(R)-2-methyl-2-(4-((1-(1-(4-(trifluoromethyl)benzyl)-1H-1,2,3-triazol-4-yl)ethoxy)carbonyl)phenylthio)propanoic acid
2-methyl-2-(4-(((1-(4-(trifluoromethyl)benzyl)-1H-1,2,3-triazol-4-yl)methoxy)carbonyl)phenylthio)propanoic acid
(R)-2-(4-((1-(1-(4-tert-butylbenzyl)-1H-1,2,3-triazol-4-yl)propan-2-yloxy)carbonyl)phenylthio)-2-methylpropanoic acid
(S)-2-(4-((3-(1-(4-tert-butylbenzyl)-1H-1,2,3-triazol-4-yl)-1,1,1-trifluoropropan-2-yloxy)carbonyl)phenylthio)-2-methylpropanoic acid
(R)-2-(4-((3-(1-(4-tert-butylbenzyl)-1H-1,2,3-triazol-4-yl)-1,1,1-trifluoropropan-2-yloxy)carbonyl)phenylthio)-2-methylpropanoic acid
(R)-2-methyl-2-(4-((1-(1-(4-(trifluoromethyl)benzyl)-1H-1,2,3-triazol-4-yl)propan-2-yloxy)carbonyl)phenylthio)propanoic acid
(S)-2-methyl-2-(4-((1-(1-(4-(trifluoromethyl)benzyl)-1H-1,2,3-triazol-4-yl)propan-2-yloxy)carbonyl)phenylthio)propanoic acid
(R)-2-methyl-2-(4-((1,1,1-trifluoro-3-(1-(4-(trifluoromethyl)benzyl)-1H-1,2,3-triazol-4-yl)propan-2-yloxy)carbonyl)phenylthio)propanoic acid
(S)-2-methyl-2-(4-((1,1,1-trifluoro-3-(1-(4-(trifluoromethyl)benzyl)-1H-1,2,3-triazol-4-yl)propan-2-yloxy)carbonyl)phenylthio)propanoic acid
(S)-2-methyl-2-(4-((2,2,2-trifluoro-1-(1-(4-methylbenzyl)-1H-1,2,3-triazol-4-yl)ethoxy)carbonyl)phenylthio)propanoic acid
(R)-2-methyl-2-(4-((2,2,2-trifluoro-1-(1-(4-methylbenzyl)-1H-1,2,3-triazol-4-yl)ethoxy)carbonyl)phenylthio)propanoic acid
(R)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid
(S)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid
4-(1-Carboxy-1-methyl-ethylsulfanyl)-benzoic acid (S)-2-[1-(4-tert-butyl-benzyl)-1H-1,2,3-triazol-4-yl]-1-methyl-ethyl ester
4-(1-Carboxy-1-methyl-ethylsulfanyl)-benzoic acid (R)-1-[1-(4-tert-butyl-benzyl)-1H-1,2,3-triazol-4-yl]-ethyl ester
4-(1-Carboxy-1-methyl-ethylsulfanyl)-benzoic acid (R)-1-[1-(4-trifluoromethylsulfanyl-benzyl)-1H-1,2,3-triazol-4-yl]-ethyl ester
4-(1-Carboxy-1-methyl-ethylsulfanyl)-benzoic acid (R)-1-[1-(4-trifluoromethoxy-benzyl)-1H-1,2,3-triazol-4-yl]-ethyl ester
4-(1-Carboxy-1-methyl-ethylsulfanyl)-benzoic acid (R)-2-[1-(4-tert-butyl-benzyl)-1H-1,2,3-triazol-4-yl]-1-methyl-ethyl ester
4-(1-Carboxy-1-methyl-ethylsulfanyl)-benzoic acid (R)-1-[1-(3-trifluoromethyl-benzyl)-1H-1,2,3-triazol-4-yl]-ethyl ester
4-(1-Carboxy-1-methyl-ethylsulfanyl)-benzoic acid (R)-1-[1-(3,5-bis-trifluoromethyl-benzyl)-1H-1,2,3-triazol-4-yl]-ethyl ester
4-(1-Carboxy-1-methyl-ethylsulfanyl)-benzoic acid (R)-1-[1-(3,5-difluoro-benzyl)-1H-1,2,3-triazol-4-yl]-ethyl ester
4-(1-Carboxy-1-methyl-ethylsulfanyl)-benzoic acid 2-[1-(4-tert-butyl-benzyl)-1H-1,2,3-triazol-4-yl]-ethyl ester
4-(1-Carboxy-1-methyl-ethylsulfanyl)-benzoic acid 2-[1-(4-methyl-benzyl)-1H-1,2,3-triazol-4-yl]-ethyl ester 4-(1-Carboxy-1-methyl-ethylsulfanyl)-benzoic acid (R)-1-methyl-2-[1-(4-trifluoromethyl-benzyl)-1H-1,2,3-triazol-4-yl]-ethyl ester
4-(1-Carboxy-1-methyl-ethylsulfanyl)-benzoic acid (S)-1-methyl-2-[1-(4-trifluoromethyl-benzyl)-1H-1,2,3-triazol-4-yl]-ethyl ester
2-(4-(((1-(4-tert-butylbenzyl)-5-oxo-4-propyl-4,5-dihydro-1H-1,2,4-triazol-3-yl)methoxy)carbonyl)phenoxy)-2-methylpropanoic acid
2-(4-(((4-hexyl-1-(4-methylbenzyl)-5-oxo-4,5-dihydro-1H-1,2,4-triazol-3-yl)methoxy)carbonyl)phenoxy)-2-methylpropanoic acid
2-(4-(((1-(4-tert-butylbenzyl)-5-oxo-4-propyl-4,5-dihydro-1H-1,2,4-triazol-3-yl)methoxy)carbonyl)phenylthio)-2-methylpropanoic acid
2-(4-(((4-hexyl-1-(4-methylbenzyl)-5-oxo-4,5-dihydro-1H-1,2,4-triazol-3-yl)methoxy)carbonyl)phenylthio)-2-methylpropanoic acid
2-methyl-2-(4-(((1-(4-methylbenzyl)-4-octyl-5-oxo-4,5-dihydro-1H-1,2,4-triazol-3-yl)methoxy)carbonyl)phenylthio)propanoic acid
2-(4-(((1-(4-bromobenzyl)-5-oxo-4-propyl-4,5-dihydro-1H-1,2,4-triazol-3-yl)methoxy)carbonyl)phenylthio)-2-methylpropanoic acid
2-methyl-2-(4-(((5-oxo-4-propyl-1-(4-(trifluoromethyl)benzyl)-4,5-dihydro-1H-1,2,4-triazol-3-yl)methoxy)carbonyl)phenoxy)propanoic acid
2-methyl-2-(4-(((5-oxo-4-propyl-1-(4-(trifluoromethoxy)benzyl)-4,5-dihydro-1H-1,2,4-triazol-3-yl)methoxy)carbonyl)phenoxy)propanoic acid
2-methyl-2-(4-(((5-oxo-4-propyl-1-(4-(trifluoromethylthio)benzyl)-4,5-dihydro-1H-1,2,4-triazol-3-yl)methoxy)carbonyl)phenylthio)propanoic acid
2-(2-methoxy-4-(((1-(4-methylbenzyl)-4-octyl-5-oxo-4,5-dihydro-1H-1,2,4-triazol-3-yl)methoxy)carbonyl)phenoxy)-2-methylpropanoic acid
2-(4-(((1-(4-tert-butylbenzyl)-4-hexyl-5-oxo-4,5-dihydro-1H-1,2,4-triazol-3-yl)methoxy)carbonyl)phenylthio)-2-methylpropanoic acid
2-(4-(((1-(4-tert-butylbenzyl)-4-heptyl-5-oxo-4,5-dihydro-1H-1,2,4-triazol-3-yl)methoxy)carbonyl)phenylthio)-2-methylpropanoic acid
2-(4-(((1-(4-tert-butylbenzyl)-4-heptyl-5-oxo-4,5-dihydro-1H-1,2,4-triazol-3-yl)methoxy)carbonyl)phenoxy)-2-methylpropanoic acid
2-(4-(((4-heptyl-1-(4-methylbenzyl)-5-oxo-4,5-dihydro-1H-1,2,4-triazol-3-yl)methoxy)carbonyl)phenylthio)-2-methylpropanoic acid
2-(4-(((4-heptyl-1-(4-methylbenzyl)-5-oxo-4,5-dihydro-1H-1,2,4-triazol-3-yl)methoxy)carbonyl)phenoxy)-2-methylpropanoic acid
2-(4-(((4-ethyl-1-(4-methylbenzyl)-5-oxo-4,5-dihydro-1H-1,2,4-triazol-3-yl)methoxy)carbonyl)phenylthio)-2-methylpropanoic acid
2-(4-(((1-(4-tert-butylbenzyl)-4-ethyl-5-oxo-4,5-dihydro-1H-1,2,4-triazol-3-yl)methoxy)carbonyl)phenylthio)-2-methylpropanoic acid
2-(4-(((4-ethyl-5-oxo-1-(4-(trifluoromethyl)benzyl)-4,5-dihydro-1H-1,2,4-triazol-3-yl)methoxy)carbonyl)phenylthio)-2-methylpropanoic acid
2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid
2-(4-(((1-(4-(1,1,1,3,3,3-hexafluoro-2-methoxypropan-2-yl)benzyl)-4-hexyl-5-oxo-4,5-dihydro-1H-1,2,4-triazol-3-yl)methoxy)carbonyl)phenylthio)-2-methylpropanoic acid
2-(4-(((4-hexyl-5-oxo-1-(4-(perfluoropropan-2-yl)benzyl)-4,5-dihydro-1H-1,2,4-triazol-3-yl)methoxy)carbonyl)phenylthio)-2-methylpropanoic acid
2-(4-(((1-(4-tert-butylbenzyl)-4-heptyl-5-oxo-4,5-dihydro-1H-1,2,4-triazol-3-yl)methoxy)carbonyl)-2-methoxyphenoxy)-2-methylpropanoic acid
2-methyl-2-(4-(((1-(4-methylbenzyl)-5-oxo-4,5-dihydro-1H-1,2,4-triazol-3-yl)methoxy)carbonyl)phenoxy)propanoic acid
2-(4-(((1-(4-tert-butylbenzyl)-5-oxo-4,5-dihydro-1H-1,2,4-triazol-3-yl)methoxy)carbonyl)phenoxy)-2-methylpropanoic acid
2-methyl-2-(4-(((1-(4-methylbenzyl)-5-oxo-4,5-dihydro-1H-1,2,4-triazol-3-yl)methoxy)carbonyl)phenylthio)propanoic acid
2-(4-(((1-(4-tert-butylbenzyl)-5-oxo-4,5-dihydro-1H-1,2,4-triazol-3-yl)methoxy)carbonyl)phenylthio)-2-methylpropanoic acid
2-methyl-2-(4-(((1-(4-methylbenzyl)-5-oxo-4,5-dihydro-1H-1,2,4-triazol-3-yl)methoxy)carbonyl)phenoxy)propanoic acid
2-(4-(((1-(4-tert-butylbenzyl)-5-oxo-4,5-dihydro-1H-1,2,4-triazol-3-yl)methoxy)carbonyl)phenoxy)-2-methylpropanoic acid
2-methyl-2-(4-(((4-methyl-1-(4-methylbenzyl)-5-oxo-4,5-dihydro-1H-1,2,4-triazol-3-yl)methoxy)carbonyl)phenoxy)propanoic acid
2-(4-(((1-(4-tert-butylbenzyl)-4-methyl-5-oxo-4,5-dihydro-1H-1,2,4-triazol-3-yl)methoxy)carbonyl)phenoxy)-2-methylpropanoic acid
2-methyl-2-(4-(((1-(4-methylbenzyl)-5-oxo-4,5-dihydro-1H-1,2,4-triazol-3-yl)methoxy)carbonyl)phenylthio)propanoic acid
2-(4-(((1-(4-tert-butylbenzyl)-5-oxo-4,5-dihydro-1H-1,2,4-triazol-3-yl)methoxy)carbonyl)phenylthio)-2-methylpropanoic acid
2-methyl-2-(4-(((4-methyl-1-(4-methylbenzyl)-5-oxo-4,5-dihydro-1H-1,2,4-triazol-3-yl)methoxy)carbonyl)phenylthio)propanoic acid
2-(4-(((1-(4-tert-butylbenzyl)-4-methyl-5-oxo-4,5-dihydro-1H-1,2,4-triazol-3-yl)methoxy)carbonyl)phenylthio)-2-methylpropanoic acid
2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
(R)-2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
(S)-2-methyl-2-(4-((1-(1-(4-methylbenzyl)-5-oxo-4,5-dihydro-1H-1,2,4-triazol-3-yl)ethoxy)carbonyl)phenylthio)propanoic acid
(R)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
(S)-2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
(S)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
(R)-2-methyl-2-(4-((1-(1-(4-methylbenzyl)-5-oxo-4,5-dihydro-1H-1,2,4-triazol-3-yl)ethoxy)carbonyl)phenylthio)propanoic acid
4-(1-Carboxy-1-methyl-ethylsulfanyl)-benzoic acid (S)-1-[1-(4-methyl-benzyl)-5-oxo-4-propyl-4,5-dihydro-1H-1,2,4-triazol-3-yl]-ethyl ester
4-(1-Carboxy-1-methyl-ethylsulfanyl)-benzoic acid (R)-1-[1-(4-methyl-benzyl)-5-oxo-4-propyl-4,5-dihydro-1H-1,2,4-triazol-3-yl]-ethyl ester
4-(1-Carboxy-1-methyl-ethylsulfanyl)-benzoic acid 1-[1-(4-tert-butyl-benzyl)-5-oxo-4-propyl-4,5-dihydro-1H-1,2,4-triazol-3-yl]-2,2,2-trifluoro-ethyl ester
4-(1-Carboxy-1-methyl-ethylsulfanyl)-benzoic acid (S)-1-[1-(4-tert-butyl-benzyl)-5-oxo-4-propyl-4,5-dihydro-1H-1,2,4-triazol-3-yl]-ethyl ester
4-(1-Carboxy-1-methyl-ethylsulfanyl)-benzoic acid (R)-1-[5-oxo-4-propyl-1-(4-trifluoromethyl-benzyl)-4,5-dihydro-1H-1,2,4-triazol-3-yl]-ethyl ester
4-(1-Carboxy-1-methyl-ethylsulfanyl)-benzoic acid (R)-1-[1-(4-tert-butyl-benzyl)-5-oxo-4-propyl-4,5-dihydro-1H-1,2,4-triazol-3-yl]-ethyl ester
4-(1-Carboxy-1-methyl-ethoxy)-benzoic acid (S)-1-[5-oxo-4-propyl-1-(4-trifluoromethyl-benzyl)-4,5-dihydro-1H-1,2,4-triazol-3-yl]-ethyl ester
4-(1-Carboxy-1-methyl-ethoxy)-benzoic acid (S)-1-[1-(4-tert-butyl-benzyl)-5-oxo-4-propyl-4,5-dihydro-1H-1,2,4-triazol-3-yl]-ethyl ester
4-(1-Carboxy-1-methyl-ethoxy)-benzoic acid (R)-1-[5-oxo-4-propyl-1-(4-trifluoromethyl-benzyl)-4,5-dihydro-1H-1,2,4-triazol-3-yl]-ethyl ester
4-(1-Carboxy-1-methyl-ethoxy)-benzoic acid (R)-1-[1-(4-tert-butyl-benzyl)-5-oxo-4-propyl-4,5-dihydro-1H-1,2,4-triazol-3-yl]-ethyl ester
4-(1-Carboxy-1-methyl-ethylsulfanyl)-benzoic acid (S)-1-[5-oxo-4-propyl-1-(4-trifluoromethyl-benzyl)-4,5-dihydro-1H-1,2,4-triazol-3-yl]-ethyl ester
4-(1-Carboxy-1-methyl-ethylsulfanyl)-benzoic acid 1-(4-methyl-benzyl)-5-oxo-4,5-dihydro-1H-1,2,4-triazol-3-ylmethyl ester
4-Carboxymethylsulfanyl-benzoic acid 1-(4-tert-butyl-benzyl)-4-hexyl-5-oxo-4,5-dihydro-1H-1,2,4-triazol-3-ylmethyl ester
4-(1-Carboxy-1-methyl-ethoxy)-benzoic acid 1-[4-heptyl-1-(4-methyl-benzyl)-5-oxo-4,5-dihydro-1H-1,2,4-triazol-3-yl]-ethyl ester
4-(1-Carboxy-1-methyl-ethylsulfanyl)-benzoic acid 1-[4-heptyl-1-(4-methyl-benzyl)-5-oxo-4,5-dihydro-1H-1,2,4-triazol-3-yl]-ethyl ester

11. Compounds according to claim 10 that are
2-methyl-2-(4-(((1-(4-(methylthio)benzyl)-1H-pyrazol-4-yl)methoxy)carbonyl)phenylthio)propanoic acid
(R)-2-methyl-2-(4-((1-(3-(4-methylbenzyl)-1-propyl-1H-pyrazol-5-yl)ethoxy)carbonyl)phenoxy)propanoic acid
(S)-2-methyl-2-(4-((1-(3-(4-methylbenzyl)-1-propyl-1H-pyrazol-5-yl)ethoxy)carbonyl)phenoxy)propanoic acid
(R)-2-methyl-2-(4-((2,2,2-trifluoro-1-(3-(4-methylbenzyl)-1-propyl-1H-pyrazol-5-yl)ethoxy)carbonyl)phenoxy)propanoic acid
(S)-2-methyl-2-(4-((2,2,2-trifluoro-1-(3-(4-methylbenzyl)-1-propyl-1H-pyrazol-5-yl)ethoxy)carbonyl)phenoxy)propanoic acid
(R)-2-methyl-2-(4((1-(3-(4-methylbenzyl)-1-propyl-1H-pyrazol-5-yl)ethoxy)carbonyl)phenylthio)propanoic acid
(S)-2-methyl-2-(4-((1-(3-(4-methylbenzyl)-1-propyl-1H-pyrazol-5-yl)ethoxy)carbonyl)phenylthio)propanoic acid
(R)-2-methyl-2-(4-((2,2,2-trifluoro-1-(3-(4-methylbenzyl)-1-propyl-1H-pyrazol-5-yl)ethoxy)carbonyl)phenylthio)propanoic acid
(S)-2-methyl-2-(4-((2,2,2-trifluoro-1-(3-(4-methylbenzyl)-1-propyl-1H-pyrazol-5-yl)ethoxy)carbonyl)phenylthio)propanoic acid
4-(1-Carboxy-1-methyl-ethoxy)-benzoic acid 5-(4-methyl-benzyl)-2-propyl-2H-pyrazol-3-ylmethyl ester
4-(1-Carboxy-1-methyl-ethylsulfanyl)-benzoic acid 5-(4-methyl-benzyl)-2-propyl-2H-pyrazol-3-ylmethyl ester
(R)-2-methyl-2-(4-((1-(1-(4-methylbenzyl)-1H-1,2,3-triazol-4-yl)ethoxy)carbonyl)phenylthio)propanoic acid
2-methyl-2-(4-((2-(1-(4-(trifluoromethyl)benzyl)-1H-1,2,3-triazol-4-yl)ethoxy)carbonyl)phenylthio)propanoic acid
(R)-2-methyl-2-(4-((1-(1-(4-(trifluoromethyl)benzyl)-1H-1,2,3-triazol-4-yl)ethoxy)carbonyl)phenylthio)propanoic acid
(R)-2-(4-((1-(1-(4-tert-butylbenzyl)-1H-1,2,3-triazol-4-yl)propan-2-yloxy)carbonyl)phenylthio)-2-methylpropanoic acid
(S)-2-(4-((3-(1-(4-tert-butylbenzyl)-1H-1,2,3-triazol-4-yl)-1,1,1-trifluoropropan-2-yloxy)carbonyl)phenylthio)-2-methylpropanoic acid
(R)-2-(4-((3-(1-(4-tert-butylbenzyl)-1H-1,2,3-triazol-4-yl)-1,1,1-trifluoropropan-2-yloxy)carbonyl)phenylthio)-2-methylpropanoic acid
(S)-2-methyl-2-(4-((2,2,2-trifluoro-1-(1-(4-methylbenzyl)-1H-1,2,3-triazol-4-yl)ethoxy)carbonyl)phenylthio)propanoic acid
(R)-2-methyl-2-(4-((2,2,2-trifluoro-1-(1-(4-methylbenzyl)-1H-1,2,3-triazol-4-yl)ethoxy)carbonyl)phenylthio)propanoic acid
(R)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid
(S)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid
4-(1-Carboxy-1-methyl-ethylsulfanyl)-benzoic acid (S)-2-[1-(4-tert-butyl-benzyl)-1H-1,2,3-triazol-4-yl]-1-methyl-ethyl ester
2-(4-(((1-(4-tert-butylbenzyl)-4-hexyl-5-oxo-4,5-dihydro-1H-1,2,4-triazol-3-yl)methoxy)carbonyl)phenylthio)-2-methylpropanoic acid
2-(4-(((1-(4-tert-butylbenzyl)-4-ethyl-5-oxo-4,5-dihydro-1H-1,2,4-triazol-3-yl)methoxy)carbonyl)phenylthio)-2-methylpropanoic acid
2-(4-(((4-ethyl-5-oxo-1-(4-(trifluoromethyl)benzyl)-4,5-dihydro-1H-1,2,4-triazol-3-yl)methoxy)carbonyl)phenylthio)-2-methylpropanoic acid
2-methyl-2-(4-((1-(1-(4-methylbenzyl)-5-oxo-4-propyl-4,5-dihydro-1H-1,2,4-triazol-3-yl)ethoxy)carbonyl)phenylthio)propanoic acid
2-methyl-2-(4-(((1-(4-methylbenzyl)-5-oxo-4,5-dihydro-1H-1,2,4-triazol-3-yl)methoxy)carbonyl)phenoxy)propanoic acid
2-(4-(((1-(4-tert-butylbenzyl)-5-oxo-4,5-dihydro-1H-1,2,4-triazol-3-yl)methoxy)carbonyl)phenoxy)-2-methylpropanoic acid
2-methyl-2-(4-(((1-(4-methylbenzyl)-5-oxo-4,5-dihydro-1H-1,2,4-triazol-3-yl)methoxy)carbonyl)phenylthio)propanoic acid
2-(4-(((1-(4-tert-butylbenzyl)-5-oxo-4,5-dihydro-1H-1,2,4-triazol-3-yl)methoxy)carbonyl)phenylthio)-2-methylpropanoic acid
2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
(R)-2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
(S)-2-methyl-2-(4-((1-(1-(4-methylbenzyl)-5-oxo-4,5-dihydro-1H-1,2,4-triazol-3-yl)ethoxy)carbonyl)phenylthio)propanoic acid
(R)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
(S)-2-methyl-2-(4-((2,2,2-trifluoro-1-(1-(4-methylbenzyl)-5-oxo-4-propyl-4,5-dihydro-1H-1,2,4-triazol-3-yl)ethoxy)carbonyl)phenylthio)propanoic acid
(S)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
(R)-2-methyl-2-(4-((1-(1-(4-methylbenzyl)-5-oxo-4,5-dihydro-1H-1,2,4-triazol-3-yl)ethoxy)carbonyl)phenylthio)propanoic acid
4-(1-Carboxy-1-methyl-ethylsulfanyl)-benzoic acid (S)-1-[1-(4-methyl-benzyl)-5-oxo-4-propyl-4,5-dihydro-1H-1,2,4-triazol-3-yl]-ethyl ester
4-(1-Carboxy-1-methyl-ethylsulfanyl)-benzoic acid (R)-1-[1-(4-methyl-benzyl)-5-oxo-4-propyl-4,5-dihydro-1H-1,2,4-triazol-3-yl]-ethyl ester
4-(1-Carboxy-1-methyl-ethylsulfanyl)-benzoic acid 1-[4-heptyl-1-(4-methyl-benzyl)-5-oxo-4,5-dihydro-1H-1,2,4-triazol-3-yl]-ethyl ester

12. Compounds according to claim 11 that are
(R)-2-methyl-2-(4-((1-(3-(4-methylbenzyl)-1-propyl-1H-pyrazol-5-yl)ethoxy)carbonyl)phenoxy)propanoic acid
(S)-2-methyl-2-(4-((1-(3-(4-methylbenzyl)-1-propyl-1H-pyrazol-5-yl)ethoxy)carbonyl)phenoxy)propanoic acid
(R)-2-methyl-2-(4-((2,2,2-trifluoro-1-(3-(4-methylbenzyl)-1-propyl-1H-pyrazol-5-yl)ethoxy)carbonyl)phenoxy)propanoic acid
(S)-2-methyl-2-(4-((2,2,2-trifluoro-1-(3-(4-methylbenzyl)-1-propyl-1H-pyrazol-5-yl)ethoxy)carbonyl)phenoxy)propanoic acid
(R)-2-methyl-2-(4-((1-(3-(4-methylbenzyl)-1-propyl-1H-pyrazol-5-yl)ethoxy)carbonyl)phenylthio)propanoic acid
(S)-2-methyl-2-(4-((1-(3-(4-methylbenzyl)-1-propyl-1H-pyrazol-5-yl)ethoxy)carbonyl)phenylthio)propanoic acid
(R)-2-methyl-2-(4-((2,2,2-trifluoro-1-(3-(4-methylbenzyl)-1-propyl-1H-pyrazol-5-yl)ethoxy)carbonyl)phenylthio)propanoic acid
(S)-2-methyl-2-(4-((2,2,2-trifluoro-1-(3-(4-methylbenzyl)-1-propyl-1H-pyrazol-5-yl)ethoxy)carbonyl)phenylthio)propanoic acid
4-(1-Carboxy-1-methyl-ethoxy)-benzoic acid 5-(4-methyl-benzyl)-2-propyl-2H-pyrazol-3-ylmethyl ester
4-(1-Carboxy-1-methyl-ethylsulfanyl)-benzoic acid 5-(4-methyl-benzyl)-2-propyl-2H-pyrazol-3-ylmethyl ester
(R)-2-methyl-2-(4-((1-(1-(4-methylbenzyl)-1H-1,2,3-triazol-4-yl)ethoxy)carbonyl)phenylthio)propanoic acid
2-methyl-2-(4-((2-(1-(4-(trifluoromethyl)benzyl)-1H-1,2,3-triazol-4-yl)ethoxy)carbonyl)phenylthio)propanoic acid
(R)-2-methyl-2-(4-((1-(1-(4-(trifluoromethyl)benzyl)-1H-1,2,3-triazol-4-yl)ethoxy)carbonyl)phenylthio)propanoic acid
(S)-2-methyl-2-(4-((2,2,2-trifluoro-1-(1-(4-methylbenzyl)-1H-1,2,3-triazol-4-yl)ethoxy)carbonyl)phenylthio)propanoic acid p1 (S)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid
4-(1-Carboxy-1-methyl-ethylsulfanyl)-benzoic acid (S)-2-[1-(4-tert-butyl-benzyl)-1H-1,2,3-triazol-4-yl]-1-methyl-ethyl ester
(S)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
4-(1-Carboxy-1-methyl-ethylsulfanyl)-benzoic acid (S)-1-[1-(4-methyl-benzyl)-5-oxo-4-propyl-4,5-dihydro-1H-1,2,4-triazol-3-yl]-ethyl ester
4-(1-Carboxy-1-methyl-ethylsulfanyl)-benzoic acid (R)-1-[1-(4-methyl-benzyl)-5-oxo-4-propyl-4,5-dihydro-1H-1,2,4-triazol-3-yl]-ethyl ester

13. Compounds according to claim 12 that are
(R)-2-methyl-2-(4-((1-(1-(4-methylbenzyl)-1H-1,2,3-triazol-4-yl)ethoxy)carbonyl)phenylthio)propanoic acid
2-methyl-2-(4-((2-(1-(4-(trifluoromethyl)benzyl)-1H-1,2,3-triazol-4-yl)ethoxy)carbonyl)phenylthio)propanoic acid
(R)-2-methyl-2-(4-((1-(1-(4-(trifluoromethyl)benzyl)-1H-1,2,3-triazol-4-yl)ethoxy)carbonyl)phenylthio)propanoic acid
(S)-2-methyl-2-(4-((2,2,2-trifluoro-1-(1-(4-methylbenzyl)-1H-1,2,3-triazol-4-yl)ethoxy)carbonyl)phenylthio)propanoic acid
(S)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid
4-(1-Carboxy-1-methyl-ethylsulfanyl)-benzoic acid (S)-2-[1-(4-tert-butyl-benzyl)-1H-1,2,3-triazol-4-yl]-1-methyl-ethyl ester
(S)-2-methyl-2-(4-((2,2,2-trifluoro-1-(1-(4-methylbenzyl)-5-oxo-4,5-dihydro-1H-1,2,4-triazol-3-yl)ethoxy)carbonyl)phenylthio)propanoic acid
4-(1-Carboxy-1-methyl-ethylsulfanyl)-benzoic acid (S)-1-[1-(4-methyl-benzyl)-5-oxo-4-propyl-4,5-dihydro-1H-1,2,4-triazol-3-yl]-ethyl ester
4-(1-Carboxy-1-methyl-ethylsulfanyl)-benzoic acid (R)-1-[1-(4-methyl-benzyl)-5-oxo-4-propyl-4,5-dihydro-1H-1,2,4-triazol-3-yl]-ethyl ester

14. Compounds according to claim 13 that are
(R)-2-methyl-2-(4-((1-(1-(4-methylbenzyl)-1H-1,2,3-triazol-4-yl)ethoxy)carbonyl)phenylthio)propanoic acid
2-methyl-2-(4-((2-(1-(4-(trifluoromethyl)benzyl)-1H-1,2,3-triazol-4-yl)ethoxy)carbonyl)phenylthio)propanoic acid
(R)-2-methyl-2-(4-((1-(1-(4-(trifluoromethyl)benzyl)-1H-1,2,3-triazol-4-yl)ethoxy)carbonyl)phenylthio)propanoic acid
(S)-2-methyl-2-(4-((2,2,2-trifluoro-1-(1-(4-methylbenzyl)-1H-1,2,3-triazol-4-yl)ethoxy)carbonyl)phenylthio)propanoic acid
(S)-2-methyl-2-(4-((2,2,2-trifluoro-1-(1-(4-(trifluoromethyl)benzyl)-1H-1,2,3-triazol-4-yl)ethoxy)carbonyl)phenylthio)propanoic acid

15. A pharmaceutical composition comprising a compound of claim 1 and at least one pharmaceutically acceptable solvent, carrier, excipient, diluent or glidant.

16. A method of treating atherosclerosis hypercholesterolemia, primary hypercholesterolemia or mixed dyslipidemia, hypertriglyceridemia, Frederickson Types IV or V hyperlipidemia, the method comprising administering a therapeutically effective amount of a compound or salt of claim 1 to a patient in need of such treatment.

17. A method of activating PPARα, the method comprising administering a therapeutically effective amount of a compound or salt of claim 1 to a patient in need of such treatment.
